(19) United States
(12) Patent Application Publication
(10) Pub. No.: US 2020/0149145 A1
(43) Pub. Date: May 14, 2020

(54) NANOSTRUCTURED TITANIUM-BASED COMPOSITIONS AND METHODS TO FABRICATE THE SAME

(71) Applicant: The Board of Trustees of the University of Illinois, Urbana, IL (US)

(72) Inventors: Jean Paul ALLAIN, Champaign, IL (US); Alethia BARNWELL, Champaign, IL (US); Akshath R. SHETTY, Mumbai (IN); Ana Fatima CIVANTOS FERNANDEZ, Urbana, IL (US); Yadir TORRES, Sevilla (ES); Juan Jose PAVON, Columbia (CO)

(73) Assignee: The Board of Trustees of the University of Illinois, Urbana, IL (US)

(21) Appl. No.: 16/592,195

(22) Filed: Oct. 3, 2019

Related U.S. Application Data
(63) Continuation of application No. PCT/US18/26567, filed on Apr. 6, 2018.
(60) Provisional application No. 62/483,105, filed on Apr. 7, 2017.
(60) Provisional application No. 62/483,074, filed on Apr. 7, 2017.
(60) Provisional application No. 62/556,048, filed on Sep. 8, 2017.
(60) Provisional application No. 62/556,120, filed on Sep. 8, 2017.

(51) Int. Cl. C22F 3/00; A61L 27/50; C22C 14/00; A61L 27/06

(52) U.S. Cl. CPC C22F 3/00; A61L 27/06; C22C 14/00; A61L 27/50

(57) ABSTRACT

Provided herein are methods for the controlled, independent modification of the surface of titanium-based materials and compositions generated thereby. The methods allow for the alteration of multiple surface characteristics including generation of precise nanostructures, morphology, crystallography and chemical composition for increased biocompatibility, for example, osseointegration, osseoconduction, cell adhesion, cell proliferation, mechanical properties (e.g. elasticity, modulus, surface texture, porosity), hydrophobicity, hydrophilicity, steric hindrance, anti-inflammatory properties and/or anti-bacterial properties.

### CROSS-REFERENCE TO RELATED APPLICATIONS

This application claims the benefit of and priority to U.S. Provisional Application No. 62/483,105, filed Apr. 7, 2017; U.S. Provisional Application No. 62/483,074, filed Apr. 7, 2017; U.S. Provisional Application No. 62/556,120, filed Sep. 8, 2017 and U.S. Provisional Application No. 62/556,048, filed Sep. 8, 2017, which are each hereby incorporated in their entirety to the extent not inconsistent herewith.

### BACKGROUND OF INVENTION

Titanium and titanium alloys are widely recognized as advantageous materials for biological implants, including bone implants. Titanium-based materials have been commonly used as biological implants due to a beneficial balance between biomechanical properties and in vivo biocompatibility. Titanium-based materials are the preferred materials for a variety of implants, including dental implants, joint replacements, pacemakers and a variety of other medical applications and procedures.

Unprocessed, naturally-surfaced titanium-based implants suffer from a number of known disadvantages. First, solid commercially pure titanium and a number of titanium alloys are biomechanically incompatible, primarily due to elasticity and modulus mismatch between the implant and adjacent tissues, which may aggravate unwanted bone resorption in host tissues. Second, because titanium-based materials are bioinert, the body frequently grows a thin fibrous tissue over their surface after they are introduced into a patient. The fibrous tissue hinders osseointegration and may increase the risk of the implant loosening and bone fracture.

Modern implant producers have attempted to address these issues in a number of ways. Regarding biomechanical incompatibility, implants have been developed to improve mechanical mismatch, or reduce elastic modulus mismatch, by introducing pores in the implant via a number of known metallurgical processes. While pores reduce the modulus of the implant, careful control of the pore size and morphology is required for osseointegration, especially at the surface of the implant, to improve bone ingrowth and fatigue resistance. To address the growth of fibrous tissue, implants with surface modifications have been developed, utilizing topographical or chemical changes. Because cell surfaces naturally contain nanofeatures linked to the extracellular matrix, nanotopographical alterations to implants are important for cell adhesion and response.

Plasma-based techniques have been used for both generating surface porosity and modifying surface topology. For example, U.S. Pat. No. 6,582,470 describes modifying porous titanium surfaces by exposing the surface to a reactive plasma gas. While these techniques improve the biomechanical properties and cell adhesion, they provide little control over surface reactions and have difficulty in reliably fabricating structures smaller than about 50 nm. By providing precise porous and/or nanopatterned regions to the implant, including tailored to the specific application of the implant, biomechanical stresses may be reduced and cell adhesion increased, resulting in longer implant life, faster patient recovery times and reduced risk of implant tissue damage, complications and infections.

Conventional surface treatment of Ti surfaces for biomedical applications such as integration with bone have focused mostly on techniques that require strong chemical etchants and high-temperature processes. These approaches generally result in large toxic chemical waste streams and high-cost in manufacturing. Other approaches induce physical changes with methods such as sand-blasting or grit-blasting that result in kinetic roughening of the Ti surface with little control over the nanoscale topography, its specific dimensions, chemistry, composition and surface charge density.

Nanopatterned surfaces have been obtained mostly by bottom-up and top-down techniques on model materials given the difficulty in high-fidelity control of clinically-relevant surfaces and of complex 3D systems. Furthermore, no current nanoscale modification method exists that can control both surface chemistry and topography independently.

Thus, it can be seen from the foregoing that there remains a need in the art for surface modification of titanium-based implants, including at the nanometer level to improve biomechanical compatibility, associated functional deployment and implant lifetime and success rate, including by well controlled cellular adhesion to implant surface, morphology and behavior as well as high-fidelity control of the spatio-temporal immune-response behavior of the biomedical implant interface with the body. Tunability of the implant surface to engender specific cell and immune-system response that ultimately results in an interface that can trigger these effects over specific times and reproducibly is critically desirable.

### SUMMARY OF THE INVENTION

Provided herein are methods for the controlled, independent modification of the surface of titanium-based materials and compositions generated thereby. The methods allow for the alteration of multiple surface characteristics including generation of precise nanostructures, morphology, crystallography, chemical hybridizations and chemical composition for increased biocompatibility, for example, osseointegration, osseoconduction, cell adhesion, cell proliferation, enhanced local mechanical properties (elasticity, modulus, surface texture, porosity), hydrophobicity, hydrophilicity, steric hindrance, modulating-immuno response, anti-inflammatory properties and/or anti-bacterial properties. The surface of the composition may be modified by independently controlling parameters (e.g. incident angle, fluence, flux, energy, species, etc.) of one or more directed energetic particle beams, providing more control and increased bioactivity over conventional kinetic roughing techniques.

The provided compositions are modified to increase multiple biological properties or functions, including modification of multiple properties in a single region or domain and creating multiple regions or domains with biological advantages within a single composition. The provided methods are precise, allowing for the controlled generation of specific nanostructures across multiple domains. Further, precise changes to crystallography or morphology are possible, including changes to grain structure and the generation of metastable states. The provided methods also allow for specific modification of chemical composition, for instance, accurate creation of one or more alloys different from adjacent domains or the original underlying substrate. Irradiation-driven compositional variation such as one element over another at the surface differing from the sub-surface can be tuned to specific concentrations.

In aspect, provided is a titanium-containing composition comprising: a titanium or titanium alloy substrate having a surface; wherein the surface has a plurality of nanoscale domains characterized by a surface geometry providing a selected multifunctional bioactivity; wherein each of the nanoscale domains has at least one lateral spatial dimension selected over the range of 3 nm to 1 □m and a vertical spatial dimension less than 500 nm. In a second aspect, provided is a titanium-containing composition comprising: a titanium or titanium alloy substrate having a surface; wherein the surface has a plurality of nanoscale domains characterized by a surface geometry providing a selected multifunctional bioactivity; wherein the nanoscale domains are generated by exposing the surface to one or more directed energetic particle beam characterized by one or more beam properties.

In an embodiment, for example, the selected multifunctional bioactivity is with respect to an in vivo or in vitro activity with respect to a plurality of biological or physical processes relative to a titanium or titanium alloy substrate surface not having the plurality of nanoscale domains characterized by the nanofeatured surface geometry. In embodiments, the in vivo or in vitro activity is an enhancement in cell adhesion activity, cell shape activity, cell proliferation activity, cell migration activity, cell differentiation activity, anti-bacterial activity, bactericidal activity, anti-inflammatory activity, osseointegration activity, biocorrosion activity, cell differentiation activity, immuno-modulating activity during acute or chronic inflammation or any combination of these. In an embodiment, the enhancement of in vivo or in vitro activity is equal to or greater than 100%. In an embodiment, the in vivo or in vitro activity is a decrease in an immune response, for example, a decrease in the immune response equal to or greater than 200% in a period selected from the range of 24 to 48 hours.

The wetting characteristics of a solid material is ultimately the result of surface free energy. In all forms of condensed matter, there is a significant difference in the energy distribution of surface atoms and atoms within the bulk of a material. Each atom within the bulk material is surrounded by other neighboring atoms that all exert multi-directional pulling forces that collectively balance into a net force of zero. Unlike bulk atoms, surface atoms are exposed to the environment and subject to the one-sided pulling force of their inner material atom interfaces and external contacting influences. In the conditions of an unbalanced net force, surface atoms have a higher energy state whose “excess energy” creates an internal contracting pressure designated as the surface tension or free energy. Contact angle analysis essentially accesses the equilibrium force between the surface tension of the liquid molecules and a solid material surface in terms of cohesion and adhesion energy. Cohesion free energy is defined as the free energy change per unit area in the process of bringing two like materials together to form a continuous body like the contracting forces pulling the liquid surface into a droplet. Adhesion free energy is the free energy change in the process of bringing two unlike bodies together. When the liquid surface tension exceeds the material tension, one obtains a high cohesion energy between the liquid molecules that results in a beaded liquid droplet and overall poor wetting of a material surface. A large contact angle is observed in unfavorable wetting conditions. A higher material surface tension correlates to a larger adhesion free energy, in which the material's inward attractive forces dominate the intermolecular forces of a liquid to spread and wet the surface. Naturally, a small contact angle is observed in favorable wetting conditions. Because of this, contact angle continues to be one of the accurate methods of characterizing material wettability.

The surface geometry of the titanium-based material may be altered in a variety of beneficial ways to provide the desired biocompatibility, each independently providing specific biological functions. Surface geometry may be simultaneously altered in multiple aspects over a selected surface area and selected depth, allowing for the efficient generation of compositions with enhanced bioactivity, including alterations to both inter-pore and intra-pore areas.

In an embodiment, the surface geometry is a spatial distribution of relief features, recessed features, localized regions characterized by a selected composition, phase, crystallographic texture, or any combination of these. In embodiments, the surface geometry is a periodic or semi-periodic spatial distribution of the nanoscale domains. In embodiments, the surface geometry is provided between and within pores of the substrate. In embodiments, the surface geometry is a selected topology, topography, morphology, texture or any combination of these.

In embodiments, for example, each of the nanoscale domains are characterized by a vertical spatial dimension less than or equal to 250 nm, less than or equal to 100 nm, less than or equal to 50 nm or, optionally, selected over the range of 10 nm to 250 nm, 10 nm to 100 nm or 10 nm to 50 nm. In embodiments, the nanoscale domains comprise nanowalls, nanorods, nanoplates, nanoripples or any combination thereof having lateral spatial dimensions selected over the range of 10 to 1000 nm and vertical spatial dimensions of less than or equal to 250 nm. In an embodiment, for example, the nanowalls, nanorods, nanoplates or nanoripples are inclined towards a direction oriented along a selected axis relative to the surface. In embodiments, the nanowalls, nanorods, nanoplates or nanoripples are separated from one another by a distance of less than 100 nm.

In an embodiment, for example, the nanoscale domains comprise discrete crystallographic domains. In an embodiment, the crystallographic domains are characterized as an α+β annealed alloy. In embodiments, the nanoscale domains characterized by a chemical composition different from the bulk phase of the titanium or titanium alloy substrate.

The provided surface geometries allow for specific, designed increases in biofunctionality of the modified surfaces and/or domains. The described increased bioactivity allows for the fabrication of compositions and implants that decrease bacterial growth or inflammation, improve biomechanical compatibility between the material and in situ tissue, promote functional deployment and increase implant lifetime and success rate.

In embodiments, the surface geometry provides an enhancement in vivo or in vitro activity with respect to cell adhesion proliferation activity and migration greater than or equal to 100%. In an embodiment, the surface geometry provides an enhancement in vivo or in vitro activity with respect to anti-bacterial activity and bactericidal activity greater than or equal to 100%. In embodiments, the surface geometry provides an enhancement of a selected physical property of the substrate, for example, hydrophillicity, hydrophobicity, surface free energy, surface charge density or any combination of these. In embodiments, the enhancement of selected physical property is equal to or greater than 25%.

The provided compositions and methods include a variety of titanium-based substrates or implants known as useful in medical procedures or as medical devices. The provided titanium substrate may include titanium-based materials preprocessed to have porosity or surface characteristics to further increase biofunctionality.

In embodiments, the titanium or titanium alloy substrate is a biocompatible substrate, for example, mesoporous, microporous, or nanoporous substrates. Advantageously, the described systems and methods may generate nanostructures and nanopatterns on the surface of the pore, between pores or both. In embodiments, the titanium or titanium alloy substrate comprises commercially pure titanium metal (cpTi), Ti6Al4V alloy or a combination thereof. In an embodiment, for example, the titanium or titanium alloy substrate comprises a component of a medical device. In an embodiment, the medical device is a dental implant, a joint, hip or shoulder replacement, pedicle screw, syringe, needle, scalpel, or other surgical rod, plate or spinal injury instrument device.

The energetic particle beam(s) provided herein may be individually controlled to promote specific self-assembly of nanostructures, topography and/or topography and/or to alter chemical composition, morphology or crystallography. In the embodiments utilizing multiple beams, each beam may be independently controlled by one or more beam parameters to achieve the desired biofunctionality. Given the option of multiple beams each controlled by one or more independent parameters, complex surface alterations are possible.

In an embodiment, the directed energetic particle beam is a broad beam, focused beam, asymmetric beam, reactive beam or any combination of these. In embodiments, for example, the one or more beam properties is intensity, fluence, energy, flux, incident angle, ion composition, neutral composition, ion to neutral ratio or any combinations thereof.

In an aspect, provided is a method of fabricating a bioactive titanium-containing substrate, the method comprising: i) providing the titanium or titanium alloy substrate having a substrate surface; and ii) directing a directed energetic particle beam onto the substrate surface, thereby generating a plurality of nanoscale domains on the surface; wherein the directed energetic particle beam has one or more beam properties selected to generate the plurality of nanoscale domains characterized by a surface geometry providing a selected multifunctional bioactivity. In an embodiment, the directed energetic particle beam is a broad beam, focused beam asymmetric beam or any combination of these. In embodiments, for example, the step of directing the directed energetic particle beam onto the substrate surface comprises directed irradiation synthesis (DIS), directed plasma nanosynthesis (DPNS), Direct Seeded Plasma Nanosynthesis (DSDPNS), Direct Soft Plasma Nanosynthesis (DSPNS) or any combination of these.

In an embodiment for certain applications, the step of directing the directed energetic particle beam onto the substrate surface is achieved using a method other than directed irradiation synthesis (DIS). For example, the invention, includes methods of fabricating a bioactive titanium-containing substrate wherein directed plasma nanosynthesis (DPNS), direct seeded plasma nanosynthesis (DSPNS) or any combination of these techniques is used to carry out the step of directing the directed energetic particle beam onto the substrate surface to generate a plurality of nanoscale domains characterized by a surface geometry providing a selected multifunctional bioactivity. Accordingly, one of skill in the art will readily understand that certain applications and materials of the invention are achieved using methods that do not include processing via directed irradiation synthesis (DIS).

In an aspect, provided is a method of fabricating a bioactive titanium-containing substrate, the method comprising: i) providing the titanium or titanium alloy substrate having a substrate surface; and ii) directing a first directed energetic particle beam and a second directed energy particle beam onto the substrate surface, thereby generating a plurality of nanoscale domains on the surface; wherein the first directed energetic particle beam has one or more first beam properties and the second directed energetic particle beam has one or more second beam properties; and wherein at least one of the first beam properties is different than at least one of the second beam properties and the first beam properties and the second beam properties are independently selected to generate the plurality of nanoscale domains characterized by a surface geometry providing a selected multifunctional bioactivity.

In embodiments, the one or more beam properties is intensity, fluence, energy, flux, incident angle, ion composition, neutral composition, ion to neutral ratio or any combinations thereof. In an embodiment, the directed energetic particle beam comprises one or more ions, neutrals or combinations thereof. In an embodiment, the ions are Kr ions or Ar ions. In embodiments, aid one or more beam properties comprise incident angle and the incident angle is selected from the range of 0° to 80°. In embodiments, the one or more beam properties comprise fluence and the fluence is selected from the range of 1×10¹⁶ cm⁻² to 1×10¹⁹ cm⁻² or optionally 1×10¹⁶ cm⁻² to 1×10²⁰ cm⁻². In an embodiment, for example, the one or more beam properties comprise energy and the energy is selected from the range of 0.05 keV to 10 keV, or optionally, 0.1 keV to 10 keV. The use of energetic particle beams allows for substrate quench rates that are greater than traditional thermal and/or chemical processing methods. Quench rates may, for example, be nearly instantaneous as the directed particle beams may athermally interact with the substrate. In embodiments, the provided energetic particle beams provide a quench rate selected from the range of 10¹¹ K/s to 10¹⁴ K/s (degrees Kelvin per second). In embodiments, for example, the substrate is quenched in less than or equal to 10 μs, or optionally, less than or equal to 1 ns.

The described compositions, systems and methods may be beneficial in any other applications in tunability of surface interfaces is desirable, such as tuning surface chemistry, topography, crystallographic structure or physical properties to support specific functions. For example, the provided compositions and methods may be utilized to achieve desired functionality in healthcare applications, aseptic processing, food and beverage production, consumer products and industrial processes.

Bioactivated titanium may be useful in a wide range of healthcare related applications, not limited to implants but expanding to a variety of medical devices. For example, bioactive titanium may be used in aseptic processing for the production of pharmaceuticals, vaccines, food and beverage or medicals devices, for example, by increasing antibacterial properties. Further, bioactive titanium can implemented in surgical instruments, dental instruments, and biosensors, including in vivo sensors and those integrated with or applied to tissues.

Surface modified titanium may also be useful in non-biological applications such as consumer products or industrial processes. For example, it may be used as a catalyst or catalyst support in a chemical reactor or processing device. Further, surface modified titanium may have enhanced heat transfer properties beneficial for use in heat exchangers, HVAC applications, insulators or other applications in which control of heat transfer is desirable.

As noted herein, the titanium compositions may be rendered anti-bacterial by the treatments described herein. On many surfaces exposed to the environment, there is the risk that a microbial biofilm may form on a surface. The compositions of the invention may be used together with any surface. The surface is not limited and includes any surface on which a microorganism may occur, particularly a surface exposed to water or moisture. Treating surfaces to avoid films of antimicrobial compounds or manufacturing with them the working surfaces of laboratories (clinical, microbiological, water analysis, food), of businesses handling fresh food (butchers, fishmongers, etc.), of hospital buildings and health centers, to mention just a few examples, guarantees the suitable hygienic conditions for development of the work and eliminates the risk of contamination and infections.

Such inanimate surfaces exposed to microbial contact or contamination include in particular any part of: food or drink processing, preparation, storage or dispensing machinery or equipment, air conditioning apparatus, industrial machinery, e.g. in chemical or biotechnological processing plants, storage tanks and medical or surgical equipment. Any apparatus or equipment for carrying or transporting or delivering materials, which may be exposed to water or moisture is susceptible to biofilm formation. Such surfaces will include particularly pipes (which term is used broadly herein to include any conduit or line). Representative inanimate or abiotic surfaces include, but are not limited to food processing, storage, dispensing or preparation equipment or surfaces, tanks, conveyors, floors, drains, coolers, freezers, equipment surfaces, walls, valves, belts, pipes, air conditioning conduits, cooling apparatus, food or drink dispensing lines, heat exchangers, boat hulls or any part of a boat's structure that is exposed to water, dental waterlines, oil drilling conduits, contact lenses and storage cases. As noted above, medical or surgical equipment or devices represent a particular class of surface on which a biofilm may form. This may include any kind of line, including catheters (e.g. central venous and urinary catheters), prosthetic devices e.g., heart valves, artificial joints, false teeth, dental crowns, dental caps and soft tissue implants (e.g. breast, buttock and lip implants). Any kind of implantable (or “in-dwelling”) medical device is included (e.g. stents, intrauterine devices, pacemakers, intubation tubes, prostheses or prosthetic devices, lines or catheters). An “in-dwelling” medical device may include a device in which any part of it is contained within the body, i.e. the device may be wholly or partly in-dwelling. Plastic materials with antimicrobial properties can also be used in manufacturing handles, handlebars, handgrips and armrests of public transport elements, in rails and support points in places widely used, in the manufacturing of sanitary ware for public and mass use, as well as in headphones and microphones of telephones and audio systems in public places; kitchen utensils and food transport, all with the purpose of reducing the risk of propagation of infections and diseases.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1. Surface characteristics of Ti6Al4V samples before DIS processing: a) Surface prepared up to mirror finishing; b) Biphasic (α (hcp) is grey and β (bcc) is white) structure of the alloy revealed after polishing and metallographic acid etching.

FIG. 2. As received micro-structures of rough and porous+polished cpTi samples before Ar+irradiation a) Rough as-machined condition; b) Bulk machined samples after polishing and metallographic etching; c) Porous and polished samples; d) Porous and polished after metallographic etching.

FIG. 3. SEM images of a porous+polished cpTi sample (S1 (CPTi) lowest porosity) after Ar+DIS with an incident angle of 60 degrees: a) Overview of DIS nano-patterning in which is evident nano-rods inside scrathces; b) Nano-rods on flat surfaces with same orientation; c) Some nano-ripples perpendicular to ion beam direction; d) Details of oriented nano-rods; e) Nano-rods with difference orientations (crystallographic orientations); f) and g) Nano-rods inside scratch and nano-ripples on the flat zone; h) and i) Details of oriented nano-rods.

FIG. 4. SEM images of a rough cpTi samples after Ar+DIS with different incident angles: a) to d) Normal incidence; e) and f) Off-normal incidence of 45°; g) to i) Off-normal incidence of 75°.

FIG. 5. AFM analysis of porous and rough cpTi samples after Ar+DIS: a) Porous sample with roughness rms=0.57 nm; average height of 6.7 nm; b) Porous sample with roughness rms=3.49 nm; average height of 18.1 nm; c) Rough sample after incidence angle of 45° with roughness rms=73.35 nm; average height of 185 nm; d) Rough sample after incidence angle of 75° with roughness rms=22.07 nm; average height of 52.42 nm.

FIG. 6. Master-diagram that illustrates semi-quantitative and phenomenological relationships between DIS processing on samples of cpTi with theirs surface structural modifications and free surface energy responses.

FIG. 7. Fluorescent images showing cell nucleoids: a) the DNA presents strand breaks that are spreaded as a comet tail (positive control); b) negative nucleoids are compact and correspond to untreated cells; (c-f) nucleoids depevopeded by cells exposure to tested materials.

FIG. 8. Data distribution for percentage of DNA in tail of HASMCs cultured in the presence of irradiated metal samples. Notice that the variation range for the tested materials remains no higher than 8% and closer to the negative control.

FIG. 9. SEM images showing the evolution of surface nano-patterning of Ti6Al4V samples for different incidence angles with Ar+irradiation.

FIG. 10 Cytotoxicity results presented as the cell survival rate in terms of incidence angle of irradiation for the Ti6Al4V alloy. A control sample without exposure to Ar+irradiation was used on a polished Ti6Al4V alloy surface.

FIG. 11. SEM images showing the evolution of cells/nano-structured surfaces interactions and morphology changes for different incidence angles with Ar+irradiation.

FIG. 12. Map of SEM images showing the nano-scale relationship between structural and cells morphology evolutions for different incidence angles with Ar+irradiation.

FIG. 13. Master-diagram that relates different regimes of interactions between atoms and irradiation ions (diffusive and erosive) with the associated nano-patterning for different incidence angles with Ar+: red curve is a proposed formation behavior for nano-ripples and green curve is a proposed one for nano-rods formation (adapted from BH model).

FIG. 14. Ternary-diagram that relates angles of incidence, surface nano-ripples and a cells interaction criteria (filopodia activity) for irradiation with Ar+: desired relationship between nano-ripples and filopodia activity is obtained around 30° incidence angle.

FIG. 15. Figure above is 30-deg Ar+1-keV irradiation. This could be a special figure and also for 60-degree irradiation to support claims made in paragraph above.

FIG. 16. a) Long nano-walls and b) short nano-walls (MAG×80,000).

FIG. 17. a) Narrow nano-cones and b) wide nano-cones (MAG×50,000).

FIG. 18. Nano-ripples (MAG×120,000).

FIG. 19. Nano-walls (MAG×30,000).

FIG. 20. a) Narrow nano-cones; b) wide nano-cones and c) Round plate formation.

FIG. 21. Nano-walls of different lengths.

FIG. 22. a) Narrow nano-cones and b) wide nano-cones.

FIG. 23. a) Long nano-walls and short nano-walls.

FIG. 24. a) Narrow nano-cones and b) wide nano-cones.

FIG. 25. a) Small nano-walls with smooth surface and b) small nano-walls at high resolution.

FIG. 26. a) Narrow nano-cones at low mag and b) narrow nano-cones at high resol.

FIG. 27. Initial nano-wall formation at high resolution.

FIG. 28. Initial nano-cone formation at high resol.

FIG. 29. SEM images showing the evolution of surface nano-patterning of Ti6Al4V samples for different incidence angles with Ar+irradiation.

FIG. 30. Parameter: Fluence 1×10¹⁸ cgs (Ar)_02; a) Survey scans and b) core scans.

FIG. 31. Parameter: Fluence 7.5×10¹⁷ cgs (Ar)_10; a) Survey scans and b) core scans.

FIG. 32. Parameter: Fluence 5×10¹⁷ cgs (Ar)_09; a) Survey scans and b) core scans.

FIG. 33. Parameter: Fluence 2.5×10¹⁷ cgs (Ar)_08; a) Survey scans and b) core scans.

FIG. 34. Parameter: Fluence 1×10¹⁷ cgs (Ar)_07; a) Survey scans and b) core scans.

FIG. 35. AFM analysis of porous cpTi samples after Ar+DPNS: a) Porous sample with roughness rms=0.57 nm; average height of 6.7 nm; b) Porous sample with roughness rms=3.49 nm; average height of 18.1 nm.

FIG. 36. Fluorescent images showing cell nucleoids: the DNA presents strand breaks that are spreaded as a comet tail (positive control); negative nucleoids are compact and correspond to untreated cells; Ti-virgin and porous cpTi nucleoids developed by cells exposure to tested materials.

FIG. 37. Data distribution for percentage of DNA in tail of HASMCs cultured in the presence of irradiated metal samples. Notice that the variation range for the tested materials remains no higher than 8% and closer to the negative control.

FIG. 38. IGNIS facility at the UIUC.

FIG. 39. Pro-inflammatory and anti-inflammatory events in the body after insertion of an implant.

FIG. 40. High-resolution SEM of Ti-based surface showing nanostructured columns that can be varied along various Ti grains that may enhance the adhesion properties of cells.

FIG. 41. (left) Nanotopography of advanced 7.5×10¹⁷ cm⁻² fluence irradiation with Ar+ions at 1-keV and 60-degree incidence. (right) Early stage nanopatterning at 10¹⁷ cm⁻² fluence.

FIG. 42. (left) striking nanopatterning rich in a variety of patterns directed by grain orientation effects. By combining both the grain size and texture with DIS one could obtain very specific patterning systems to enhance cell adhesion properties, (right) Pillar-like structures are synthesized with about 200-400 nm pillar structures becomes an exiting anti-bacterial surface similar to the surface structure of cicada wings (see FIG. 47).

FIG. 43. (top Left) The advantage is to have nanostructures that can be filled with a therapeutic compound in this case in Ti-based implant biomaterials (figure from: V-P. Lehto et al. 2013, Wiley & Sons) (top Right) Cicada (Psaltoda claripennis) shown in a) and its wing in b) with an SEM image of the wing surface (marker at 2-um) showing nanostructures of about 200-nm height with 100-nm diameter at base and 60-nm at cap (e.g. a nanopillar) spaced about 170-nm apart (from: E. P. Ivanova et al. Small 2012, 8 (16) 2489-2494. (bottom Left) SEM showing bacteria dying over the cicada wing nanostructure topography. (bottom Right) The DIS-synthesized nanostructure demonstrating a factor 100× surface area likely more potent anti-bacterial properties.

FIG. 44. Contact angle results of an unirradiated cpTi surface (S0; 53.03±0.06) and after irradiation with Ar+; the surface wettability increased significantly after irradiation (lower contact angle, 10×): S1(0-degree incidence) (40%); S2 (30-deg) (70%); S3 (60-deg) (20%); S4 (80-deg) (10%); S6 (70%).

FIG. 45. Alkaline phosphatase (ALP) expression induced on a polished Ti6Al4V alloy sample, two krypton irradiated alloy samples, and 3 control tissue plate cell cultures. Titanium DIS parameters: Ti—Kr-03—Gas: Kr, Angle: 0°, Energy: 1000 eV, Fluence: 1×10¹⁸ cgs and Ti—Kr-05—Gas: Kr, Angle: 0°, Energy: 1000 eV, Fluence: 1×10¹⁸ cgs. Notice sample Ti—Kr-05 had significantly higher ALP expression than all other titanium alloy surfaces.

FIG. 46. ELISA cytokine measurements of macrophage TNF-α secretion stimulated by 24-hour cell culture in ten different culture conditions, including a LPS positive tissue plate cell medium, a tissue plate cell medium, a polished titanium surface, and eight krypton and argon irradiated titanium surfaces in total. Despite the lack of linearity between nanostructure formation surface area, shape, size and cytokine secretion, most titanium surfaces appear to elicit a strong pro-inflammatory pathway of activation. Notice contrasting surfaces of polished titanium and sample Ti—Ar-10 induced high amounts of TNF-α that exceeded the cytokine secretion in the positive LPS control. Titanium DIS parameters: Ti—Ar-10—Gas: Ar, Angle: 60°, Energy: 1000 eV, Fluence: 7.5×10¹⁷ cgs, Ti—Ar-09—Gas: Ar, Angle: 60°, Energy: 1000 eV, Fluence: 5.0×10¹⁷ cgs, Ti—Ar-08—Gas: Ar, Angle: 60°, Energy: 1000 eV, Fluence: 2.5×10¹⁷ cgs, Ti—Ar-07—Gas: Ar, Angle: 60°, Energy: 1000 eV, Fluence: 1.0×10¹⁷ cgs, Ti—Ar-02—Gas: Ar, Angle: 60°, Energy: 1000 eV, Fluence: 1.0×10¹⁸ cgs, Ti—Kr-05—Gas: Kr, Angle: 0°, Energy: 1000 eV, Fluence: 1×10¹⁸ cgs, and Ti—Kr-03—Gas: Kr, Angle: 0°, Energy: 1000 eV, Fluence: 1×10¹⁸ cgs.

FIG. 47. ELISA cytokine measurements of macrophage IL-10 secretion stimulated by 24-hour cell culture in ten different culture conditions, including a LPS positive tissue plate cell medium, a tissue plate cell medium, a polished titanium surface, and eight krypton and argon irradiated titanium surfaces in total. Notice all titanium samples induced very small secretions of IL-10 at 24 hours in contrast to the tissue culture plate cell mediums. Titanium DIS parameters: Ti—Ar-10—Gas: Ar, Angle: 60°, Energy: 1000 eV, Fluence: 7.5×10¹⁷ cgs, Ti—Ar-09—Gas: Ar, Angle: 60°, Energy: 1000 eV, Fluence: 5.0×10¹⁷ cgs, Ti—Ar-08—Gas: Ar, Angle: 60°, Energy: 1000 eV, Fluence: 2.5×10¹⁷ cgs, Ti—Ar-07—Gas: Ar, Angle: 60°, Energy: 1000 eV, Fluence: 1.0×10¹⁷ cgs, Ti—Ar-02—Gas: Ar, Angle: 60°, Energy: 1000 eV, Fluence: 1.0×10¹⁸ cgs, Ti—Kr-05—Gas: Kr, Angle: 0°, Energy: 1000 eV, Fluence: 1×10¹⁸ cgs, and Ti—Kr-03—Gas: Kr, Angle: 0°, Energy: 1000 eV, Fluence: 1×10¹⁸ cgs.

FIG. 48. ELISA cytokine measurements of macrophage TNF-α secretion stimulated by 48-hour cell culture in ten different culture conditions, including a LPS positive tissue plate cell medium, a tissue plate cell medium, a polished titanium surface, and eight krypton and argon irradiated titanium surfaces in total. Notice at 48 hours of cell culture, the majority of the titanium surfaces exhibit a decrease in their TNF-α concentrations. This may indicate the potential end of the pro-inflammatory activity of the macrophage. The LPS tissue plate culture is the only testing condition to exhibit a peaking increase in TNF-α secretion. Titanium DIS parameters: Ti—Ar-10—Gas: Ar, Angle: 60°, Energy: 1000 eV, Fluence: 7.5×10¹⁷ cgs, Ti—Ar-09—Gas: Ar, Angle: 60°, Energy: 1000 eV, Fluence: 5.0×10¹⁷ cgs, Ti—Ar-08—Gas: Ar, Angle: 60°, Energy: 1000 eV, Fluence: 2.5×10¹⁷ cgs, Ti—Ar-07—Gas: Ar, Angle: 60°, Energy: 1000 eV, Fluence: 1.0×10¹⁷ cgs, Ti—Ar-02—Gas: Ar, Angle: 60°, Energy: 1000 eV, Fluence: 1.0×10¹⁸ cgs, Ti—Kr-05—Gas: Kr, Angle: 0°, Energy: 1000 eV, Fluence: 1×10¹⁸ cgs, and Ti—Kr-03—Gas: Kr, Angle: 0°, Energy: 1000 eV, Fluence: 1×10¹⁸ cgs.

FIG. 49. ELISA cytokine measurements of macrophage IL-10 secretion stimulated by 48-hour cell culture in ten different culture conditions, including a LPS positive tissue plate cell medium, a tissue plate cell medium, a polished titanium surface, and eight krypton and argon irradiated titanium surfaces in total. Titanium DIS parameters: Ti—Ar-10—Gas: Ar, Angle: 60°, Energy: 1000 eV, Fluence: 7.5×10¹⁷ cgs, Ti—Ar-09—Gas: Ar, Angle: 60°, Energy: 1000 eV, Fluence: 5.0×10¹⁷ cgs, Ti—Ar-08—Gas: Ar, Angle: 60°, Energy: 1000 eV, Fluence: 2.5×10¹⁷ cgs, Ti—Ar-07—Gas: Ar, Angle: 60°, Energy: 1000 eV, Fluence: 1.0×10¹⁷ cgs, Ti—Ar-02—Gas: Ar, Angle: 60°, Energy: 1000 eV, Fluence: 1.0×10¹⁸ cgs, Ti—Kr-05—Gas: Kr, Angle: 0°, Energy: 1000 eV, Fluence: 1×10¹⁸ cgs, and Ti—Kr-03—Gas: Kr, Angle: 0°, Energy: 1000 eV, Fluence: 1×10¹⁸ cgs. Notice, polished titanium is the only sample to induce a significant increase in IL-10 secretion that exceeds its TNF-α secretion, while all other testing conditions show a decrease in IL-10.

FIG. 50. ELISA cytokine measurements of macrophage TNF-α secretion stimulated by 24-hour cell culture in ten different culture conditions (see description in FIG. 52).

FIG. 51. ELISA cytokine measurements of macrophage TNF-α secretion stimulated by 48-hour cell culture in ten different culture conditions (see description in FIG. 52).

FIG. 52. ELISA cytokine measurements of macrophage TNF-α secretion stimulated by 72-hour cell culture in ten different culture conditions, including a LPS positive tissue plate cell medium, a tissue plate cell medium, a polished titanium surface, and eight krypton and argon irradiated titanium surfaces in total. Titanium DIS parameters: Ti—Ar-10—Gas: Ar, Angle: 60°, Energy: 1000 eV, Fluence: 7.5×10¹⁷ cgs, Ti—Ar-09—Gas: Ar, Angle: 60°, Energy: 1000 eV, Fluence: 5.0×10¹⁷ cgs, Ti—Ar-08—Gas: Ar, Angle: 60°, Energy: 1000 eV, Fluence: 2.5×10¹⁷ cgs, Ti—Ar-07—Gas: Ar, Angle: 60°, Energy: 1000 eV, Fluence: 1.0×10¹⁷ cgs, Ti—Ar-02—Gas: Ar, Angle: 60°, Energy: 1000 eV, Fluence: 1.0×10¹⁸ cgs, Ti—Kr-05—Gas: Kr, Angle: 0°, Energy: 1000 eV, Fluence: 1×10¹⁸ cgs, and Ti—Kr-03—Gas: Kr, Angle: 0°, Energy: 1000 eV, Fluence: 1×10¹⁸ cgs. Notice, 72 hours of cell culture resulted in sample Ti—Ar-02 finally stimulating TNF-α secretion after initial measurements of zero at all previous time points. In contrast to the majority of the testing conditions, finely nanostructured samples Ti—Ar-07 and Ti—Ar-08 exhibited an increase in TNF-α.

FIG. 53. LPS supplemented tissue plate cell culture and tissue plate cell culture TNF-α concentrations measured over a 72-hour period. Notice both culture controls seem to follow the typical cell activation trends observed with implantation at 48 hours when host defense cells dominate the implant environment with different pro-inflammatory cytokines and enzymes.

FIG. 54. Tissue Culture Plate Control 24-72 Hour Macrophage Culture TNF-α Secretion with culture period (hrs) on the x-axis and concentration (pg/ml) on the y-axis.

FIG. 55. Titanium surface macrophage cultures observed to exhibit a decrease in TNF-α secretion over a 72-hour period similar to the LPS positive and tissue culture plate cell cultures. Titanium DIS parameters—Ti—Kr-03—Gas: Kr, Angle: 0°, Energy: 1000 eV, Fluence: 1×10¹⁸ cgs, Ti—Kr-05—Gas: Kr, Angle: 0°, Energy: 1000 eV, Fluence: 1×10¹⁸ cgs, Ti—Ar-09—Gas: Ar, Angle: 60°, Energy: 1000 eV, Fluence: 5.0×10¹⁷ cgs, and Ti—Ar-10—Gas: Ar, Angle: 60°, Energy: 1000 eV, Fluence: 7.5×10¹⁷ cgs. Although polished titanium and sample Ti—Ar-10 appeared to induce the more robust pro-inflammatory responses initially, notice both samples showed the greatest decrease in TNF-α secretion at each time point compared to other titanium samples.

FIG. 56. Tissue Culture Plate Control 24-72 Hour Macrophage Culture TNF-α Secretion with culture period (hrs) on the x-axis and concentration (pg/ml) on the y-axis.

FIG. 57. Titanium surface macrophage cultures observed to exhibit a delay or increase in TNF-α secretion over a 72-hour period in contrast to the control cultures and typical inflammatory trends observed in the body. Titanium DIS parameters—Ti—Ar-02—Gas: Ar, Angle: 60°, Energy: 1000 eV, Fluence: 1.0×10¹⁸ cgs, Ti—Ar-07—Gas: Ar, Angle: 60°, Energy: 1000 eV, Fluence: 1.0×10¹⁷ cgs, and Ti—Ar-08—Gas: Ar, Angle: 60°, Energy: 1000 eV, Fluence: 2.5×10¹⁷ cgs. Compared to all the TNF-α positive supernatants at 24 hours, sample Ti—Ar-08 had the lowest TNF-α secretion, but notice this same sample had the highest TNF-α secretion at 72 hours that exceeded the amount of the LPS positive control for pro-inflammatory cytokines. Sample Ti—Ar-02 seems to display a delay in macrophage activation and cytokine secretion.

FIG. 58. Contact angle results of an unirradiated titanium alloy versus titanium alloys irradiated at an increasing fluence from 1.0×10¹⁷ cgs to 1.0×10¹⁸ cgs. Contact angle measurements: Polished Titanium—76.1±3.9, Ti—Ar-07-82.3±6.7, Ti—Ar-08-78.4±4.8, Ti—Ar-09-74.1±7.3, Ti—Ar-10-81.3±2.8, and Ti—Ar-02-68.7±2.2. Notice the argon irradiation appears to cause a small decrease in surface wettability.

FIG. 59 a-c. Flat, clustered platelets and nano-ripples formed on the surface. Such structures are formed using the following DIS parameter—Gas: O, Angle: 60°, Energy: 1000 eV, Fluence: 1×10¹⁸ cgs.

FIG. 60 a-b. SEM image displaying the drastic effect of incident angle on nanostructure formation with oxygen ion irradiations. Ti—O-13 (FIG. 60a)—smooth titanium alloy w/minimal impurities. Ti—O-14 (FIG. 60 b)—flat, clustered platelet and nano-rippled titanium surface. Such structures are formed using the following DIS parameter: Ti—O-13—Gas: O, Angle: 0°, Energy: 1000 eV, Fluence: 1×10¹⁸ cgs and Ti-0-14—Gas: O, Angle: 60°, Energy: 1000 eV, Fluence: 1×10¹⁸ cgs.

FIG. 61 a-d. SEM images displaying the drastic effect of incident angle on nanostructure formation with krypton ion irradiations. Ti—Kr-15 (FIG. 61a-b)—smooth titanium surface with various regions of fine nano-walls & ripples. Ti—Kr-16 (FIG. 61 c-d)—titanium surface with different direction oriented nano-walls in protruding grain boundaries. Such structures are formed using the following DIS parameter: Ti—Kr-15—Gas: Kr, Angle: 0°, Energy: 1000 eV, Fluence: 1×10¹⁸ cgs and Ti—Kr-16—Gas: Kr, Angle: 60°, Energy: 1000 eV, Fluence: 1×10¹⁸ cgs.

FIG. 62 a-d. Different direction-oriented nano-walls and nano-cones formed on the surface. Such structures are formed using the following DIS parameter: Ti—Kr-17—Gas: Kr, Angle: 60°, Energy: 1000 eV, Fluence: 7.5×10¹⁷ cgs.

FIG. 63 a-c. Large, stacked nano-cones and thin nano-walls formed on the surface. Such structures are formed using the following DIS parameter: Ti—Kr-18—Gas: Kr, Angle: 60°, Energy: 1000 eV, Fluence: 5.0×10¹⁷ cgs.

FIG. 64 a-d. Long nano-walls and short nano-walls formed on the surface. Such structures are formed using following DIS parameters—Gas: Ar, Angle: 0°, Energy: 1000 eV, Fluence: 1×10¹⁸ cgs.

FIG. 65 a-b. Long nano-walls and short nano-walls formed inside pores. Such structures are formed using following DIS parameters—Gas: Ar, Angle: 0°, Energy: 1000 eV, Fluence: 1×10¹⁸ cgs,

FIG. 66 a-b. Long nano-walls and short nano-walls formed on the walls of the pores. Such structures are formed using following DIS parameters—Gas: Ar, Angle: 0°, Energy: 1000 eV, Fluence: 1×10¹⁸ cgs.

FIG. 67 a-b. Long nano-walls and short nano-walls in a tilted view of the surface. Such structures are formed using following DIS parameters—Gas: Ar, Angle: 0°, Energy: 1000 eV, Fluence: 1×10¹⁸ cgs.

FIG. 68 a-b. Long nano-walls and short nano-walls formed on the surface. FIG. 68 c. Nano-cones along with nano-walls formed on the surface. Such structures are formed using following DIS parameters—Gas: Ar, Angle: 60°, Energy: 1000 eV, Fluence: 1×10¹⁸ cgs.

FIG. 69 a-b. Long nano-walls and short nano-walls formed in the pores. Such structures are formed using following DIS parameters—Gas: Ar, Angle: 60°, Energy: 1000 eV, Fluence: 1×10¹⁸ cgs.

FIG. 70 a. Titled view of surface Nano-cones FIG. 70 b. Tilted view of surface nano-walls. Such structures are formed using following DIS parameters—Gas: Ar, Angle: 60°, Energy: 1000 eV, Fluence: 1×10¹⁸ cgs.

FIG. 71 a-b. Nano-walls and nano-cone formed on the surface. Such structures are formed using following DIS parameters—Gas: Ar, Angle: 60°, Energy: 500 eV, Fluence: 1×10¹⁸ cgs.

FIG. 72 a-c. Narrow and wide nano-cones formed on the surface. Such structures are formed using following DIS parameters—Gas: Ar, Angle: 60°, Energy: 750 eV, Fluence: 1×10¹⁸ cgs.

FIG. 73. Nano-cones inside the pores. Such structures are formed using following DIS parameters—Gas: Ar, Angle: 60°, Energy: 750 eV, Fluence: 1×10¹⁸ cgs.

FIG. 74 a-c. Long nano-walls and short nano-walls formed on the surface. Such structures are formed using following DIS parameters—Gas: Ar, Angle: 0°, Energy: 1000 eV, Fluence: 1×10¹⁸ cgs.

FIG. 75 a-c. Long nano-walls and short nano-walls formed in the pores. Such structures are formed using following DIS parameters—Gas: Ar, Angle: 0°, Energy: 1000 eV, Fluence: 1×10¹⁸ cgs.

FIG. 76 a-c. Long nano-walls and short nano-walls formed on the walls of the pores. Such structures are formed using following DIS parameters—Gas: Ar, Angle: 0°, Energy: 1000 eV, Fluence: 1×10¹⁸ cgs.

FIG. 77 a-c. Long nano-walls and short nano-walls along with nano-cones formed on the surface. Such structures are formed using following DIS parameters—Gas: Ar, Angle: 60°, Energy: 1000 eV, Fluence: 1×10¹⁸ cgs.

FIG. 78. Surface morphology of porous Ti showing micro (blue arrow) and macro (red arrow) pores obtained by space-holder technique, using different space-holder percentage of NaCl (FIGS. 78 A-E) and NH₄HCO₃ (FIG. 78 F).

FIG. 79. Control 60% Ti showing macro and micro pores at a) low magnification and b) high magnification. c) Surface of untreated 60% Ti at high magnification. d) Nano walls obtained on the surface of 60% Ti irradiated using the following DIS parameters—Gas: Ar, Angle: 60⁰, Energy: 1000 eV, Fluence: 1×10¹⁸ cgs. e) Nano walls obtained on the surface of 60% Ti irradiated using the following DIS parameters-Gas: Ar, Angle: 0°, Energy: 1000 eV, Fluence: 1×10¹⁸ cgs.

FIG. 80. SEM image of before and after of a) control and b) irradiated 60% Ti sample. The DIS experimental parameters are as follows: Gas: Ar, Angle: 60⁰, Energy: 1000 eV, Fluence: 1×10¹⁸ cgs.

FIG. 81 SEM images of middle part of a commercial titanium alloy dental implant. The studied area is polished Titanium alloy (without SLA treatment) localized in the middle part. DPNS shows to be effective and develops small nanofeatures in the polished part. In addition, this complex topography which combines different planes, angles, pores does not suppose any obstacle to the development of nanoplatelets due to DPNS surface modification.

FIG. 82 SEM images of middle part of a commercial titanium alloy dental implant with the special focused on the SLA pretreated surface. The studied area is the SLA treated middle part in which DPNS shows to be effective as well and could develop small nanofeatures with similar morphology and size than in the previous polished area. This fact opens new frontiers in which complex medical devices can be improved without using chemical and toxic compounds.

FIG. 83 SEM images of lower part of commercial titanium alloy dental implants. The Argon ions of DPNS surface modification arrives at this area showing the presence of similar nanofeatures previously describe in FIGS. 81 and 82. Once more, DPNS shows to be effective in the surface modification of the 3D structures. In this case, the SLA modification of SLA does not create any obstacle modifying in deeper the dental implant surface at the nano-scale order.

FIG. 84 SEM images of middle part of a commercial titanium alloy dental implant SEM images of the upper part of commercial titanium alloy dental implant. The studied area is polished Titanium alloy (without SLA treatment) after DPNS processing. Small nanostructures are presented in this area due to DPSN treatment.

FIG. 85 SEM images of irradiated phosphate Ti6Al4V samples with high energy (1 Kev) and fluences. Both samples were irradiated using similar DPNS conditions but with two different incidence angles (0 and 60 degrees) and short times (18 and 20 mins of ion beam exposure respectively. It is remarkable the different surfaces of phosphate coatings developed on Ti6Al4V alloy samples which were obtained using higher energies and fluences. In that sense, the incidence angle plays an important role in the size and the morphology of the nanofeatures, and at normal incidence angle, the nanofeatures with nanoplatelet morphology present smaller size compared to the nanosharp pillars or nanocones found in the off-normal irradiation sample.

FIG. 86 SEM images of irradiated phosphate Ti6Al4V samples with medium energy (750 eV) and high fluences. Both samples were irradiated using similar DPNS conditions but with two different incidence angles (0 and 60 degrees) and longer times (30 and 40 mins of ion beam exposure). It is remarkable the different surfaces of phosphate coatings developed on Ti6Al4V samples which were obtained using medium energies and high fluences. In that sense, the incidence angle plays an important role, even much more than in the previous figure, because the nanofeatures found are totally different in size and in morphology. At 30 mins of irradiation time, the nanostructures develop present long shape similar as worm-like, however, using 40 mins and 60 degrees of incidence angle, the interaction with the grain and mineral component is different and produce nano-sharp structures similar as high energies (see FIG. 85).

FIG. 87 SEM images of phosphate Ti6Al4V samples irradiated with low energy (500 ev) and high fluences. Both samples were irradiated using similar DPNS conditions but with two different incidence angles (0 and 60 degrees) and longer times (32 and 50 mins of ion beam exposure). It is remarkable the different surfaces of phosphate coatings developed on Ti6Al4V samples which were obtained using low energies and high fluences. Again, the incidence angle plays an important role in both surfaces. With 60 degrees almost there is no nanofeatures and the surface is smooth. At normal incidence angle, the surface present similar nanofeatures found in FIG. 86 in PTi30 mins, worm-like structures but with lower density probably due to lower energies.

FIG. 88 Summarizes SEM images of phosphate Ti6Al4V samples irradiated at normal incidence angle (0) and high fluences. Three phosphate ti alloy samples were irradiated using similar DPNS conditions but with three different energies (1 KeV, 750 eV, and 500 eV) as well three times of ion beam exposure (18, 30 and 40 mins). The nanostructures develop are consistent with the time of DPNS treatment, longer times increase worm-like nano features due to the chemical interaction with the surface but at short times the nanofeatures are smaller and more elongated similar to nanoripples.

FIG. 89 Summarizes SEM images of phosphate Ti6Al4V samples irradiated at off-normal incidence angle (60 degrees) and high fluences. Three phosphate ti alloy samples were irradiated using similar DPNS conditions but with three different energies (1 KeV, 750 eV, and 500 eV) as well three times of ion beam exposure (20, 40 and 50 mins). The nanostructures develop do not follow the same behavior described in FIG. 86. At higher times the surface becomes smoother at short times of exposure. The modification using 60 degrees of incidence angle produce high nano-sharp cones at higher and medium energies but using lower energies, the nanofeatures disappear due to the highly oblique angle that ion beam crashed.

FIG. 90 Comparative SEM images of phosphate Ti6Al4V alloy samples irradiated at 60 degrees incidence angle with high energies (1 KeV), Three phosphate Ti6Al4V alloy samples were irradiated using similar DPNS conditions but with three different fluences high, medium and low which correspond to 1E18, 7E17, and 2.5E17 cm⁻². It should point it out that even the time of DPNS treatment was similar in the three samples using 20, 22, and 24 mins respectively, the nanofeatures develop were similar in shape and size. Notice, a slight increase in nanofeatures density using low fluences which were presented with sharper ends but the width was higher at long fluences.

FIG. 91 Comparative SEM images of phosphate Ti6Al4V alloy samples irradiated at 0 and 60 degrees and high energies (1 KeV) but with medium and low fluences (7E17, and 2.5E17 cm⁻²). Three phosphate Ti6Al4V alloy samples were irradiated using two types of incidence angles. At 60 degrees the nanofeatures present nano-sharp cones shape while at 0 degrees are more similar to a worm like structures. At lower fluences, with 60 degrees the nano cones become sharper due to the effect of the ion beam incidence angle.

FIG. 92 SEM images of phosphate Ti alloy samples irradiated with high energies (1 KeV) and low fluences (2.5E17). Both samples were irradiated with different incidence angle (0 and 60 degrees) and different time 28 and 24 mins. The nanofeatures were totally different in terms of morphology and size. At 0 degrees the surface seems to present a pore-like structures and using 60 degrees more nanosharpcones.

FIG. 93 Cell morphology evaluation of macrophages: Analysis of phenotype polarization. SEM images of macrophages J7741A seeded on the Ti surface at 72 h of cell incubation. The first row represents the general cell morphology of these immune cells, which the majority present round cell shape according to M0 phenotype. In order to get in depth the analysis of the cell morphology, a higher magnification study (second and third rows) was developed. Star-like cell shape was marked by yellow arrows finding a more developed M1 phenotype at lower and medium fluence, 2.5E17 and 5.0E17 cm⁻² respectively. Even in these images it was appreciated it a wide variety in cell morphology, a slightly tendency of rounded cell shape was found at higher fluence (1 E18 cm⁻²) observing with lower filopodia density which matched with M0 or M2 phenotype. These description were also confirmed at higher magnification, evaluating single cells and observing closer the cytoplasmic structures (filopodia and lamellipodia) in which smooth and lower fluences such as Polished Ti and 2.5E17 cm⁻² showed the M1 activation state. In this case, it was necessary to further analyses the filopodia at higher magnifications, which were shown in the next figure.

FIG. 94 Comparative results of filopodia analysis of macrophages. SEM images of the filopodia analysis of macrophages (J7741A) seeding on irradiated Titanium alloy samples at 72 h of incubation. The analyzed filopodia expression are was marked by yellow square in order to facilitate the studied area. The phenotype expression M1 and M2 is also related with filopodia activation and subsequently with cell morphology. In these images it is confirmed how smooth and lower fluences induce an increased in filopodia and cytoplasmic projections which was found to be more elongated that in higher fluences.

FIG. 95 24-72 hour macrophage culture ELISA cytokine Results. Cytokine quantification of TNF alpha and IL10 which are the most representative cytokines from the different phenotypes, M1 and M2 respectively. Macrophages were cultured on top of the irradiated Ti by DPNS and media was taken at 24, 48 and 72 h to measure the cytokine released using Elisa sandwich. Besides the overlap of the standard deviation, the results reveal interesting information about the immune response of DPNS treatment. First of all, TFNF alpha values increases with the incubation time as it was expected, however, great differences were found between fluences. At 24 h, all substrates were recognize as titanium by macrophages which expressed and released similar cytokine expression levels (TNF alpha≈100 pg/mL). At 48 and 72 hours of incubation, the TNF alpha levels increased around 300-400 pg/mL observing the highest concentration in low fluences and smoother surfaces, polished Titanium. In Panel B, it is shown the IL10 results at 24, 48, and 72 h. The cytokine expression showed different released prolife. At 24 h, similar Il10 was quantified in the media of all the specimens around 200-300 pg/mL. Compared to TNF alpha results, Il10 levels showed an increased at short time of incubation, however, it didn't increase with culture time as TNF alpha did, and at 48 h the results decreased except for the internal references (controls TCP ad LPS). At 72 h it was observed similar cytokine expression as found at 24 h, however the long standard deviation made harder the analyses of these results. For that reason, TNF alpha were evaluated again in next experiment and the ratio between cytokines was analyzed. From the table, it was observed that M2 phenotype was similar between samples but, with clear predominance for medium to higher fluences.

FIG. 96 Quantification of TNF alpha cytokine expression of macrophages (J7741A). Cells were cultured on top of the irradiated Ti modified by DPNS and media was taken at 24, 48 and 72 hours to measure the cytokine released using Elisa sandwich. In these repeated experiments, the TNF alpha values were similar as the levels obtained in the previous experiment (see FIG. 93), reaching 150 pg/mL at the first 24 h, and increasing the cytokine released at 48 and 72 h, reaching values of 250 and 350 pg/mL respectively. Here, in this figure, it is observed an increase of TNF alpha expression in lower and medium fluences ranging from 2.5 e17 to 7.5E17 cm⁻². Similarly, the polished Ti results were lower than than lower and medium fluences but it did not follow the same trend for the irradiated Ti using higher fluences as it observed in the right panel (B) in which polished and 1.0E18 run with similar (m trend) and in parallel, even the higher fluence reached out lower results than the polished Titanium. Also in this experiment two more samples were irradiated with different gas species (Nitrogen and Oxygen) and one commercial Ti reference referred as the “SLA” type surface. These samples reached the lowest levels of TNF alpha expression. Nitrogen sample offered the lowest and most delayed immune response since it did not activate the M1 phenotype of the macrophages, much more than SLA, with values around 25 pg/mL in NTi-1.0E18 compared to 100 pg/mL in SLA type surface at 72 h of cell incubation.

FIG. 97 Comparative results of cell morphology and CCR7 M1 phenotype expression of macrophages (Actin and DAPI analysis of cell cytoskeleton and nuclei by confocal microscopy): Argon fluence study. Confocal images of the Immunostaining experiment of cell cytoskeleton, cell nuclei and CCR7 surface cell marker of macrophages growing on Ti alloy samples. The purpose of this figure is to set up the colors obtained and its relationship with macrophages activation. In red color, due to Texas red phalloidin staining, it is represented actin fibers which are related to cell morphology and adhesion process. IN blue it is marked the DNA content by using Hoechst and in green the cell surface marker CCR7 which is frequently expressed in macrophages with M1 phenotype. In these images the polished titanium showed a three independent channels obtained from confocal microscopy. Merging the first two panel we are capable to analyze the cell morphology, and as we expected in PTi cells were round to star-like cell shape and including the green channel we are capable to distinguish the macrophages with M1 phenotype, related to engulfment and cleaning process.

FIG. 98 Comparative results of cell morphology of macrophages (Actin and DAPI analysis of cell cytoskeleton and nuclei by confocal microscopy): Argon fluence study. Cell morphology analysis of macrophages (J7741A) at 72 h of cell incubation. Confocal images of the immunostaining experiment were taken in order to evaluate the cell cytoskeleton (actin fibers appears in red) and the cell nuclei (DNA content in blue). One of the assumptions related to immune response to biomaterials is the relationship between the polarized phenotype and the cell morphology of macrophages. In this figure, cell cytoskeleton was evaluated to find a relationship between cell shape and phenotype expressed of macrophages growing on DPNS Ti alloy surfaces. It is noticeable, in general, the round cell shape in all the samples observing the cell nuclei as the majority of the total cell volume and the actin fibers expressed in low proportion. The rounded cells are related to non-activated macrophages (M0) while start-like shapes (as it is observed in low fluences 2.5 E 17) has been described as M1 phenotype (see white arrows) and elongated cell shape has been related to M2 phenotype. Regarding cell morphology of lower fluences such 2.5E17 it was observed a more variety in cell shape, finding star-like shape, elongated, polygonal, and round morphologies. However, due to the coexistence with other cytoskeleton morphologies in the same condition, it was difficult to analyze clearly the phenotype expressed. Following the same screening, the cell shape found in 5.0 and 7.5 E17 fluences are totally rounded with similar cell density and higher fluence showed similar cell variety than low fluence. Besides the cell morphology of M1 macrophages, it was also detected the presence of macrophages with two nucleus, which is common of initial foreign body giant cells (FBGCs) (see white arrows). These FBGCs are in charge of engulfing and removing particle debris and bacteria in the damage area, therefore, are key factors in inflammation process and in particular in foreign body reaction (FBR) due to biomaterials implantation. All the samples, including Polished Ti, showed a slightly presence of FBGCs similar as the control reference.

FIG. 99 Comparative results of cell morphology and macrophages (Actin and DAPI analysis of cell cytoskeleton and nuclei by confocal microscopy): Argon fluence study. Confocal images of FBGCs found in macrophages growing onto irradiated Ti substrate at 72 h of cell incubation. As it has been described before, the main function of these immune cells are related to cleaning of necrotic tissue, dead cells, particle debris or even bacteria. They are protagonist of the FBR of biomaterials, which try to fight against the surfaces releasing proteolytic enzymes and acidifying the pH with the aim to damage and isolated the implantable device. If these cells stay longer periods the inflammation process becomes chronic which finally promote the rejection of the biomaterial. In these images the presence of these FBGCs are low but in the case of low fluence it was found developed FBGCs with 5 to 6 nucleus for one cell.

FIG. 100 Comparative results of M1 phenotype of macrophages (Actin and DAPI analysis of cell cytoskeleton and nuclei by confocal microscopy): Argon fluence study. Evaluation of the M1 phenotype expression by confocal microscopy. CCR7 is constitutively expressed by M1 polarized macrophages and it is used as reference for M1 phenotype evaluation. In this images, clearly, the highest expression was found in Polished Titanium followed by slightly expression in higher fluence and medium. However the signal obtained for the irradiated titanium was lower than PTi assuming that CCR7 is also expressed in M0 and M2 macrophages in a low proportion.

FIG. 101 Evaluation of cell differentiation process of pre-osteoblast cells growing on Ti6Al4V substrates irradiated with different fluence in DIS. Alkaline phosphatase (ALP) enzyme is one of the most famous early bone markers expressed during osteoblast differentiation process and has become a key factor to compared titanium surfaces with different treatments. ALP was measured at 4, 7, and 14 days of pre-osteoblast MC3T3E1 cultured on Ti6Al4V alloy sample following the protocol guidelines. In panel A, ALP results are expressed as pg/mins/ml as well panel B, but in the later, ALP is represented as linear behavior to make easier the trend and relationship between conditions. At 4 days, the ALP levels reached remained low, closed to 250 pg/mins/mL and no differences between fluence were found. However, in this context, increasing the incubation time to 7 days, ALP content raised up to nearly 1200 pg/mins/mL, in which polished Ti showed the poorest levels (855 pg/mins/mL). At 14 days, ALP expressed was higher as it was expected, and there was no differences betweem fluence, highlighting the lower fluence (2.5E17) as the samples which showed higher content (2400 pg/mins/mL).

FIG. 102 Evaluation of cell differentiation of osteoblasts cells MC3T3E1 growing into irradiated Ti6Al4V alloy in DIS. Quantification of ALP enzyme was expressed in pg/mins/mL at 4 and 14 days on Ti alloy samples irradiated with three different gas species, inert and reactive ion beams (argon, krypton, nitrogen, and oxygen). Although ALP levels were similar between surfaces irradiated using different gas species, the levels achieved at short time of incubation were similar to those found in FIG. 9 (250 pg/mins/mL), however, at 14 days there were another different scenario. At longer times, the nitrogen surfaces enhanced the ALP levels much more than the other gas species. Argon irradiated by 60 degrees and Oxygen by normal incidence angle also achieved high ALP values more than Krypton (at 0 and 60 degrees).

FIG. 103 Comparative results of osteoblast proliferation (cell metabolic activity): Argon fluence study. Cell adhesion and proliferation of pre-osteoblast growing on irradiated Ti6Al4V alloy samples. Cell metabolic activity were expressed as relative fluorescence units of Alamarblue resulting an increased in metabolic activity from 24 to 96 h as we expected. In addition, at 24 h TiN_60 achieved better metabolic activity in adhesion state and at 96 h the highest values were found in TiKr_0. Two way ANOVA revealed strong significance differences between incubation times and surfaces, finding p≤0.05, p≤0.01, and p≤0.001 represented by *, **,*** respectively.

FIG. 104 Comparative roughness of irradiated Titanium alloy (Surface roughness analysis by AFM): Incidence angle study. AFM 3D images of Ti alloy samples irradiated with low fluences 2.5E17 and different incidence angles. AFM measurements allow the quantification of roughness surface of the DPNS samples analyzing Ra roughness and RMS (root mean square) of the samples. As it is observed through these images, the surface modification is achieved in any DPNS samples which showed nanofeatures and an increased Ra values compared to the controls polished Titanium (PTi). The highest roughness values are found in high oblique incidence angle (80° degrees, Ra=31.14 nm) followed by 60 and 30 degrees and finally 0 incidence angle. Even for the latter, PTi showed the lowest Ra (7.9 nm).

FIG. 105 Comparative results of human aortic smooth muscle cells in cell adhesion process (Cell metabolic measurement at 24 hours): Incidence angle study. Cell viability results of human aortic smooth muscle cells (HASMCs) growing on Ti alloy substrates at 24 h of cell incubation. Cell viability results are presented as cell metabolic activity in terms of incidence angle for the irradiated Ti6Al4V alloy disc. Control samples used were without exposure to Ar+irradiation on a polished Ti6Al4V alloy surface (CPTI) and tissue culture plastic (TCP).

FIG. 106 Cell morphology evaluation of human aortic smooth muscle cells: Analysis sand quantification of filopodia prolongations at 24 hours. Analysis of filopodia and lamellipodia cell structures by SEM and its quantification by image J. SEM images show the evolution of cells/nano-structured surfaces interactions and morphology changes for different incidence angles with Ar+irradiation. The bottom row corresponds to the higher magnification of the red squares region of the upper SEM images. Quantification of cell adhesion structures. Filopodia density (A) and filopodia length (B) was evaluated in Ti6Al4V samples after Ar+irradiation with different incidence angles. Ti6Al4V without irradiation was used as a control (CPTI). Data shown were expressed in mean plus standard deviation finding significant differences in terms of filopodia density between the control sample and Ti6Al4V S2 (30°) and indicated it by asterisks (P<0.05).

FIG. 107 Confocal imaging of actin cytoskeleton and cell nuclei of HASMCs at 24 h after cell seeding. Actin was visualized by phalloidin-TRICT and nuclei by DAPI (Hoechst). Actin fibers and cell nuclei images are presented in gray scale to reserve maximum contrast. Cell morphology, found on Ti6Al4V treated by DPNS, shows greater actin cytoskeleton development with a cell shape more spread than Ti control and higher interaction with the surface and with surrounding cells. Cells appear more spread than on control sample, polished Ti, in which cells presents an elongated cytoskeleton but with reduced interactions with cells and the surface.

FIG. 108 XPS patterns of porous titanium samples using different concentration of salt (NaCl) and Amonium bicarbonate (BA) as space holder.

FIG. 109 Table with the description of the chemical compounds and their percentages in each sample obtained by XPS. Zn2p peak and Cu2p peak increase in the samples with lower amount of salt while oxygen peak and Titaniu peaks appear homogeneous in the samples ranging from 29 to 34 and from 1 to 5.87 in O1s and Ti2p peaks respectively. The carbon layer previous to irradiation by DPNS was quite high around 60%.

FIG. 110 X-ray diffraction patterns of space holder samples using Salt A (FIG. 110A) and Ammonium Bicarbonate BA (FIG. 110B).

FIG. 111 Evaluation of Youngs modulus and porosity index (%) of the porous titanium samples with both treatments. Increasing the porosity in the samples the stiffness decreased as it is shown in 70% the Youngs modulus is 453 (N/mm²), the lowest found in all the samples.

FIG. 112 The porosity index was evaluated using Micro-CT analysis of the whole sample. The images obtained revealed the distribution, density, and morphology of the pores in the titanium samples.

FIG. 113 SEM images of the different porous Ti samples with irradiation by DPNS.

FIG. 114 Table with the all conditions used to modify porous Ti surfaces with DPNS.

FIG. 115 SEM image of the control surface of porous titanium. This image shows the smooth surface outside the pore.

FIG. 116 SEM image of 60% porous NaCl Ti samples before and after irradiation using normal incidence angle by DPNS. Notice the nanotopography changes before and after DPNS, highlighting the presence of small nanoplatelets in the pore surface.

FIG. 117 SEM image of 60% porous NaCl Ti samples before and after irradiation using off-normal incidence angle (60 degrees) by DPNS. Notice the nanotopography changes before and after DPNS, highlighting the presence of small nano peaks in the pore surface.

FIG. 118 SEM image of 30% and 50% porous NaCl Ti samples irradiated with normal and off-normal incidence angle (0 and 60 degrees respectively). The lower row showed higher magnification of the yellow circle which corresponds to nano ripples at the surface of both samples.

FIG. 119 SEM image of 30% and 50% porous NaCl Ti samples irradiated with normal and off-normal incidence angle (0 and 60 degrees, respectively). The lower row showed higher magnification of the upper row, to confirm the presence of nanofeatures inside the pore.

FIG. 120 SEM image of 50% porous NaCl Ti samples irradiated with off-normal incidence angle (60 degrees). Nanofeatures were found inside the pit as nanoripples, similar as 30% Ti.

FIG. 121 SEM image of 50% porous NaCl Ti samples irradiated with off-normal incidence angle (60 degrees). Different morphology of nanofeatures were found in these samples, from nanorounds to nanoripples. Notice that in some areas it was detected a smoother effect probably due to the oblique incidence angle.

FIG. 122 SEM image of 30% and 50% porous NaCl Ti samples irradiated with normal and off-normal incidence angle (0 and 60 degrees, respectively) as well different energies (500, 750, and 1 Kev, respectively. The lower row showed higher magnification of the upper row, to confirm the presence of nanofeatures inside the pore. At lower energies and normal incidence angle the surface seems smooth and with no nanostructures, however, using energies of 1 Kev, the nanofeatures developed cover the whole surface increasing the roughness of the sample. On the other hand, the nanofeatures were develop in all the energies in the 50% porous Ti samples due to the off normal incidence angle.

FIG. 123 SEM image of 50% porous NaCl Ti samples irradiated with off-normal incidence angle (60 degrees) and 750 ev of energy. Nanosharp peaks were developed due to the irradiation process by DPNS finding these nanostructures at the surface of the sample.

FIG. 124 SEM image of 50% porous NaCl Ti samples irradiated with off-normal incidence angle (60 degrees) and 750 ev of energy. Wide and small nanocones were observed at the surface and also inside the pit of the sample.

FIG. 125 Cell adhesion and proliferation of preosteoblast cells seeded on 30% and 50% PTi modified with 1 Key of energy and 0 and 60 degrees of incidence angle, respectively. The cell metabolic activity of MC3T3E1 increased on incubation time as expected, however, there were differences between samples. The irradiation in 30% PTi_Ar_5 samples achieved lower cell metabolic levels than the control non irradiated (30% PTi) for the three time points. Nevertheless, the 50PTi_Ar_06 achieved higher metabolic activity than its counterpart on irradiated. Notice the highest levels in 50% PTi samples compared to 30% which can be associated to a better substrates to induce cell adhesion and proliferation.

FIG. 126 Cell mineralization of pre-osteblast cells seeded onto porous Ti surfaces. This image showed the influence of incidence angle in calcium deposits, measured by alizaring red, due to the incubation of preosteoblast cells with mineralized media during 14 days. 50% PTi surfaces (irradiated and control) achieved higher levels of calcium deposits than 30% porous titanium samples. It is noteworthy to mention that the irradiated sample of 50% (50% PTi_ar_6) showed better results compared to the control surface, highlighting the strong effect of the nanofeatures in the mineralization process of osteoblast.

FIG. 127 Provides metabolic activity in UFR for 30% NaCl and 50% NaCl, at 1 day, 4 days and 7 days.

FIG. 128 Optical microscopy images of Alizarin experiment of all porous Ti samples at 14 days of cell seeding. In red color it is represent the calcium deposits by the preosteoblast cell MC3T3E1. It is remarkable the obtained results in irradiated 50% porous samples than 30% (irradiated and control) in which almost there were no red coloration on the surface.

FIG. 129 Mechanism of ion beam irradiation inside the pit of porous titanium samples by DPNS. Notice, the incidence of ion beam inside the pore and how it modifies the surface as well there is an energy deposition which subsequently continuous with the ion beam in other areas. This mechanism explains how the nanostructures are developed in other walls inside the pit even those that are not directly exposed to the ion beam.

FIG. 130 Comparative results of osteoblast differentiation (ALP Expression): Argon fluence study. At 4 days there is no differences between ion irradiated samples and controls, however, at 7 days this situation evolves, changing slightly to an increase ALP levels in irradiated samples. At 14 days of cell culture, ALP levels still, increased on time for all specimens, however, at low fluences the expression is higher than the other conditions.

FIG. 131 Comparative results of osteoblast differentiation (ALP Expression): Gas species and incident angle study.

FIG. 132 Comparative results of irradiated titanium alloy with different incidence angles (surface topography analysis by SEM): Incidence angle study.

FIG. 133 Evaluation of bacterial adhesion and biofilms formation by confocal microscopy. Live and dead assays was performed to analyze how the nanofeatures developed by DPNS can disturb and kill the bacterial adhesion process in order to reduce the biofilms formation and secondary infections. These confocal images of Polished Ti (PTi), S1-0°, S2-30°, S3-60°, S4-80°, and SLA type surface shown in green color the live E. coli and in red the dead bacteria. Regarding higher bacteria density in the surface SLA shows higher number of bacteria alive and dead as well. On the contrary, PTi, S1, and S2 showed similar behaviors, reaching lower dead bacteria compared to SLA type surface in which deade bacteria were superior. Ti6Al4V irradiated with oblique and higher oblique incidence angles (60° and 80° degrees, respectively) showed a different bacteria distribution due the nanopatterning surface and the nanofeatures morphology. S4-80° was the incidence angle with a lower ratio of live and dead cells, even better than SLA type surface. It was found lower alive bacteria density and higher dead bacteria revealing the morphology of the nanofeatures and the chemistry surface, key factors in the development of antibacterial properties. Ti6Al4V surfaces irradiated with high energy (1 kEv), low fluence (2.5E17), a neutral gas specie (Argon) and 80 degrees of incidence angle as the best antibiofouling surface.

FIG. 134 Simplified diagram of SEM images revealing the relationship between the nano-scale features, the incidence angle of Ar+ions beams and the cells adhesion structures presented in these Ti allow samples. Regarding the DPNS process, the nanostructures increase cell lamelipodia and filopodia protusions which are related to an increase in cell attachment. In addition, the nanofeatures morphology and their orientation are controled due to the different parameters of DPNS, developing nanograins and nanoripples by normal or off normal incidence angles, respectively.

FIG. 135 Cell metabolic activity macrophages seeded on porous Ti surfaces at 24 h. Macrophages exhibited similar metabolic activity in all the samples independently of the percentage of porosity or the irradiation parameters.

FIG. 136 SEM images of porous and non porous cpTi samples after Ar+using DPNS with different incident angles. PPS2 (A an B), NPS1 (C and D), NPS2 (E and F) and NPS3 (G and H) were irradiated with 60, 0, 45, and 75 degrees of incident angle respectively.

FIG. 137 Biological evaluation of cpTi samples modified with Ar+using DPNS with different incident angles. PPS2 (A an B), NPS1 (C and D), NPS2 (E and F) and NPS3 (G and H) were irradiated with 60, 0, 45, and 75 degrees of incident angle respectively.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Nanoscale domains,” as used herein, refers to features characterized by one or more structural, composition and/or phase properties having relatively small dimensions generated on the surface of a substrate. Nanoscale domains may refer to relief features and/or recessed features such as trenches, nanowalls, nanocones, nanoplates, nanocolumns, nanoripples, nanopillars, nanorods, nanowires, nanotubes and/or quantum dots. Nanoscale domains may refer to discrete crystalline domains, compositional domains, distributions of defects, and/or changes in bond hybridization. Nanoscale domains include self-assembled nanostructures. In embodiments, for example, nanoscale domains refer to surface depths or structures generated on a surface having dimensions of less than 1 μm, less than or 500 nm, less than 100 nanometers, or in some embodiments, less than 50 nm. In an embodiment, nanoscale domains refer to a domain in a thermally stable metastate.

“Surface geometry” refers to a plurality nanoscale domains positioned on the surface of a substrate. In embodiments, for example, nanofeatured surface geometry is a periodic or semi-periodic spatial distribution of nanoscale domains. For example, nanofeatured surface geometries include topology, topography, spatial distribution of compositions, spatial distribution of phases, spatial distribution of crystallographic orientations and/or spatial distribution of defects. Surface geometries of some aspects are useful for providing a selected multifunctional bioactivity, a selected physical property or a combination thereof.

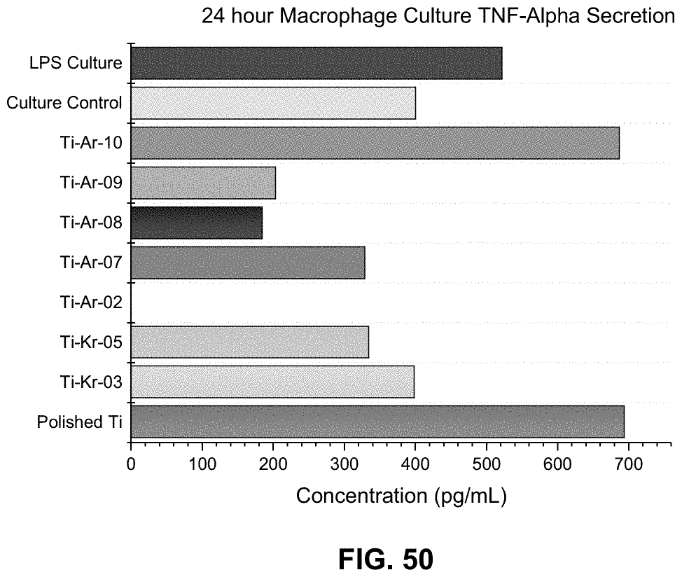

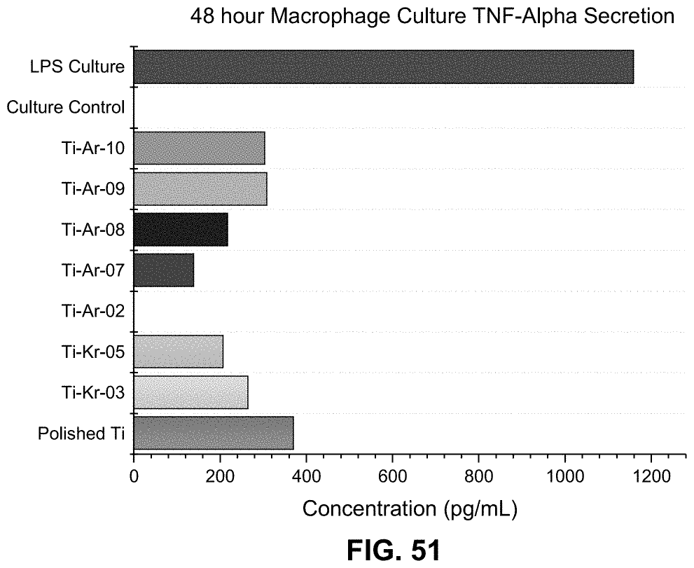

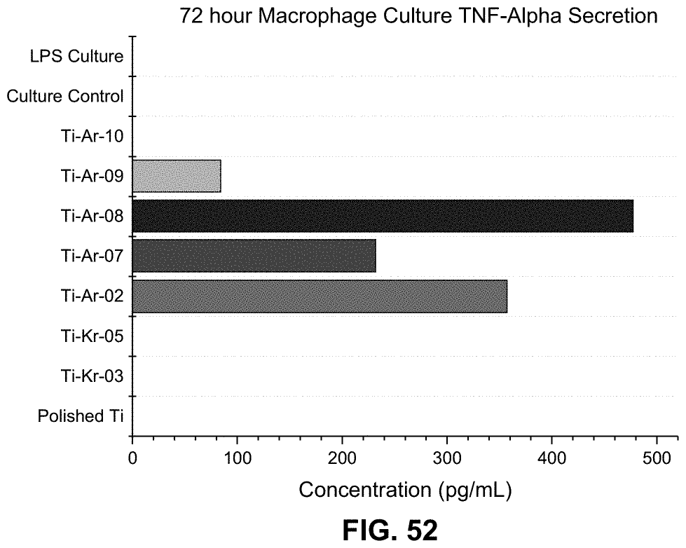

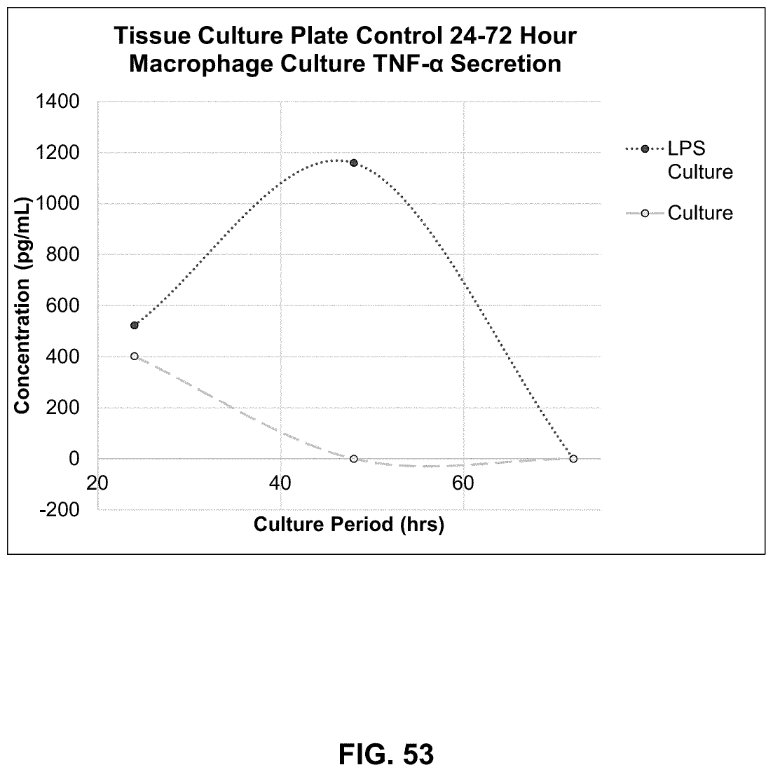

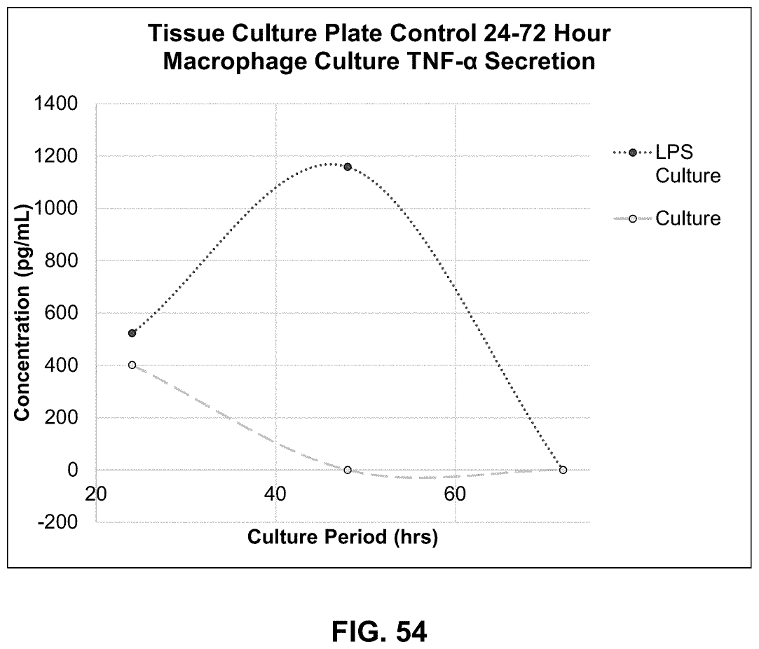

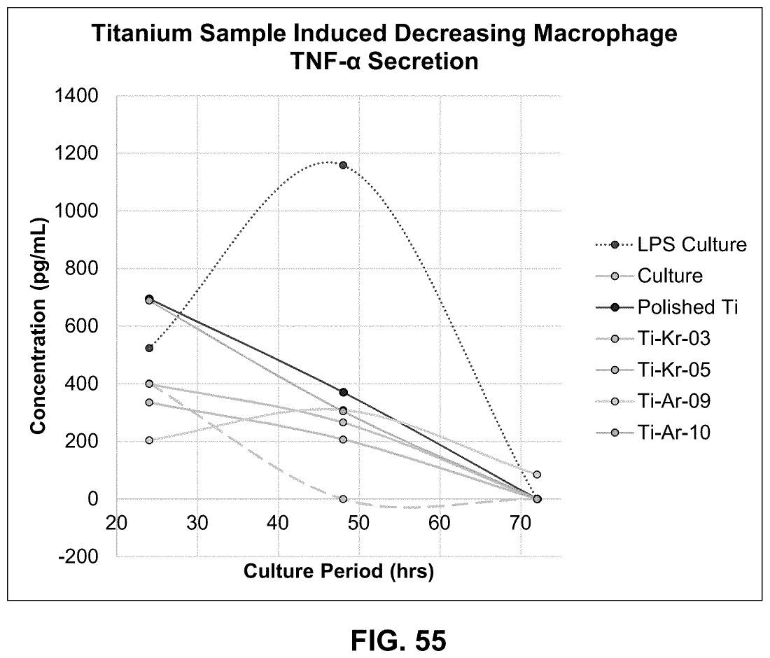

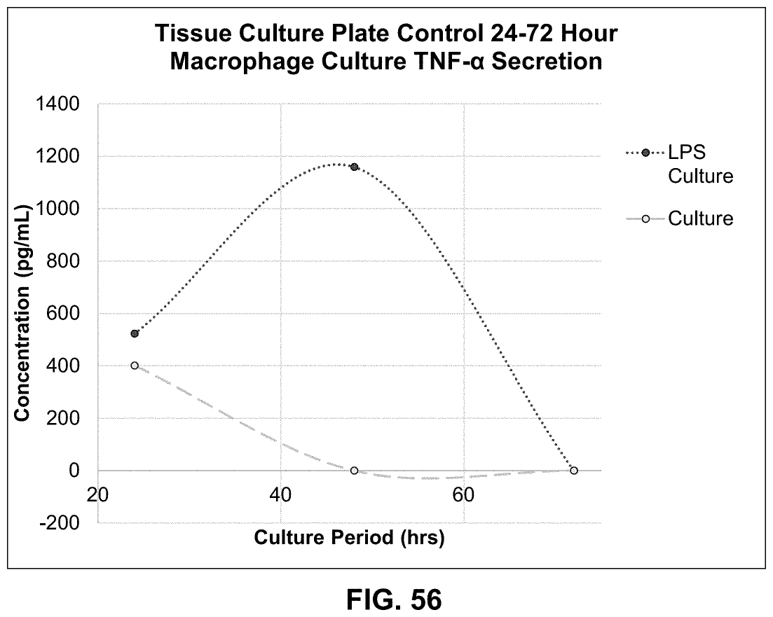

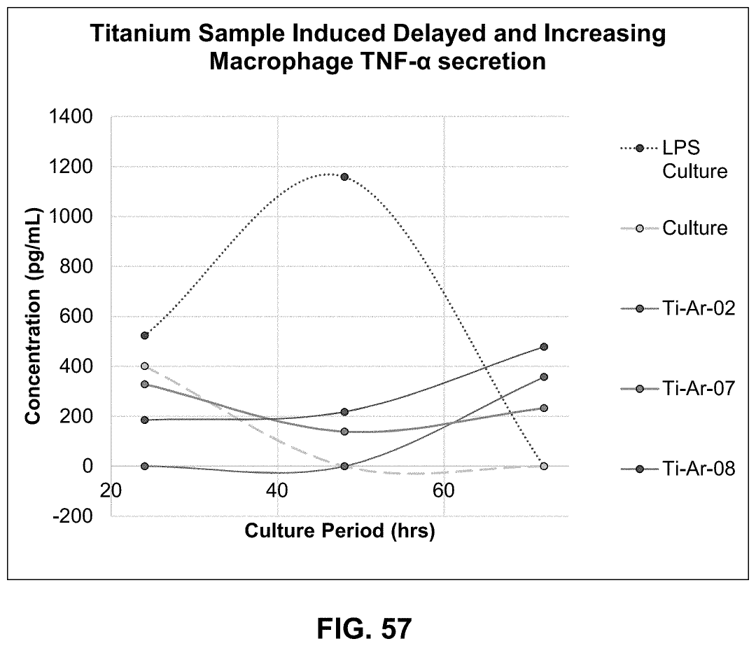

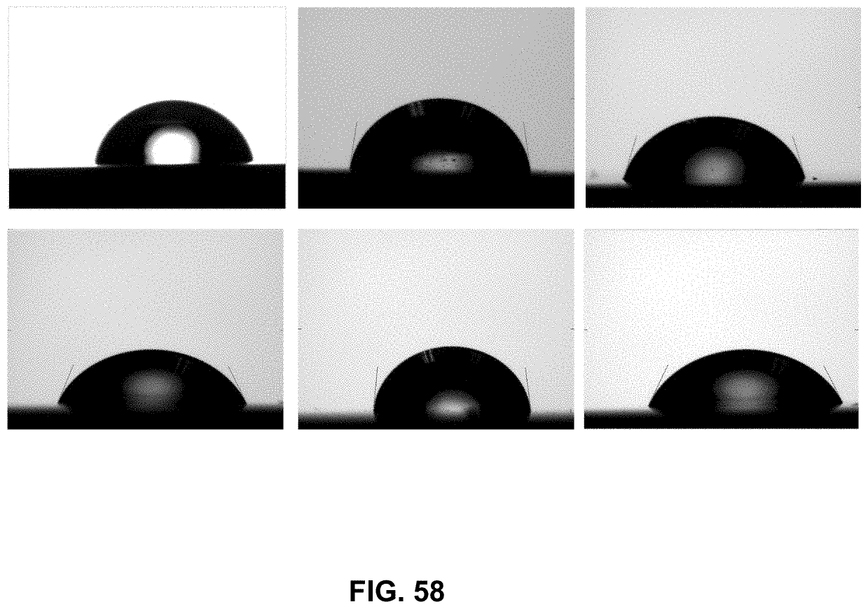

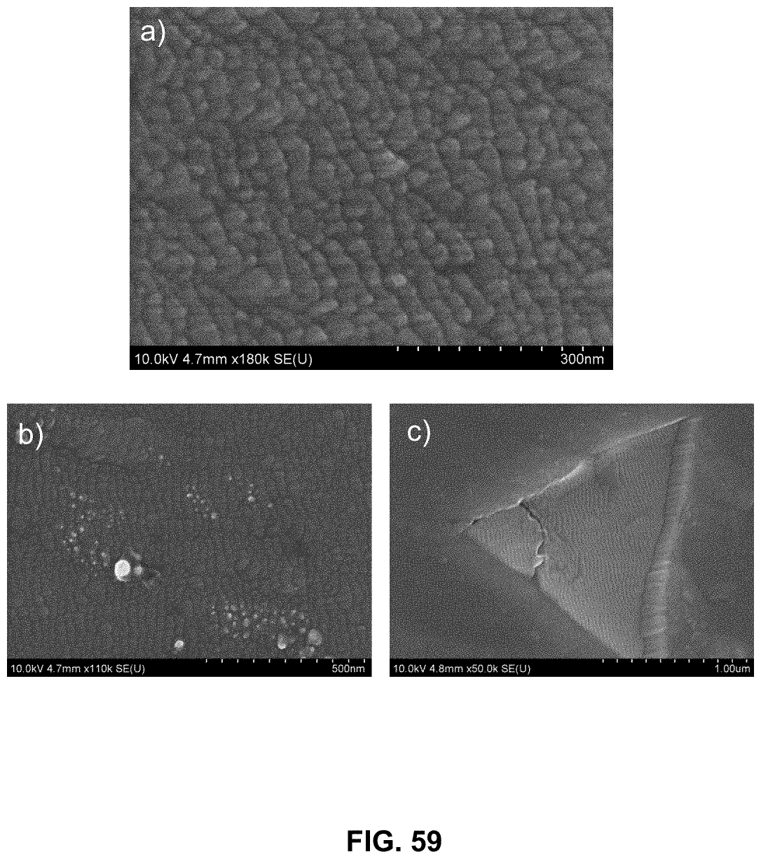

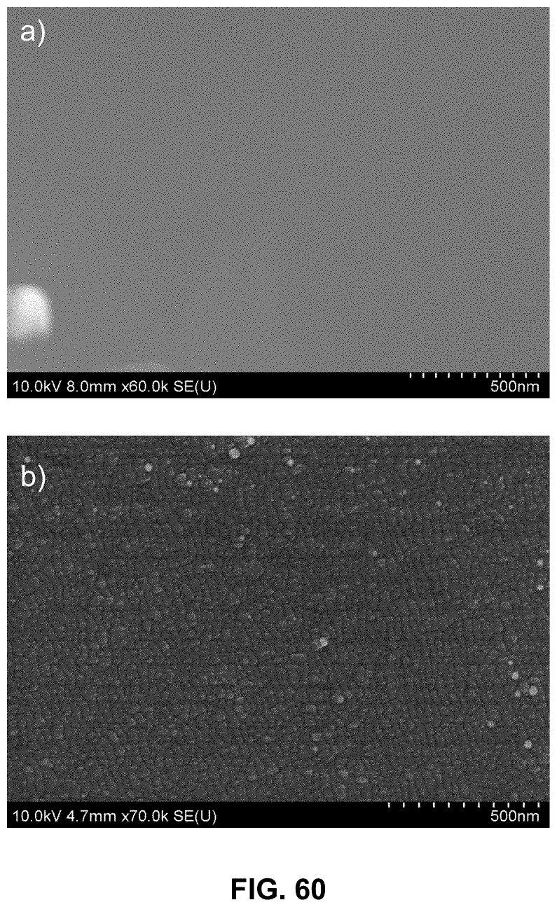

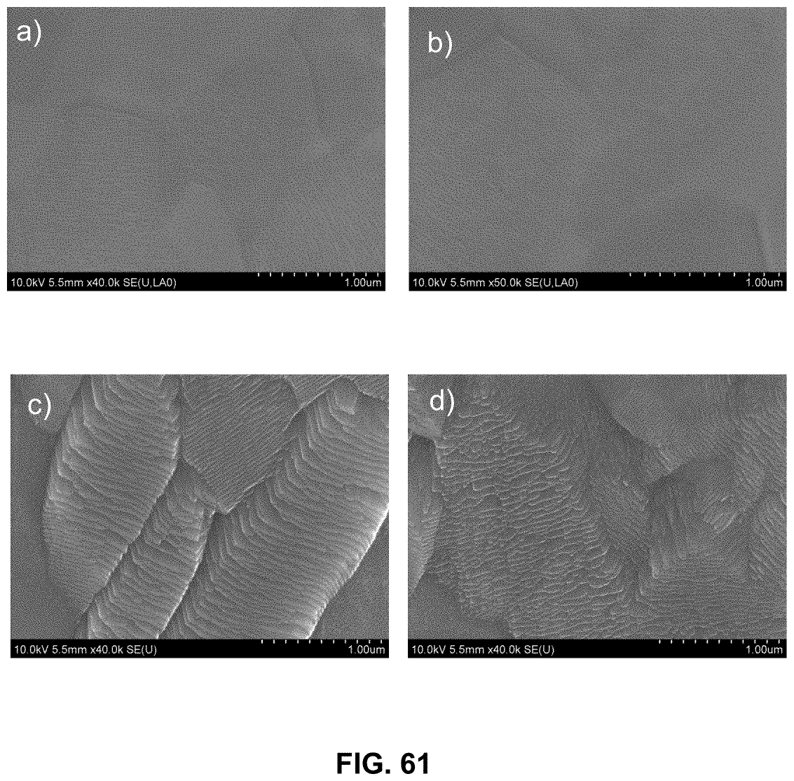

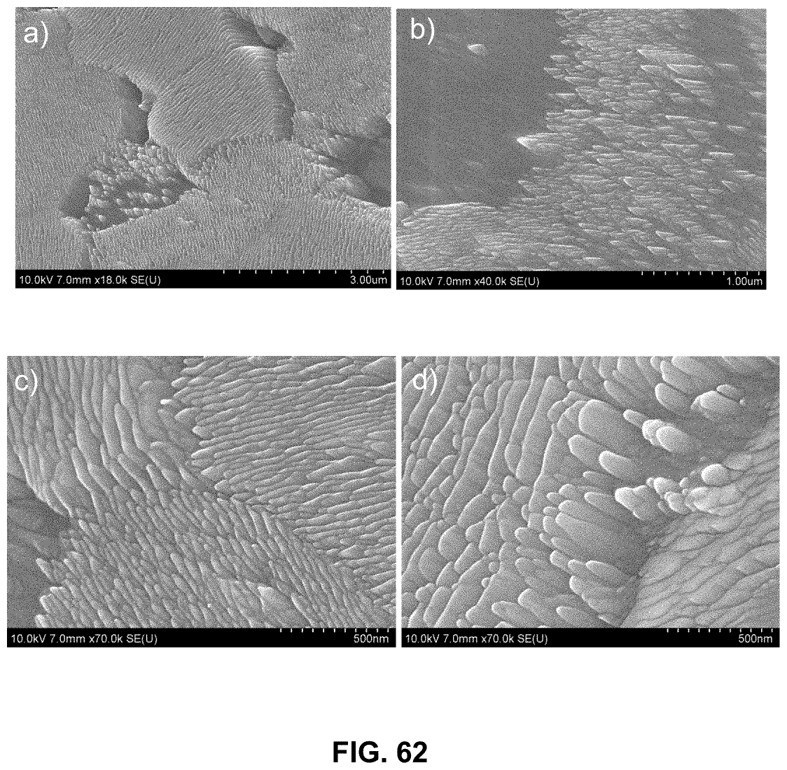

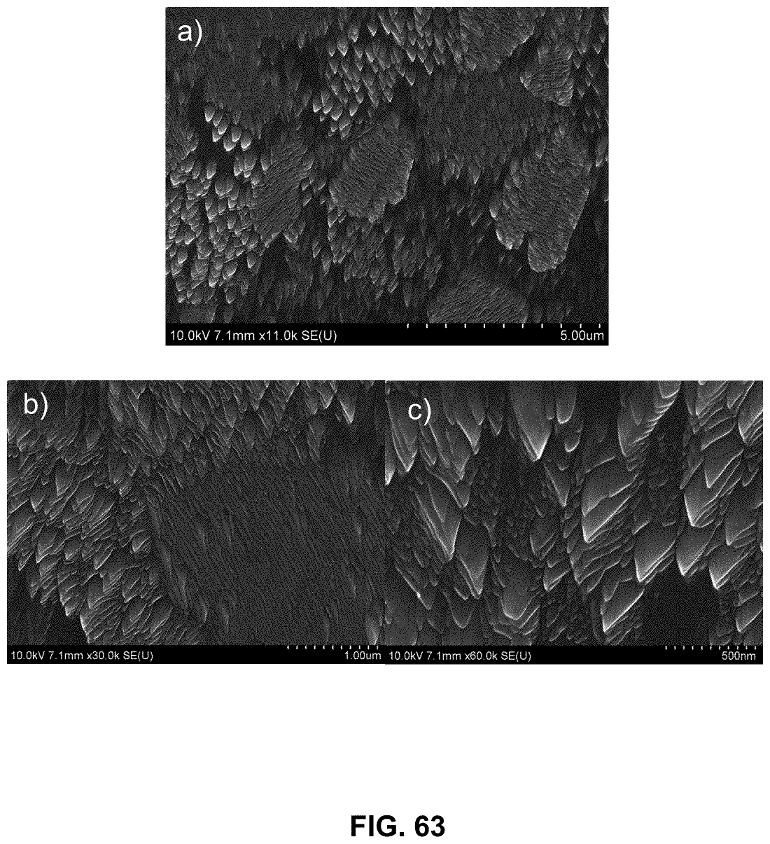

“Selected multifunctional bioactivity” refers to an enhancement of in vivo or in vitro activity with respect to a plurality of biological or physical processes. In embodiments, for example, multifunctional bioactivity is enhanced relative to a titanium or titanium alloy substrate surface not having said plurality of nanoscale domains characterized by nanoscale surface geometry. In an embodiment, for example, a selected multifunctional bioactivity is an enhancement in cell adhesion activity, cell shape activity, cell proliferation activity, cell migration activity, cell differentiation activity, anti-bacterial activity, bactericidal activity, anti-inflammatory activity, osseoconductive activity, osseointegration activity, biocorrosion activity, cell differentiation activity, immuno-modulating activity during acute or chronic inflammation or any combination of these. In an embodiment, for example, a selected multifunctional bioactivity is a modulation in the immune response to a foreign body (e.g. the implant). In an embodiment, for example, a selected multifunctional bioactivity is an enhancement or inhibition of one or more protein interactions. Macrophage cells are powerful regulators of the foreign body response that govern the inflammatory and tissue remodeling pathways of injury resolution in implantation. In the body, foreign material is a potent stimulus that triggers the surrounding tissue to produce the various signaling molecules and growth factors of macrophage recruitment and polarization. Although type 1 and type 2 T-helper cell activity continue to be the standards of cell phenotype, the fundamental polarizing states of macrophages can be classified into the three main functions of host defense, immune regulation, and tissue repair. To characterize the macrophage polarization activated in each testing condition, an ELISA kit was used to quantify the secretion of pro-inflammatory and anti-inflammatory cytokines TNF-α and IL-10 at 24, 48, and 72 hours of surface cell culture. The 24-hour cytokine results are presented in Table 1 and FIG. 51. TNF-α nor IL-10 secretion appeared to directly correlate with the nanostructure characteristics of increased roughness, topographical shape, size, and total surface area coverage at 24 hours as expected. For example, the extremely contrasting sample surfaces of polished titanium and sample Ti—Ar-10 induced similar macrophage secretions of TNF-α at high concentrations of 695.1 and 688.4 pg/mL. Interestingly, these two supernatant concentrations exceeded the TNF-α of the positive pro-inflammatory control of the LPS supplemented tissue plate culture medium. The argon irradiated samples Ti—Ar-10, Ti—Ar-02 and krypton irradiated sample Ti—Kr-05 were all observed to have very similar striking topographies of stacked platelets, nano-cones, and protruding grain boundaries. Yet, sample Ti—Ar-02 was only observed to induce a small concentration of IL-10 cytokines with no detectable TNF-α at 24 hours. The heavily nanostructured surface of Ti—Kr-05 was also observed to produce a comparable amount of TNF-α to the finely structured surface samples of Ti—Kr-03 and Ti—Ar-07. Its supernatant was measured to have a pro-inflammatory cytokine concentration of 335 pg/mL, while significantly smoother surface samples Ti—Kr-03 and Ti—Ar-07 had concentrations of 399.2 and 328.7 pg/mL. In regards to IL-10, polished titanium, the tissue culture plate control, and the LPS positive control produced the largest cytokine concentration. The high cytokine concentrations of TNF-α and IL-10 observed in these 3 testing conditions may indicate the presence of classically activated M1 and M2b polarized macrophages that can drive host defense and suppress inflammation. One may also be observing a happy medium with nanostructure formation and size on samples Ti—Ar-08 and Ti—Ar-09. Sample Ti—Ar-08 was irradiated at one of the middle increment parameters in the fluence study, which was observed to be the beginning parameter of increasing structural modification surface area and defined, smaller nanostructures. The smaller nanostructures resulted in the second lowest concentration of TNF-α cytokines and an upregulated secretion of IL-10 compared to smoother surface sample Ti—Ar-07. Increasing to the next increment of fluence resulted in sample Ti—Ar-09 having a larger structural modification surface area with larger nanostructures than Ti—Ar-08. Interestingly, the larger nanostructures decreased IL-10 cytokine secretion with an upregulated secretion of pro-inflammatory TNF-α. These results suggest surface nanostructuring and texturization to have an immunosuppressive influence that can be undone with larger, disruptive topographies. Despite the proposals made, it is clear the samples activated a robust, pro-inflammatory pathway in the macrophages at 24 hours. FIG. 52 and Table 2 present the 48-hour macrophage cytokine secretion results. At 48 hours, the surface testing conditions displayed a decreased TNF-α concentration. This may have indicated the beginning resolution of the pro-inflammatory pathway activated at 24 hours. However, the anti-inflammatory cytokine secretion was also observed to decrease at 48 hours with the majority of the sample supernatants measuring zero concentrations of IL-10. The LPS supplemented tissue plate culture was only the testing condition to have a peaking 1158.6 pg/mL concentration of TNF-α, which is a common result observed with the macrophage and leukocyte response to infection in the body. Interestingly, polished titanium was the only sample possibly displaying a complete reversal in macrophage polarization with its IL-10 secretion growing to significantly exceed the TNF-α cytokine concentration. The 24-hour macrophage culture had an approximate TNF-α concentration of 695.1 pg/mL compared to the 228 pg/mL concentration of IL-10 on polished titanium. At 48 hours, polished titanium cultured macrophages had TNF-α and IL-10 concentrations of 370.5 and 918 pg/mL respectively. One can conclude that the macrophages at 48 hours are beginning to have a transition in polarization and phenotype. Table 3 and FIGS. 53(a) and (b) exemplify the differences in TNF-α secretion over the total culture period of 72 hours. As one can observe in the Table 3, the 72 hour supernatant measurements displayed a zero concentration of TNF-α for polished titanium, samples Ti—Kr-03, Ti—Kr-05, Ti—Ar-10, and the tissue plate culture controls. Despite some inconsistency between its 24 and 48 hour measurements, sample Ti—Ar-09 overall showed a significant decrease in its TNF-α secretion. The decrease observed with these samples potentially indicate the resolution of the pro-inflammatory pathway of the macrophages. Conversely, finer structured samples Ti—Ar-07 and Ti—Ar-08 showed an overall increase in TNF-α cytokine secretion at 72 hours that went against the conventional progression of the inflammatory pathway towards tissue repair in the body. Interestingly, sample Ti—Ar-02 displayed significant TNF-α secretion after producing supernatants with a zero concentration of the pro-inflammatory cytokine at 24 and 48 hours of cell culture. From these results, it appears that Ti—Ar-02 may possess some topographic and chemical surface properties that delayed macrophage attachment and activation of the foreign body response. Unlike the rougher, nanostructured surfaces, samples Ti—Ar-07 and Ti—Ar-08 may have topographical and chemical properties that prolong pro-inflammatory activity. FIGS. 54, 55, and 56 provide a visual explanation of these results with the LPS supplemented and cell culture medium tissue plate controls serving as the conventional pathway of inflammation. Several in-vitro studies have shown metallic implants to induce a high initial secretion of pro-inflammatory cytokines in the first 24 hours of cell material interaction. Once cell adhesion occurs, macrophages are polarized into a pro-inflammatory state that generates high amounts of cytokines and growth factors to promote its proliferation and recruitment of other immune cells to destroy pathogens and damaged cell debris in the environment. It is proposed that only the newly formed macrophages secrete the cytokines and molecular signals that eventually direct the activation pathways toward wound-healing after 24 hours in-vitro. The work of Anderson et al with stainless steel and chromium cobalt observed microrough surfaces to have greater pro-inflammatory cytokine secretion in 1 day compared to smoother surfaces. However, by day 2, the macrophages migrated freely over the smooth surfaces and polarized into host defense phenotypes with high pro-inflammatory cytokine secretion rates exceeding that of the rough surfaces. Initially, the rougher surfaces indicated a poor success rate for implantation. However, after 2 days, the surfaces induced significant amounts of anti-inflammatory signaling molecules. Because the foreign body response is a highly transient process depending on the natural stimuli of the environment, one cannot conclude that robust, pro-inflammatory activity at one time point means implant failure. In fact, Anderson et. al proposed an initially high pro-inflammatory cytokine secretion to be characteristic of a biomaterial capable of effectively preventing onsite infection with early implantation. The key is determining the properties that can modulate the inflammatory pathways toward a favorable resolution of the foreign body response. Osteoblasts are the principal operating cells of the tissue synthesis and mineralization processes responsible for bone generation. Previous research performed has discovered each stage of osteogenesis to correlate with the expression of certain genes in mesenchymal stem cells and osteoblasts. Some of the markers include collagen I, alkaline phosphatase, osteopontin, osteonectin, and osteocalcin. Many in-vivo and in-vitro studies have found these specific protein and gene markers to play a critical role in the regulation and execution of the bone cellular building pattern and incorporation of key elements. To characterize the bone supporting growth potential of the titanium samples, a p-nitrophenyl phosphate (pNPP) substrate solution was used to characterize the expression of alkaline phosphatase (ALP). The results are summarized in FIG. 50. As one can observe, sample Ti—Kr-05 induced the osteoblast culture to have a significantly higher ALP expression than any of the surface testing conditions simulated, which in this experiment consisted only of polished titanium and sample Ti—Kr-03.

“Directed energetic particle beam,” as used herein, refers to a stream of accelerated particles. In embodiments, the directed energetic particle beam is generated from low-energy plasma. In some embodiments, directed energetic particle beam is a focused or broad ion beams capable of delivering a controlled number of ions to a precise point or area upon a substrate over a specified time. Directed energetic particle beam may include ions and additional non-ionic particles including subatomic particles or neutral atoms or molecules. In embodiments, directed energetic particle beams provide individual ions to the target location. Examples of directed energetic particle beams include focused ion beams, broad ion beams, thermal beams, plasma generated beams, optical beams and radiation beams.

“Beam property” or “beam parameter” refer to a user or computer controlled property of beam, for example, an ion beam. Beam parameter may refer to incident angle with a target substrate, fluence, energy, flux, beam composition and ion species. Beam parameters may be adjusted to provide selected interactions between the beam and the target substrate to generate specific nanostructures or enhance specific properties of the substrate. Beam parameters may be controlled by a variety of means, including adjustments to electromagnetic devices in communication with the beam, adjusting the gas or energy source used to generate the beam or physical positioning of the beam in reference to the target.

“Vertical spatial dimension” refers to a measure of the physical dimensions of a nanoscale domain perpendicular or substantially perpendicular to the planar or contoured surface of a substrate. In embodiments, vertical spatial dimension refers to a height or depth of a nanoscale domain or the mean depth of a surface modification, for example, a crystalline or compositional domain.

“Lateral spatial dimension” refers to a measure of the physical dimensions of a nanoscale domain parallel or substantially parallel to the planar or contoured surface of a substrate.

“Titanium or Titanium alloy substrate” refers to any substrate composed of titanium including commercially pure titanium and Ti6Al4V as described herein. In some embodiments, titanium alloys may refer to alloys containing titanium but in which titanium is not the primary component. In other embodiments, titanium alloys refer to alloys in which titanium represents more than 25%, or optionally 50%, of the alloy. Titanium and titanium alloys may include a titanium oxide layer, including on the surface being modified.

“Porosity” or “porous titanium” refers to substrates or titanium surfaces having individual or networked voids at or near the surface of the substrate. Porosity may be nanoscale, microscale or larger. As described herein, substrates may have porosity prior to any plasma treatment (e.g. porosity formed during substrate formation such as sintering). In some embodiments, pores may be formed, enlarged or altered by the treatment of directed plasma, including forming nanopatterns on interior pore surfaces or walls between individual pores.

“Multiplexing” refers to simultaneously modifying the target substrate in more than one way, for example, by providing two or more directed particle beams at the substrate having different properties, for example, to generate or modify at least one nanoscale domain (e.g. nanoscale features, crystalline domains, compositional domains, distributions of defects, changes in bond hybridization. In some embodiments, for example, a single directed particle beam may have one or more beam properties to generate or modify multiple nanoscale domains on the substrate. In embodiments, multiple direction particle beams are generated from the same plasma source.

The invention may be further understood by reference to the following non-limiting Examples that expand on certain aspects and embodiments of the invention.

### Example 1: Directed Irradiation Synthesis (DIS) of Rough and Porous Titanium Implants for Bone Tissue Repair

### Abstract

It is recognized that medical grade titanium alloys, like commercially pure titanium (cpTi), are the best metallic biomaterials for bone replacement, primarily due to their excellent balance between biomechanical properties and in-vivo biocompatibility response. However, they have two important disadvantages: stress shielding and subsequent bone resorption due to stiffness mismatch with respect to bone, and the lack of osseointegration due to fibrous tissue around implants. Porous cpTi implants have been demonstrated to be an alternative for stress shielding and different techniques of surface modification like controlled roughness have been implemented for improvements of cpTi osseointegration. In this work we have modified samples of both rough and powder metallurgy (PM) porous cpTi by directed irradiation synthesis (DIS) with the aim of evaluating the capability to produce surface nano-patterning. These samples were tested by contact angle testing, structurally characterized by scanning electron microscopy (SEM) and atomic force microscopy (AFM), and biologically tested through the response of human aortic smooth muscle cells (HASMCs). Nano-patterning of porous+polished cpTi samples were highly successful with some samples sensitive to initial surface porosity, being more effective on the flat polished areas. In contrast, initial micro-roughness of conventional machined cpTi samples was an important obstacle to nano-patterning, allowing only influence on vertical nano-roughness, with an important smoothing effect for off-normal beam incidence. In general, DIS processing generated important improvements on both smoothing and surface free energy reflected in drastic reductions of contact angle measurements. Results presented not only have produced nano-structuring on porous Ti-based surfaces, but also produced advantageous smoothing of rough surfaces without any detrimental effect on the original biocompatibility of cpTi implants.

### Introduction

Bone tissue damage is typically represented by joints replacements, fractures, dental implants, and bone diseases related to osteoporosis and cancer. The broad spectrum of bone degradation issues become bone tissue problems as a public health problem, as was recognized some years ago by the World Health Organization [1]. Most of the current clinical treatments for tissue bone correspond to tissue substitution, i.e. replacements approaches for which the biomaterials are mostly of first and second generation. Using these combined biomaterials substitutes have shown reasonable success through translational and clinical levels. However, several failure statistics of current joint replacements indicate that this remains a technological challenge and warrants a multidisciplinary effort to offer new clinical alternatives with the highest in-vivo performance and reliability. To that end, some medical grade of titanium (Ti) alloys, like commercially pure Ti (cpTi), have proved to be the best biomaterials for clinical success of bone replacement due to its excellent balance between biomechanical and in-vivo biocompatibility. However, cpTi exhibits the following disadvantages that are direct consequences of being a 1^{st} generation biomaterial [2]: 1. Biomechanical incompatibility reflected in the elastic mismatch with respect to hosting tissue, and the consequent bone resorption around the implants; 2. Being bio-inert, cpTi implants are surrounded by a thin fibrous tissue, which can often reduce osseointegration with an associated risk of loosening or fracture of bone and/or implant. Biointerface improvements are necessary to avoid the existence of the fibrous tissue or to reduce the loosening risks due to its presence.

In regards to the first problem, it is desirable to design new implants and prostheses with a lower stiffness than those currently used, which would address the stress-shielding problem without any important detrimental effect on mechanical strength. Several reported studies have focused on development of new implants with a bone-matching modulus, such as porous materials [3,4]. To that end, there are some manufacturing processes, among which are highlighted: the electron beam melting process [5], creep expansion of argon-filled pores [6], directional aqueous freeze casting [7], rapid prototyping techniques [8], laser-engineered net shaping [9], electric current activated/assisted sintering techniques [10,11], conventional and non-conventional powder metallurgy (PM) [12] and space-holder techniques [13]. From a conventional PM point of view, controlling compacting pressure, sintering temperature and time, could enable a suitable porosity for stress shielding reduction. Improvement of bone ingrowth and osseointegration requires that pore size and morphology be controlled, especially at the surface, which is also critical for the fatigue resistance of the implant. Optimum PM conditions, which ensures a desired balance between a low stiffness (reduced stress-shielding) and a high mechanical strength (fatigue resistance) have been established [14]. In regards to non-conventional PM alternatives for stress-shielding reduction, the loose sintering process (LSP), in which there is no used any compaction pressure, has emerged as an attractive route to produce porous Ti implants with high porosity and the aim of successfully replacing cancellous bone with an extraordinary low Young's modulus of around 0.5 to 1 GPa. To that end, a detailed comparison between space-holder and loose sintering techniques is available [15], in which they were able to properly optimize a suitable mechanical balance for a Ti implant used in highly porous bones.

In regards to alternatives about minimizing and/or avoiding fibrous tissue and lack of osseointegration, they are basically based on modifications of topographical and/or chemical properties of cpTi surfaces. With respect to topographical modifications, state-of-the art focuses on controlling surface kinetic roughness to improve osseointegration (i.e. by methods such as: chemical etching, sand-blasting, laser ablation, etc. . . . ). Most of these approaches have in common that a vertical parameter of roughness 5.0 microns has an important positive effect on osteoblast adhesion, and in further in-vivo osseointegration [16,17]. Despite limited success, many works can be found about surface chemistry modifications of cpTi by changing its bio-inert character to a bioactive one through different treatments: bioactive ceramic coatings like hydroxyapatite (HA) and Bioglass® [18], and thermo-chemical conversions of titanium oxide layer to a bioactive layer of sodium titanate [17]. In-vivo success of these treatments has been limited due to some functional problems such as: lack of adhesion, brittleness, phases instability, etc.

Surface nanostructuring of conventional biomaterials has emerged as one of the most important and effective approaches to convert them to advanced bioactive materials. This is the consequence of a nano-feature's ability to effectively have some influence on surrounding biological environment at the molecular nano-scale level. Since the cells in their natural environment are surrounded by nanoscale features linked to their extracellular matrix (ECM), the nanotopographical parameters become an important part in design of biomaterials for tissue formation and repair. Accordingly, several studies suggest that a remarkably small modification in surface nanotopography could support mesenchymal stem cell growth and development, indicating that changes in such nanotopographical features can have a direct influence in the adhesion/tension balance to permit self-renewal or targeted differentiation [19]. Biointerface topography and, in particular, nanoscale features can affect cell behavior and integrin-mediated cell adhesion, and is now evident from studies with fabricated topographical features [20]. The mechanisms that mediate cellular reaction with nanoscale surface structures are not well understood [21]. A direct result of the influence of the surface topography, or even an indirect one, may also be correlated to its ability to influence the composition, orientation, or conformation of the adsorbed ECM components [22,23].

The drawbacks of conventional nano-patterning techniques have mainly been attributed to their physical limitations in fabricating structures smaller than about 50-nm. Therefore, bottom-up techniques that rely on self-assembly, self-organization, and local patterning, have become technologies capable of pattern biocompatible surface nanostructures. Irradiation-driven systems have been explored in moderate energy regimes dominated by knock-on atom displacement regimes for semiconductor metallization microstructure control [24], engineering of nanostructured carbon [25], compositional patterning of immiscible alloys [26]. Directed Irradiation Synthesis (DIS) introduces a synthesis process that is scalable to high-volume manufacturing by virtue of its intrinsic large-area simultaneous exposure of materials surfaces and interfaces. Broad-beam ions combined with rastered focused ions and gradient ion-beam profiles are sequenced and/or combined with reactive and/or non-reactive thermal beams that control the surface topography, chemistry and structure at the micro and nano-scale [27].

There are no reported works that examine the capability of ion-beam based modification of nano-patterning rough and porous Ti (e.g. within and inside pores) with cell stimulation and tissue growth. Only a few works have addressed modification and testing on flat surfaces of TiO₂ [28] in contact with rat aortic endothelial cells adhesion, and upregulation of osseospecific proteins-osteopontin and osteocalcin of human osteoprogenitor cells cultured on Ti [29]. Therefore, the aim of this work is to test the hypothesis, for the first time, that rough and porous cpTi samples can be nano-patterned in such way that the biological response of the surface can be favorably influenced. To that end, treated samples were characterized in order to establish relationships with DIS conditions, and with surface energy and structural properties as well. These new surfaces were also biologically evaluated by using human aortic smooth muscle cells (HASMCs) for cytotoxicity assessment. Overall analysis of DIS influence on initial rough and porous cpTi samples enabled qualification of the real potential of our technology for nano-patterning and generating a positive biological response.

### Materials and Methods.

Manufacturing of Porous and Rough cpTi Samples

The powder of cpTi (SE-JONG Materials Co. Ltd., Korea) used for the blends was manufactured by a hydrogenation/dehydrogenation process. The particle size distribution corresponded to 10, 50 and 90% passing percentages, of 9.7, 23.3 and 48.4 μm, respectively. The chemical composition of the powder used was equivalent to cpTi Grade IV according to the ASTM F67-00 Standard [30]. CpTi has an apparent density of 1.30±0.01 g/cm³ (28.8±0.1%) and a tap density of 1.77±0.04 g/cm³ (39.2±0.8%). The blends of cpTi powder were prepared using a Turbula® T2C blender for 40 min to ensure good homogenization. In order to address irradiation influence on porous cpTi samples with different porosities, here we are comparing the loose sintering technique (without any compaction pressure) with the conventional PM one via a low compaction pressure. The compacting step was carried out using an Instron 5505 universal machine to apply the pressure used of 100 MPa, according to reported optimum results [14]. The compacting loading rate was 6 kN/s, dwelling time was 2 min and unloading time was 15 s for decreasing load up to 150 N. The sintering process was performed in a Carbolyte® STF 15/75/450 ceramic furnace with a horizontal tube at 1250° C. for 2 h using high vacuum (≈5×10⁻⁵ mbar). Diameter of compaction die (8 mm) and powder mass were selected to obtain samples in which the effect of compaction pressure was minimized [14]. Surface treatment of cpTi rough samples was achieved by conventional machining of discs (diameter of 2 cm, thickness of 0.5 cm) that were provided by City of Hope of Cancer (, Duarte, Calif.).

Directed Irradiation Synthesis (DIS) of Porous and Rough cpTi Samples.

Grinding and polishing of cpTi porous samples were performed, before DIS processing. In contrast, original rough cpTi samples were irradiated as received. DIS experiments were performed at Radiation Surface Science and Engineering Laboratory (RSSEL) at School of Nuclear Engineering, Purdue University, through the facilities of Particle and Radiation Interaction with Soft and Hard Matter (PRIHSM) system, which was originally developed and set-up by Prof. Jean Paul Allain [27]. DIS conditions of different rough and porous cpTi samples appear summarized in Table 1; they were chosen after an exhaustive revision of our previous DIS nano-structuring results on metals [27, 31], as well as from some other revised works on Ti, TiO₂, Cu, Au, and Ag [32-42]. Argon (Ar+) source at 1 and 0.5 keV was used to irradiate cpTi samples; several angles of incidence and fluences were also evaluated, as is shown in Table 1.

TABLE 1
Irradiation Parameters (Ar⁺) on cpTi Samples

| | Energy | Flux | Fluence | Incidence | Time |
|---|---|---|---|---|---|
| Samples | (keV) | (ions/cm²) | (cm⁻²) | Angle (°) | (min) |
| S1 (cpTi) | 1 | 3.0E14 | 1.0E17 | 60 | 2.0 |
| S2 (cpTi) | 1 | 5.0E14 | 2.5E17 | 60 | 2.0 |
| S3 (cpTi) | 0.5 | 7.2E14 | 5.0E17 | 0 | 18 |
| S4 (cpTi) | 0.5 | 3.2E14 | 5.0E17 | 45 | 27 |
| S5 (cpTi) | 0.5 | 7.7E14 | 3.5E17 | 75 | 76 |

Structural and Surface Free Energy Characterization of cpTi Samples Modified by DIS

Surface free energy of irradiated samples was evaluated by contact angle testing with deionized water through a Rame-Hart Goniometer Model 500-Advanced contact angle goniomter/tensiometer with DROPimage Advanced Software. We performed the sessile method of contact angle analysis (where the sample was static and not moving after the drop was placed on it). All measurements were performed with deionized water (to not have any type of interaction with the surface). The water dropper was far enough away from the surface that the water did not touch the sample until it left the dropper. The dropper was kept at the same distance from each sample surface. The water droplets had 3 pL of water on each sample. Morphological features of samples were detailed analyzed by Scanning Electron Microscopy, SEM (Philips XL40 field emission, FEI, Hillsboro, Oreg., USA). Atomic force microscopy (AFM) was also used for detailed morphological and topographical characterization of irradiated cpTi surfaces, by using an AFM Veeco Dimension 3000 (Santa Barbara, Ca) on AC Mode using cantilever Bruker DNP-10. The scanned area was 1 μm square over samples of both titanium rough and porous samples.

Biological evaluation of cpTi samples modified by DIS.

The cells used for biological assessment of treated and un-treated samples were human aortic smooth muscle cells (HASMCs, Lifetechnology Cat # C0075C). To that end, cells morphology changes with culture time, and cytotoxicity assays test on modified surfaces via Comet Assay® testing, were performed. Changes in cells morphology in terms of culture time was observed by using optical microscopy (OM) analysis. The cell line used was the choose model to validate the potential for tissue growth and regeneration of the new surfaces. Cells were grown at 37° C. with >95% Rh and CO₂ gas exchange until they were nearly confluent. HASMCs were seeded on the top of the samples for 24 hours under normal culturing conditions (37° C., 95% air, 5% CO₂, 95% humidity). HASMCs with a cellular density of 5×10³ live cells per cm², and viability of 86% were cultured in a 7 well tissue culture dish in the presence of the analytes. Two samples labeled as NegCtl and PosCtl corresponded to cells growing alone and used as controls in the assay. All the samples were incubated under normal culturing conditions (37.0° C., 95% air, 5% CO2, 95% humidity) for 24 hours. Then, after 48 hours, visual inspection of HASMCs growing in the presence of the tested analytes was performed during the incubation period under bright field illumination 30 utilizing a Nikon inverted Diaphot fluorescent microscope with 10× and 20× objectives (Nikon Instruments, Melville, N.Y.). The Comet Assay® was carried out according to manufacturer's recommendations (cat. #4250-050-K, Trevigen, Inc., Gaithersburg, Md.). Finally, all the experimental measurements are presented as the mean+standard derivation, which were analyzed using Origin Pro 8.6. One-way ANOVA was performed to compare the mean of the samples, and at the 0.05 level, the population means were considered to be significantly different.

### Results and Discussion

Structural Characterization of as-Received (AR) and DIS Treated cpTi Samples

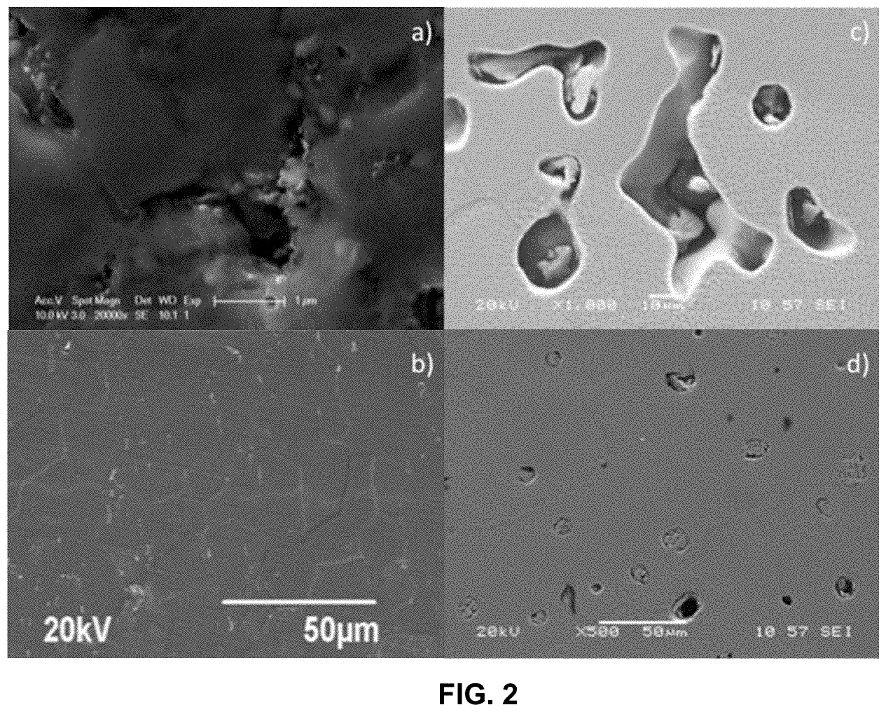

Microstructure of un-irradiated cpTi, rough and porous, samples correspond to a conventional medical grade cpTi (commercial Ti) (FIG. 2); the rough cpTi microstructure consists of mostly equiaxial grains of a phase (hcp), some of them twinned due to work hardening by cold rolling. However, same a phase grains appear clearly less twinned in the porous cpTi structure due to evidently much less effect of cold work (compacting step) because much of the twinning disappears during sintering step of the PM process. Obviously, any twinning will be completely absent in loose sintering samples.

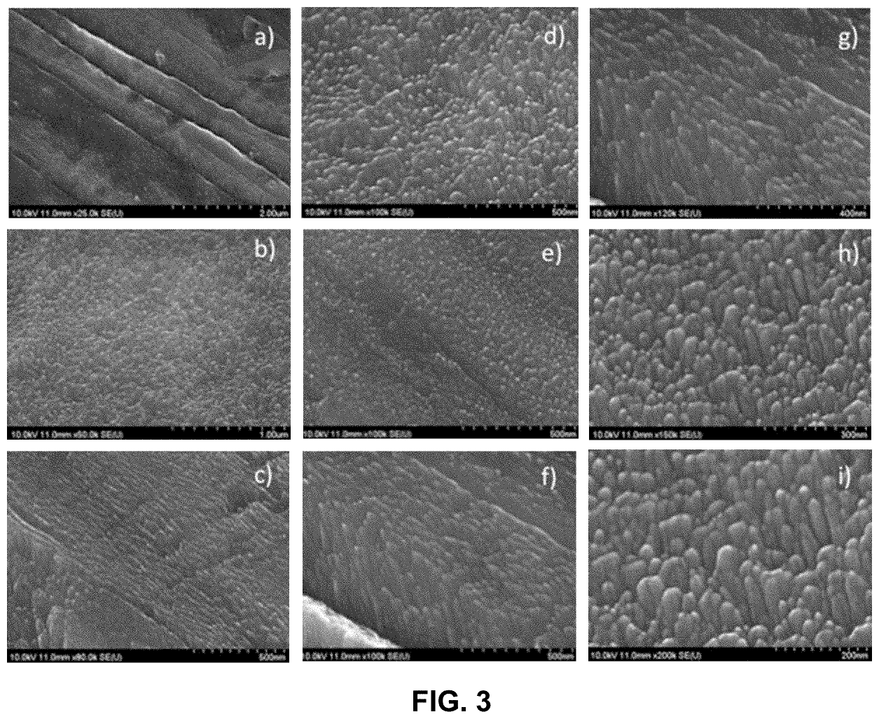

Surface structural modifications and nano-structuring due to DIS of porous cpTi samples are summarized in FIG. 3. The influence of DIS on lower porosity cpTi (FIG. 3a) is reflected one specific nano-patterning characteristic. For the case of low porosity cpTi, short oriented nano-rods preferentially-oriented in the same orientation of the ion-beam direction is obtained. However, some of them appear with a switch of direction, most probably associated to different crystallographic direction of a phase (hcp) grains. This synthetized nano-patterning appears partially as some nano-ripples or with a mixed structure between nano-rods and nano-ripples. The prevalence of nano-rods indicates that the incident angle has the same value of the transition angle or slightly higher (diffusive to erosive regime; rod+ripples to 100% nano-rods). The observed general effect of DIS nano-patterning is the consequence of cpTi microstructure as a monophasic alloy of a phase (hcp); the whole coherency between nano-rods and nano-ripples with respect to ion beam direction would indicate that surface grains are texturized; i.e. grains in the same direction that could be the consequence of the polishing operation which is also verified because of the consistency with polishing scratch directions. It is relevant the high throwing power of this DIS nano-patterning, which is reflected in the capability to create nano-rods inside the pores, and inside the scratches as well (see details in FIGS. 3a, 3c and 3f). Not only nano-rods, but also nano-columns can be easily appreciated inside the gaps-scratches, in a similar way as it is normally observed inside a trench during practice FIB technique. Those columns from the bottom of the scratch seem to grow approximately perpendicular to surface; however, those nano-rods far away from the gap, appear more elongated and they tend to be more parallel to the surface.

Nano-structuring of porous cpTi samples depicted in FIG. 3 are consistent with some previous studies about Ar+ion beam irradiation on cpTi [44] however these materials were not porous and the resultant topography were limited to 10-20-nm ripples spaced about 20-nm apart. Similar to that work, we obtained mostly nano-rods and some mixed areas with nano-ripples for an incident angle of 60°. This similarity can be explained in terms of the incident angle, which appears slightly higher than the Transition Angle (TA) between mostly nano-ripples formation (normal incidence, diffusive regime) and straight and long nano-rods (highly off-normal incidence, grazing incidence, of around 80°; erosive regime). Note that the high nano-patterning effectiveness observed here is mainly due to DIS on porous cpTi (100% α-phase, hcp), is in alignment with previous work using ion-beam sputtering on Ti6Al4V [44]. Other work included the use of high-energy 30-keV Ga+irradiation resulting in similar ripple structures but lacking any of the biological response control obtained in the work presented here [43,45]. These results showed partial nano-patterning due to DIS due to also partial percentage of a phase (hcp). Results indicate that this phase has a favorable strong response to DIS nano-patterning. In regards to high efficiency of DIS nano-structuring, no matter the porosity of cpTi samples, it could be associated to a favorable effect of previous polishing of samples, which have pores confined at the surface. The mixed surface of flat and porous areas seems not to mean any obstacle to DIS efficiency for surface nano-patterning of cpTi. It must be noticed that DIS nano-patterning was basically the same on porous cpTi samples with highest porosity. The positive response of outer pores to DIS nano-patterning can be explained in terms of interactions between remote (nominal) incident angle (60°) and local incident angles inside the pores; in this context, flat polished zones responded in a conventional mixed way of nano-rods+nano-ripples. However, the curved zones inside the pores are more sensitive to create nano-rods, which would mean that ion beam on curved surface helps to stimulate the erosive regime, when is used an intermediate incident angle like 60°. These observed tendencies are in well agreement with that observed in previous works about ion bombardment on porous solids, as well as with some theoretical predictions about ion beam nano-structuring, which are discussed below.

From the theoretical point of view, the first advances for understanding ion beam sputtering (IBS) nano-structuring was made by Sigmund [46] as he showed that local surface minima (“valleys”) should be eroded at a faster rate than local maxima (“peaks”); i.e., the sputtering rate depends on the local surface curvature, leading to a surface instability, which is the origin of the nano-structuring process. Based on this work, Bradley and Harper (BH) proposed later the first continuum model describing ripples formation [47]. In such a way, sputtering can be used to alter the surface morphology due to its dependence on the local surface curvature [48]. Depending on the irradiation conditions (angle of incidence, energy, surface curvature, etc.), it can induce either surface smoothening or roughening. These competing processes may be used to design the surface geometry important for certain applications. The velocity of erosion is therefore faster for the trough than for the crest. In the case of metals, the build-up of a regular pattern is produced by two different mechanisms, which lead to a similar surface instability: the surface curvature dependence of the ion sputtering and the presence of an extra energy barrier whenever diffusing adatoms try to descend step edges. In the case of a surface porosity mostly isolated and confined at the surface, several incidence angles can be locally experienced inside the pores for a fixed remote incident angle; this will be traduced in a curvature dependence of the surface nano-patterning. Therefore, we can speculate about a sequence of patterns inside the pores for an off-normal ion incidence like 60°; for an ideal semi-circular closed pore with a diameter of the same order of the grain size that would be: 1. Initially, a nano-rod domain region produced due to a semi-grazing incidence, which would be associated to a predominant erosive regime; 2. A second domain region would consist of a mixed pattern of rods and ripples due to its similarity with remote conditions due to 60° incidence (diffusive plus erosive regimes); 3. A nano-rippled region appears due to normal incidence conditions (diffusive regime); 4. Once again, a mixed rods+ripples zone due to occurrence of a local incidence angle similar to that off-normal remote angle. Note that some pores in FIG. 3 appear mostly with nano-rods inside, due to certain prevalence of erosive conditions. This positive response of both flat and curved zones of porous cpTi samples to DIS nano-structuring is strongly associated to smoothness of initial micro-topographical features.

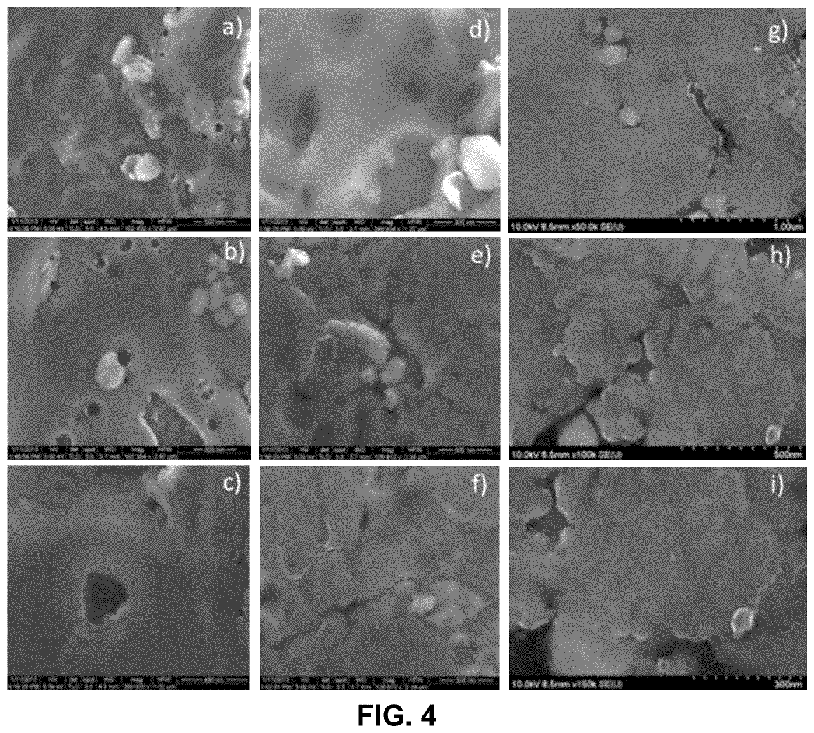

DIS on rough cpTi is performed through different incident angles, in order to test the influence on surface structuring; a low energy Ar+ion particle beam is extracted and applied to the samples with normal and off-normal incidences (0°, 45° and 75°). Images with lowest magnifications basically don't show any notorious micro-scale difference due to normal Ar+incidence with respect to untreated surface (see FIG. 2). However, when we move to a higher magnification (see FIG. 4a to 4c) it is appreciable that a slight proportion of nano-features response after DIS, which are clearly evident after normal DIS; there are a few zones with nano-ripples that can be observed and some new nano-holes that were absent in the bulk of starting rough surface as well. Occurrence of nano-ripples is not surprising for cpTi under normal incidence on a flat surface. They are normally associated to the prevalence of diffusive regimes, which is normally found for normal incidence. This is in agreement with that observed previously by Quian et al. [43] due to high-energy Ga+incidence by using FIB with normal incidence on cpTi, where they obtained 100% nano-ripples on cpTi grains. However, in addition to the use of ion energy of 30 keV, they also polished the samples before irradiation, in contrast to our original rough machined samples. But Ga-induced impurities also hampered the pure Ti property of their interface. On the other hand, these few nano-rippled zones are also consistent with that observed by Riedel et al. [49]; as in our case, they didn't polish their Ti6Al4V samples, by using a rotating sample holder and a normal incident source of Ar+ with different levels of low energy ions. They also observed partial nano-ripples and some effect of creating holes that they related to an etching effect of ion bombardment. However, the nano-ripples that they observed corresponded to ions energy far away higher than we used in this work (1100 eV against 500 eV in Table 1). In order to explain our results under normal incidence, as we described from Sigmund [46] and Bradley-Harper models [47], surface curvature of the irradiated substrate play a role in the final nano-structuring response; therefore, for original rough cpTi surfaces with a vertical roughness parameter of around 20 to 300 nm, it is rather hardly expected any effect of important percentage of diffusive regime on the surface, which allows to produce massive nano-ripples. Those roughness parameters imply a weakening of any potential diffusive mechanism in concordance with what is predicted by HB model. In contrast, a very low vertical roughness parameter like that one exhibited previously by flat parts of porous samples of round 5 nm, effectively ensures a high nano-patterning response due to ion incidence. Consistently with this statement, note that those few nano-ripples obtained in our rough samples due to normal incidence are preferentially located at the “valleys” of the profile; this is, once again, consistent with more favorable sites to reproduce the conditions for a diffusive regime due to normal incidence.

A similar insensitivity to Ar+normal incidence on initially rough surfaces has been also observed on different irradiated materials, like Cu samples that were observed by Hino et al. [49]; despite they were not focused on any nano-ripple formation, the surface became rough compared with that one before the irradiation, after treatment with the incident angle of 0°. In contrast, the surfaces were observed to become smooth with an increasing of incidence angle. This is reasonably consistent with our results here, as it will be discussed later about AFM analysis of irradiated surfaces. In addition, we can consider other two factors that can avoid effective nano-rippling during Ar+normal incidence on original rough surfaces: 1. Crystalline phase is not directly exposed to broad ion beam; then, this is a clear obstacle to any atom diffusion driving force due to impacting ions; 2. Irregularities associated to roughness can clearly allow some interference phenomenon between protrusions, which will reduce any driving diffusion nano-patterning effect due to impacting ions.

Nano-scale holes observed due to normal incidence could be related with increased roughness above mentioned, which is also and indicative of highest erosive effect due to interaction between normal incidence and initially rough surface. This Ar+normal incidence capability to create controlled nano-holes were previously reported by Li et al. [50]; in their work, they were able to produce those nano-holes by using a thin insulating solid-state membrane in which those samples had big symmetry hales in counterpart of the irradiated part, in such a way that a thin neck between the big pores and the irradiated part was removed by the ion beam. Therefore, within the same line of reasoning, our nano-holes due to Ar+normal incidence on a rough cpTi samples can be the consequence of a certain kind of empty space or big pore below the some thin outer irradiated layer; as in the work of Li et al [50]. It must be noticed also that, in addition to those created circular-shape holes, it can be appreciated some embryonic or nucleated pre-holes, which appear as a depressions similar to typical nano-voids which are normally observed after ductile fracture of metals. In search of another reasonable explanation, by looking the morphology of the voids produced, another source for this specific damage can rely on supersaturating defects (displacement damage) and accumulation of implanted inert gases (Ar+) [51]. Formation of voids and bubbles during ion beam interaction with materials addresses many important issues. In fact, formation of a three-dimensional (3-D) void-lattice has demonstrated the earliest example of self-organization in material processing. This is the key concept of the “bottom-up” technology used for modern ultra-small scale device formation.

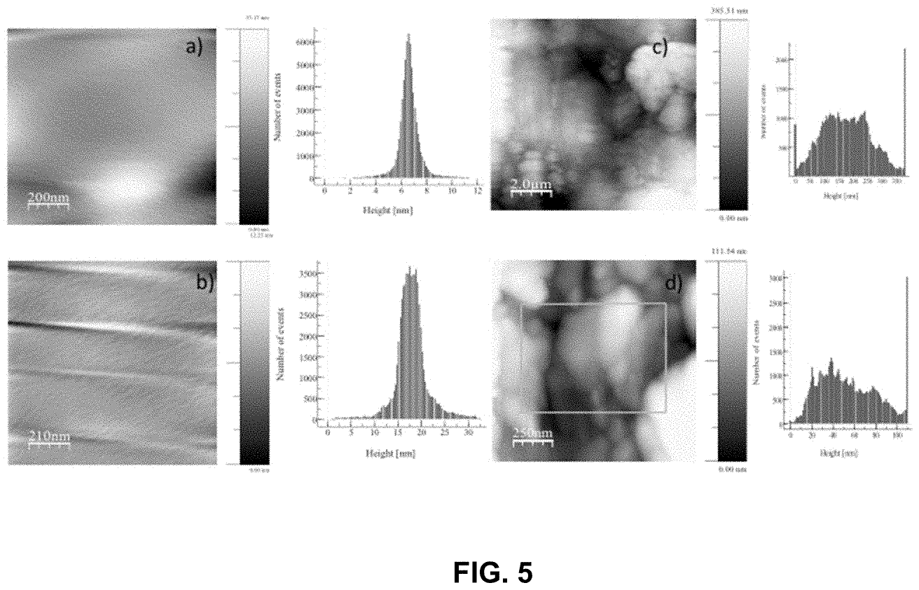

Off-normal incidence on rough cpTi samples was performed through irradiations of 45° and 75° incidence angles (see FIG. 5e to 5i); as in the case of normal incidence, it is not appreciated any important surface change at micro-scale level. For highest magnifications, despite there is not any appreciable nano-feature due to off-normal DIS, there is an evident smoothened effect; this effect totally opposite to that rougher, nano-holes, and a few nano-rippling for normal incidence. Reduced presence of “valleys” and “peaks” (protrusions) due to this off-normal incidence is also a confirmation of the smoothened effect. Importantly, that effect was even higher for the increased off-normal incidence (75°), (see FIG. 5g to 5i). Other authors like Hino et al [49] already observed this tendency of highest surface smoothening for highest off-normal incidence; they studied incidence angle influence of Ar+irradiation on highly pure Cu. As we mentioned before, they determined that surface was observed to become smooth with an increased incident angle. They observed the smoothest surface for an incident angle of 70°. Here, according to both theoretical predictions and our own observations in FIG. 5, we can speculate about the following: Ion bombardment can be used to alter the surface morphology due to its dependence on the local surface curvature [48]. Smoothing effect can be attributed to a loss of the balance between factors that control nano-patterning due to ion beam incidence; according to BH model [47], curvature dependent roughening and surface smoothing: because of the highest factor of curvature dependent roughening, smoothing must prevalence in order to re-establish the balance. As a consequence, small irregularities on a relatively smooth surface may result enhanced by ion bombardment. According to BH model [47], when height variation is far away to be an initial smooth surface, BH balance is unstable and, therefore, smoothened effect will be acting for off-normal incidence special for grazing angles.

AFM analysis has quantification the surface features due to DIS on both porous and rough cpTi (see FIG. 5); firstly, for samples with the lowest porosity, AFM images confirm the nano-patterning previously observed by SEM. Again, this is uniformly present at the flat zones due to previous mirror polishing. AFM images allowed us hardly appreciate mixed nano-patterning of nano-rods and nano-ripples. Roughness quantification is also presented in the same FIG. 5, in which can be appreciated the nano-scale of the vertical main roughness parameter of irradiated porous samples of 0.57 and 3.49 nm. Note the presence of polishing scratches due to mechanical polishing. Topographical and nano-patterning aspect of highest porosity samples (FIG. 5b), are similar to lowest porosity ones; however, quantification of roughness parameter shows that mean height is higher. Unfortunately, there are not any reported work about AFM characterization of cpTi irradiated in similar conditions; however, by comparison with some similar work on irradiation on cpTi [33], Cadmium telluride (CT) and Zn-doped (˜4 at %) CT (CZT) crystals [52], and Au, Pt, Ag, Cu and Co under Ar+ion beam sputtering at grazing incidence [53], our mean height nano-scale values are in well agreement with that.

FIGS. 5c and 5d present higher roughness parameters of rough irradiated surfaces with respect to irradiated porous ones. It is noticeable that vertical roughness parameter and mean height of the profiles are larger than same parameters of porous cpTi after irradiation. This AFM analysis also confirms that we did not create any nano-patterning, as reported in previous work of normal incidence on rough surface of Cu [49]. AFM results of off-normal incidence on same kind of rough surfaces showed some important differences; the reduction of both roughness parameter and mean height was not so drastic from normal to 45° incidence angle; however, from AFM image is evident the smoothing effect due to off-normal incidence in a similar way to what was observed by SEM, and in those previous similar works as well [49]. The flattening and smoothing effect is also evident due to lack of any debris as it was observed before for normal incidence. The change in roughness profile is evidently more drastic for highest off-normal angle (75°), in such a way that both roughness parameter and mean vertical height were importantly lower (22.07 and 55.42, respectively); these changes were also evident from the strongest smoothened effect observed in the AFM image, as well as the clear absence of any roughening debris.

Surface Free Energy Evaluation of as-Received (AR) and DIS Treated cpTi Samples

The wettability is fundamental for the cellular adhesion and, consequently, for the success of osseointegration and bone tissue growth, since the blood is the first tissue that reaches the implant, and 90% of its plasma is composed by water [54], the evaluation of the surface wettability can be accomplished through the determination of contact angle. First of all, we compare the contact angle of our untreated sample with some value of Ti already reported; to that end, we used some previous works about Ar+bombardment on cpTi [55] and on Riedel [45]. From that comparison, as-received surfaces (porous, polished and rough) presented similar values of free surface energies of simply AR machined Ti6Al4V samples [45], as well as values of cpTi mirror polished [55]: our samples with 53.03±0.06, against 54.55±6.54 for Ti6Al4V and 53.46±4 for cpTi II. The contact angle measurements after irradiation of porous cpTi samples, reflected an important change with respect to control one (see Table 2); both types of porous samples (lowest and highest porosity) demonstrated important reduction of contact angle (reduced hydrophobicity) after Ar+irradiation with 60° incidence angle. In case of lowest surface porosity, contact angle was of 13.45±4.51 (reduction of 74.64%), and for highest porosity the contact angle was of 28.15±5.21 (reduction of 46.92%). It is important to notice that those reduced values of contact angle after DIS were obtained despite the initial porosity of samples, and the initial roughness of rough samples as well. Despite there are reported some works about influence of ion irradiation on contact angle, this is the first time that such a reduction is reported due to off-normal Ar+incidence on Ti. With respect to those previous works on Ti6Al4V and cpTi II, there does not appear to be any examples of measurable change on contact angle due to normal Ar+incidence on machined Ti6Al4V; with respect to effect on cpTi II, a reduction of about 62.6% due to normal incidence of 1.5 keV has been reported (compared with our value of 1 keV).

TABLE 2
Results of contact angles testing (DI
water) on irradiated cpTi samples

| | Incidence | Mean | |
|---|---|---|---|
| Samples | Angle (°) | Value | STDEV |
| Untreated cpTi | — | 53.03 | 0.06 |
| S1 (cpTi) | 60° | 13.45 | 4.51 |
| S2 (cpTi | 60° | 28.15 | 5.21 |
| S3 (cpTi) | 0° | 33.3 | 3.5 |
| S4 (cpTi) | 45° | 32.1 | 4.2 |
| S5 (cpTi) | 75° | 25.6 | 3.8 |

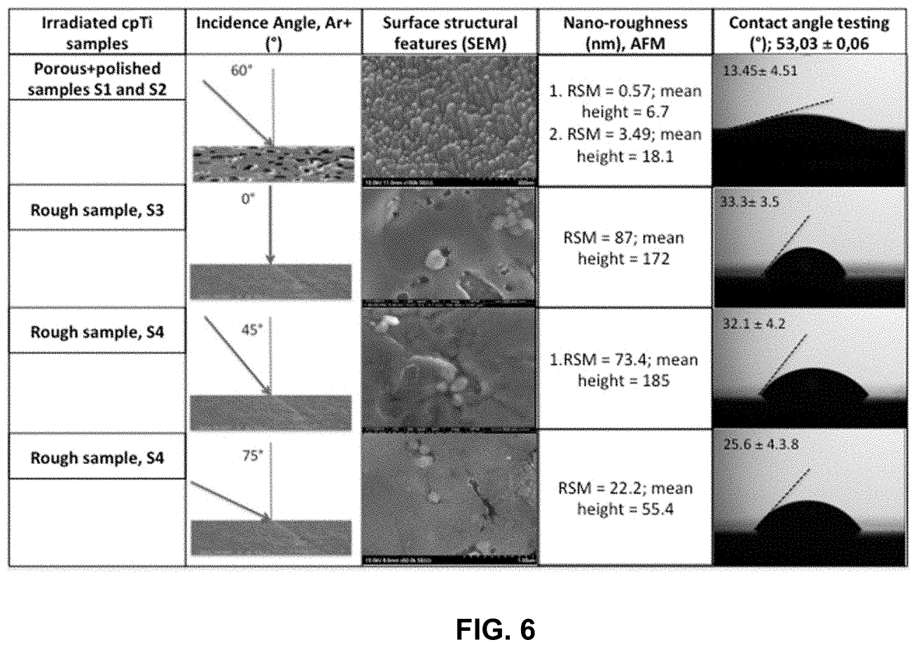

In summary, our results have shown a contact angle reduction due to DIS on both porous and rough samples; DIS nano-structuring of porous samples implied lowest contact angle for middle incidence angle of around 60 degrees; in addition, smoothing of rough samples due to normal and off-normal incidences implied also a drastic reduction of contact angles. In order to rationale this tendency, we must first try to understand the factors that determine the contact angle response of a surface and its relation with the surface free energy. The molecular interactions of water with a surface can be characterized by three mechanisms: covalent bonding, electrostatic interaction, and electromagnetic interaction [56, 57]. The amount of interaction that a liquid has with a surface can be described as a balance of free energies and the subsequent surface tension relating to each interface (solid-liquid, liquid-gas, gas-solid). To better describe wetting on real surfaces with roughness and imperfections, two models have been developed based on modifications to Young's equation; these are the Wenzel [58] and the Cassie-Baxter models [59]. The difference in these modes of analysis is based on assumptions that the liquid will react to surface irregularities in two distinct manners. In the Wenzel regime, although the surface is roughened, the liquid remains capable of complete contact with the solid beneath. In contrast, the Cassie-Baxter model assumes the roughness of the surface prevents complete contact between the liquid and solid by trapping gas between the two phases. According to these models, we can summarize that contact angle dependence about following factors: liquid properties, topographical parameters, surface chemistry, surface crystalline structure, surface crystalline defects, surface residual stress, surface micro-curvature, contact time, temperature and environmental pressure. Within this framework and by considering the relationships that we have established through our own master-diagram in FIG. 6, above contact angle response to irradiated surfaces can be explained as follows: 1. The lowest roughness of the irradiated surfaces with respect to control one plays an important role on the contact angle reduction with an evident correlation between both surface parameters. This can be easily verified by observing the tendency of roughness parameters with respect to tendency of contact angle values (FIG. 6). However, it must be also emphasized that there must be other factors that can be actually influencing importantly in that contact angle reduction, specifically associated to porous and polished samples. By following the reduction tendency exhibited by irradiated rough samples, it would not be expected such a drastic contact angle reduction observed on irradiated porous and polished samples. Obviously, it would be reasonable that previous polishing had a strong influence on that contact angle reduction; however that deviation with respect to that expected values from rough samples results opens the question of a possible influence of DIS surface modifications: basically nano-structuring and surface chemistry related; according to Wenzel regime and also because of the rather improbable chemical changes, it would be rationally expected that DIS nano-patterning of porous and polished samples has an actual influence on contact angle reduction.

Biological Assessment of as-Received (AR) and DIS Treated cpTi Samples

As it is well recognized from biological response of biomaterials surfaces, free surface energy of biomaterials reflected in their contact angle values plays a determinant role in the biological environment response. By considering the effect of multiple surface properties in contact angle results, here we can establish important relationships with surface modifications due to ion irradiation processing of cpTi surfaces. In that context, osseointegration is one of the good examples in which, besides the roughness, the surface tension is a parameter that interferes on it [60]. It permits a higher or smaller scattering of liquid onto the metallic surface. The human blood contains about 90% of water, thus the capability of water adsorption by the surface, known like wettability, is a fundamental parameter to the success of cellular adhesion and, consequently, to the osseointegration [54]. It is commonly accepted that blood compatibility is improved when the hydrophilicity of a surface is increased (unless the surfaces are superhydrophobic) [61-63].

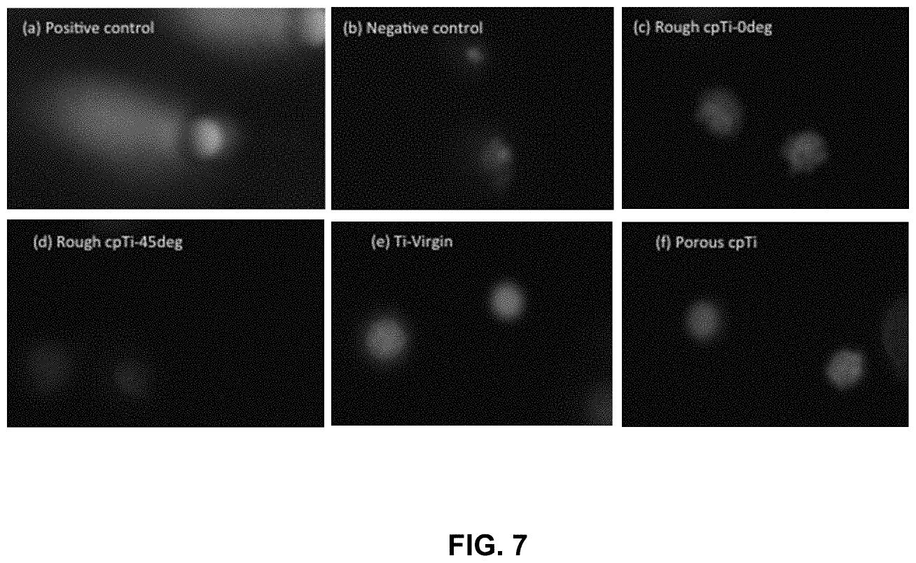

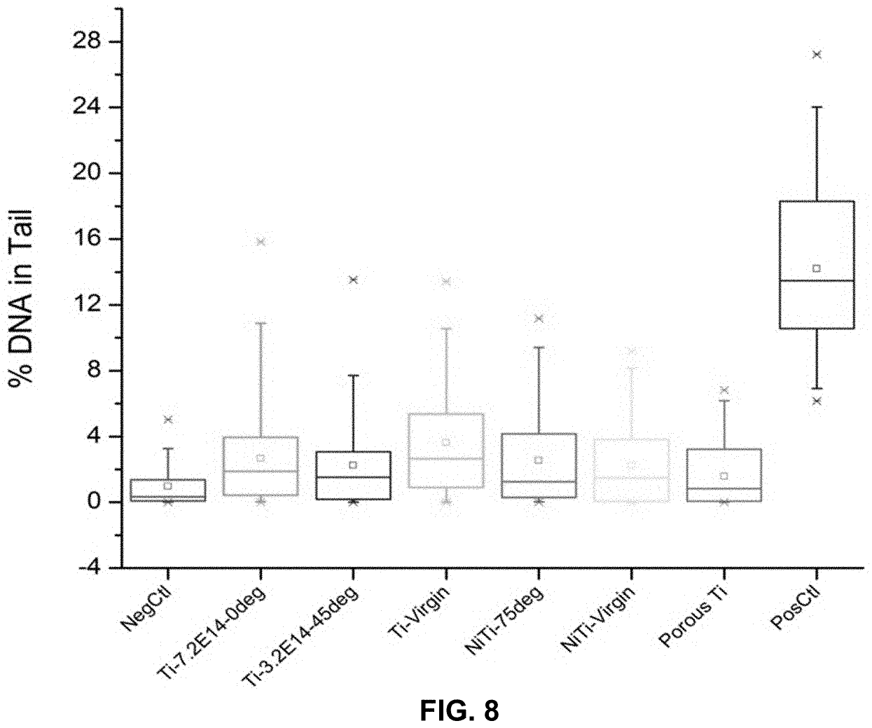

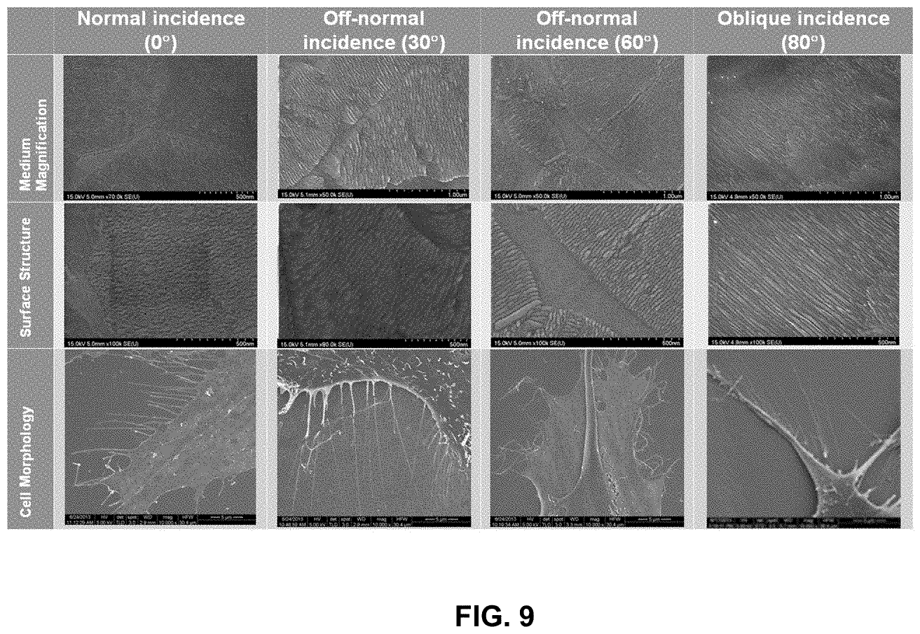

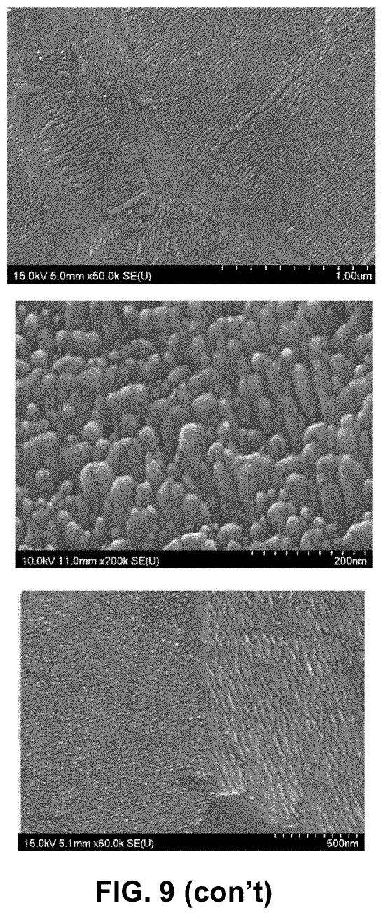

Cytotoxicity assessment of irradiated samples was performed via Comet Assay@ testing, in search of evaluating the potential geno-toxicological effect in vitro on human aortic smooth muscle cells (HASMCs) cultured in the presence of the evaluated specimens. FIG. 7a shows the characteristic shape of nucleoid exhibiting DNA damage (positive results for the assay) after treatment with hydrogen peroxide (H₂O₂). The tail of the nucleoid corresponds to DNA strand breaks produced by exposure with the toxic agent. FIG. 7b shows negative results for DNA damage in untreated cells (cells growing alone and without exposure to any toxic agent), which is characterized by compact nucleoids. FIGS. 7c-7f show the results obtained for the tested materials (untreated, and porous and rough cpTi irradiated samples). Notice that these nucleoid shapes are closer to negative control samples instead the positive ones. Virgin untreated sample in FIG. 7e corresponds to cpTi biocompatible surface without irradiation and use for comparative purposes. These samples show similar nucleoid shapes to their irradiated counterparts (FIGS. 7c, 7d and 7f). The potential DNA damage produced on HASMCs after exposure to the tested analytes was quantified using Comet Score™ software (Tri Tek Corp., Sumerduck, Va.). The results were compared with those obtained for the control samples (positive and negative controls). Positive results for DNA damage are characterized for high percentage of DNA in tail close or higher than the values for the positive control sample. Tables 3 and 4 summarize the results of percentage of DNA in tail obtained for controls and treated and untreated cpTi surfaces. At 0.05 levels, the tested materials means are significantly different to the positive control (Table 4), thus indicating no detrimental effects in HASMC's DNA induce by the tested materials under the experimental conditions outlined in this report. FIG. 8 display the data distribution in Tables 3 and 4. The range of variation for the tested samples is lower than the positive control (treated with H₂O₂). It indicates that irradiated cpTi surfaces did not induce DNA damage on HASMCs cultured in the presence of these materials under the experimental conditions tested in this work. Therefore, here we can estate that we tested the potential adverse effects on HASMC genetic material as a result of exposure to irradiated cpTi surfaces through the Comet Assay®. The results depicted in the present work suggest that irradiated cpTi samples (porous, polished and rough) with Argon under different conditions do not produce detectable DNA damage in HASMCs. In regards to HASMCs morphology changes during time in contact with those different surfaces, that was performed by visual inspection of HASMCs growing in the presence of the tested materials, during the incubation period under bright field illumination utilizing a Nikon inverted Diaphot fluorescent microscope with 10× and 20× objectives (Nikon Instruments, Melville, N.Y.). Periods of observations were 0, 24 and 48 hours, and this preliminary characterization of cells behavior by co-culture on the surfaces has shown that irradiated cpTi surfaces does not have any cytotoxicity effect on the HASMCs.

TABLE 3
Percentage of DNA in tail parameter after performing Comet-Assay
on HASMCs cultured in the presence of irradiated metal surfaces.

| | | | Standard |
|---|---|---|---|
| Sample | N Analysis | Mean | Deviation |
| Negative Control | 45 | 0.96 | 1.21 |
| Rough cpTi-0deg | 45 | 2.67 | 3.15 |
| Rough cpTi-45deg | 45 | 2.24 | 2.62 |
| Ti-Virgin | 45 | 3.63 | 3.38 |
| Porous cpTi | 45 | 1.57 | 1.95 |
| Positive Control | 45 | 14.20 | 5.06 |

TABLE 4
One way ANOVA was performed to compare the mean
of each tested sample with the control ones.

| | | Sum of | Mean | | |
|---|---|---|---|---|---|
| | DF | Squares | Square | F Value | Prob > F* |
| Model | 7 | 5798.4 | 828.34 | 89.14 | 0 |
| Error | 352 | 3270.9 | 9.29 | | |
| Total | 359 | 9069.3 | | | |
| | | Sum of | Mean | | |
| | DF | Squares | Square | F Value | Prob > F |
| Model | 2 | 5019.4 | 2509.72 | 243.53 | 0 |
| Error | 132 | 1360.3 | 10.30 | | |
| Total | 134 | 6379.8 | | | |

*At the 0.05 level, the population means are significantly different.

By comparing with previous works about biological assessment of Ar+irradiated Ti samples, our results are in well agreement with those reports; firstly, for the case of cpTi II after normal incidence [55], they found that proliferation tests showed that irradiated surfaces allowed a significant increase in the number of cells, compared to that data obtained in untreated surfaces. Their evaluations of the biological response in vitro in the new biomaterial surface showed that the behavior of pre-osteoblasts cells MC3T3-E1 was influenced by the topography, roughness and wettability of the titanium surface submitted to the argon-ion bombardment. Secondly, with respect to biological evaluations of Ar+irradiated Ti6Al4V under normal incidence [45], it was observed that Ar+beam etching had a largely positive impact on the cellular interaction over the as-received substrates. Calcein AM staining and SEM imaging confirmed that there was an increase in early cell spreading and mobility. Most importantly with respect to our cytotoxicity results, they also found that the ion beam etched substrates had slightly lower but comparable MTT absorbance to the untreated as-received substrates, but the ALP values of the etched were higher. Biocompatibility is, at least, equal to well-known biocompatibility of conventional bio-inert cpTi surface; with respect to cell behavior parameters, by considering our improvements in nano-structuring, roughness parameters and contact angle, we can reasonably expect that cells factors like adhesion, proliferation, migration, and differentiation will be also improved with the surfaces obtained here. However, it is important to point out at this moment that our results reported here are new in the sense that is the first time is shown relationships between initial surface micro-topography of cpTi (porous, polished and rough), irradiation conditions, surface nano-structure, roughness, surface free energy and basic biocompatibility assessment; they are related not only with their behavior and adhesion, but also with their further potential to stimulate tissue growth.

### CONCLUSIONS

Characterization of surface properties and preliminary biological evaluation of porous, polished and rough cpTi samples after low energy Ar+irradiation, has allowed us to highlight the following findings about their potential use for bone tissue growth:

1. Irradiation with Ar+ of porous, polished cpTi samples under off-normal incidence (60 degrees) produced a general nano-pattering corresponding to conditions of mixed diffusive and erosive regimes: mostly nano-rods and some curved nano-ripples. These nano-features appeared mostly with same orientation, which would indicate surface texturing of α-(hcp) phase, most probably associated to plastic strain of the surface micro-structure during polishing. Interestingly, surface porosity was not an obstacle for nano-structuring; even nano-pattering of pores inside was possible, which seems to be related with different angles that can be formed between the remote incident angle and the pore's curvature. This capability of Ar+irradiation to nano-patterning both flats and curved zones of porous cpTi implants opens, for the first time, a real opportunity to address successfully two of the most important issues of Ti implants: stress shielding by controlled bulk porosity, and improvements of osseointegration by converting the whole surface in a third generation one, by a controlled surface nano-patterning.

2. Micro-roughness of original rough cpTi samples was an enough obstacle to successfully nano-structuring after low energy Ar+irradiations with different incident angles. However, it was observed some important effects on micro and nano-topographical parameters of irradiated samples, depending on the incident angle. Normal incidence was the only condition that produced small proportion of nano-ripples; however, its most important effect was increasing of roughness and some production of circumferential-shape holes. These features are assumed to be mostly the consequence of irradiation of some thin plastic deformed layer, and/or due to creation of irradiation voids and bubbles. Small nano-ripples are due to small diffusive effect, whilst the increased roughness is most likely related with a process highly erosive. As long as off-normal angle was increased, an important smoothing effect was evident; in addition, the closest angle to grazing incidence had the strongest smoothened effect, which was appreciated from SEM analysis.

3. Detailed analysis and quantification of irradiated surfaces by AFM allowed us to confirm structural and roughness modifications due to low energy Ar+ion beams. Porous, polished cpTi samples exhibited a very smooth surface reflected in a few nano-meters parameter of nano-roughness; this is mostly a consequence of the polishing process. Interestingly, the initial smooth surface was not negatively affected by DIS nano-patterning; in contrast, it seems to generate an even smoother irradiated surface. Initial rough samples showed high roughness sensitivity to irradiation; in such a way that raised off-normal incident reduced vertical roughness parameters, due to smoothing effect above mentioned.

4. Surface free energy estimations of treated samples via contact angle testing served us to establish some relationships with structural modifications due to irradiation processing. Despite contact angle has a complex functional dependence on several factors, our results here were clearly sensitive to nano-roughness vertical parameter; therefore, the lowest nano-roughness parameter implied a lower contact angle value. Moreover, it relevant that the smoothing effect of DIS on rough samples allows similar values of porous, polished and irradiated samples, especially for the incident angle close to grazing incidence. With respect to influence of DIS nano-patterning on roughness and contact angle results, which can only be analyzed for porous, polished samples, it seems that higher nano-structuring not only implied a lower roughness, but also meant a lower contact angle, with a reduction of about 50% of initial value.

5. Preliminary biological assessment by Comet Assay® testing of both porous, polished and rough cpTi irradiated samples has shown that Ar+ions beams has not any toxicological detrimental effect on initial biocompatible surface. HASMCs morphology behavior in contact modified surface, and in terms of time, also suggests that these new surfaces will in fact improve In Vivo tissue stimulation. This can be stated from the consistency with several previous results, as well as because those important improvements observed on surface nano-structuring, controlled roughness and reduced free surface energy. In summary, phenomenological relationships and trends determined here allowed us to have a new insight about positive influence of Ar+irradiation on both porous and rough cpTi surfaces in order to not only improve osseointegration, but also to effectively promote bone tissue growth and repair.

### REFERENCES

[1] Lars Lidgren, The Bone and Joint Decade 2000-2010, Bulletin of the World Health Organization 2003, 81 (9), p. 69.
[2] Advances in Regenerative Medicine: Role of Nanotechnology and Engineering Principles, Chapter 12. Materials Surface Effects on Biological Interactions, Josep A. Planell, Melba Navarro, George Altankov, Conrado Aparicio, Elisabeth Engel, Javier Gil, Maria Pau Ginebra, and Damien Lacroix, V. P. Shastri et al. (eds.), pp. 233, Springer, The Netherlands, 2007.
[3] Gibson L J, Ashby M F (1997) Cellular solids: structure and properties, 2nd edn. University Press, Cambridge 15.
[4] Banhart J (2001) Prog Mater Sci 46:559.
[5] Parthasarathy J, Starly B, Raman S, Christensen A (2010) J Mech Behav Biomed Mater 3:249.
[6] Oppenheimer S M, Dunand D C (2009) Mater Sci Eng, A 523:70.
[7] Chino Y, Dunand D C (2008) Acta Mater 56:105.
[8] Ryan G E, Pandit A S, Apatsidis D P (2008) Biomaterials 29:3625.
[9] Krishna B V, Bose B, Bandyopadhyay A (2007) Acta Biomater 3:997.
[10] An Y B, Oh N H, Chun Y W, Kim Y H, Kim D K, Park J S, Kwon J-J, Choi K O, Eom T G, Byun T H, Kim J Y, Reucroft P J, Kim K J, Lee W H (2005) Mater Lett 59:2178.
[11] Orru' R, Licheri R, Locci A M, Cincotti A, Cao G (2009) Mater Sci Eng R Rep 63:127.
[12] Oh I H, Nomura N, Masahashi N, Hanada S (2003) Scr Mater 49:1197.
[13] Wen C E, Mabuchi M, Yamada Y, Shimojima K, Chino Y, Asahina T (2001) Scripta Mater 45:1147.
[14] Torres Y, Pavon J J, Nieto I, Rodriguez J A (2011) Metall Mater Trans B 42:891.
[15] Yadir Torres, Sheila Lascano, Jorge Bris, Juan Pav6n, Jose A. Rodriguez, Development of porous titanium for biomedical applications: A comparison between loose sintering and space-holder techniques, Materials Science and Engineering C 37 (2014) 148-155.
[16] Boyan B D, Schwartz Z (1999) Modulation of osteogenesis via implant surface design. In: Davies J E (ed) Bone engineering. Em2 Inc, Toronto, p 232.
[17] Aparicio Badenas C, Gil F X (2005) Tratamientos de superficie sobre titanio comercialmente puro para la mejora de la osteointegracio n de los implantes dentales. In: Universitat Polite'cnica de Catalunya. Departament de Cie'ncia dels Materials i Enginyeria Metallu'rgica. http://hdl.handle.net/10803/6044. Accessed 15 Apr. 2005.
[18] Hench, L. L., “Bioceramics: From Concept to Clinic”, J. Am. Ceram. Soc., 74, 1487-1510, (1991).
[19] McMurray, R. J., N. Gadegaard, P. M. Tsimbouri, K. V. Burgess, L. E. McNamara, R. Tare, K. Murawski, E. Kingham, R. O. Oreffo, and M. J. Dalby, 2011, Nanoscale surfaces for the long-term maintenance of mesenchymal stem cell phenotype and multipotency: Nature materials, v. 10, p. 637-644.
[20] Nanotopographical modification: a regulator of cellular function through focal adhesions, Manus Jonathan Paul Biggs, R. Geoff Richards, Matthew J. Dalby, Nanomedicine: Nanotechnology, Biology, and Medicine 6 (2010) 619-633.
[21] Dalby, M. J., N. Gadegaard, R. Tare, A. Andar, M. O. Riehle, P. Herzyk, C. D. Wilkinson, and R. O. Oreffo, 2007, The control of human mesenchymal cell differentiation using nanoscale symmetry and disorder: Nature materials, v. 6, p. 997-1003.
[22] Andersson, A.-S., J. Brink, U. Lidberg, and D. S. Sutherland, 2003, Influence of systematically varied nanoscale topography on the morphology of epithelial cells: NanoBioscience, IEEE Transactions on, v. 2, p. 49-57.
[23] Martines, E., K. Seunarine, H. Morgan, N. Gadegaard, C. D. Wilkinson, and M. O. Riehle, 2005, Superhydrophobicity and superhydrophilicity of regular nanopatterns: Nano letters, v. 5, p. 2097-2103.
[24] Harper, J., and K. Rodbell, 1997, Microstructure control in semiconductor metallization: Journal of Vacuum Science & Technology B: Microelectronics and Nanometer Structures, v. 15, p. 763-779.
[25] Krasheninnikov, A., and F. Banhart, 2007, Engineering of nanostructured carbon materials with electron or ion beams: Nature materials, v. 6, p. 723-733.
[26] Vo, N., R. Averback, P. Bellon, S. Odunuga, and A. Caro, 2008, Quantitative description of plastic deformation in nanocrystalline Cu: Dislocation glide versus grain boundary sliding: Physical Review B, v. 77, p. 134108.
[27] Allain, J., M. Nieto, M. Hendricks, P. Plotkin, S. Harilal, and A. Hassanein, 2007, IMPACT: A facility to study the interaction of low-energy intense particle beams with dynamic heterogeneous surfaces: Review of Scientific Instruments, v. 78, p. 113105-113105-14.
[28] Lu J, Rao M P, MacDonald N C, Khang D, Webster T J. Improved endothelial cell adhesion and proliferation on patterned titanium surfaces with rationally designed, micrometer to nanometer features. Acta Biomater 2008; 4:192-201.
[29] Sjostrom T, Dalby M J, Hart A, Tare R, Oreffo R O, Su B. Fabrication of pillar-like titania nanostructures on titanium and their interactions with human skeletal stem cells. Acta Biomater 2009; 5:1433-41.
[30] ASTM F67-00 (2002) Standard specification for unalloyed titanium for surgical implant applications.
[31] Nanostructured Biointerfaces, Jean Paul Allain, M. Echeverry, S. Arias, J. Pavón, Chapter in: 2013 CMOS Emerging Technologies Research Symposium Book, submitted.
[32] Chan W L, Chason E. Kinetics of ion-induced ripple formation on Cu (001) surfaces.
[33] H. X. Qian, W. Zhou. Self-organization of ripples on Ti irradiated with focused ion beam. Applied Surface Science 258 (2012) 1924-1928.
[34] Carsten Busse, Cemal Engin, Henri Hansen, Udo Linke, Thomas Michely, Herbert M. Urbassek. Adatom formation and atomic layer growth on Al(111) by ion bombardment: experiments and molecular dynamics simulations. Surface Science, Volume 488, Issue 3, 10 Aug. 2001, Pages 346-366
[35] J. Naumann, J. Osing, A. J. Quinn, I. V. Shvets. Morphology of sputtering damage on Cu(111) studied by scanning tunneling microscopy. Surface Science, Volume 388, Issues 1-3, 23 Oct. 1997, Pages 212-219
[36] G Costantini, S Rusponi, F Buatier de Mongeot, C Boragno and U Valbusa. Periodic structures induced by normal-incidence sputtering on Ag(110) and Ag(001): flux and temperature dependence
[37] O Malis, J D Brock, R L Headrick, M S Yi, J M Pomeroy. Ion-induced pattern formation on Co surfaces: An x-ray scattering and kinetic Monte Carlo study. Physical Review B, 2002—APS
[38] M. V. Ramana Murty, T. Curcic, A. Judy, and B. H. Cooper. X-Ray Scattering Study of the Surface Morphology of Au(111) during Ar+Ion Irradiation. Physical review letters volume 80, number 21. 25 May 1998
[39] Matthias Kalff, George Comsa, Thomas Michely. Temperature dependent morphological evolution of Pt(111) by ion erosion: destabilization, phase coexistence and coarsening. Surface Science, volume 486, Issues 1-2, 1 Jul. 2001, Pages 103-135
[40] P. Karmakar, G. F. Liu, J. A. Yarmoff. Sputtering-induced vacancy cluster formation on TiO₂ (110). Physical review B 76, 193410 (2007).
[41] Sebastiaan van Dijken, Dennis de Bruin, and Bene Poelsema. Kinetic Physical Etching for Versatile Novel Design of Well Ordered Self-Affine Nanogrooves. Phys. Rev. Lett. 86, 4608-4611 (2001)
[42] Tim Luttrell and Matthias Batzill. Nanoripple formation on TiO2 (110) by low-energy grazing incidence ion sputtering. Phys. Rev. B 82, 035408 (2010).
[43] Self-organization of ripples on Ti irradiated with focused ion beam, H. X. Qian, W. Zhou, Applied Surface Science 258 (2012) 1924-1928.
[44] New Nanostructured Biointerfaces of Ti6Al4V by Directed Irradiation Synthesis (DIS) for Cells Stimulation: Analysis of Their Tissue Growth Potential, Juan Pavon, Osman El-Atwani, Sandra Arias, Emily Gordon, Lisa M. Reece, Jean Paul Allain, submitted.
[45] Ion beam etching titanium for enhanced osteoblast response, Nicholas A. Riedel, John D. Williams, Ketul C. Popat, J Mater Sci (2011) 46:6087-6095.
[46] Sigmund P (1969) Theory of sputtering. I. Sputtering yield of amorphous and polycrystalline targets. Phys Rev 184: 383-416.
[47] Bradley, R. M., Harper, J. M. E.: Theory of ripple topography induced by ion bombardment. J. Vac. Sci. Technol. A 6(4), 2390-2395 (1988).
[48] Vajo J J, Doly R E, Cirlin E H (1996) Influence of O₂⁺ energy, flux, and fluence on the formation and growth of sputtering-induced ripple topography on silicon. J Vac Sci Technol A 14: 2709-2720.
[49] Smoothing of polycrystalline copper with rough surface by oblique argon-ion irradiation, Hino, T.; Nakai, T.; Nishikawa, M.; Hirohata, Y.; Yamauchi, Y., Journal of Vacuum Science & Technology B Microelectronics and Nanometer Structures, 24(4): 1918-1921, 2006-07.
[50] Ion-beam sculpting at nano-metre length scales, Jiali Li, Derek Stein, Ciaran McMullan, Daniel Branton, Michael J. Aziz & Jene A. Golovchenko, NATURE, VOL 412, 12 Jul. 2001.
[51] Fundamentals of Radiation Materials Science, Metals and Alloy, Gary S. Was, Springer-Verlag Berlin Heidelberg, 2007, p. 343.
[52] Self-organized surface nano-patterns on Cd(Zn)Te crystals induced by medium-energy ion beam sputtering, R Gago, L Vazquez, F J Palomares, F Agullo-Rueda, M Vinnichenko, V Carcelen, J Olvera, J L Plaza and E Dieguez, J. Phys. D: Appl. Phys. 46 (2013) 455302 (10pp).
[53] Ion beam sputtering induced ripple formation in thin metal films, P. Karmakar, D. Ghose, Surface Science 554 (2004) L101-L106.
[54] B. Kasemo, Surf. Sci. 500 (2002) 656.
[55] Influence of argon-ion bombardment of titanium surfaces on the cell behavior, J. C. Sa, R. A. de Brito, C. E. Moura, N. B. Silva, M. B. M. Alves, C. Alves Junior, Surface & Coatings Technology 203 (2009) 1765-1770.
[56] I. A. Ryzhkin, V. F. Petrenko, J Phys Chem B, 101 (1997) 6267-6270.
[57] A. Dotan, H. Dodiuk, C. Laforte, S. Kenig, J Adhes Sci Technol, 23 (2009) 1907-1915.
[58] R. N. Wenzel, Ind Eng Chem, 28 (1936) 988-994.
[59] A. B. D. Cassie, S. Baxter, T Faraday Soc, 40 (1944) 0546-0550.
[60] J. M. Schakenraad, H. J. Busscher, C. R. H. Wildevuur, J. Arends, J. Biomed. Mater. Res. 45 (1999) 140.
[61] C. J. Lin, Y. Yang, Y. K. Lai, Q. Q. Zhang, K. Wu, L. H. Zhang, P. F. Tang, Colloid Surface B, 79 (2010) 309-313.
[62] F. Shen, E. Zhang, Z. J. Wei, Mat Sci Eng C-Mater, 30 (2010) 369-375.
[63] S. Takemoto, T. Yamamoto, K. Tsuru, S. Hayakawa, A. Osaka, S. Takashima, Biomaterials, 25 (2004) 3485-3492.

### Example 2: Nanostructured Ti6Al4V Biointerfaces by DIS for Endothelial Cell Stimulation

### Abstract

Degradation and damage of human tissues are one of the most important public health problems that often compromise the patient's quality of life. Multi-disciplinary teams from government to academic/industrial networks routinely confront this challenge seeking to develop practical treatments and repair. For damage to bone tissue, it is widely recognized that medical grade titanium alloys, such as Ti6Al4V, is the best biomaterial for bone repair due to an optimal balance between its biomechanical and biocompatible properties. Several cases in the literature exist that test the hypothesis of using Ti6Al4V for growth stimulation of other tissues different than bone by some type of surface modification. In this work we have obtained, for the first time, high-fidelity control of surface nanostructures on medical grade Ti6Al4V by using ion-beam sputtering (IBS) resulting in control of cell shape, adhesion and proliferation. These surfaces were biologically tested through the response of human aortic smooth muscle cell (HASMC) line as a cell model. This cell line was specifically used for cytotoxic assessment through yellow tetrazolium MTT method, and for evaluating morphological and adhesion changes of cells, in contact with the new surfaces. Cell behavior indicated not only an unaltered biocompatibility of the new Ti-alloy nanostructured surfaces, but also important dramatic control of filopodia and lamellipodia activity of cells. These are good indicators of improvement of cell adhesion and proliferation. Improvements of cell attachment, especially in regards to filopodia activity, were directly related with nano-ripples structure geometry and, therefore, with incidence angle, which followed a diffusive regime during ion-beam irradiation.

### Introduction

One of the fundamental premises in substitutive medicine is that once a certain level of deterioration of any organ or tissue has been reached, a more effective protocol is to remove it and replace it rather than attempt to heal it. In that context, damaged tissues are currently treated from two conventional medical paradigms: 1) organ transplants and 2) tissue/organ replacement with a biomaterial; i.e. via auto-grafts or allograft routes [1]. Tissue engineering (TE) and regenerative medicine (RM) are two concepts clearly related but inherently different. While TE focuses on engineering methods to produce new tissue from a variety of cellular sources, RM focuses on the use of biomaterials for the regeneration of tissues and organs [2]. RM, in the widest sense, is concerned with the restoration of impaired function of cells, tissues, and organs either by biological replacement, e.g. with tissues cultured in-vitro, or by providing the stimulus for the body's own reparative and regenerative mechanisms [4]. Simultaneously, biomimetics and bioinspired concepts have emerged based on the principle of construction of artificial materials that attempt to imitate the tissue they are implanted in and are actively interacting with its cells [6]. The repair and substitution of bone require both non-degradable and degradable materials that should be able to integrate and form a direct bond with the tissue; such is the case for osseointegration. Within this context, pure titanium (Ti) and some of its alloys, and some bioactive bioceramics as well, are the conventional biomaterials that have shown the highest clinical success [2]. Ti is widely recognized to be the preferred biomaterial for bone replacement due to its excellent balance between biomechanical and biocompatibility response. However, titanium suffers from the intrinsic growth of a thin fibrous tissue interface [2].

Last decade has been arguably a definitive period for the role nanotechnology can play on advanced biomaterials. In that sense, surface nanostructuring of conventional 1^{st} and 2^{nd} generation biomaterials, has emerged as one of the most important and effective approaches to convert them to a 3^{rd} generation biomaterial, as described herein. Meaning that as a consequence of nano-features fabricated on the biomaterial in question it imparts the function to effectively influence surrounding biological environment at a molecular nano-scale level. Several studies demonstrated different morphology configurations at the nanoscale can have a strong and direct influence over cellular behavior; indeed, it's possible to appreciate that cells prefer texturized surfaces in comparison with smooth ones, depending on cell type [6]. Biointerface topography and, in particular, nanoscale features can affect cell behavior and integrin-mediated cell adhesion, and is now evident from studies with fabricated topographical features [3]. The extent to which nanotopography influences cell behavior in-vitro remains unclear, and investigation on this phenomenon is still underway. The processes that mediate the cellular reaction with nanoscale surface structures are also not well understood [7]. For example, it is not clear if this influence derives directly from surface topography, or perhaps indirectly with surface structures possibly affecting the composition, orientation, or conformation of the adsorbed ECM (extra-cellular matrix) components [8, 9].

A few papers have reported converting Ti into a third generation biomaterial and those papers do not appear to describe modifying Ti surfaces for regenerative medicine. Most of them have shown improvements of cell adhesion due to interactions with nano-patterned surfaces, as regulators of cellular functions through focal adhesion. Results include: increased adhesion and formation of human mesenchymal stem cells [10], improvements of rat aortic endothelial cells adhesion on TiO₂ [11], upregulation of osteospecific proteins—osteopontin and osteocalcin of human osteoprogenitor cells cultured on Ti [10]. Conventional processing of materials for surface nanostructuring has been dominated by the development of advanced top-down fabrication techniques that include lithography-based techniques. Some of them include focused-beam lithographies using electron or ion energetic particles and scanning probe lithographies [12, 13]. The drawbacks of these conventional techniques have mainly been attributed to their physical limitations in fabricating structures smaller than about 50-nm and also limited to the modification of a few classes of materials. Therefore, bottom-up techniques that rely on self-assembly, self-organization, and local patterning, have become emergent technologies capable of patterning biocompatible surface nanostructures. Ion beams can be used to induce patterned structures with unique topography at the nano-scale by means of sputtering and other surface-related processes [14-22]. Irradiation-driven systems have been explored in similar moderate energy regimes dominated by knock-on atom displacement regimes for semiconductor metallization microstructure control [23], engineering of nanostructured carbon [24], and compositional patterning of immiscible alloys [25]. One important limitation in current nanomanufacturing approaches is a dependence on naturally self-ordered processes that balance kinetic and thermodynamic dissipative forces in the absence of irradiation therefore requiring very high temperature processes [26]. Consequently, many of the desired biomaterial properties that require a combination of metal alloy and soft material interfaces cannot be processed with conventional bottom-up techniques. Directed Irradiation Synthesis (DIS) and Directed Plasma Nanosynthesis (DPNS) address this limitation by introducing a synthesis process that is scalable to high-volume manufacturing by virtue of its intrinsic large-area simultaneous exposure of materials surfaces and interfaces. One subset of DIS is ion-beam sputtering (IBS) and is the methodology used in the work reported here. Advanced in-situ synthesis methods have been recently developed by Allain et al. to elucidate ion-irradiation mechanisms that can manipulate surface chemistry and surface morphology to ultimately synthesize functional coatings for 3D scaffold systems [27].

The aim of the work reported here is to examine the role surface nanostructuring of Ti6Al4V by IBS can have in the stimulation of cells and tissues, other than bone, in order to provide important cues for tissue regeneration. We report a systematic study where we have successfully nanostructured medical grade Ti6Al4V and conducted detailed characterization establishing processing conditions and correlating them to surface and biomaterial properties. These new nanostructured surfaces were biologically evaluated by using human aortic smooth muscle cells (HASMCs) for proliferation and cell/surface adhesion. This analysis allowed us to determine connections with processing, structure, surface energy, and biointerface properties. Biological response of these new surfaces has also lead us, for the first time, to establish correlations between nanostructuring by IBS and cell stimulation, as well as to show the real potential of these new surfaces to favorably stimulate cells and tissues different than bone.

### Materials and Methods

Ion Beam Sputtering (IBS) of Ti6Al4V Samples

Medical grade Ti6Al4V alloy (ASTM F136, F1472) samples were used for surface modification by IBS. Samples were initially prepared by grinding and polishing up to mirror finish, before exposure to irradiation processing. IBS synthesis conditions for various Ti6Al4V samples are summarized in Table 5. Conditions were selected after an exhaustive revision of our previous DIS nano-structuring results on metals [27, 28], as well as from some other revised works on Ti, TiO₂, Cu, Au, and Ag [29-40]. Argon (Ar+) source at 1 keV was used to irradiate discs of Ti6Al4V; several angles of incidence and fluences were also evaluated, as shown in Table 5

TABLE 5
Irradiation Parameters (1-keV Ar⁺) on Ti6AI4V Samples

| | Energy | Flux (ions | Fluence | Incidence | |
|---|---|---|---|---|---|
| Samples | (keV) | sec⁻¹cm⁻² | (cm⁻²) | angle (°) | Time (s) |
| S1 (Ti6) | 1.0 | 6.51E14 | 2.5E17 | 0° | 384.0 |
| S2 (Ti6) | 1.0 | 3.29E14 | 2.5E17 | 30° | 758.8 |
| S3 (Ti6) | 1.0 | 6.44E14 | 2.5E17 | 60° | 387.9 |
| S4 (Ti6) | 1.0 | 6.55E14 | 2.5E17 | 80° | 381.6 |

Structural and Energy Characterization of IBS Modified Surfaces

Surface energy of irradiated samples was evaluated by contact angle testing with deionized water through a Rame-Hart Goniometer Model 500-Advanced contact angle goniomter/tensiometer with DROPimage Advanced Software. We performed the sessile method of contact angle analysis (where the sample was static and not moving after the drop was placed on it). All measurements were performed with deionized water (to not have any type of interaction with the surface). The water dropper was far enough away from the surface that the water did not touch the sample until it left the dropper. The dropper was kept at the same distance from each sample surface. The water droplets had ˜3 μL of water on each sample. Morphological features of samples were analyzed by Scanning Electron Microscopy, SEM (Philips XL40 field emission, FEI, Hillsboro, Oreg., USA).

Cell Morphology Via SEM

HASMCs morphology was observed using a scanning electron microscope (SEM). For this, cells were culture for 24 hours on un-irradiated and irradiated Ti6Al4V samples, and subsequently, fixed and dehydrated using 10% formalin and increasing concentrations of ethanol (30, 50, 70, 80, 90 and 100%) respectively. Finally, the samples were subjected to critical point drying and coated with gold to be observed by SEM.

Cell Proliferation Via MTT

HASMCs (ThermoFisher Scientific, MA) were seeded on the top of un-irradiated and irradiated Ti6Al4V for 24 hours under normal culturing conditions (37° C., 95% air, 5% CO₂, 95% humidity) at a density of 5×10³ cells/cm². Afterward, yellow tetrazolium MTT colorimetric test was carried out and the mitochondrial activity was read at 570 nm according to the manufacturer's instructions (Sigma Aldrich, M0).

### Results and Discussion

Surface Nano-Structural Characteristics of IBS Ti6Al4V Samples

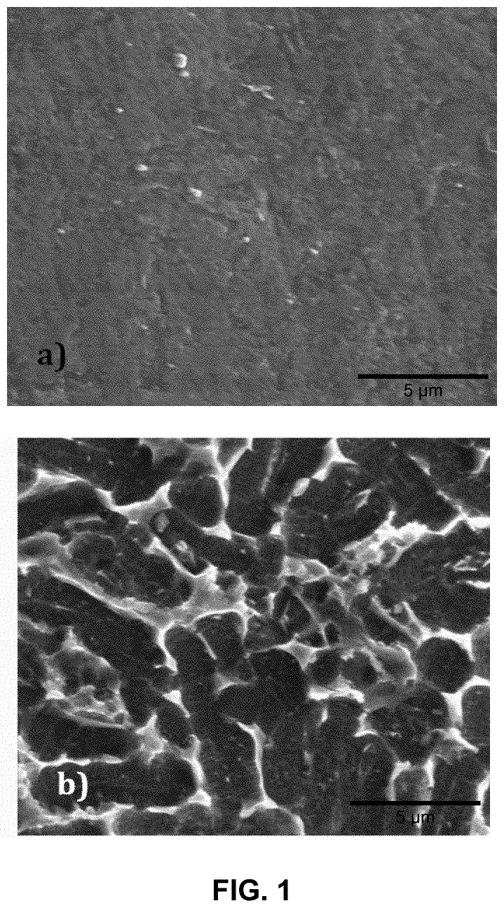

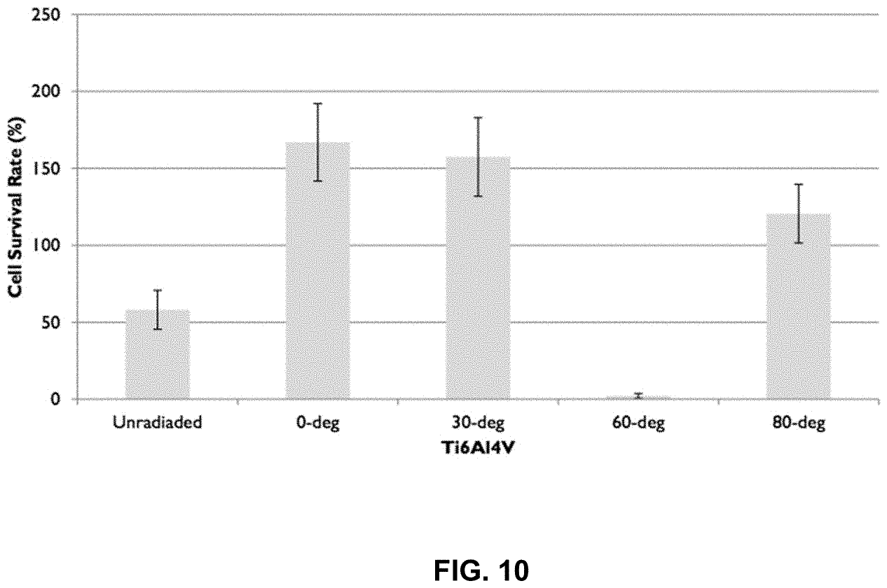

Considering the microstructure of unirradiated Ti6Al4V samples after proper etching procedures, one can observe a conventional α+β mill-annealed alloy (see FIG. 1) consisting of a phase (hcp), equiaxial grains and Widmanstatten plates, dispersed in an untransformed β matrix (bcc). This microstructure is the consequence of both heating and milling at the α+β thermodynamically stable region, and further slow cooling, allowing β−α transformation. This combination of phases and constituents results in an excellent balance between mechanical strength, toughness, ductility and fatigue resistance [43]. Surface structural modifications and nanostructuring due to IBS of Ti6Al4V are summarized in FIG. 10. Intrinsic to the IBS modification is its ability to only modify the first few 100's of nm and therefore not affect the optimized mechanical properties mentioned described above. The first observation relates to the effect normal incidence has on the modified surface. At normal incidence an organized dot-like structure formed alongside “broken” nanoscale ripples on the original grains is observed. Comparison with the original surface microstructure, the self-organized dot-like structures appear concentrated in the a phase (grains and Widmanstatten plates), whilst the more irregular damage seems to be preferentially located within 3 phase matrix. Furthermore, the nanostructures at normal incidence appear to have some preferential growth depending on grain orientation. This type of self-organized pattern at normal incidence appears to be corroborated with previous results from a variety of materials including: amorphous materials [44, 45], single crystals [46, 47], and polycrystalline metals, under normal incidence of Ar+[30, 48, 49]. From those previous papers, a diffusive regime is explained to be predominant for normal incidence. However, normal incidence and low energy processes in some types of materials (e.g. Si) have resulted in the smoothening of the surface. Evidence for a resistance to patterning is found in the normal incidence case as well as more oblique angles for certain grains. This could be evidence of a balance between mass redistribution mechanisms that drive adatoms on the surface to recombine with irradiation-driven surface vacancies leading to smooth surfaces. The fact that smooth surfaces only occur under certain grain orientations suggests that there is also a structure-driven relaxation mechanism coupled to the irradiation-driven mechanisms that lead to self-organized nanostructures. As the incident angle is increased, the stabilization of surface ripples is observed and is corroborated by previous data on “off-normal” irradiation [29-40, 44-49]. These incipient nano-ripples can be also explained due to the activation of the terrace diffusion barrier and of the Ehrlich-Schwoebel barrier [45, 50], which opens the possibility for the development of the surface instability connected to ripple formation. At normal incidence, the features produced by ion sputtering reflect the surface symmetry and are aligned along energy preferred crystallographic orientation [45] and enhanced surface recombination, which can lead to partial nano-dot and nano-ripple formation or in some cases complete smoothening of the surface. With respect to previous work consisting of Ar+bombardment on Ti alloys, there are a few reported results. Riedel et al [49] performed normal irradiation with Ar+ on Ti6Al4V rough samples by using a rotator holder followed by evaluation of cell response. Comparing to this work we find similar resistance to nanopatterning along specific grain orientation or microstructure phases.

In FIG. 10 are also depicted structures obtained from off-normal incidence starting with 0=30°. This change in the incident angle results in a predominant elongated rippled nanostructure at the irradiated surface mostly associated to the response of a (hcp) phase of the alloy. Given its polycrystallinity the observed nano-ripples were not perfectly perpendicular to the direction of irradiation. Most of them clearly appear with a shift of direction, which is a consequence of the coupling of incident energy deposition at the near surface along with surface diffusion being correlated to the movement of atoms associated with a particular crystallographic orientation [45]. Interestingly, grains of the same a (hcp) phase were not transformed to nano-ripples, exhibiting a type of elongated nano-grains (short nano-rods). These nanostructures can be assumed as an intermediate nanostructure phase between curved “elongated” nano-ripples and long nano-rods in the same direction of the ion beam (see discussion later) consistent with an erosive-dominated and enhanced surface diffusion regime as described by the Bradley and Harper (BH) model [51]. Another important point is the difference in response of metallic surfaces compared to semiconductor materials to ion irradiation. Metals have both higher diffusivity and the intrinsic non-directional nature of the metallic bond results in both resistance to amorphization and consequently a more sensitive dependence to crystallographic orientation. The formation of a pattern is thus dictated by: the surface curvature dependence of ion-induced sputtering and the presence of an extra energy barrier whenever diffusing adatoms try to descend step edges [45]. Formation of nano-ripples on Ti6Al4V due to off-normal ions incidence is consistent with those assumed mechanisms on metals: diffusive processes are mainly responsible for the formation of regular structures. However, here we have two new findings, which warrant brief discussion: those nano-rods observed in some grains of a (hcp) phase, and the clear insensitivity of p (bcc) phase in Ti6Al4V alloys to nano-structuring. These features (or the lack thereof) can be attributed to the influence of crystallographic orientation in conjunction with corresponding a (hcp) phase and 3 (bcc) phase.

Previous work in off-normal Ar+irradiation on Ti alloys reported by Quian et al [30] consisted of a study that used the focused ion beam (FIB) technique with relatively high-energy 30-keV Ga+ions on polished commercially pure Ti (cpTi) to induce topographical patterning. Focused Ga+ions were rastered at various FIB incidence angles over a wide range of doses and at room temperature. Results presented evidence of nanoscale ripple formation at normal incidence, which was strikingly different than well-known irradiation experiments of normal incidence bombardment of semiconductors. Examination by Quian et al. demonstrated the important role that crystallographic orientation has on pattern formation and in particular the role of surface diffusion in the intrinsic anisotropic hcp structure of titanium. In addition, contrary to prior observations of ripple structure direction dependence with incident angle, results demonstrated that regardless of the incident angle, the important factor was the crystallographic orientation of each grain for the direction and type of pattern (e.g. ripple vs dot) formed at normal and near-normal incidence. In one instance with incident angle of 30°, they also observed curved nano-ripples and fragmented rods, consistent with our irradiation protocols of Ti6Al4V alloys. Furthermore, as the incident ion-beam angle was systematically increased, the importance of the incident ion-beam direction on the type and direction of the nanopattern became stronger. However, the key difference between the work by Qian et al. and that presented here is the high-fidelity control not of the ripple structure but more importantly of the nanowall systems that ultimately plays a key role in the modulating immuno-response of macrophage phenotype and cell adhesion.

Current atomistic computational modeling by Yang et al. [reference PRB 2015 and NIMB 2014] have demonstrated the role increasing incident ion-beam angle has on ripple nanopattern formation and discovered a correlation to erosion-dominant mechanisms that increased with an increase in ion-beam incident angle. The same models also demonstrated that as the incident ion-beam angle decreased to near-normal incidence, the erosive component would decrease and atomic diffusive mechanisms would take over influence mass redistribution during irradiation and ultimately dictating pattern formation. In this case, surface anisotropy and crystallographic orientation would therefore dominate in the onset of ion-induced pattern formation. Therefore, we conclude that surface nanostructuring due to ion-beam sputtering (IBS) on Ti alloys is controlled by two mechanisms: surface and sub-surface atomic diffusion mechanism (diffusive or mass-redistribution regime), which is crystallographic orientation and surface anisotropy dependent, and an erosive mechanism (erosive regime), which is incidence-angle dependent at incident angles greater than about 30-degrees for Ti alloys. We conclude, for example, that the mixed pattern of curved nano-ripples and elongated rods is the consequence of a competition between these two mechanisms. Moreover, we discovered this morphological trend with incident ion-beam angle is dominant for the α-phase in the Ti6Al4V alloy meanwhile for the 3 (bcc) phase appears insensitive to this trend, as discussed later in this paper. The interaction and competition between the two mechanisms discussed above induces pattern formation observed in FIG. 10 and influences the resultant surface properties such as hydrophilicity and hydrophobicity (see next section).

In FIG. 10 the nano-structure features observed produced by an increase of the ion-beam incident angle to 60° are very similar to those observed for 30°: nano-ripples with same orientation within a grain, but with different directions for different grains; there are also some nano-rods, confirming the mixed regime above described. Due to the increased incident angle, it appears that curved ripples are even more important than in the case of the 30° incident ion-beam angle. Again, this mixed pattern formation indicates the competition between those above-mentioned mechanisms: a diffusive regime along preferential crystallographic directions and erosive regime with nanostructure's wave vector aligned parallel the incident ion-beam. The Ti β (bcc) phase still appears insensitive to any nano-structuring due to ion irradiation at the 60° incidence.

At the highest incident angle (e.g. 80° incidence), a new trend in the resulting nano-pattern is obtained (see FIG. 10). Namely the α-phase of Ti6Al4V alloy was not the only phase material constituent responding to Ar+ion irradiation, but also the Ti β (bcc) phase. At this grazing incidence new nanoscale ripples are observed in both phases. The ripples in both phases are found to have their wave vector aligned with the ion-beam direction. The transition angle between perpendicular and parallel wave-vector ripple formation with respect to the incident ion-beam direction is therefore found to be between 60° and 80° incidence. This transition is also indicative of an observed transition for Ti-based alloys between surface and sub-surface diffusive mechanisms to erosion-dominated mechanisms dominant at grazing incidence. Furthermore, at grazing incidence the separation distance between ripples decreases resulting in well-aligned ripple structures that are about 50-nm thin and with lengths close to 0.1-0.5 μm. The morphology and direction of ripple formation are in agreement with prior results on semiconductor and single crystal materials at grazing incidence [29-44]. These results are also in agreement with the erosive regime described by the Bradley-Harper model and more recently by Yang and Allain, using atomistic simulations of Si nanopatterning [refs here]. Another important result here is that the dominating erosive processes found during grazing incidence irradiation can be achieved at room temperature. These conditions inhibit thermally-activated diffusion processes, which tend to smooth the surface and to orient the nanostructures along the preferential thermodynamic orientations [52, 53]. This specific response indicates that, under erosive sputtering conditions, it is possible to grow nanostructures, which can be aligned along thermodynamically unfavored directions.

Biological Behavior with the Nano-Structured Ti6Al4V Surfaces Synthesized by DIS

In this section an evaluation of the surface morphology modification by IBS on the Ti alloy samples is correlated to in-vitro biological testing to determine effects on cell behavior. Table 6 lists the contact angle results for samples irradiated at each listed incident ion angle measured with respect to the sample surface normal. Considering the statistics from Table 6, we note that very little change in contact angle and possibly in surface energy of the material for each irradiation condition is measured. Since the contact angles are similar, this presumes that the substrates have comparable surface free energies. However, one must be careful not to confuse strong wetting as a necessary condition for cellular adhesion. There can be other important factors and especially with inorganic systems such as metals, where intrinsic passivated oxide layers can provide for a biocompatible interface and result in positive adhesion properties for surface-cell interactions [Allain Ch. 2 Nanostructured Biointerfaces book]. With respect to these studies, no changes in adhesion in nanostructured Ti6Al4V would be expected with irradiation from surface free energy arguments. Interestingly, these results of unaltered contact angle after irradiation are consistent with that observed by Riedel et al. [48, 49], in which they performed normal Ar+etching of Ti6Al4V. The goal with these studies was to engineer surfaces on Ti6Al4V that could improve mesenchymal stem cell (MSC)-material interaction properties. The work by Riedel et al. demonstrated an overall improvement in performance of MSC interaction, though not dramatic, after ion-induced nanopatterning of the Ti alloy surfaces. The implications of these results compared to the work presented here will be discussed below.

TABLE 6
Results of contact angles testing (DI)
water on irradiated Ti6AI4V samples.

| | Incidence | Mean | Standard |
|---|---|---|---|
| Samples | angle (°) | value | deviation |
| S1 (Ti6) | 0° | 82.59 | 4.34 |
| S2 (Ti6) | 30° | 85.96 | 6.37 |
| S3 (Ti6) | 60° | 85.25 | 4.15 |
| S4 (Ti6) | 80° | 82.93 | 5.07 |

Therefore, by considering the parameters that determine free surface energy, this unaltered contact angle results can be explained as follows: the fact that contact angle basically is unaffected due to DIS, would indicate that both experimentally observed surface nano-structure and any potential surface chemistry change are not influencing surface energy; with respect to other influencing factors like crystalline system and defects, crystallographic orientation and residual stress, are weakly affected by DIS.

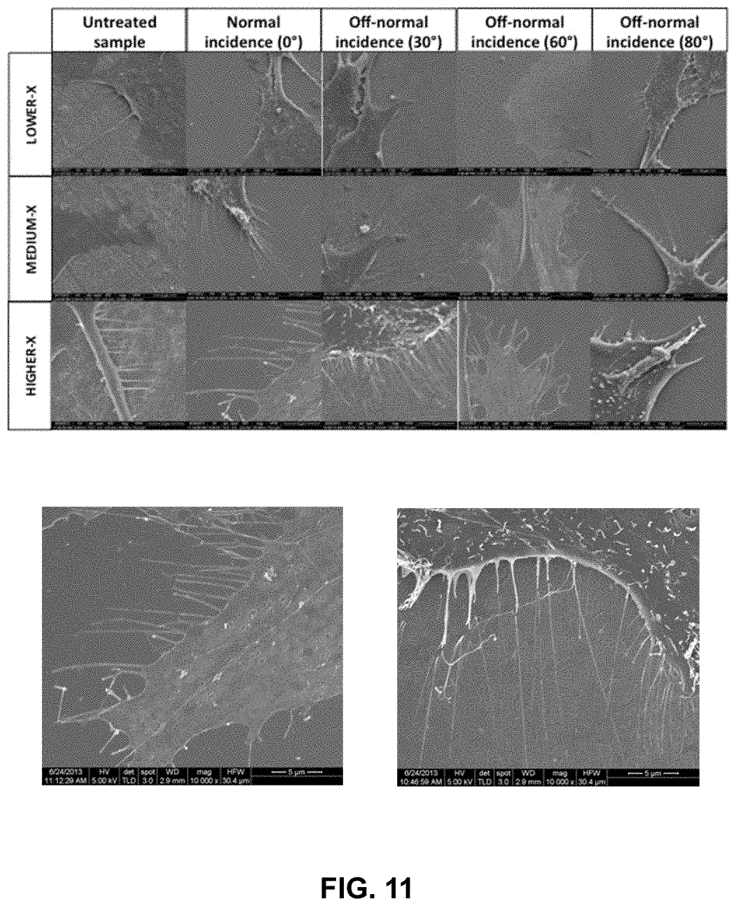

Cytotoxicity assessment of samples modified by IBS consisted of in-vitro MTT colorimetric tests (see FIG. 11), compared to a control using endothelial cells on both untreated Ti6Al4V and using the well bottom. The cell survival rate is presented for each irradiation case except for 60-degree ion incidence derived from a simple calculation of the wavelength difference from absorbance signals from a Biorad reader [42] used during MTT colorimetric testing. MTT results in FIG. 11 show high mitochondrial activity of HASMCs growing on irradiated Ti6Al4V. All of the treated samples presented cell survival percentages of factors 2-3 higher (e.g. 100-150%) compared to the control (untreated sample around 60%). This is in contrast to the results by Riedel et al., which used identical irradiation conditions as those here with the only key difference consisting of the cell line used (e.g. MSCs compared to HASMCs in this work). These results have two conclusions: 1) the cytotoxicity tests indicate improved biocompatibility of the post-irradiated surface conditions of the Ti6Al4V, and 2) the endothelial cell line (and similar mature cellular phenotypes) would be more sensitive to IBS-induced surface nanopattern formation given their propensity to better adhere to surface structures compared to more primitive cell phenotypes (e.g. MSCs). These nanostructures would provide enhanced anchor domains for the endothelial cells to interact with.

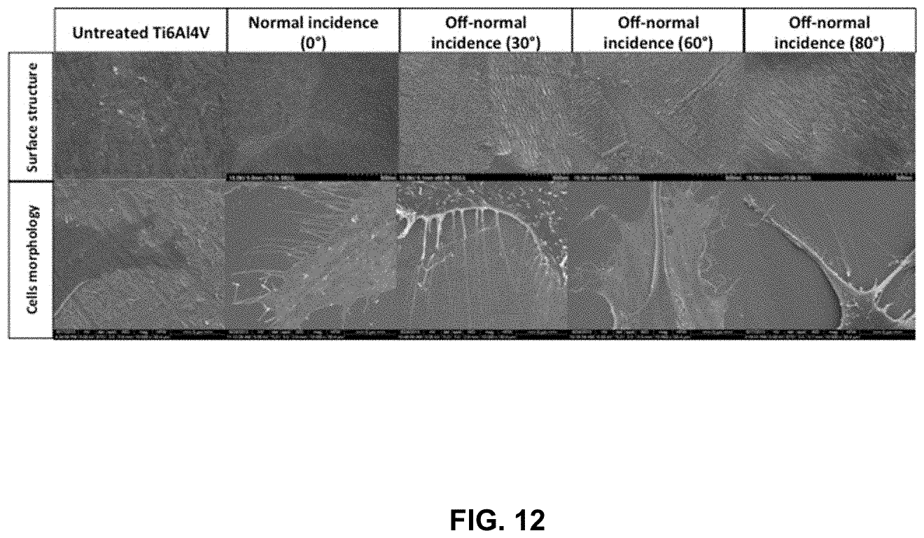

Regarding the influence of DIS surface modification of Ti6Al4V on cell/surface interactions features, all of them are included in FIG. 12; in order to correlate surface nanoscale morphology, IBS irradiation conditions and cell response. The figure is divided in three rows each defined by the magnification used with SEM, and four columns each defined by the IBS irradiation condition including the control on the first column for a sample not exposed to irradiation. The first case for the non-irradiated case the HASMCs cells are observed to spread over the intrinsic rough surface of the Ti6Al4V sample with limited dorsal surface activity and some cytoplasmic prolongations of different diameters (filopodia). Despite adhesion of these cells to the untreated surface, which conventionally has been considered acceptable, it's important to note the low number of filopodia, indicating the prevalent interaction with micro-features resulting in few focal adhesion points. This result is particularly important in that the micro-scale features, although in the spatial scale of the cell size, results in limited adhesion activity. Additional relevant aspects about lower focal adhesion points include: the clear gap between cells and substrate, as well as some holes that can be also observed in the bulk of the cells. Consequently, behavior of HASMCs cultured on untreated Ti6Al4V samples can be considered reasonably similar to that observed for similar cell phenotypes applied on polished highly biocompatible samples of cobalt-chromium (Co—Cr) alloys [54]. Comparison to the irradiated cases reveals some striking differences with the controls.

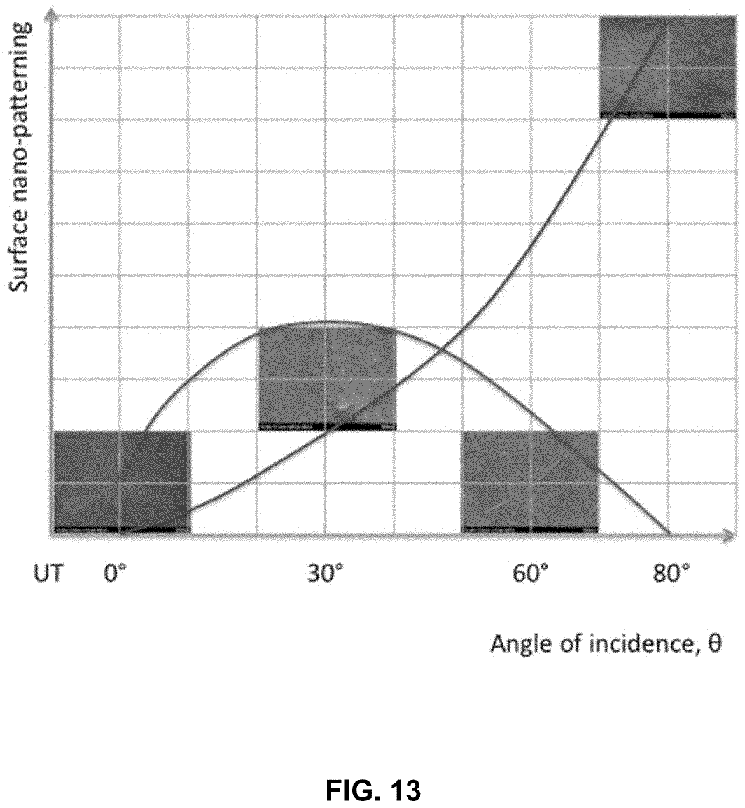

Cell morphology in contact with surfaces obtained after normal irradiation incidence showed a clear influence induced by irradiation treatment. With respect to untreated surfaces (see FIG. 12), lower magnification images enabled us to establish important changes of cell morphology and their interactions with the surface modifications. Irradiation-treated surfaces resulted in cells that appeared clearly more spread with a more irregular geometry (higher dorsal surface activity), clearly an indicator of improved cells adhesion. For SEM analysis at moderate magnification (e.g. middle-X), both dorsal surface activity (lamellipodium) and small diameter filopodia have increased with irradiation conditions at normal incidence. These improvements are found associated with an increased number of filopodia, which were not only high in number but also their diameter, on average, considerably smaller in the nanoscale range. This fact could indicate that nano-scale filopodia are strongly stimulated by the new surface nanofeatures that they are sensing (FIGS. 12 and 13). Consistent with a higher number of cell morphology features, these cells also appear with a less pronounced cell/surface vertical gap, in comparison with that observed in untreated surfaces.

A comparison between structural nano-features for normal incidence and cell morphology details (higher-magnification), allow us to point out that non-uniform distribution of nano-filopodia is due to non-uniformity of those structural nano-features (some incipient nano-ripples). Also due to the partial nano-patterning obtained with normal incidence, it is difficult to establish any relationship, from both micro and nano-scales points of view. In addition, consistent with improved adhesion, HASMCs exhibited enhanced morphology on the normal irradiated Ti6Al4V compared to a polished untreated surface.

In earlier work by Riedel et al. osteoblast cultures on irradiated Ti6Al4V samples after normal incidence with Ar+[48, 49] did not result in a measurable change in cell morphology as evidenced in the work here. In addition, it was not clear if any improvement of cells attachment was observed, contrary to the current results shown in FIGS. 12 and 13. Additional studies under irradiation conditions in this work for osteoblast proliferation and behavior is currently underway and beyond the scope of this paper. Previous work of HASMCs interactions on other biomaterials surfaces has resulted in similar cellular behavior with improved cell adhesion on nano-structured surfaces on cpTi and CoCrMo biomaterials [54]. However, these examples did not use a nanopatterned surface, instead they worked with PM nanoparticles samples without sintering; evidently, this protocol has non-negligible influences about any change of cells morphology.

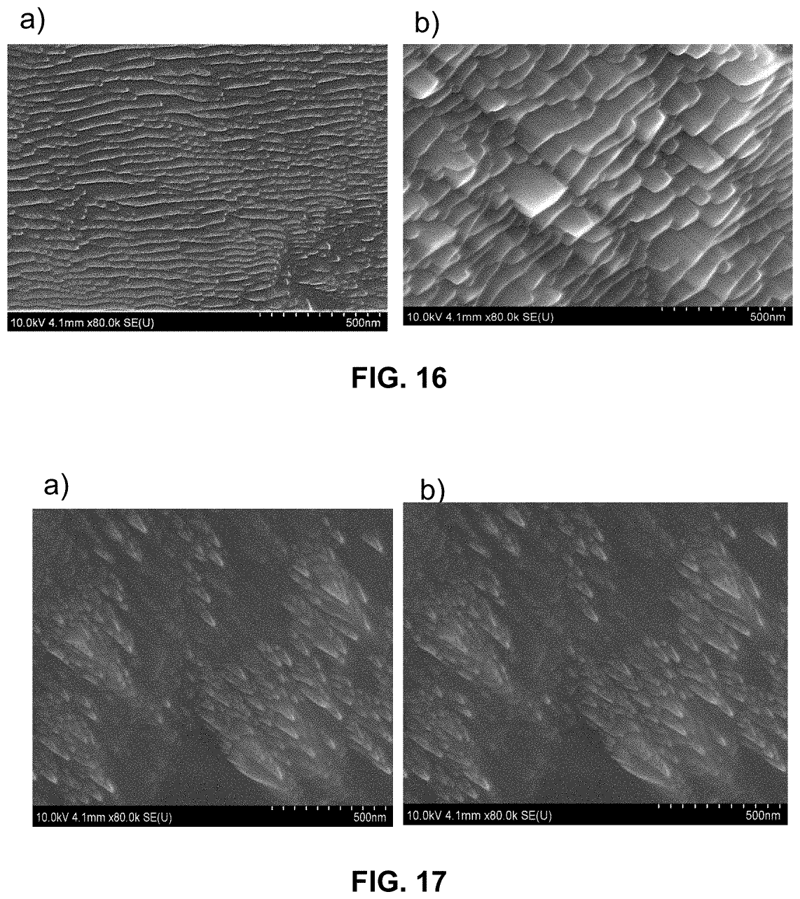

After Ti6Al4V sample irradiation with 30⁰ incidence angle, cell morphology kept basically the same trend as per normal incidence set of samples. All parameters previously observed from lowest magnification (lower-X) to highest magnification (higher-X) exhibited a growing tendency: increased lamellipodia activity, important increment of nano-filopodia, a highest spreading of cells, a better attachment, a better interaction between cells, and a whole better interaction with the nano-patterned surface. From nano-scale point of view, it was even easier to observe the improvements of cells/nano-structures interactions. Especially important are both the increment of number of nano-filopodia and the preferential site of binding with the surface; this latter result was typically observed in the grey phase of the surface (α-phase), which is predominantly nano-patterned in both nano-ripples and nano-rods. Interestingly, those nano-filopodia preferred to have nano-rippled grains as attachment sites, which could be related with high free surface energy available there for interactions and final binding. In this context, we can state at this point qualitatively that the more nano-ripples, the more nano-filopodia and, most likely, the better cell interactions and attachment to the bioactive surface (see FIG. 16).

Per definition, above increased presence of filopodia can be assumed as an important indicative of potential tissue growth of the cells in contact with the studied surfaces [55]: filopodia are temporary projections of cells membranes, and they extend and contract by the reversible assembly of actin subunits into microfilaments; it is supposed that actin polymerization is at the origin of the force propelling the cell forward. Within the same mechanism, the cell surface projects a membrane process called the lamellipodium, which is supported inside by filaments that form at the leading edge, turning into networks as they blend together. The functions of filopodia include locomotion and the capturing of nutrients. Therefore, besides all evident mechanical advantages related to filopodia, their higher number would indicate a highest actin activity and, therefore, higher mobility, and also higher capability to engulf nutrients. Indeed, in conjunction with lamellipodium activity, they indicate strong stability and interaction with surface and whole biological environment. Global cell behavior here observed indicates that untreated Ti6Al4V surfaces exhibit a clearly less adhesive force. The high dorsal activity of cells attached to DIS surfaces is also an indicator of the active state of cell phenotype development that is, at the same time, an indicator of a better differentiation response of HASMCs.

Cells morphology on surface with 60° of incidence angle exhibited similar results from both micro and nano-scales points of view (see FIGS. 12 and 13); it was observed the same high cell/surface interactions reflected in high lamellipodium activity, high interactions between cells, high number of filopodia, high spread cells, and a high overall attachment of the cell. With regards to cells nano-features, those nano-filopodia still appear preferentially attached to α-phase grains, which are the most nano-patterned ones, mostly with nano-ripples. From the micrographs there is evidence that strong crystallographic orientation governs patterning at 60⁰ incidence and this could be likely due to the maximum sputter yield as a function of incident angle for Ti from 1-keV Ar+bombardment occurring at approximately 70-degree incidence where optimized conditions for both surface diffusion and surface erosion can govern nanopatterning resulting in regions with ripples and others with nano-rods.

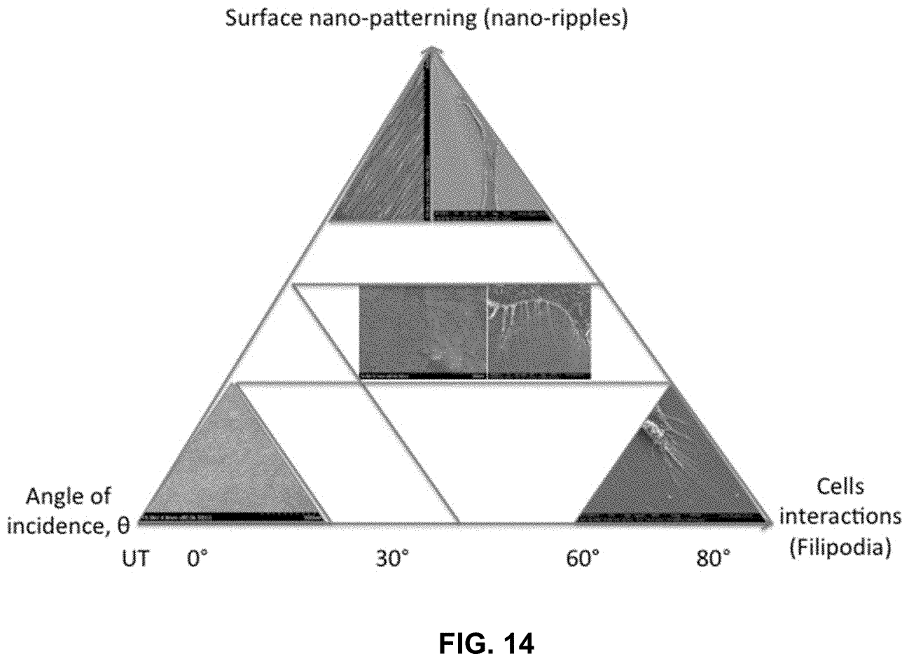

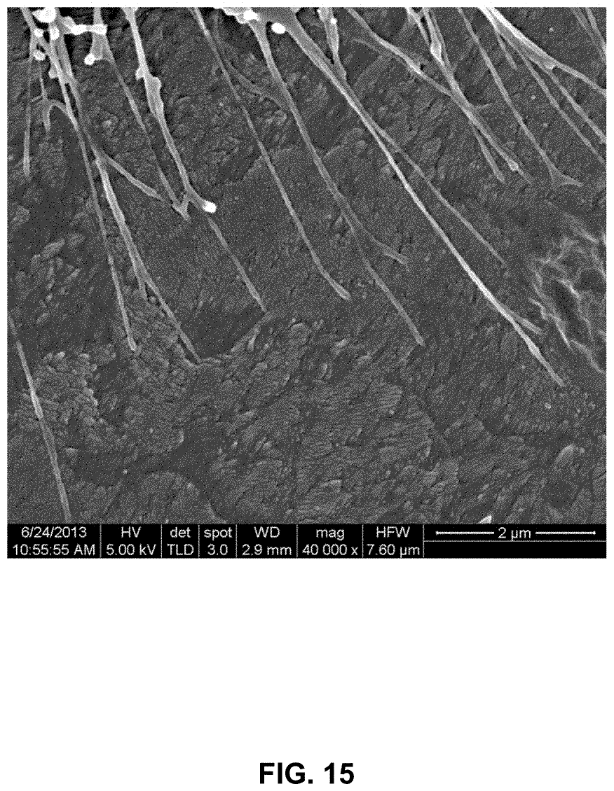

Cells/surface interactions for 80⁰ incidence angle, for which we are completely within erosive regime, micro and nano-scale features seem to indicate that positive interactions with surface show a tendency to decrease (see FIGS. 12 and 13); all above micro-level parameters seem to be reduced (lamellipodium, filopodia, interaction between cells, cells spread, and overall attachment). From nano-scale point of view, this incidence angle seems to produce a lower number of nano-filopodia; despite these are still preferentially located at α-phase, they appear mostly oblique to nano-rods direction due to new shape of those nano-features associated to erosive regime (straight and narrow elongated nano-rods). This specific response can be a consequence of the lowest free surface energy of these highly narrow nano-rods. Global analysis of stated relationships between incidence angles of Ar+ions beams, surface nano-patterning, and cells/surface interactions, has allowed us to elaborate different maps and master-diagrams that are rather useful to summarize the most important phenomenological aspects here determined (FIGS. 13 to 15).

Results obtained here about improvements of cells/surface interactions due to DIS nano-patterning of Ti6Al4V can be considered in agreement with several previous efforts about study of interactions with different kind of cells with surface nano-structures obtained by other advanced techniques [3-11]; however, results reported here are new in the sense that is the first time is shown a real relationship between ion irradiation nano-structure and some micro and nano-scale parameters of cells; they are related not only with their behavior and adhesion, but also with their further potential to stimulate tissue growth.

The influence of surface nano-structures on cells behavior has been studied by using many surfaces and cell types, such as MSC osteoblasts, fibroblasts, and some others [2]; the goal of nanostructures is to mimic the in vivo environment for cell growth and proliferation. As it can be easily verified in those works, most of those studies tried about influence of regular, periodic, nano-patterns on different aspects of cells behavior. A variety of patterns ranging from topographical to chemical have been shown to have an effect on cell adhesion, proliferation, alignment and gene expression, demonstrating that the nano-scale of the surface plays a role in determining cell behavior. Several works have shown that integrin-ligand binding is followed by the formation of focal adhesions and actin stress fibers that enhance and strengthen the cell adhesion to the surface by the recruitment of additional proteins such as the focal adhesion kinase or FAK [56]. These findings are related to relevance of filopodia presence in contact with a nano-patterned surface with respect to potential stimulus for tissue growth. A well-spread morphology, similar to that observed (see FIGS. 12 and 13) and a well-developed cytoskeleton have been observed for a nano-island height of 13 nm [57, 58]. Membrane protrusions, like filopodia, are suggested to have a sensing role and allow cells to react to the topography [2]. In addition, cells cultured on nano-pillars or nanopits usually have reduced levels of cytoskeletal organizations, and reduced adhesion, suggesting that the cells on these structures are motile and sensing rather than settled and anchored to the surface [58]; this is a theory supported by several reports demonstrating increased migration on nano-pillars. As a general rule, the effects are more pronounced for smaller, nano-sized surface features, although there is a lack of knowledge on the basic mechanisms the cell uses to detect and respond to this nanotopography.

Finally, from a comparison of the technique implemented here of Ar+ions irradiation, as well as with surface nano-structures and the interactions with HASMCs, with some above reported results, we must remark the following before to get some final conclusions: 1. Our results are consistent with those reported improvements of cells response in contact with nano-patterned surfaces; that is reflected in observed increment of filopodia activity due to interactions with surfaces here developed. This is even more important, because of the straight relationship between filopodia and actin activity, with a potential of these attached cells for stimulation of further tissue growth; 2. This is the first work in which is detailed analyzed relationships between incidence angle and nano-patterning of Ti6Al4V, as well as with cells changes on those surfaces, from micro and nan-scale levels; 3. In contrast to those methodologies of surface nano-patterning used in those previous works (mostly limited to create regular-periodic and restricted patterns), DIS technology exposed here offers unique advantages like massive surface transformation by self-organized atoms mechanisms, produced in a few seconds of irradiation, with the potential to control also any desired surface chemistry; this features become it in an advanced process far away to be the same as conventional ion etching or bombardment.

### Conclusions

The study performed here about different nano-patterns of Ti6Al4V obtained by DIS with Ar+ with different incident angles, and their interactions with HASMCs, has allowed us to draw the following conclusions about their potential for tissue growth stimulation:

1. All incidence angles joined with rest of DIS parameters used for surface treatment by DIS of Ti6Al4V were able to produce certain surface nano-pattern, which followed three regimes depending of controlling mechanisms to produce those nano-structures: 1. Diffusive regime (normal and close to normal incidences): this was characterized by generation of incipient colonies of nano-ripples, which can be also assumed as nano-cracks colonies; these colonies are the consequence of slightly happening of a diffusive mechanism; 2. Mixed regime, diffusive+erosive (closely normal incidence up to approximately 60⁰): erosive mechanism becomes more important as the incidence angle is increased; in consequence, it is obtained a mixed nano-structure, nano-ripples and nano-rods, in which proportion of nano-rods due to erosive regime is incrementally higher as the angle is increased; 3. Erosive regime (incidence angles of 80⁰ and higher): prevalence of this mechanism is reflected in the predominant obtained nano-pattern of straight long nano-rods, which also appears very narrow and highly packed. The presence of these different nano-patterns attributed to those different regimes, can be assumed as an indicator of validity of the Bradley-Harper model for DIS conditions used to irradiate Ti6Al4V.

2. Both crystalline phases and crystallographic orientation of alloy grains were determinant for final nano-structure and its orientation, for each incidence angle. During diffusive and diffusive erosive regimes (0 to 60 angles), alpha-phase (hcp) grains were basically the only ones that responded to nano-structuring to DIS. Orientation of both nano-ripples and nano-rods within these grains exhibited different directions due to different crystallographic orientations of them. In contrast, during complete erosive regime (angles >80) both kinds of grains (hcp and bcc) responded in a similar manner, generating those mentioned long straight nano-rods. This crystal insensitive nano-patterning can be attributed to severe controlled damage produced during erosive regime.

3. Analysis of biological response of above DIS surfaces in contact with HASMCs showed important influence of the different nano-patterns obtained, with respect to fundamental parameters of cellular behavior. Firstly, interactions between HASMCs and the nano-patterned surfaces exhibited overall improvement of cells behavior reflected in a high cytocompatibility response, besides highest positive parameters with respect to untreated surfaces: higher surface dorsal activity (lamellipodiums); higher cytoplasm prolongations (filopodia), higher interactions between cells, and mostly a reduced vertical gap between cells and the surface, as well as high spread cells shape, and better interactions with surface nano-features; all reflected in a whole better cells attachment. However, a detailed analysis of surface nano-features influence on cells response allowed us to observe a real consistency with surface nano-patterns obtained with different angles, different regimes, especially with respect to filopodia activity: despite all irradiated angles used were able to produce important number of filopodia, the detailed analysis allowed us to establish that curved nano-ripples were the better features to stimulate presence of filopodia. In that sense, as long as the regime allow to obtaining those nano-ripples, it was evident increasing filopodia activity. Therefore, filopodia increase up to approximately 60° incidence; then, it seems to become stable and even start to decrease at an approximate 80° incidence angle. This filopodia response would indicate that their presence is directly related with prevalence of diffusive regime and, therefore, optimum angle for their presence would be between 30° and 60° incidence angles.

4. Despite evaluation of potential stimulation of tissue growth due to these nano-patterned Ti6Al4V surfaces requires further and more detailed studies, criteria of filopodia existence as indicator of potential stimulus for tissue growing can be assumed as a valid one. Per definition, filopodia consists of temporary projections of cells membranes, and they extend and contract by the reversible assembly of actin subunits into microfilaments. The functions of filopodia include locomotion and the capturing of nutrients. Within this context, besides all evident mechanical advantages related to filopodia, their higher number would indicate highest actin activity and, therefore, higher mobility, and also higher capability to engulf nutrients. Indeed, in conjunction with lamellipodium, they indicate strong stability and interaction with surface and whole biological environment. The high dorsal activity of cells attached to DIS surfaces is also an indicator of the active state of cell phenotype development that is, at the same time, an indicator of a better differentiation response of HASMCs.

5. Our work reported here has allowed us to establish, for the first time, different phenomenological relationships between incidence angle, nano-structures, and cells adhesion, which have been illustrated through using some new maps and master diagrams schemes. These phenomenological trends allowed us to have a new semi-quantitative tool for predictions of HASMCs response in terms of processing and surface properties, which will be an important insight for the current work in which our group in involved about developing of new multifunctional surface by DIS for proper cells stimulation for further tissue growth and repair.

6. The most important result here is that specific phenotypic behavior can be correlated to the ion-irradiation induced type of pattern morphology. In the work by Riebel et al. the irradiation-induced patterns in Ti-based alloys did not lead to a significant effect on cell-surface interaction for mesenchymal cells although cell spreading was evidenced. However, in our work nearly identical irradiation treatment of the same Ti-based alloy results in significant effects on cellular behavior (in this case endothelial cells types) even though no apparent changes are made to the contact angle properties in either experiment. This is consistent with the hypothesis that cell spreading is correlated to both a cell's ability to spread based on its phenotypic behavior and the elasto-mechanical interactions with protrusions from 2D surfaces.

### REFERENCES

[1] Biomaterials, artificial organs and tissue engineering, Edited by Larry L. Hench and Julian R. Jones, Woodhead Publishing Series in Biomaterials No. 4, Imperial College of Science, Technology and Medicine, U K, 2005.
[2] Advances in Regenerative Medicine: Role of Nanotechnology and Engineering Principles, Chapter 12. Materials Surface Effects on Biological Interactions, Josep A. Planell, Melba Navarro, George Altankov, Conrado Aparicio, Elisabeth Engel, Javier Gil, Maria Pau Ginebra, and Damien Lacroix, V. P. Shastri et al. (eds.), pp. 233, Springer, The Netherlands, 2007.
[3] Nanotopographical modification: a regulator of cellular function through focal adhesions, Manus Jonathan Paul Biggs, R. Geoff Richards, Matthew J. Dalby, Nanomedicine: Nanotechnology, Biology, and Medicine 6 (2010) 619-633.
[4] Materials in Regenerative Medicine, V. Prasad Shastri and Andreas Lendlein, Adv. Mater. 2009, 21, 3231-3234.
[5] Brown R A, Phillips J B (2007) Cell responses to biomimetic protein scaffolds used in tissue repair and engineering. Int Rev Cytol 262:75-150.
[6] Deligianni, D. D.; Katsala, N. D.; Koutsoukos, P. G. & Missirlis, Y. F. (2001). Effect of surface roughness of hydroxyapatite on human bone marrow cell adhesion, proliferation, differentiation and detachment strength. Biomaterials, 22, 1, (January 2001) 87-96, ISSN0142-9612.
[7] Dalby, M. J., N. Gadegaard, R. Tare, A. Andar, M. O. Riehle, P. Herzyk, C. D. Wilkinson, and R. O. Oreffo, 2007, The control of human mesenchymal cell differentiation using nanoscale symmetry and disorder: Nature materials, v. 6, p. 997-1003.
[8] Andersson, A.-S., J. Brink, U. Lidberg, and D. S. Sutherland, 2003, Influence of systematically varied nanoscale topography on the morphology of epithelial cells: NanoBioscience, IEEE Transactions on, v. 2, p. 49-57.
[9] Martines, E., K. Seunarine, H. Morgan, N. Gadegaard, C. D. Wilkinson, and M. O. Riehle, 2005, Superhydrophobicity and superhydrophilicity of regular nanopatterns: Nano letters, v. 5, p. 2097-2103.
[10] Sjostrom T, Dalby M J, Hart A, Tare R, Oreffo R O, Su B. Fabrication of pillar-like titania nanostructures on titanium and their interactions with human skeletal stem cells. Acta Biomater 2009; 5:1433-41.
[11] Lu J, Rao M P, MacDonald N C, Khang D, Webster T J. Improved endothelial cell adhesion and proliferation on patterned titanium surfaces with rationally designed, micrometer to nanometer features. Acta Biomater 2008; 4:192-201.
[12] “Dip-Pen” Nanolithography, Richard D. Piner, Jin Zhu, Feng Xu, Seunghun Hong, Chad A. Mirkin, Science 29 Jan. 1999: Vol. 283 no. 5402 pp. 661-663.
[13] Energy spectra of quantum rings, A. Fuhrer, S. LUscher, T. Ihn, T. Heinzel, K. Ensslin, W. Wegscheider & M. Bichler, Nature 413, 822-825 (25 Oct. 2001).
[14] Chan, W. L., and E. Chason, 2007, Making waves: Kinetic processes controlling surface evolution during low energy ion sputtering: Journal of applied physics, v. 101, p. 121301-121301-46.
[15] Erlebacher, J., M. J. Aziz, E. Chason, M. B. Sinclair, and J. A. Floro, 1999, Spontaneous pattern formation on ion bombarded Si (001): Physical review letters, v. 82, p. 2330.
[16] Facsko, S., T. Dekorsy, C. Koerdt, C. Trappe, H. Kurz, A. Vogt, and H. L. Hartnagel, 1999, Formation of ordered nanoscale semiconductor dots by ion sputtering: Science, v. 285, p. 1551-1553.
[17] Frost, F., and B. Rauschenbach, 2003, Nanostructuring of solid surfaces by ion-beam erosion: Applied Physics A, v. 77, p. 1-9.
[18] Frost, F., A. Schindler, and F. Bigl, 2000, Roughness evolution of ion sputtered rotating InP surfaces: pattern formation and scaling laws: Physical review letters, v. 85, p. 4116.
[19] Kim, T., M. Jo, Y. Kim, D. Noh, B. Kahng, and J.-S. Kim, 2006, Morphological evolution of ion-sputtered Pd (001): Temperature effects: Physical Review B, v. 73, p. 125425.
[20] Makeev, M. A., and A.-L. Barabasi, 1997, Ion-induced effective surface diffusion in ion sputtering: Applied physics letters, v. 71, p. 2800-2802.
[21] Qian, J., Y. Kang, Z. Wei, and W. Zhang, 2009, Fabrication and characterization of biomorphic 45S5 bioglass scaffold from sugarcane: Materials Science and Engineering: C, v. 29, p. 1361-1364.
[22] Ziberi, B., F. Frost, B. Rauschenbach, and T. Hoche, 2005, Highly ordered self-organized dot patterns on Si surfaces by low-energy ion-beam erosion: Applied Physics Letters, v. 87, p. 033113-033113-3.
[23] Harper, J., and K. Rodbell, 1997, Microstructure control in semiconductor metallization: Journal of Vacuum Science & Technology B: Microelectronics and Nanometer Structures, v. 15, p. 763-779.
[24] Krasheninnikov, A., and F. Banhart, 2007, Engineering of nanostructured carbon materials with electron or ion beams: Nature materials, v. 6, p. 723-733.
[25] Vo, N., R. Averback, P. Bellon, S. Odunuga, and A. Caro, 2008, Quantitative description of plastic deformation in nanocrystalline Cu: Dislocation glide versus grain boundary sliding: Physical Review B, v. 77, p. 134108.
[26] Barth, J. V., G. Costantini, and K. Kern, 2005, Engineering atomic and molecular nanostructures at surfaces: Nature, v. 437, p. 671-679.
[27] Allain, J., M. Nieto, M. Hendricks, P. Plotkin, S. Harilal, and A. Hassanein, 2007, IMPACT: A facility to study the interaction of low-energy intense particle beams with dynamic heterogeneous surfaces: Review of Scientific Instruments, v. 78, p. 113105-113105-14.
[28] Nanostructured Biointerfaces, Jean Paul Allain, M. Echeverry, S. Arias, J. Pavon, Chapter in: 2013 CMOS Emerging Technologies Research Symposium Book, submitted.
[29] Chan W L, Chason E. Kinetics of ion-induced ripple formation on Cu (001) surfaces.
[30] H. X. Qiana, W. Zhou. Self-organization of ripples on Ti irradiated with focused ion beam. Applied Surface Science 258 (2012) 1924-1928.
[31] Carsten Busse, Cemal Engin, Henri Hansen, Udo Linke, Thomas Michely, Herbert M. Urbassek. Adatom formation and atomic layer growth on Al(111) by ion bombardment: experiments and molecular dynamics simulations. Surface Science, Volume 488, Issue 3, 10 Aug. 2001, Pages 346-366
[33] J. Naumann, J. Osing, A. J. Quinn, I. V. Shvets. Morphology of sputtering damage on Cu(111) studied by scanning tunneling microscopy. Surface Science, Volume 388, Issues 1-3, 23 Oct. 1997, Pages 212-219
[34] G Costantini, S Rusponi, F Buatier de Mongeot, C Boragno and U Valbusa. Periodic structures induced by normal-incidence sputtering on Ag(110) and Ag(001): flux and temperature dependence
[35] O Malis, J D Brock, R L Headrick, M S Yi, J M Pomeroy. Ion-induced pattern formation on Co surfaces: An x-ray scattering and kinetic Monte Carlo study. Physical Review B, 2002—APS
[36] M. V. Ramana Murty, T. Curcic, A. Judy, and B. H. Cooper. X-Ray Scattering Study of the Surface Morphology of Au(111) during Ar+Ion Irradiation. Physical review letters volume 80, number 21. 25 May 1998
[37] Matthias Kalff, George Comsa, Thomas Michely. Temperature dependent morphological evolution of Pt(111) by ion erosion: destabilization, phase coexistence and coarsening. Surface Science, volume 486, Issues 1-2, 1 Jul. 2001, Pages 103-135
[38] P. Karmakar, G. F. Liu, J. A. Yarmoff. Sputtering-induced vacancy cluster formation on TiO₂ (110). Physical review B 76, 193410 (2007).
[39] Sebastiaan van Dijken, Dennis de Bruin, and Bene Poelsema. Kinetic Physical Etching for Versatile Novel Design of Well Ordered Self-Affine Nanogrooves. Phys. Rev. Lett. 86, 4608-4611 (2001)
[40] Tim Luttrell and Matthias Batzill. Nanoripple formation on TiO2 (110) by low-energy grazing incidence ion sputtering. Phys. Rev. B 82, 035408 (2010).
[41] ASM International, “Materials Properties Handbook: Titanium Alloys”, Eds. Boyer, R., Welsh, G. y Collings, E. W., ASM International, O H, (1994).
[42] Navez M, Sella Cand, Chaperot D, 1962 C. R. Acad. Sci., Paris 254, 240.
[43] Ion Beam Sputtering: A Route for Fabrication of Highly Ordered Nanopatterns, Marina Cornejo, Jens Völlner, Bashkim Ziberi, Frank Frost and Bernd Rauschenbach, F. A. Lasagni and A. F. Lasagni (eds.), Fabrication and Characterization in the Micro-Nano Range, Advanced Structured Materials, Springer-Verlag Berlin Heidelberg 2011.
[44] Surface Morphology Evolution of During Low Energy Ions Bombardment of Silicon and Gallium Antimonide, Gozden Ozaydin-Ince, Ph. Thesis, Boston University, College of Engineering, 2008.
[45] Nanostructuring surfaces by ion sputtering, U Valbusa, C Boragno, F Buatier de Mongeot, Phys.: Condens. Matter 14 (2002) 8153-8175.
[46] Plasma processing for nanostructured topographies, Nicholas Alfred Riedel, Ph.D. Thesis, Department of Mechanical Engineering, Colorado State University, Fort Collins, Colo., 2012.
[47] Ion beam etching titanium for enhanced osteoblast response, Nicholas A. Riedel, John D. Williams, Ketul C. Popat, J Mater Sci (2011) 46:6087-6095.
[48] Politi P and Villain J 1996 Phys. Rev. B545114.
[49] Bradley, R. M., Harper, J. M. E.: Theory of ripple topography induced by ion bombardment. J. Vac. Sci. Technol. A 6(4), 2390-2395 (1988).
[50] Carter, G., Nobes, M. J., Paton, F., Williams, J. S., Whitton, J. L.: Ion bombardment induced ripple topography on amorphous solids. Radiat. Eff. Defects Solids 33, 65-73 (1977).
[51] Frost, F., Schindler, A., Bigi, F.: Roughness evolution of ion sputtered rotating InP surfaces: pattern formation and scaling laws. Phys. Rev. Lett. 85(19), 4116-4119 (2000).
[52] Increased endothelial and vascular smooth muscle cell adhesion on nanostructured titanium and CoCrMo, Saba Choudhary, Mikal Berhe, Karen M Haberstroh, Thomas J Webster, International Journal of Nanomedicine 2006:1(1) 41-49 41.
[53] Aparicio Badenas C, Gil F X (2005) Tratamientos de superficie sobre titanio comercialmente puro para la mejora de la osteointegracio n de los implantes dentales. In: Universitat Polite'cnica de Catalunya. Departament de Cie'ncia dels Materials i Enginyeria Metallu'rgica. http://hdl.handle.net/10803/6044. Accessed 15 Apr. 2005.
[54] McNamara, L. E., R. J. McMurray, M. J. Biggs, F. Kantawong, R. O. Oreffo, and M. J. Dalby, 2010, Nanotopographical control of stem cell differentiation: Journal of Tissue Engineering, v. 1.
[55] Dalby, M., M. Riehle, H. Johnstone, S. Affrossman, and A. Curtis, 2002a, in vitro reaction of endothelial cells to polymer demixed nanotopography: Biornaterials, v. 23, p. 2945-2954.
[56] Dalby, M. J., G. E. Marshall, H. J. Johnstone, S. Affrossman, and M. O. Riehle, 2002b, Interactions of human blood and tissue cell types with 95-nm-high nanotopography: NanoBioscience, IEEE Transactions on, v. 1, p. 18-23.

### Example 3: Summary of Titanium Nanostructuring Approach

The following is a description of some aspects of the method to generate the compositions of matter and surfaces along with specific functions elicited by the same. A conceptual connection to the surface to be claimed can be illustrated as in Scheme 1:

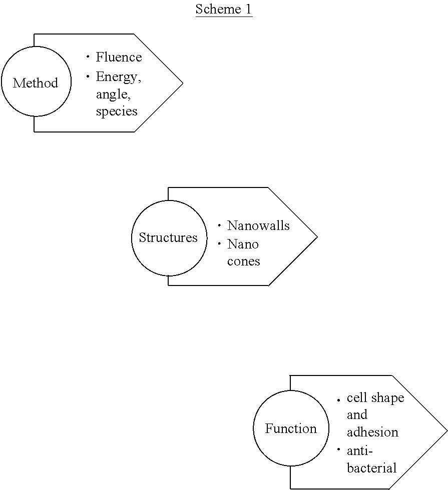

The wide scope and variety of surface structures can be synthesized by either DIS or DPNS, depending on desired function or exemplary embodiments of structure and function. For example, to elicit an immune-modulated response for macrophage phenotype that have anti-inflammatory osseoconductive properties, an exemplary embodiment of these structures would be made of medical-grade Ti alloy exposed to a particular fluence, angle of incidence, energy and species in the energetic particle beams from either DIS or DPNS methods. We organize by surfaces having structures defined with: topography, microstructure and surface composition.

1. Nanotopography:

Based on the different experimental parameters, an array of different nanostructures can be induced. The nanostructures were obtained as function of energetic particle species, fluence and incident angle with respect to the surface normal. For reasons of organization structures are organized by principal particle-beam gas species.

Microstructure needs to be defined in anatase and rutile phases.

1.1 Parameter: Gas (Kr)

a) Parallel Nano-walls (Kr): an array of (parallel) wall like structures having a height of 3 to 250 nm, with a length ranging from 10 to 40 nm, said ridge composed primarily of titanium alloy (FIGS. 16a and 16b). Such structures are formed using a method comprising of Kr ions, incident at an angle with respect to surface normal=60°, Energy: 1 keV, Fluence: 1×10¹⁸ cgs with an effective surface area of 2-5×10¹⁶ nm².

b) Nano-cones (Kr): Structures which resemble sharp-like cones. These pointed sharp regions are inclined towards the incident beam direction (FIGS. 17a and 17b). The dimension of these cones are in the range of 2 to 100 nm, with a length ranging from 10 to 40 nm. These cones consist of Ti alloy. Such structures are formed using following parameters, Gas: Kr, Angle: 60°, Energy: 1 keV, Fluence: 1×10¹⁸ cgs.

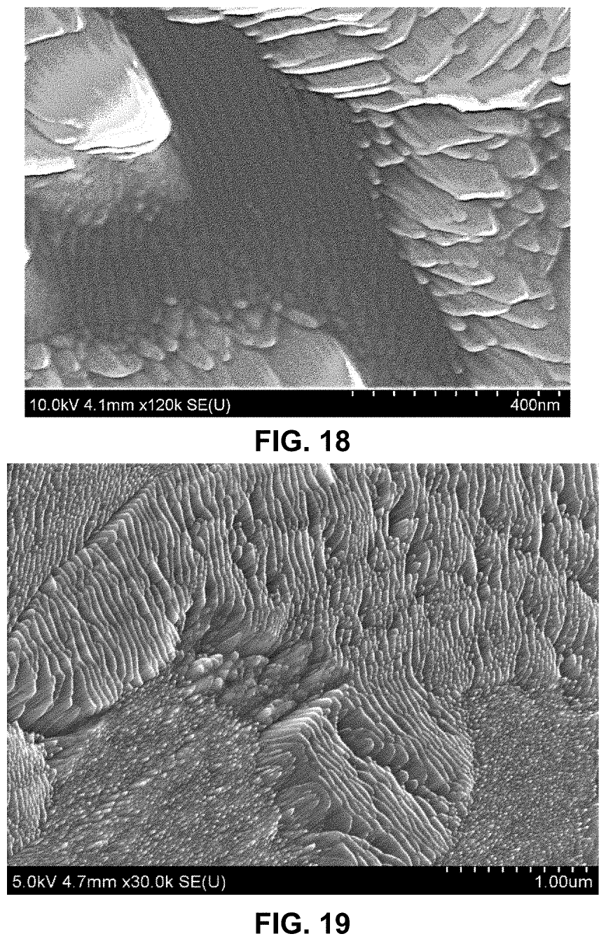

c) Nano-ripples (Kr): In the midst of nano-pillar and nano-cones, we also observe nanoripples as shown in FIG. 18. Such structures are formed using following parameters, Gas: Kr, Angle: 60°, Energy: 1 keV, Fluence: 1×10¹⁸ cgs.

1.2 Parameter: Gas (Ar)

a) Nano-walls (Ar): An array of (parallel) wall like structures having a height of 3 to 100 nm, with a length ranging from 10 to 40 nm, said ridge composed primarily of titanium alloy (FIG. 19). Such structures are formed using following parameters—Gas: Ar, Angle: 60°, Energy: 1 keV, Fluence: 1×10¹⁸ cgs.

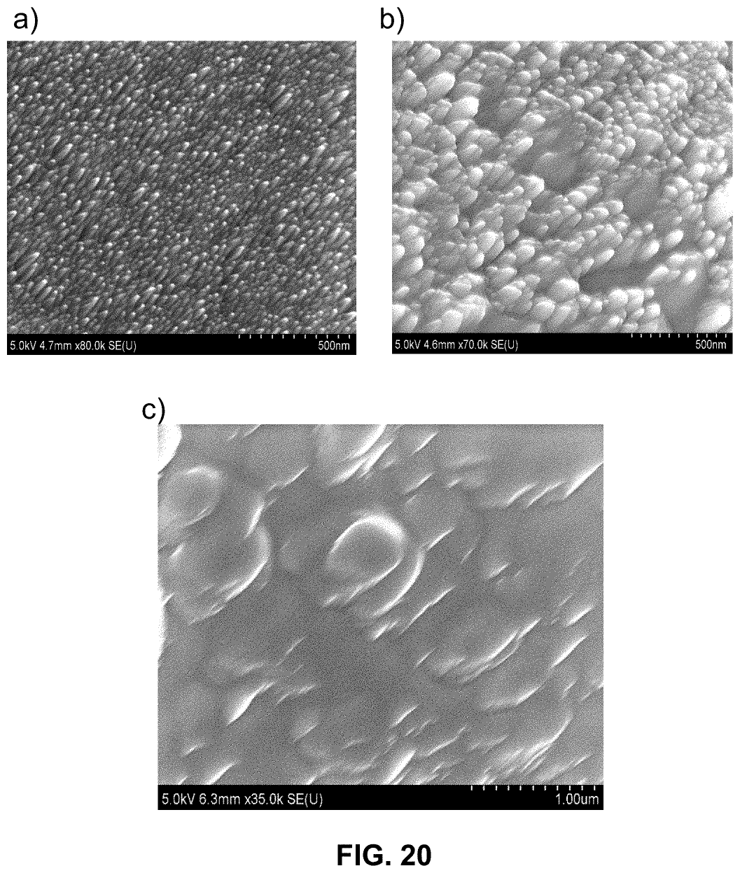

b) Nano-cones (Ar): Structures which resemble sharpe-like cones. These pointed sharp regions are inclined towards the incident beam direction (FIGS. 20a-20b). The dimension of these cones are in the range of 2 to 100 nm, with a length ranging from 10 to 40 nm. These cones consist of Ti alloy. Such structures are formed using following parameters—Gas: Ar, Angle: 60°, Energy: 1 keV, Fluence: 1×10¹⁸ cgs.

Round-plate formation (Ar): The structures resemble round plate formation (FIG. 20c). The diameter is around 50 nm. Such structures are formed using following parameters, Gas: Ar, Angle: 60°, Energy: 1 keV, Fluence: 1×10¹⁸ cgs.

1.3 Parameter: Fluence 7.5×10¹⁷ cgs (Ar)

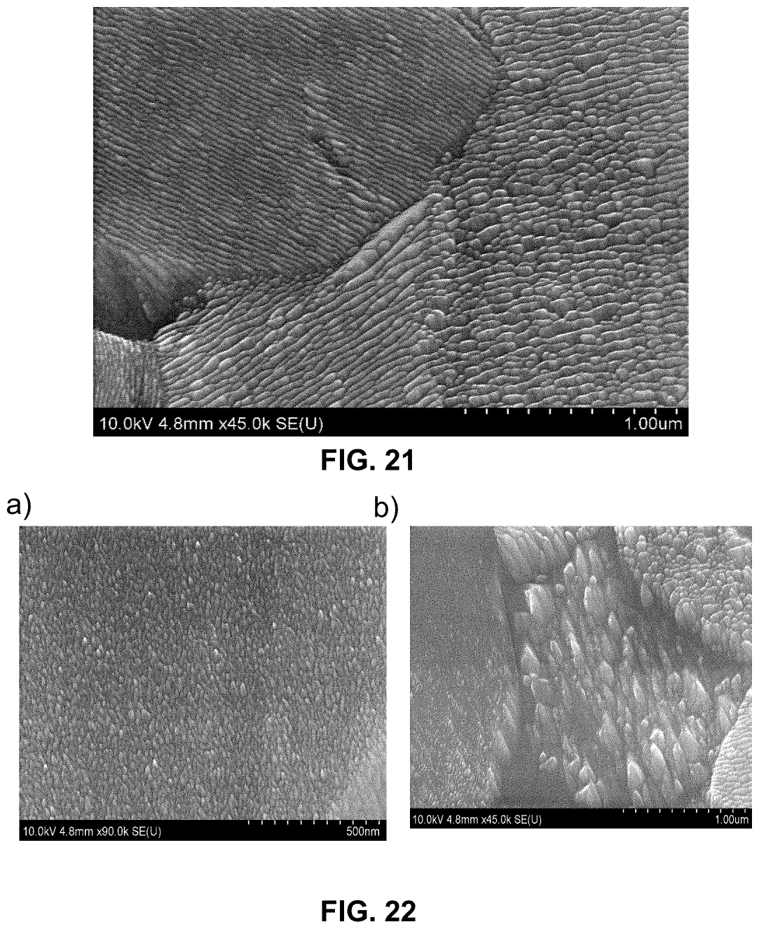

a) Nano-walls (Ar): An array of (parallel) long and short wall like structures having a height of 3 to 100 nm preferably, with a length ranging from 10 to 40 nm, said ridge composed primarily of titanium alloy (FIG. 21). Such structures are formed using following DIS parameters—Gas: Ar, Angle: 60°, Energy: 1 keV, Fluence: 7.5×10¹⁷ cgs. The walls are oriented in different directions and sizes.

b) Nano-cones (Ar): Structures consist of very narrow and wide cones (FIGS. 22a-22b). These cones consist of Ti alloy. Such structures are formed using following DIS parameters—Gas: Ar, Angle: 60°, Energy: 1 keV, Fluence: 7.5×10¹⁷ cgs.

1.4 Parameter: Fluence 5×10¹⁷ cgs (Ar)

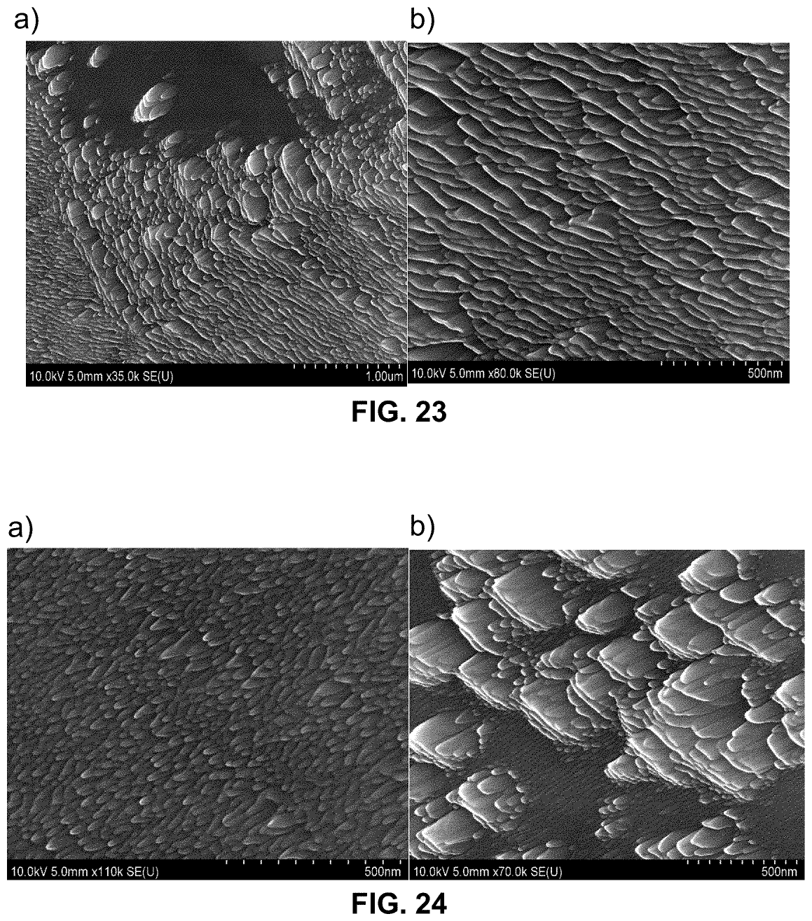

a) Nano-walls (Ar): An array of (parallel) long and short wall like structures having a height of 3 to 100 nm preferably, with a length ranging from 10 to 40 nm, said ridge composed primarily of titanium alloy (FIGS. 23a-23b). Such structures are formed using following DIS parameters—Gas: Ar, Angle: 60°, Energy: 1 keV, Fluence: 5×10¹⁷ cgs. The walls are oriented in different directions and sizes.

b) Nano-cones (Ar): Structures consist of very narrow and wide cones (FIGS. 24a-24b). These cones consist of Ti alloy. Such structures are formed using following DIS parameters—Gas: Ar, Angle: 60°, Energy: 1 keV, Fluence: 5×10¹⁷ cgs.

1.5 Parameter: Fluence 2.5×10¹⁷ cgs (Ar)

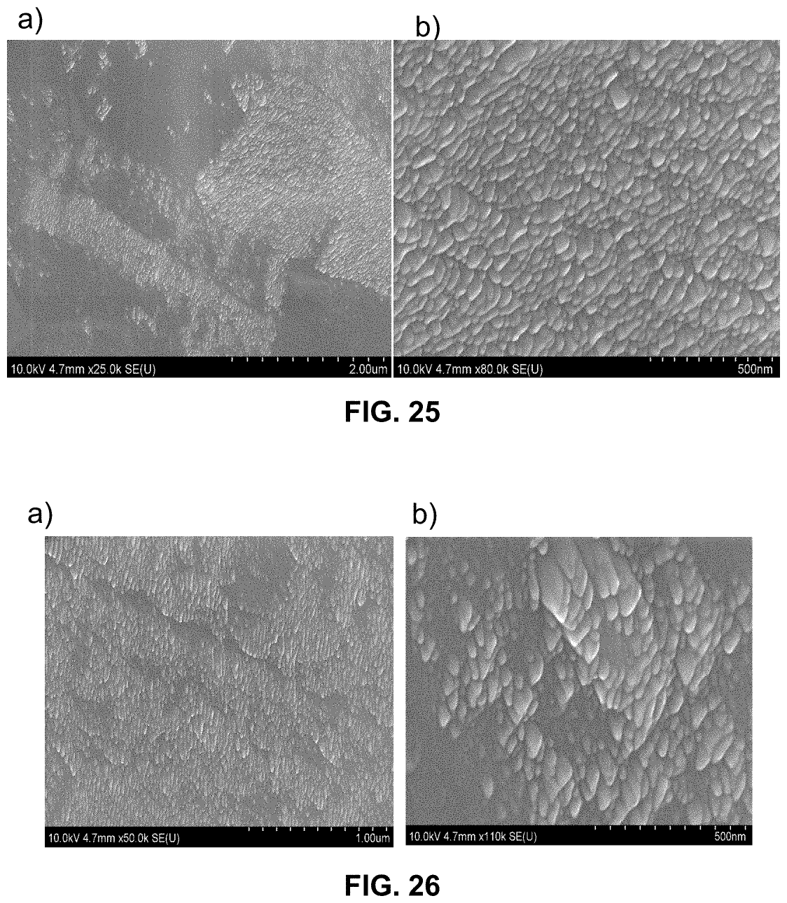

a) Nano-walls (Ar): The surface consists of smooth and nanostructured surface composed primarily of titanium alloy (FIGS. 25a-25b). Such structures are formed using following DIS parameters-Gas: Ar, Angle: 60°, Energy: 1 keV, Fluence: 2.5×10¹⁷ cgs. The walls are oriented in different directions and sizes.

b) Nano-cones (Ar): The surface consists of smooth and nanostructured surface. Structures consist of very narrow and wide cones (FIGS. 26a-26b). These cones consist of Ti alloy. Such structures are formed using following DIS parameters—Gas: Ar, Angle: 60°, Energy: 1 keV, Fluence: 2.5×10¹⁷ cgs.

1.6 Parameter: Fluence 1×10¹⁷ cgs (Ar)

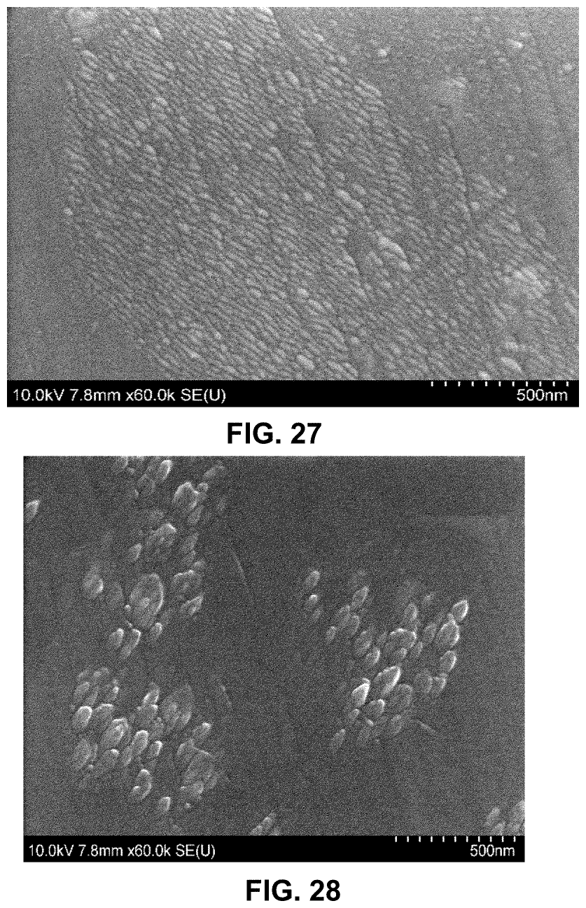

a) Nano-walls (Ar): The surface consists of fine nanostructures which are seen at specific regions on the surface. Due to their fine nature we cannot measure their dimensions. These structures are composed primarily of titanium alloy (FIG. 27). Such structures are formed using following DIS parameters-Gas: Ar, Angle: 60°, Energy: 1 keV, Fluence: 2×10¹⁷ cgs.

b) Nano-cones (Ar): Structures consist of cones which are non-uniform on the surface (FIG. 28). These cones consist of Ti alloy. Such structures are formed using following DIS parameters—Gas: Ar, Angle: 60°, Energy: 1 keV, Fluence: 5×10¹⁷ cgs.

1.7 Angles 0° to 80° (Ar)

TABLE 7
Irradiation Parameters (1-keV Ar⁺) on Ti6AI4V Samples

| | Energy | Flux (ions sec | Fluence | Incidence | |
|---|---|---|---|---|---|
| Samples | (keV) | cm⁻²) | (cm⁻²) | angle (°) | Time (s) |
| S1 (Ti6) | 1.0 | 6.51E14 | 2.5E17 | 0° | 384.0 |
| S2 (Ti6) | 1.0 | 3.29E14 | 2.5E17 | 30° | 758.8 |
| S3 (Ti6) | 1.0 | 6.44E14 | 2.5E17 | 60° | 387.9 |
| S4 (Ti6) | 1.0 | 6.55E14 | 2.5E17 | 80° | 381.6 |

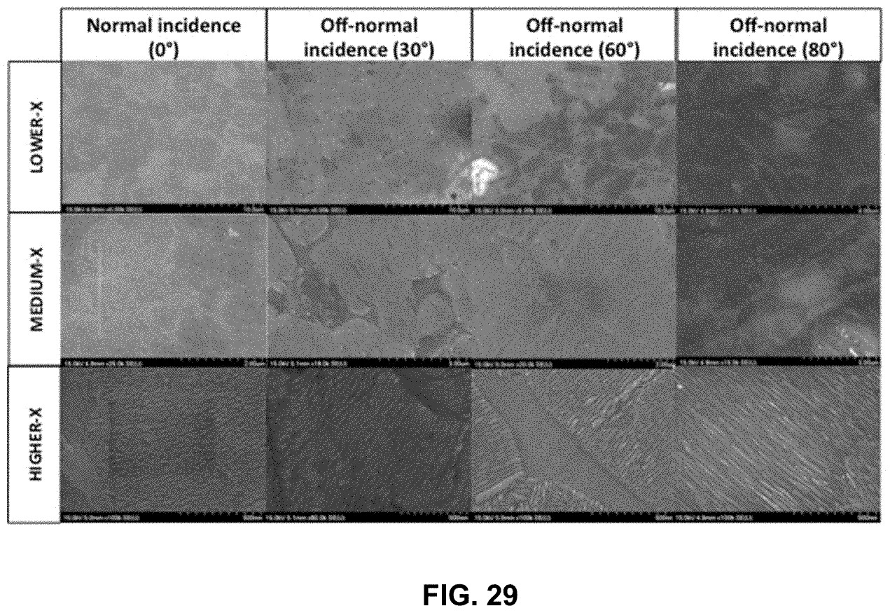

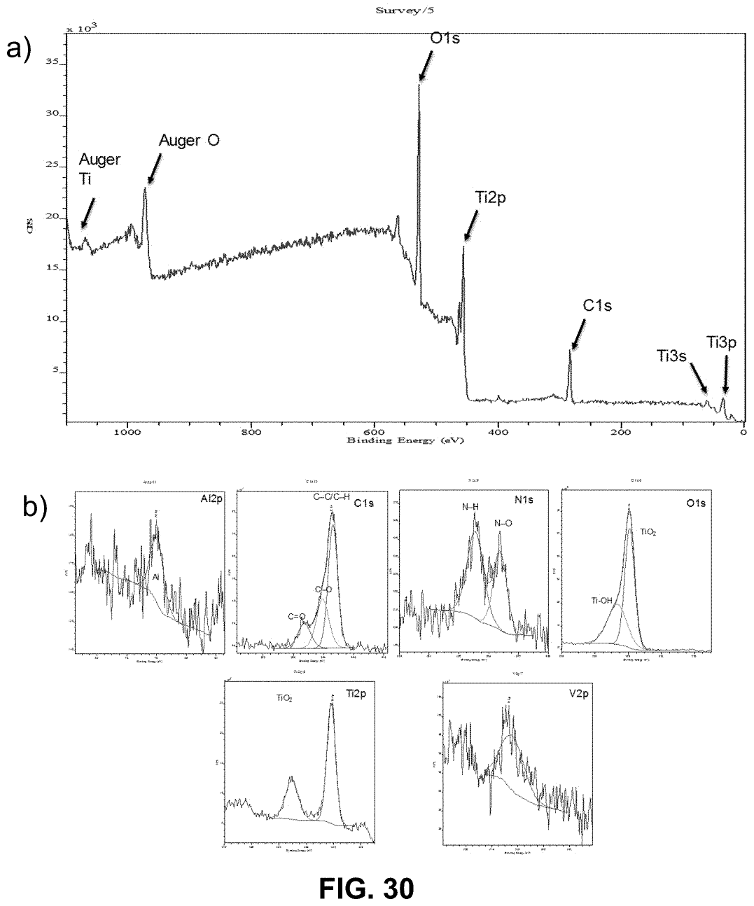

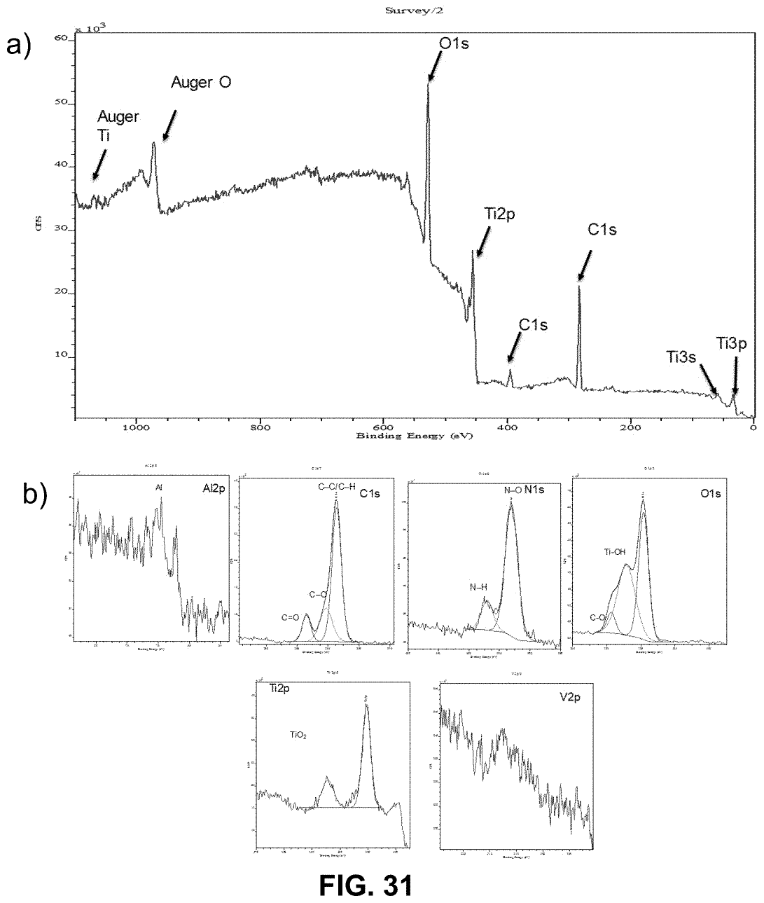

Surface structural modifications and nanostructuring due to DIS of Ti6Al4V are summarized in FIG. 29. At normal incidence an organized dot-like structure formed alongside “broken” nanoscale ripples on the original grains is observed. Comparison with the original surface microstructure, the self-organized dot-like structures appear concentrated in the a phase (grains and Widmanstatten plates), whilst the more irregular damage seems to be preferentially located within 1 phase matrix. Furthermore, the nanostructures at normal incidence appear to have some preferential growth depending on grain orientation. As the incident angle is increased, the stabilization of surface ripples is observed. These incipient nano-ripples can be also explained due to the activation of the terrace diffusion barrier and of the Ehrlich-Schwoebel barrier which opens the possibility for the development of the surface instability connected to ripple formation. At normal incidence, the features produced by ion sputtering reflect the surface symmetry and are aligned along energy preferred crystallographic orientation and enhanced surface recombination, which can lead to partial nano-dot and nano-ripple formation or in some cases complete smoothening of the surface.

In FIG. 29 the nano-structure features observed produced by an increase of the ion-beam incident angle to 60° are very similar to those observed for 30°. Due to the increased incident angle, it appears that curved ripples are even more important than in the case of the 30° incident ion-beam angle. Again, this mixed pattern formation indicates the competition between those above-mentioned mechanisms: a diffusive regime along preferential crystallographic directions and erosive regime with nanostructure's wave vector aligned parallel the incident ion-beam. The Ti β (bcc) phase still appears insensitive to any nano-structuring due to ion irradiation at the 60° incidence.

At the highest incident angle (e.g. 80° incidence), a new trend in the resulting nano-pattern is obtained (see FIG. 29). Namely the α-phase of Ti6Al4V alloy was not the only phase material constituent responding to Ar+ion irradiation, but also the Ti β (bcc) phase. At this grazing incidence new nanoscale ripples are observed in both phases. The ripples in both phases are found to have their wave vector aligned with the ion-beam direction. The transition angle between perpendicular and parallel wave-vector ripple formation with respect to the incident ion-beam direction is therefore found to be between 60° and 80° incidence. This transition is also indicative of an observed transition for Ti-based alloys between surface and sub-surface diffusive mechanisms to erosion-dominated mechanisms dominant at grazing incidence. Furthermore, at grazing incidence the separation distance between ripples decreases resulting in well-aligned ripple structures that are about 50-nm thin and with lengths close to 0.1-0.5 Cpm. Another important result here is that the dominating erosive processes found during grazing incidence irradiation can be achieved at room temperature. These conditions inhibit thermally-activated diffusion processes, which tend to smooth the surface and to orient the nanostructures along the preferential thermodynamic orientations. This specific response indicates that, under erosive sputtering conditions, it is possible to grow nanostructures, which can be aligned along thermodynamically unfavored directions.

2. Crystallographic structure:

Considering the microstructure of unirradiated Ti6Al4V samples after proper etching procedures, one can observe a conventional α+β mill-annealed alloy (see FIG. 1) consisting of a phase (hcp), equiaxial grains and Widmanstatten plates, dispersed in an untransformed β matrix (bcc). This microstructure is the consequence of both heating and milling at the α+β thermodynamically stable region, and further slow cooling, allowing β−α transformation. This combination of phases and constituents results in an excellent balance between mechanical strength, toughness, ductility and fatigue resistance. Surface structural modifications and nanostructuring due to DIS of Ti6Al4V are summarized in FIG. 29.

Intrinsic to the DIS modification is its ability to only modify the first few 100's of nm and therefore not affect the optimized mechanical properties mentioned described above. The first observation relates to the effect normal incidence has on the modified surface. At normal incidence an organized dot-like structure formed alongside “broken” nanoscale ripples on the original grains is observed. Comparison with the original surface microstructure, the self-organized dot-like structures appear concentrated in the a phase (grains and Widmanstatten plates), whilst the more irregular damage seems to be preferentially located within P phase matrix. Furthermore, the nanostructures at normal incidence appear to have some preferential growth depending on grain orientation. However, normal incidence and low energy processes in some types of materials (e.g. Si) have resulted in the smoothening of the surface. Evidence for a resistance to patterning is found in the normal incidence case as well as more oblique angles for certain grains. This could be evidence of a balance between mass redistribution mechanisms that drive adatoms on the surface to recombine with irradiation-driven surface vacancies leading to smooth surfaces. The fact that smooth surfaces only occur under certain grain orientations suggests that there is also a structure-driven relaxation mechanism coupled to the irradiation-driven mechanisms that lead to self-organized nanostructures. At normal incidence, the features produced by ion sputtering reflect the surface symmetry and are aligned along energy preferred crystallographic orientation and enhanced surface recombination, which can lead to partial nano-dot and nano-ripple formation or in some cases complete smoothening of the surface. Comparing to this work we find resistance to nanopatterning along specific grain orientation or microstructure phases.

Moreover, we discovered this morphological trend with incident ion-beam angle is dominant for the α-phase in the Ti6Al4V alloy meanwhile for the P (bcc) phase appears insensitive to this trend. The interaction and competition between the two mechanisms discussed above induces pattern formation observed in FIG. 29 and influences the resultant surface properties such as hydrophilicity and hydrophobicity.

In FIG. 29 are also depicted structures obtained from off-normal incidence starting with 8=30°. This change in the incident angle results in a predominant elongated rippled nanostructure at the irradiated surface mostly associated to the response of a (hcp) phase of the alloy. Given its polycrystallinity the observed nano-ripples were not perfectly perpendicular to the direction of irradiation. Most of them clearly appear with a shift of direction, which is a consequence of the coupling of incident energy deposition at the near surface along with surface diffusion being correlated to the movement of atoms associated with a particular crystallographic orientation. Interestingly, grains of the same a (hcp) phase were not transformed to nano-ripples, exhibiting a type of elongated nano-grains (short nano-rods). These nanostructures can be assumed as an intermediate nanostructure phase between curved “elongated” nano-ripples and long nano-rods in the same direction of the ion beam (see discussion later) consistent with an erosive-dominated and enhanced surface diffusion regime as described by the Bradley and Harper (BH) model. Another important point is the difference in response of metallic surfaces compared to semiconductor materials to ion irradiation. Metals have both higher diffusivity and the intrinsic non-directional nature of the metallic bond results in both resistance to amorphization and consequently a more sensitive dependence to crystallographic orientation. The formation of a pattern is thus dictated by: the surface curvature dependence of ion-induced sputtering and the presence of an extra energy barrier whenever diffusing adatoms try to descend step edges. Formation of nano-ripples on Ti6Al4V due to off-normal ions incidence is consistent with those assumed mechanisms on metals: diffusive processes are mainly responsible for the formation of regular structures. However, here we have two new findings, which warrant brief discussion: those nano-rods observed in some grains of a (hcp) phase, and the clear insensitivity of p (bcc) phase in Ti6Al4V alloys to nano-structuring. These features (or the lack thereof) can be attributed to the influence of crystallographic orientation in conjunction with corresponding a (hcp) phase and R (bcc) phase.

3. Surface Chemistry:

3.1 Parameter: Fluence 1×10¹⁸ cgs (Ar)_02

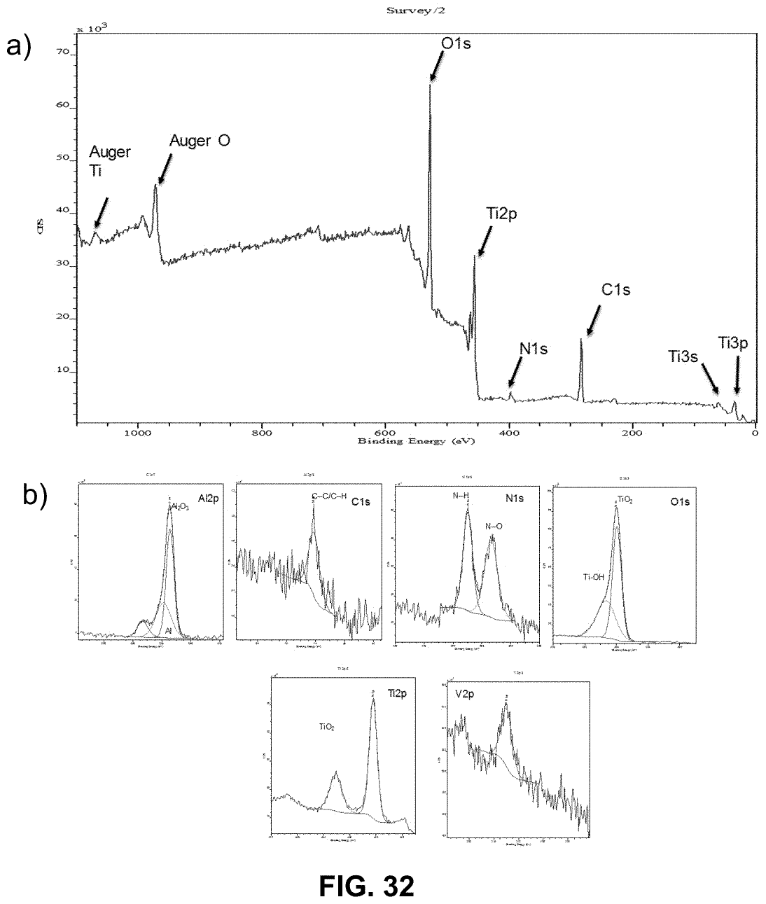

The survey scans for Ti alloy samples treated with a fluence of 1×10¹⁸ cgs for Ar irradiation at 60° is displayed in FIG. 32a. The results show the presence of Ti, C, O and N but show the presence of no contaminants. The Al2p, C1s, N1s, O1s, Ti2p, and V2p are shown in FIG. 32b. The Al2p shows the presence of only Al after irradiation. There is no change in Ti2p and C1s compared to control. The 2 peaks of O1s are assigned to Ti—OH and TiO₂. We now see a presence of V. After irradiation N1s shows the presence of N—H and N—O. The atomic concentration for control is summarized in Table 8.

TABLE 8
Atomic concentration for control Ti6Al4V

| Sample | 01s | C1s | Ti2p | N1s | V2p | Al2p |
|---|---|---|---|---|---|---|
| Ti—Ar-02 | 45.4 | 35 | 13.3 | 2.8 | 0.7 | 2 |

3.3 Parameter: Fluence 7.5×10¹⁷ cgs (Ar)_10

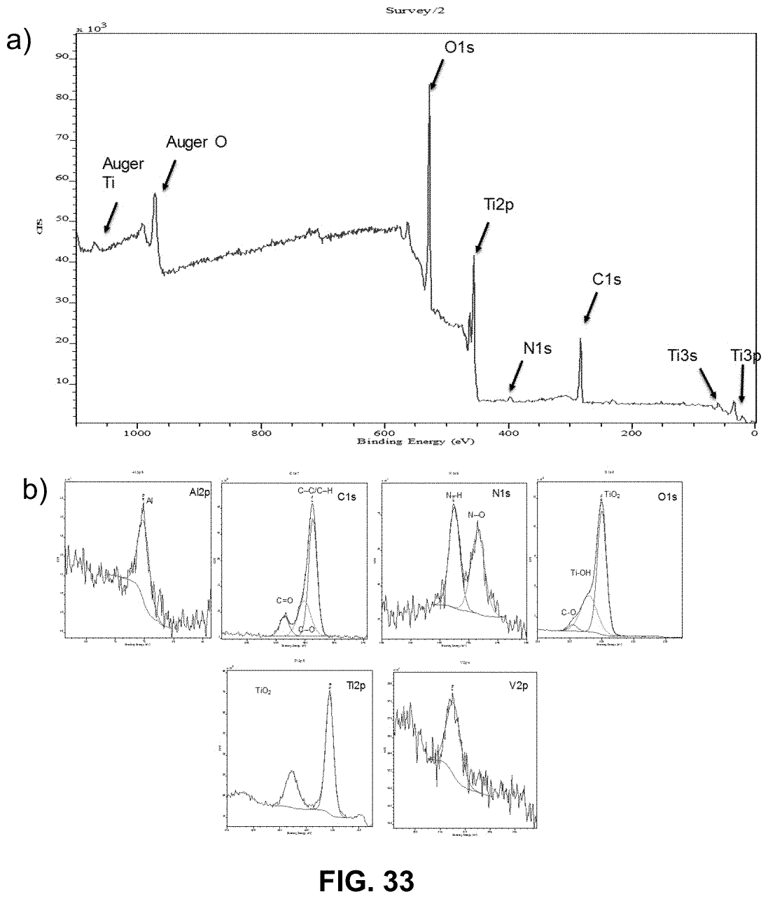

The survey scans for Ti alloy samples treated with a fluence of 7.5×10¹⁷ cgs for Ar irradiation at 60° is displayed in FIG. 33a. The results show the presence of Ti, C, O, N, V, Al. The Al2p, C1s, N1s, O1s, Ti2p, and V2p are shown in FIG. 33b. The Al2p shows the presence weak Al after irradiation. There is a minor decrease in C—O from C1s. There is no change in Ti2p. The 01s shows the presence of one new peak which is assigned to C—O. We now see a weak presence of V. We observe a rise in N—O compared to N—H. The atomic concentration for control is summarized in Table 9.

TABLE 9
Atomic concentration

| Sample | 01s | C1s | Ti2p | N1s | V2p | Al2p |
|---|---|---|---|---|---|---|
| Ti—Ar-10 | 34 | 50.5 | 5.5 | 5.7 | 1 | 3.3 |

3.4 Parameter: Fluence 5×10¹⁷ cgs (Ar)_09

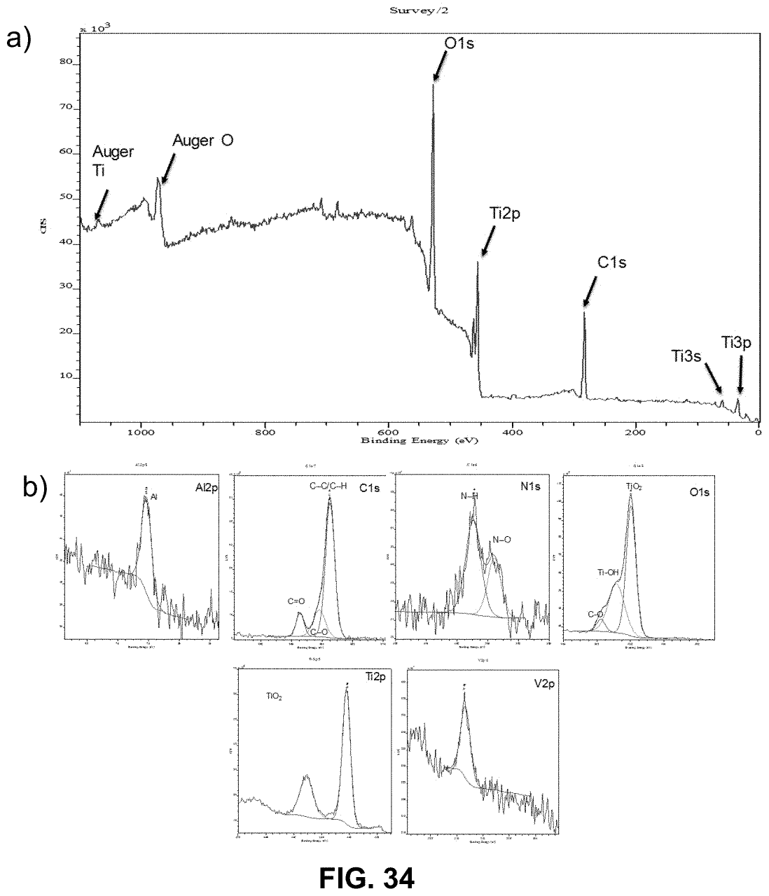

The survey scans for Ti alloy samples treated with a fluence of 5×10¹⁷ cgs for Ar irradiation at 60° is displayed in FIG. 34a. The results show the presence of Ti, C, O and N. The Al2p, C1s, N1s, O1s, Ti2p, and V2p are shown in FIG. 34b. The Al2p consist of three peaks, whereas C1s shows the presence of only one peak. There is no change in Ti2p. The O1s is composed of T-OH and Ti—O. We now see a weak presence of V. Meanwhile N1s is deconvoluted in to N—O and N—H. The atomic concentration for control is summarized in Table 10.

TABLE 10
Atomic concentration

| Sample | 01s | C1s | Ti2p | N1s | V2p | Al2p |
|---|---|---|---|---|---|---|
| Ti—Ar-09 | 42.7 | 40 | 12.1 | 3.2 | 0.2 | 1.6 |

3.5 Parameter: Fluence 2.5×10¹⁷ cgs (Ar)_08

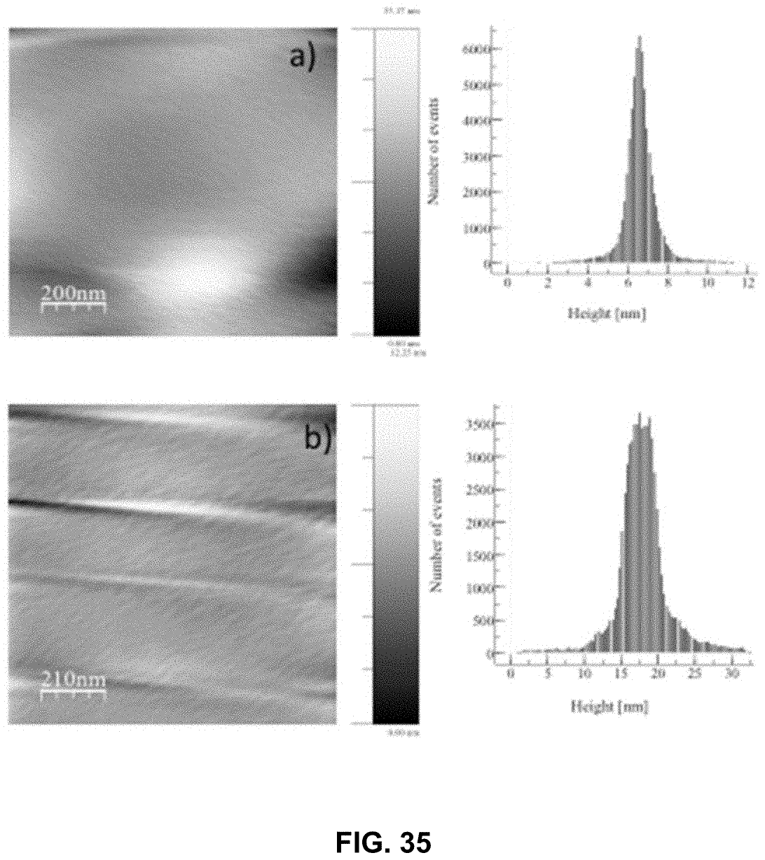

The survey scans for Ti alloy samples treated with a fluence of 2.5×10¹⁷ cgs for Ar irradiation at 60° is displayed in FIG. 35a. The results show the presence of Ti, C, O and N. The Al2p, C1s, N1s, O1s, Ti2p, and V2p are shown in FIG. 35b. The Al2p consist of only one Al peak. On the other hand, C1s is composed of three peaks. Meanwhile N1s is deconvoluted in to N—O and N—H. There is no change in Ti2p. The O1s is deconvoluted in to 3 peaks. We now see a weak presence of V. The atomic concentration for control is summarized in Table 11.

TABLE 11
Atomic concentration

| Sample | 01s | C1s | Ti2p | N1s | V2p | Al2p |
|---|---|---|---|---|---|---|
| Ti—Ar-08 | 43.2 | 39.1 | 12.6 | 2.2 | 0.5 | 2.07 |

3.6 Parameter: Fluence 1×10¹⁷ cgs (Ar)_07

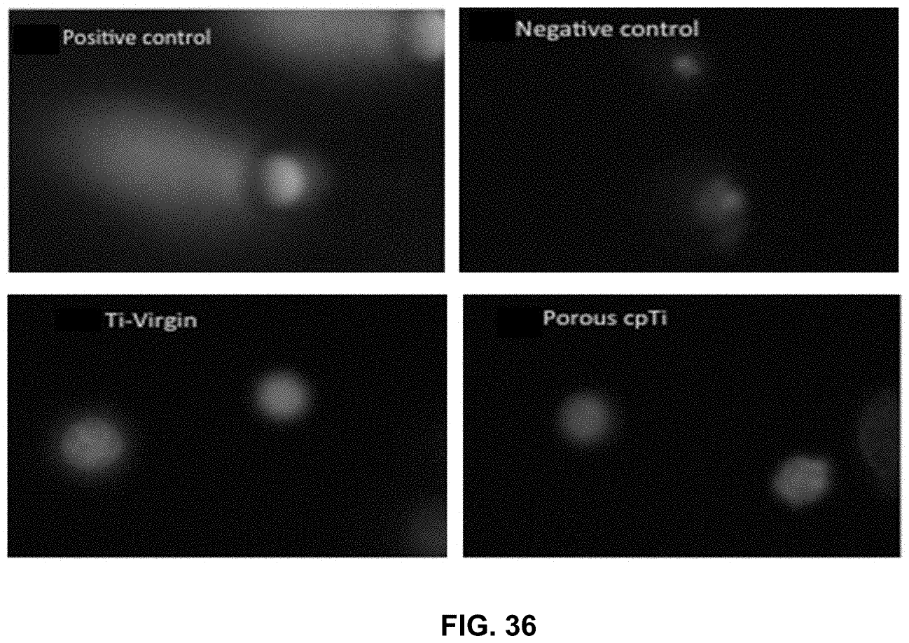

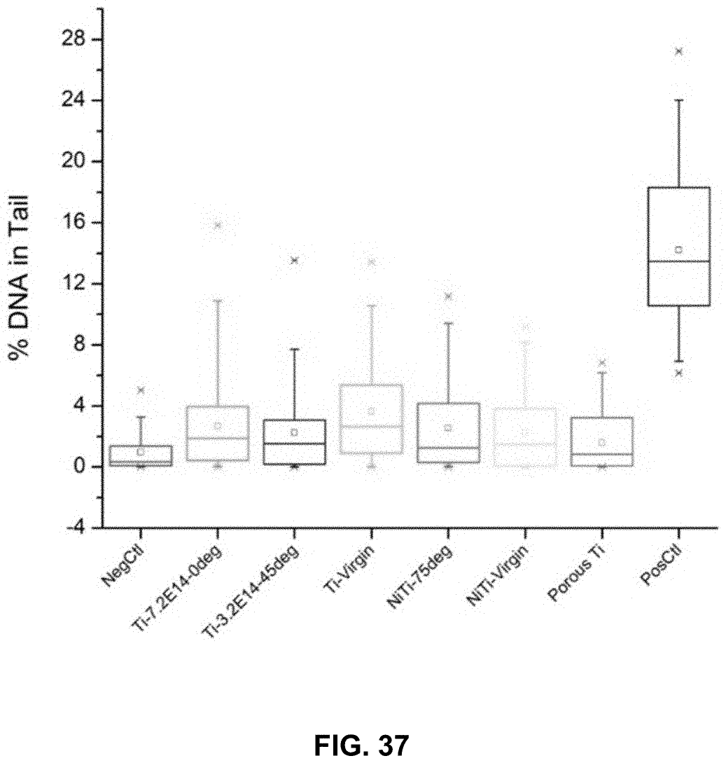

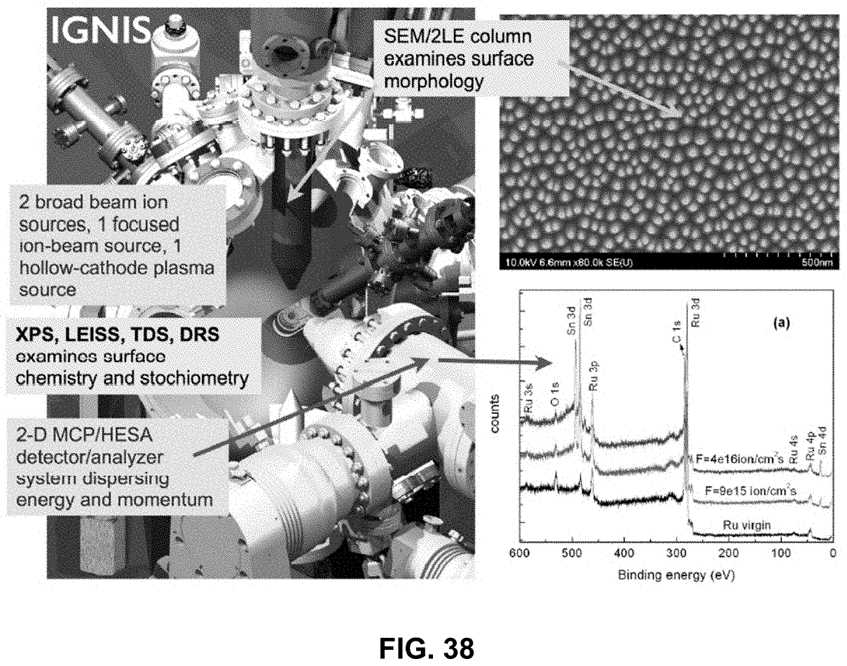

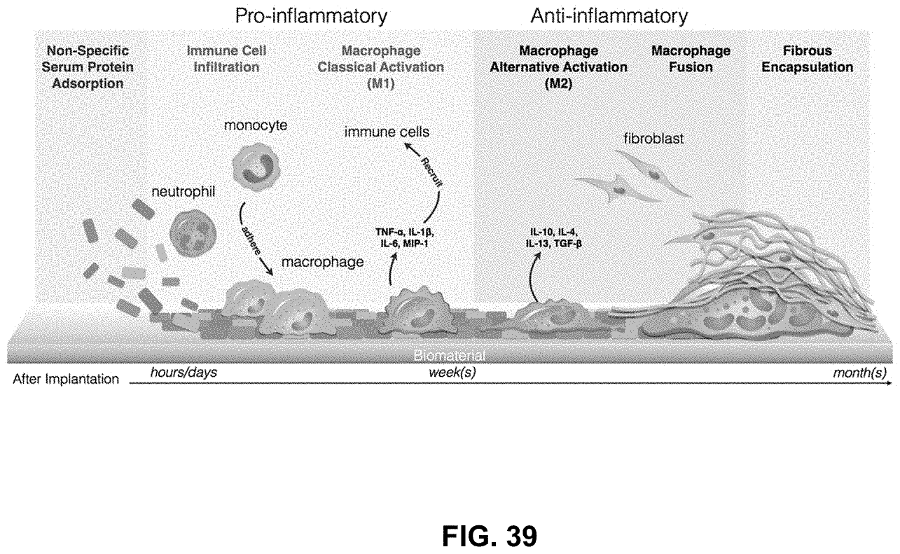

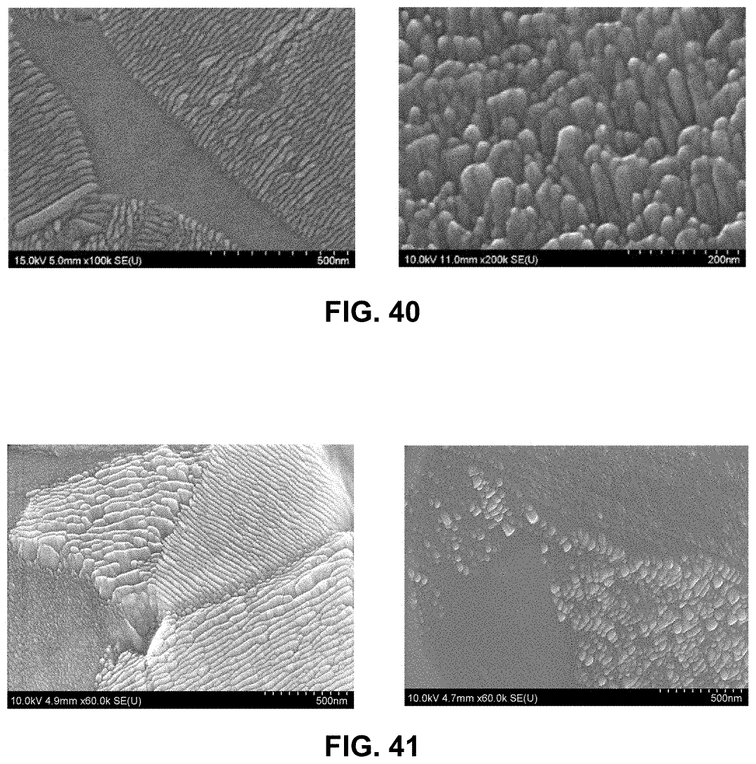

The survey scans for Ti alloy samples treated with a fluence of 1×10¹⁷ cgs for Ar irradiation at 60⁰ is displayed in FIG. 36a. The results show the presence of Ti, C, O and N. The Al2p, C1s, N1s, O1s, Ti2p, and V2p are shown in FIG. 36b. The Al2p consist of only one Al peak. On the other hand, C1s is made up of three peaks. Meanwhile N1s is consists of N—O and N—H. There is no change in Ti2p. The O1s is deconvoluted in to 3 peaks. The atomic concentration for control is summarized in Table 12.

TABLE 12
Atomic concentration

| Sample | 01s | C1s | Ti2p | N1s | V2p | Al2p |
|---|---|---|---|---|---|---|
| Ti—Ar-07 | 41 | 44.1 | 11.28 | 1.6 | 0.24 | 1.6 |

### Example 4: Nanostructured Bioactivated Porous Titanium Implants for Bone Tissue and Vascular Repair and Methods to Fabricate the Same

### Brief Overview

Described a modified powder metallurgy (PM) porous cpTi (commercially-pure) material that is transformed by directed plasma nanosynthesis (DPNS) with the aim to provide three primary bioactive functions: a) enhanced soft tissue and bone tissue integration, b) anti-bacterial interfaces and c) ultra-hydrophilic properties within porous structures enabling enhanced protein and drug adhesion. We demonstrate the nano-structuring capability of DPNS on intra-porous and inter-porous surfaces without any detrimental effect on the original biocompatibility of porous cpTi.

The provided materials are designed for integration with bone or soft tissue (e.g. ligament, tendon, vascular, gum, etc. . . . ). The degree of tissue integration is dependent on the nanostructure design in-between pores and within pores of cpTi. In some embodiments, the invention provides methods capable of inducing nanostructure formation within and between Ti-based nano to microscale pores. Porosity serves two purposes: 1) to provide anchors for cell adhesion and 2) to provide protein/drug payload delivery. Control of surface chemistry and porosity is important in many biomedical application areas including: biosensors, drug delivery, tissue engineering, cell culturing and wound healing. Most methods to control porosity and surface chemistry is limited by chemical-based processes that when produced at industrial scales translate in significant toxic waste, which can result in about 30-40% of total fabrication costs. This has the potential to disrupt this cost by providing for a non-chemical method of fabrication with DPNS providing for nanostructure control of surfaces within and in-between pores. The application for this nanostructured porous cpTi material include: joint replacements, hip and shoulder fractures, dental implants, and bone diseases related like osteoporosis and cancer.

Most current clinical treatments for bone tissue regeneration correspond to tissue substitution, i.e. replacement approach for which the biomaterials are both biocompatible and bioactive. Although there have been some clinical successes with conventional materials, several failure statistics of current joint replacements indicate that are challenged by the bioinert nature of most of these materials including Ti-based biomaterials. Some medical grade titanium (Ti) alloys, like commercially pure Ti (cpTi), have proved to be the best biomaterials for clinical success of bone replacement due to its excellent balance between biomechanical and in vivo biocompatibility. However, cpTi exhibits the following disadvantages that are direct consequences of being a 1st generation biomaterial: 1) Biomechanical incompatibility reflected in the elastic mismatch with respect to hosting tissue, and the consequent bone resorption around the implants; 2) Being bio-inert, cpTi implants are surrounded by a thin fibrous tissue, which can often reduce osseointegration with an associated risk of loosening or fracture of bone and/or implant; improvement of the biointerface is necessary to avoid the existence of the fibrous tissue or to reduce the loosening risks due to its presence.

In regards to first problem, it is desirable to design new implants and prostheses with a lower stiffness than those currently used, which would allow the stress-shielding problem to be solved or reduced without any significant detrimental effect on mechanical strength. Several reported studies have dedicated to development of new implants with a bone-matching modulus, such as porous materials; to that end, there are some manufacturing processes, among which include: the electron beam melting process, creep expansion of argon-filled pores, directional aqueous freeze casting, rapid prototyping techniques, laser-engineered net shaping, electric current activated/assisted sintering techniques, conventional and non-conventional powder metallurgy (PM) and space-holder techniques. From a conventional PM point of view, controlling compacting pressure, sintering temperature and time, could help reach a suitable porosity to reduce stress shielding. Improvement of bone ingrowth and osseointegration requires that pores size and morphology be controlled, especially in the surface, which is also important for the fatigue resistance of the implant. In regards to non-conventional PM alternatives for stress-shielding reduction, loose sintering process, without compaction pressure, has emerged as an attractive route to produce porous Ti implants with a high porosity, with the aim of successfully replacing cancellous bone with an extraordinary low Young's modulus of around 0.5 to 1 GPa. To that end, we have recently published a detailed comparison between space-holder and loose sintering techniques, in which they were able to properly optimize a suitable mechanical balance for a Ti implant for highly porous bones.

In regards to alternative biomaterials to minimize and/or avoid fibrous tissue formation and lack of osseointegration, they are basically based on modifications of topographical and/or chemical properties of cpTi surfaces. Surface nanostructuring of conventional biomaterials has emerged as one of the most important and effective manners to convert them in advanced biomaterials; this is the consequence of nano-features capability to effectively have some influence on surrounding biological environment at molecular nano-scale level. Since the cells in their natural environment are surrounded by nanoscale features linked to their extracellular matrix (ECM), the nanotopographical parameters become an important part in design of biomaterials for tissue formation and repair. Accordingly, several studies suggest that a remarkably small modification in surface nanotopography could result in mesenchymal stem cell growth and development, indicating that changes in such nanotopographical features had a direct influence in the adhesion/tension balance to permit self-renewal or targeted differentiation. Biointerface topography and, in particular, nanoscale features can affect cell behavior and integrin-mediated cell adhesion, and is now evident from studies with fabricated topographical features. The processes that mediate the cellular reaction with nanoscale surface structures are not well understood and may be addressed; a direct result of the influence of the surface topography, or even indirect one, when the surface structure affects the composition, orientation, or conformation of the adsorbed ECM components.

The drawbacks of conventional nano-patterning techniques have mainly been attributed to their physical limitations in fabricating structures smaller than about 50-nm. Therefore, bottom-up techniques that rely on self-assembly, self-organization, and local patterning, have become technologies capable of pattern biocompatible surface nanostructures. Irradiation-driven systems have been explored in moderate energy regimes dominated by knock-on atom displacement regimes for semiconductor metallization microstructure control, engineering of nanostructured carbon, and compositional patterning of immiscible alloys. Directed plasma nanosynthesis (DPNS) introduces a synthesis process that is scalable to high-volume manufacturing by virtue of its intrinsic large-area simultaneous exposure of materials surfaces and interfaces.

There are no reported works on ion and plasma-induced irradiation nano-patterning with intra-porous and inter-porous surface modification of porous Ti with applications to cell stimulation and tissue growth. The methodology with DPNS can target specific regions in, around and in-between pores that enable specific tailored functionalities from drug delivery to soft issue integration enabling a highly versatile and tunable biomaterial for advanced biosensing and protein/drug adhesion/delivery applications.

Provided is a new biomaterial that introduced multiple functions to porous cpTi-based biomaterials. Also provided is a method or process of fabrication using DPNS to induce nanostructures in-between and inside pores. This process not only introduces nanostructuring as described above but also can refine pore size.

Described is a process and arrangement to generate patterned structures and unique topography at the nanoscale in-between and inside pores while eliminating the shortcomings of conventional chemical-based approaches. Besides reducing significantly toxic chemical waste processes the use of DPNS on nanostructuring porous cpTi biomaterials avoids costly high-temperature cycles necessary in conventional surface modification techniques.

### Results and Discussion

Structural characterization of as-received (AR) and DPNS-treated cpTi samples

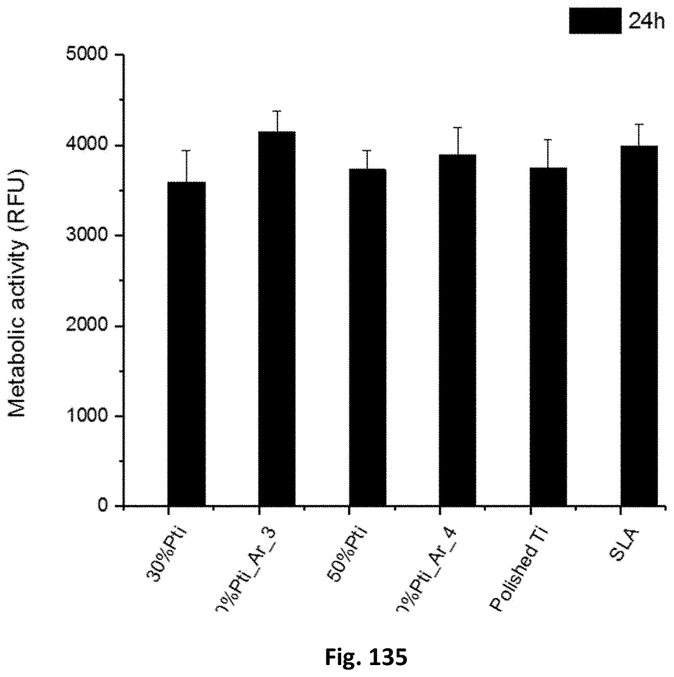

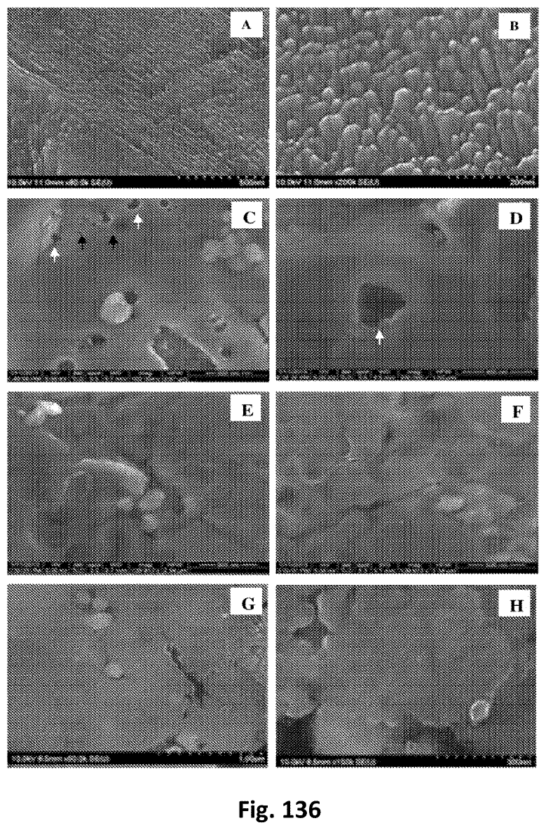

Surface structural modifications and nano-structuring due to DPNS of porous cpTi samples are summarized in FIGS. 135a-135h. The influence of DPNS on lower porosity cpTi (FIG. 135a) is reflected in a general nano-patterning; it mostly corresponds to short oriented nano-rods, preferentially oriented in the same orientation of the ions beam direction. However, some of them appear with a switch of direction, most probably associated to different crystallographic direction of a phase (hcp) grains. This synthetized nano-patterning appears partially as some nano-ripples or with a mixed structure between nano-rods and nano-ripples. The prevalence of nano-rods indicates that the incident angle has the same value of the transition angle or slightly higher (diffusive to erosive regime; rods and ripples to 100% nano-rods). The observed general effect of DPNS nano-patterning is the consequence of cpTi microstructure as a monophasic alloy of a phase (hcp); the whole coherency between nano-rods and nano-ripples with respect to ion beam direction would indicate that surface grains are texturized; i.e. grains in the same direction that could be the consequence of the polishing operation which is also verified because of the consistency with polishing scratch directions. It is relevant the high throwing power of this DPNS nano-patterning, which is reflected in the capability to create nano-rods inside the pores, and inside the scratches as well (see details in FIGS. 135a and 135b). Not only nano-rods, but also nano-columns can be easily appreciated inside the gaps-scratches, in a similar way as it is normally observed inside a trench during practice FIB technique. Those columns from the bottom of the scratch seem to grow approximately perpendicular to surface; however, those nano-rods far away from the gap, appear more elongated and they tend to be more parallel to the surface.

Nano-structuring of porous cpTi samples depicted in FIGS. 135a-h are consistent with some previous studies about Ar+ion beam irradiation on cpTi; similar to that work, we obtained mostly nano-rods and some mixed areas with nano-ripples for an incident angle of 60°. This similarity can be explained in terms of the incident angle, which appears slightly higher than transition angle (TA) between mostly nano-ripples formation (normal incidence, diffusive regime) and straight and long nano-rods (highly off-normal incidence, grazing incidence, of around 80°; erosive regime). Note that high nano-patterning effectiveness observed here due to DPNS on porous cpTi (100% a phase, hcp), is in agreement similar experiments of DPNS on Ti6Al4V; they showed partial nano-patterning due to DPNS due to also partial percentage of a phase (hcp). Our results here indicate that this phase has a favorable strong response to DPNS nano-patterning. In regards to high efficiency of DPNS nano-structuring, no matter the porosity of cpTi samples, it could be associated to a favorable effect of previous polishing of samples, which have pores confined at the surface. The mixed surface of flat and porous areas seems not to mean any obstacle to DPNS efficiency for surface nano-patterning of cpTi. It must be noticed that DPNS nano-patterning was basically the same on porous cpTi samples with highest porosity. The positive response of outer pores to DPNS nano-patterning can be explained in terms of interactions between remote (nominal) incident angle (60°) and local incident angles inside the pores; in this context, flat polished zones responded in a conventional mixed way of nano-rods+nano-ripples. However, the curved zones inside the pores are more sensitive to create nano-rods, which would mean that ion beam on curved surface helps to stimulate the erosive regime, when is used an intermediate incident angle like 60°. These observed tendencies are in well agreement with that observed in previous works about ion bombardment on porous solids, as well as with some theoretical predictions about ion beam nano-structuring.

AFM analysis has allowed us a to quantify the surface features due to DPNS on porous rough cpTi (see FIGS. 5a-5b); firstly, for samples with the lowest porosity, AFM images confirm the nano-patterning previously observed by SEM. Again, this is uniformly present at the flat zones due to previous mirror polishing. AFM images allowed us hardly appreciate mixed nano-patterning of nano-rods and nano-ripples. Roughness quantification is also presented in the same FIGS. 5a-5b, in which can be appreciated the nano-scale of the vertical main roughness parameter of irradiated porous samples of 0.57 and 3.49 nm. Note the presence of polishing scratches due to mechanical polishing. Topographical and nano-patterning aspect of highest porosity samples (FIG. 5b), are similar to lowest porosity ones; however, quantification of roughness parameter shows that mean height is higher. Unfortunately, there does not appear to be any reported work about AFM characterization of cpTi irradiated in similar conditions.

Surface Free Energy Evaluation of as-Received (AR) and DPNS-Treated cpTi Samples

The wettability is fundamental for the cellular adhesion and, consequently, for the success of osseointegration and bone tissue growth, since the blood is the first tissue that reaches the implant, and 90% of its plasma is composed by water, the evaluation of the surface wettability can be accomplished through the determination of contact angle. The contact angle measurements after irradiation of porous cpTi samples reflected an important change with respect to control one (see Table 13); both kind of porous samples (lowest and highest porosity) showed important reduction of contact angle (reduced hydrophobicity) after Ar+irradiation with 60° incidence angle. In case of lowest surface porosity, contact angle was of 13.45±4.51 (reduction of 74.64%), and for highest porosity the contact angle was of 28.15±5.21 (reduction of 46.92%). It is important to notice that those reduced values of contact angle after DPNS were obtained despite the initial porosity of samples and the initial roughness of rough samples as well. Despite there are reported some works about influence of ion irradiation on contact angle, this is the first time that such a reduction is reported due to off-normal Ar+incidence on Ti.

TABLE 13
Results of contact angle testing (DI
water) on irradiated cpTi samples

| | Incidence | Mean | |
|---|---|---|---|
| Samples | Angle (°) | Value | ST DEV |
| Untreated cpTi | — | 53.03 | 0.06 |
| S1 (cpTi) | 60° | 13.45 | 4.51 |
| S2 (cpTi) | 60° | 28.15 | 5.21 |
| S3 (cpTi) | 0° | 33.3 | 3.5 |
| S4 (cpTi) | 45° | 32.1 | 4.2 |
| S5 (cpTi) | 75° | 25.6 | 3.8 |

Biological Assessment of as-Received (AR) and DPNS-Treated cpTi Samples

As it is well recognized from biological response of biomaterials surfaces, free surface energy of biomaterials reflected in their contact angle values plays a determinant role in the biological environment response. By considering the effect of multiple surface properties in contact angle results, here we can establish important relationships with surface modifications due to ion irradiation processing of cpTi surfaces. In that context, osseointegration is one of the good examples in which, besides the roughness, the surface tension is a parameter that interferes on it. It permits a higher or smaller scattering of liquid onto the metallic surface. The human blood contains about 90% of water, thus the capability of water adsorption by the surface, known like wettability, is a fundamental parameter to the success of cellular adhesion and, consequently, to the osseointegration. It is commonly accepted that blood compatibility is improved when the hydrophilicity of a surface is increased (unless the surfaces are superhydrophobic).

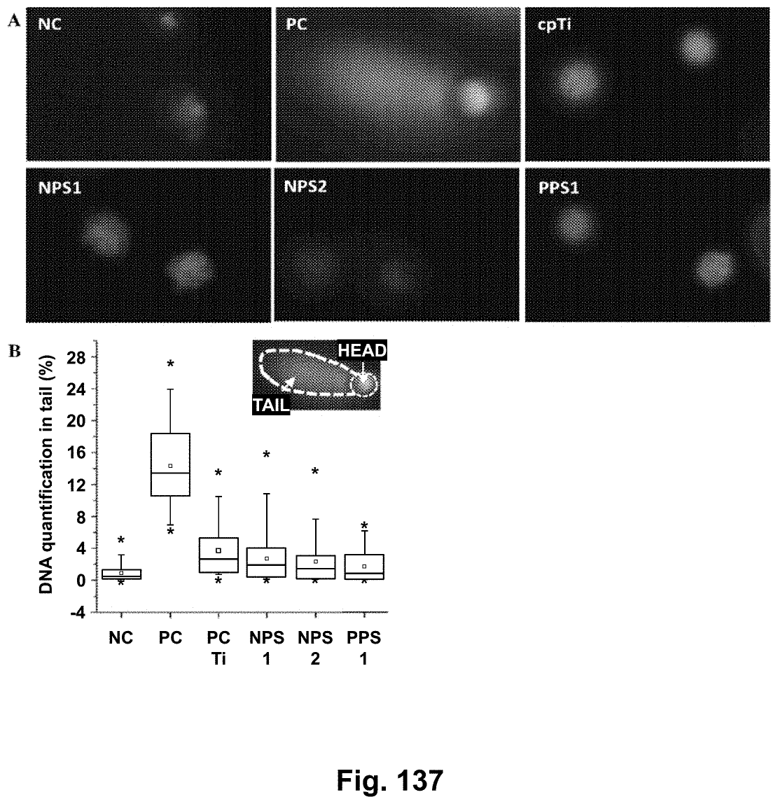

Cytotoxicity assessment of irradiated samples was performed via Comet Assay@ testing, in search of evaluating the potential geno-toxicological effect in vitro on human aortic smooth muscle cells (HASMCs) cultured in the presence of the evaluated specimens. FIG. 137a shows the characteristic shape of nucleoid exhibiting DNA damage (positive results for the assay) after treatment with hydrogen peroxide (H₂O₂). The tail of the nucleoid corresponds to DNA strand breaks produced by exposure with the toxic agent. FIG. 137a shows negative results for DNA damage in untreated cells (cells growing alone and without exposure to any toxic agent), which is characterized by compact nucleoids. Remaining figures show the results obtained for the tested materials (untreated, and porous irradiated samples). Notice that these nucleoid shapes are closer to negative control samples instead the positive ones. Virgin untreated sample in FIG. 137a. also corresponds to cpTi biocompatible surface without irradiation and use for comparative purposes. These samples show similar nucleoid shapes to their irradiated counterpart. The potential DNA damage produced on HASMCs after exposure to the tested analytes was quantified using Comet Score™ software (Tri Tek Corp., Sumerduck, Va.). The results were compared with those obtained for the control samples (positive and negative controls). Positive results for DNA damage are characterized for high percentage of DNA in tail close or higher than the values for the positive control sample. Tables 14 and 15 summarize the results of percentage of DNA in tail obtained for controls and treated and untreated cpTi surfaces. At 0.05 levels, the tested materials means are significantly different to the positive control (Table 15), thus indicating no detrimental effects in HASMC's DNA induce by the tested materials under the experimental conditions outlined in this report. FIG. 137b display the data distribution in Tables 14 and 15. The range of variation for the tested samples is lower than the positive control (treated with H₂O₂). It indicates that irradiated cpTi surfaces did not induce DNA damage on HASMCs cultured in the presence of these materials under the experimental conditions tested in this work. Therefore, here we can estate that we tested the potential adverse effects on HASMC genetic material as a result of exposure to irradiated cpTi surfaces through the Comet Assay®. The results depicted in the present work suggest that irradiated cpTi samples (porous+polished and rough) with Argon under different conditions do not produce detectable DNA damage in HASMCs. In regards to HASMCs morphology changes during time in contact with those different surfaces, that was performed by visual inspection of HASMCs growing in the presence of the tested materials, during the incubation period under bright field illumination utilizing a Nikon inverted Diaphot fluorescent microscope with 10× and 20× objectives (Nikon Instruments, Melville, N.Y.). Periods of observations were 0, 24 and 48 hours, and this preliminary characterization of cells behavior by co-culture on the surfaces has shown that irradiated cpTi surfaces does not have any cytotoxicity effect on the HASMCs.

TABLE 14
Percentage of DNA in tail parameter after performing Comet-Assay
on HASMCs cultured in the presence of irradiated metal surfaces.

| | | | Standard |
|---|---|---|---|
| Sample | N Analysis | Mean | Deviation |
| Negative Control | 45 | 0.96 | 1.21 |
| Rough cpTi-0deg | 45 | 2.67 | 3.15 |
| Rough cpTi-45deg | 45 | 2.24 | 2.62 |
| Ti-Virgin | 45 | 3.63 | 3.38 |
| Porous cpTi | 45 | 1.59 | 1.95 |
| Positive Control | 45 | 14.2 | 5.06 |

TABLE 15
One-way ANOVA was performed to compare the mean
of each tested sample with the control ones.

| | | Sum of | Mean | | |
|---|---|---|---|---|---|
| | DF | Squares | Square | F Value | Prob > F* |
| Model | 7 | 5798.4 | 828.3 | 89.1 | 0 |
| Error | 352 | 3270.9 | 9.29 | | |
| Total | 359 | 9069.3 | | | |
| | | Sum of | Mean | | |
| | DF | Squares | Square | F Value | Prob > F |
| Model | 2 | 5019.4 | 2509.7 | 243.5 | 0 |
| Error | 132 | 1360.3 | 10.30 | | |
| Total | 134 | 6379.8 | | | |

*At the 0.05 level, the population means are significantly different.

Biocompatibility is, at least, equal to well-known biocompatibility of conventional bio-inert cpTi surface; with respect to cell behavior parameters, by considering our improvements in nano-structuring, roughness parameters and contact angle, we can reasonably expect that cells factors like adhesion, proliferation, migration, and differentiation will be also improved with the surfaces obtained here. However, it is important to point out at this moment that our results reported here are new in the sense that is the first time is shown relationships between initial surface micro-topography of porous cpTi, irradiation conditions, surface nano-structure, roughness, surface free energy and basic biocompatibility assessment; they are related not only with their behavior and adhesion, but also with their further potential to stimulate tissue growth.

### Prior Methods, Apparatus, Developments and Publications

DPNS of Porous and Rough cpTi Samples.

Experiments were performed at Radiation Surface Science and Engineering Laboratory (RSSEL) at the University of Illinois at Urbana Champaign, which is originally developed and designed by Prof. Jean Paul Allain (FIG. 41). DPNS conditions of different cpTi samples are summarized in Table 1 (see Example 1).

Manufacturing of Porous cpTi Samples

The powder of cpTi (SE-JONG Materials Co. Ltd., Korea) used for the blends was manufactured by a hydrogenation/dehydrogenation process. The particle size distribution corresponded to 10, 50 and 90% passing percentages, of 9.7, 23.3 and 48.4 μm, respectively. The chemical composition of the powder used was equivalent to cpTi Grade IV according to the ASTM F67-00 Standard. CpTi has an apparent density of 1.30±0.01 g/cm³ (28.8±0.1%) and a tap density of 1.77±0.04 g/cm³ (39.2±0.8%). The blends of cpTi powder were prepared using a Turbula® T2C blender for 40 min to ensure good homogenization. In order to address irradiation influence on porous cpTi samples with different porosities, here we are comparing the loose sintering technique (without any compaction pressure) with the conventional PM one via a low compaction pressure. The compacting step was carried out using an Instron 5505 universal machine to apply the pressure used of 100 MPa. The compacting loading rate was 6 kN/s, dwelling time was 2 min and unloading time was 15 s for decreasing load up to 150 N. The sintering process was performed in a Carbolyte® STF 15/75/450 ceramic furnace with a horizontal tube at 1250° C. for 2 h using high vacuum (=5×10⁵ mbar). Diameter of compaction die (8 mm) and powder mass were selected to obtain samples in which the effect of compaction pressure was minimized. Structural and surface free energy characterization of cpTi samples modified by DPNS

Surface free energy of irradiated samples was evaluated by contact angle testing with deionized water through a Rame-Hart Goniometer Model 500-Advanced contact angle goniomter/tensiometer with DROPimage Advanced Software. We performed the sessile method of contact angle analysis (where the sample was static and not moving after the drop was placed on it). All measurements were performed with deionized water (to not have any type of interaction with the surface). The water dropper was far enough away from the surface that the water did not touch the sample until it left the dropper. The dropper was kept at the same distance from each sample surface. The water droplets had ˜3 μL of water on each sample. Morphological features of samples were detailed analyzed by Scanning Electron Microscopy, SEM (Philips XL40 field emission, FEI, Hillsboro, Oreg., USA). Atomic force microscopy (AFM) was also used for detailed morphological and topographical characterization of irradiated cpTi surfaces, by using an AFM Veeco Dimension 3000 (Santa Barbara, Ca) on AC Mode using cantilever Bruker DNP-10. The scanned area was 1 μm square over samples of both titanium rough and porous samples.

Biological Evaluation of cpTi Samples Modified by DPNS.

The cells used for biological assessment of treated and un-treated samples were human aortic smooth muscle cells (HASMCs, Lifetechnology Cat # C0075C). To that end, cells morphology changes with culture time, and cytotoxicity assays test on modified surfaces via Comet Assay® testing, were performed. Changes in cells morphology in terms of culture time was observed by using optical microscopy (OM) analysis. The cell line used was the choose model to validate the potential for tissue growth and regeneration of the new surfaces. Cells were grown at 37° C. with >95% Rh and CO2 gas exchange until they were nearly confluent. HASMCs were seeded on the top of the samples for 24 hours under normal culturing conditions (37° C., 95% air, 5% C02, 95% humidity). HASMCs with a cellular density of 5×10³ live cells per cm2, and viability of 86% were cultured in a 7 well tissue culture dish in the presence of the analytes. Two samples labeled as NegCtl and PosCtl corresponded to cells growing alone and used as controls in the assay. All the samples were incubated under normal culturing conditions (37.0° C., 95% air, 5% C02, 95% humidity) for 24 hours. Then, after 48 hours, visual inspection of HASMCs growing in the presence of the tested analytes was performed during the incubation period under bright field illumination utilizing a Nikon inverted Diaphot fluorescent microscope with 1× and 20× objectives (Nikon Instruments, Melville, N.Y.). The Comet Assay® was carried out according to manufacturer's recommendations (cat. #4250-050-K, Trevigen, Inc., Gaithersburg, Md.). Finally, all the experimental measurements are presented as the mean+standard derivation, which were analyzed using Origin Pro 8.6. One-way ANOVA was performed to compare the mean of the samples, and at the 0.05 level, the population means were considered to be significantly different.

Based on the different experimental parameters, an array of different nanostructures can be induced on porous Ti. The nanostructures were obtained as function of angles and energy. Using directed irradiated synthesis, along with the surface these nanostructures can also be grown inside pores as well as along the pore walls

### 1. Sample: 30% Ti_Ar_05

### Angle=0°

Fluence (cm⁻²)=1.00E+18
Energy (eV)=1000

### Parameter: Angle (0°)

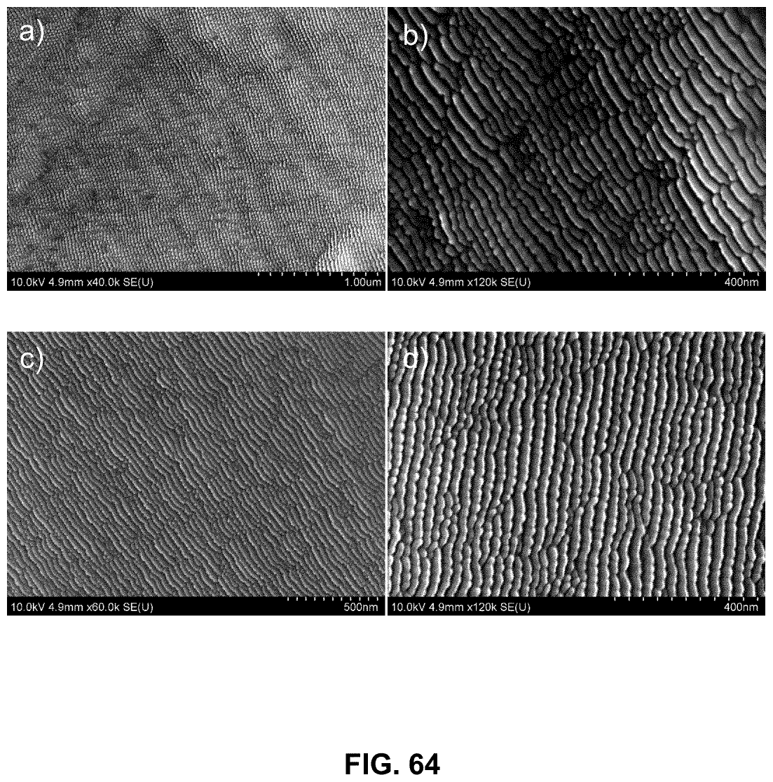

Surface Nano-walls (Ar): An array of (parallel) wall like structures having a height of 10 to 100 nm preferably, with a length ranging from 10 to 40 nm, said ridge composed primarily of Ti (FIGS. 64 a-d). Such structures are formed using following DIS parameters—Gas: Ar, Angle: 0°, Energy: 1000 eV, Fluence: 1×10¹⁸ cgs and are formed on the surface of the titanium substrate.

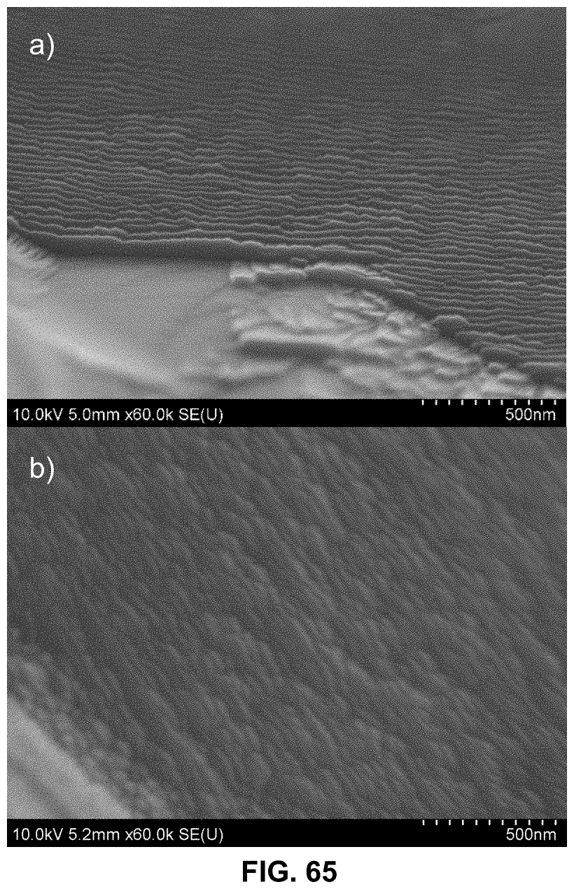

Inside Pore Nano-walls (Ar): Inside the pores, an array of (parallel) wall like structures having a height of 10 to 100 nm preferably, with a length ranging from 10 to 40 nm, said ridge composed primarily of Ti (FIGS. 65 a-b). Such structures are formed using following DIS parameters—Gas: Ar, Angle: 0°, Energy: 1000 eV, Fluence: 1×10¹⁸ cgs.

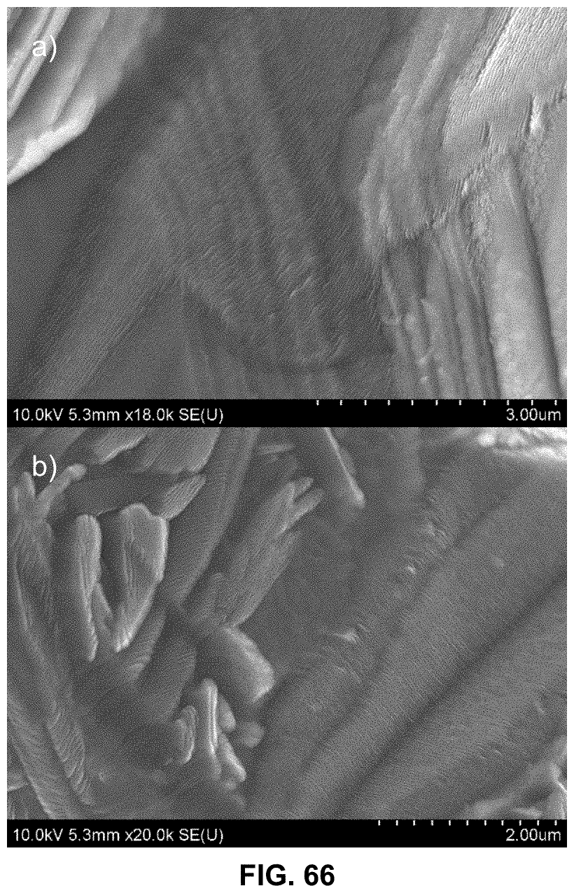

Walls of Pore Nano-walls (Ar): Along the walls of pores, an array of (parallel) wall like structures having a height of 3 to 100 nm preferably, with a length ranging from 10 to 40 nm, said ridge composed primarily of porous Ti (FIGS. 66 a-b). Such structures are formed using following DIS parameters-Gas: Ar, Angle: 0°, Energy: 1000 eV, Fluence: 1×10¹⁸ cgs.

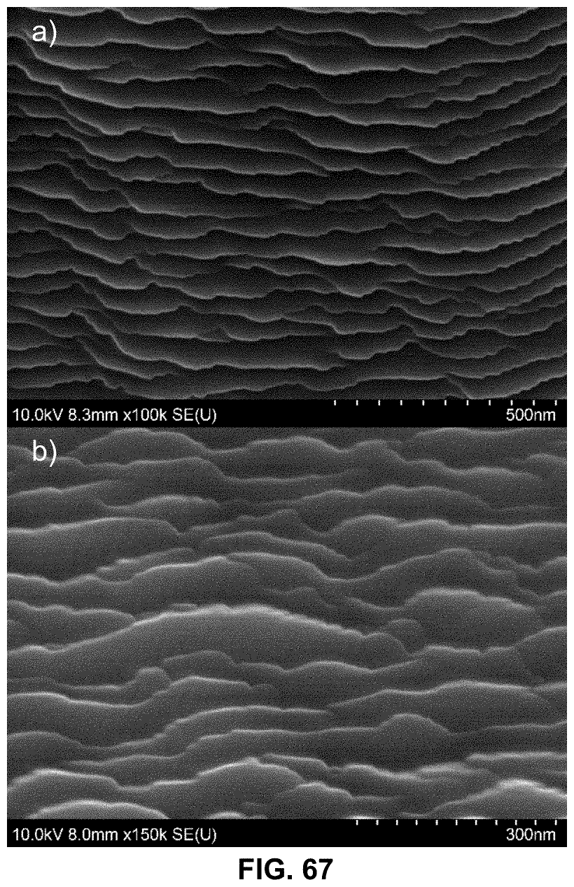

Tilted view of surface Nano-walls (Ar): When the sample is tilted at a particular angle under scanning electron microscope, an array of (parallel) wall like structures having a height of 10 to 100 nm preferably, with a length ranging from 10 to 40 nm, said ridge composed primarily of porous Ti (FIGS. 67 a-b). Such structures are formed using following DIS parameters—Gas: Ar, Angle: 0°, Energy: 1000 eV,

### Fluence: 1×10¹⁸ cgs.

### 2. Sample: 50% Ti_Ar_06

### Angle=60°

Fluence (cm⁻²)=1.00E+18
Energy (eV)=1000

### Parameter: Angle (60°)

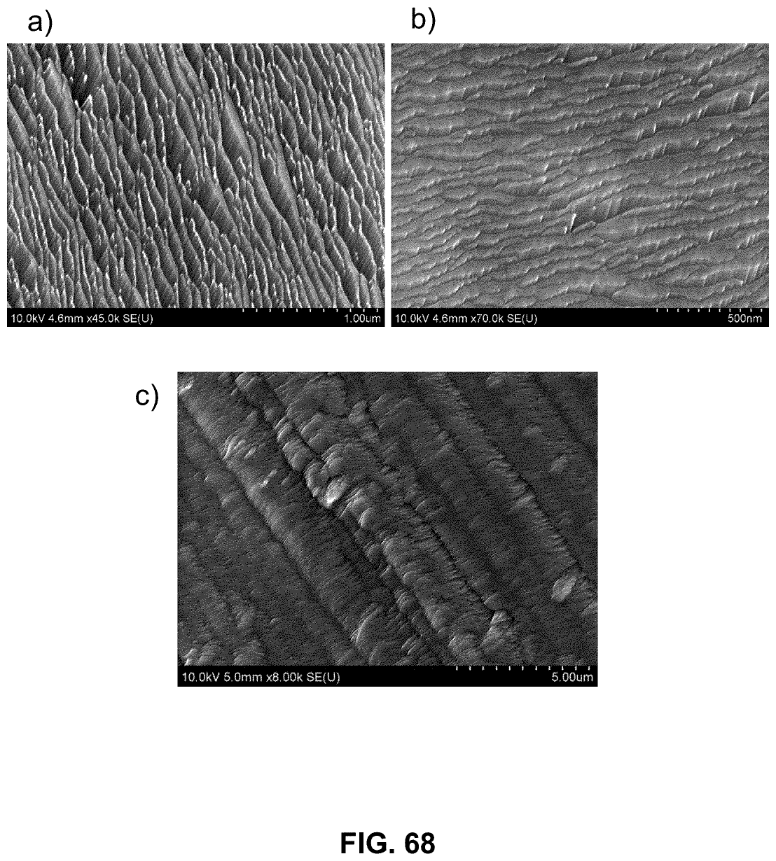

Surface Nano-walls and nano-cones (Ar): On the surface, we observe 2 different kind of nanostructures: 1) an array of (parallel) wall like structures having a height of 3 to 100 nm preferably, with a length ranging from 10 to 40 nm, said ridge composed primarily of Ti (FIGS. 68 a-b). 2) Wide and narrow nano-cones ranging between 2 to 100 nm preferably, with a length ranging from 10 to 40 nm (FIG. 68 b). Such structures are formed using following DIS parameters—Gas: Ar, Angle: 60°, Energy: 1000 eV, Fluence: 1×10¹⁸ cgs.

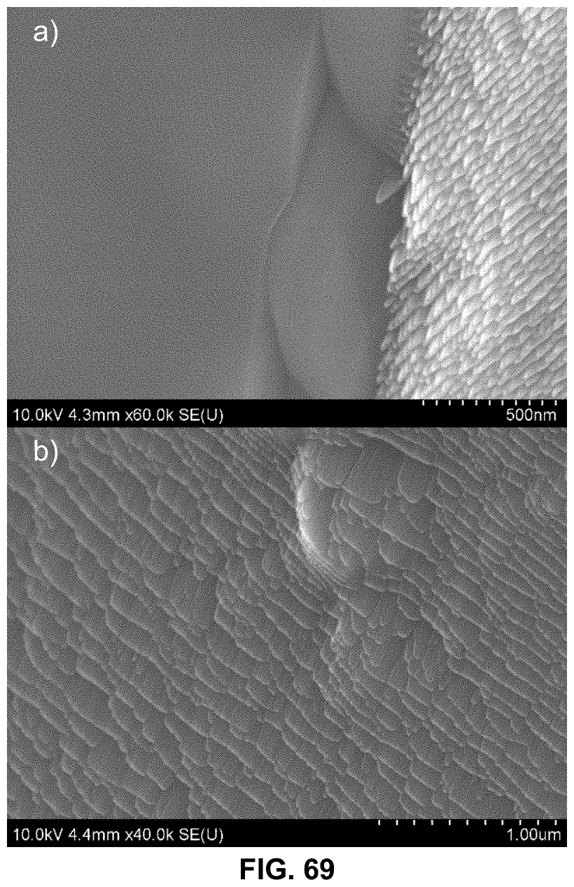

Pore Nano-walls and nano-cones (Ar): Inside the pores, we observe nano walls as well sharp nanocones. Nano-walls: an array of (parallel) wall like structures having a height of 10 to 100 nm preferably, with a length ranging from 10 to 40 nm. Nano cones: The dimension of these cones are in the range of 2 to 50 nm preferably, with a length ranging from 10 to 40 nm said ridge composed primarily of porous Ti (FIGS. 69 a-b). Such structures are formed using following DIS parameters—Gas: Ar, Angle: 60°, Energy: 1000 eV, Fluence: 1×10¹⁸ cgs.

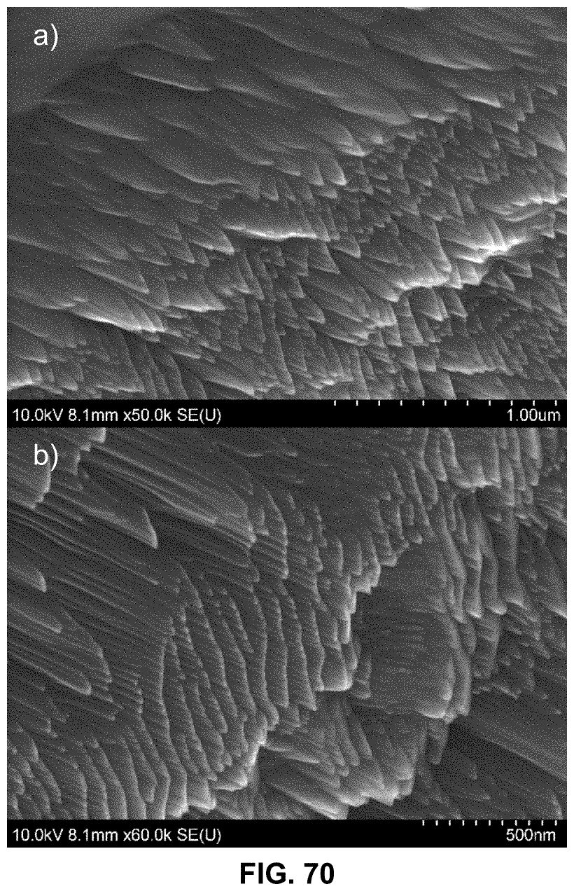

Tilted view of surface Nano-walls and nano-cones (Ar): When the sample is titled under the scanning electron microscope, an array of (parallel) wall like structures having a height of 3 to 100 nm preferably, with a length ranging from 10 to 40 nm, said ridge composed primarily of Ti (FIG. 70 a). The dimension of these cones are in the range of 2 to 100 nm preferably, with a length ranging from 10 to 40 nm (FIG. 70 b). Such structures are formed using following DIS parameters—Gas: Ar, Angle: 60°, Energy: 1000 eV, Fluence: 1×10¹⁸ cgs.

3. Sample: 50% Ti_Ar_07

### Angle=60°

Fluence (cm⁻²)=1.00E+18
Energy (eV)=500

### Parameter: Energy (500 eV)

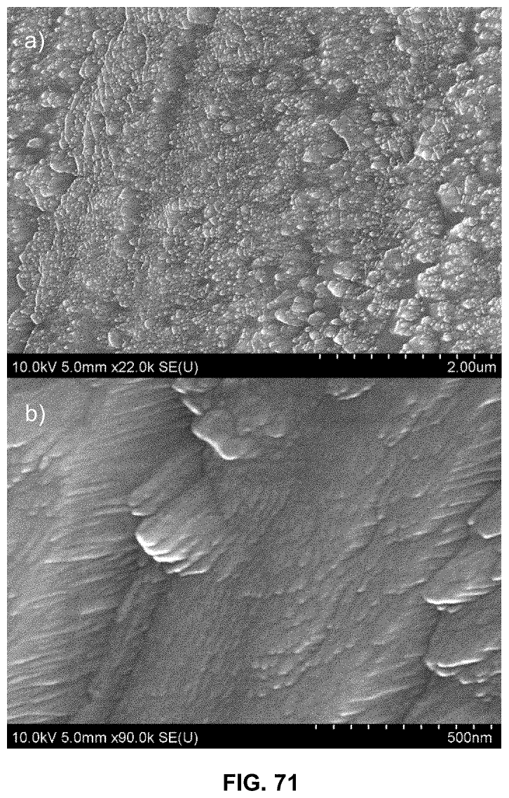

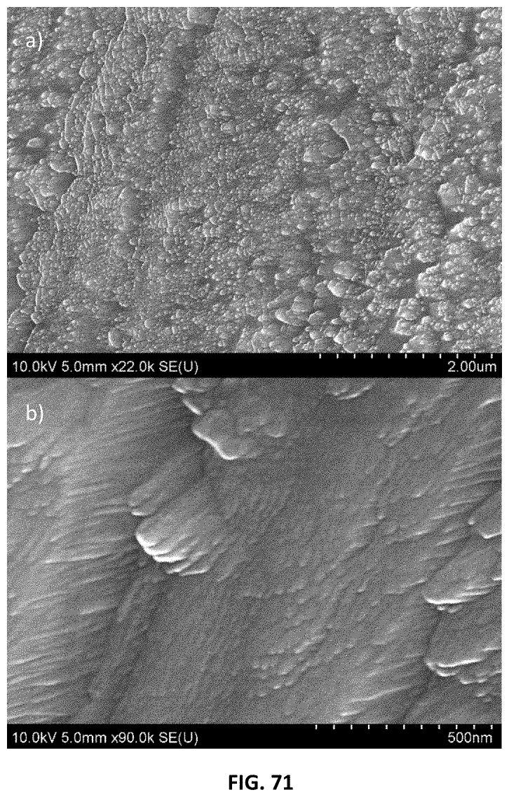

Surface nano-walls and nano-cones (Ar): At incidence energy of 500 eV, we see small nano cones and nano walls. The dimension of nano cones are in the range of 2 to 50 nm preferably, with a length ranging from 2 to 40 nm. In case of nano walls an array of (parallel) wall like structures having a height of 3 to 50 nm preferably, with a length ranging from 10 to 40 nm, said ridge composed primarily of Ti. Such structures are formed using following DIS parameters—Gas: Ar, Angle: 60°, Energy: 500 eV, Fluence: 1×10¹⁸ cgs (See FIG. 71).

### 4. Sample: 50% Ti_Ar_08

### Angle=60°

Fluence (cm⁻²)=1.00E+18
Energy (eV)=750

### Parameter: Energy (750 eV)

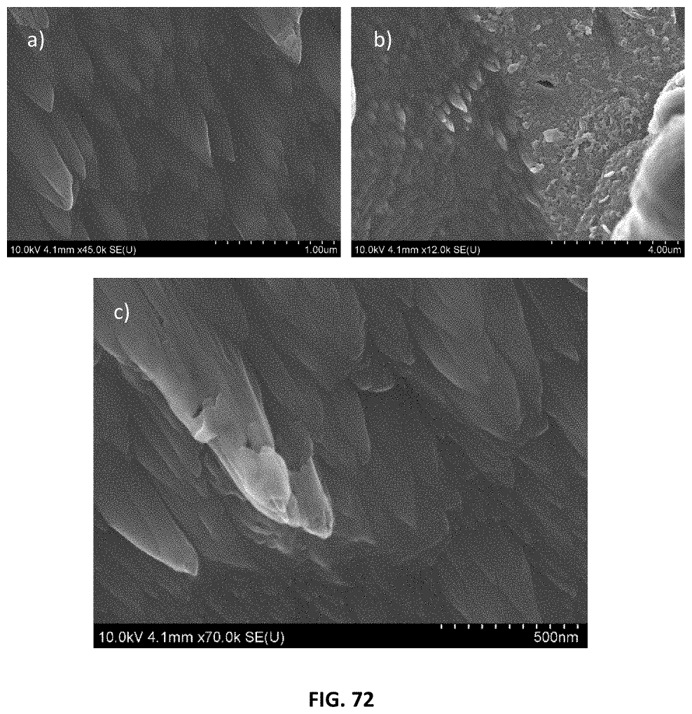

Surface Nano-cones (Ar): Structures which resemble like sharp cones. These pointed sharp regions are inclined towards the incident beam direction (FIG. 72). The dimension of these cones are in the range of 2 to 100 nm preferably, with a length ranging from 10 to 40 nm. These cones consist of Ti. Such structures are formed using following DIS parameters—Gas: Ar, Angle: 60°, Energy: 750 eV, Fluence: 1×10¹⁸ cgs.

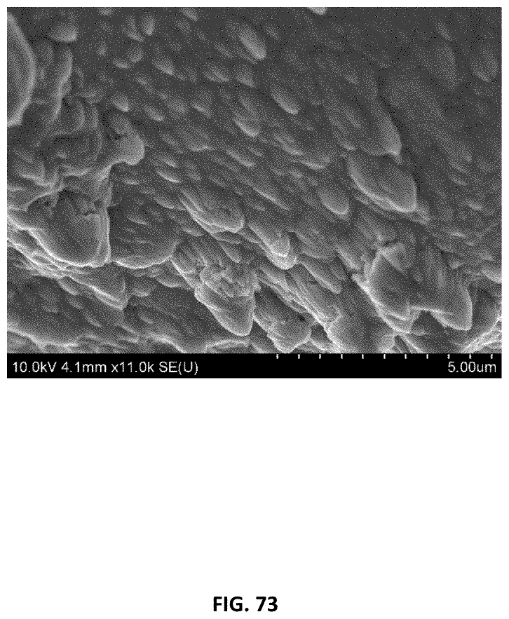

Pore Nano-cones (Ar): Inside the pores, nano cones can seen with dimensions of height of 3 to 100 nm preferably, with a length ranging from 10 to 40 nm, said ridge composed primarily of Ti (FIG. 73). Such structures are formed using following DIS parameters—Gas: Ar, Angle: 60°, Energy: 750 eV, Fluence: 1×10¹⁸ cgs.

### 5. Sample: 60% Ti_Ar_01

### Angle=0°

Fluence (cm⁻²)=1.00E+18
Energy (eV)=1000

### Parameter: Angle (0°)

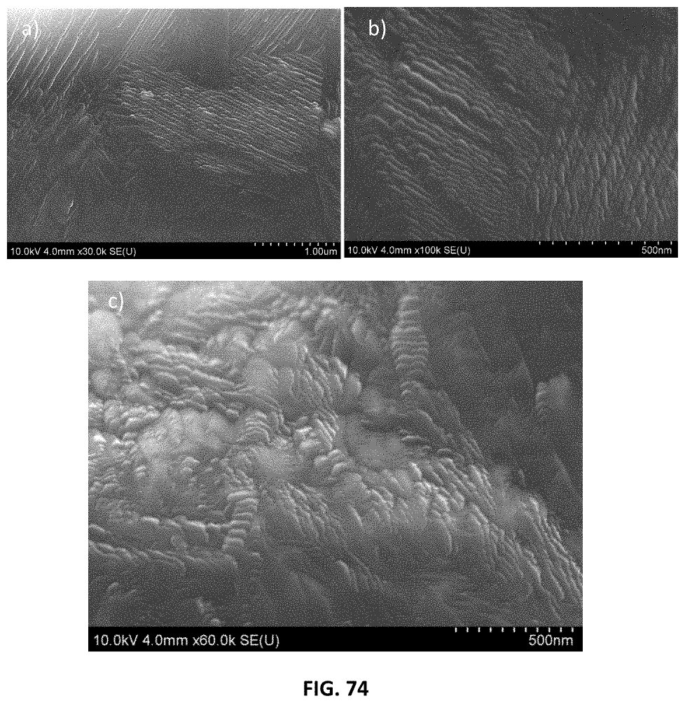

Surface Nano-walls (Ar): An array of (parallel) wall like structures having a height of 3 to 60 nm preferably, with a length ranging from 10 to 40 nm, said ridge composed primarily of Ti (FIGS. 74 a-c). Such structures are formed using following DIS parameters—Gas: Ar, Angle: 0°, Energy: 1000 eV, Fluence: 1×10¹⁸ cgs.

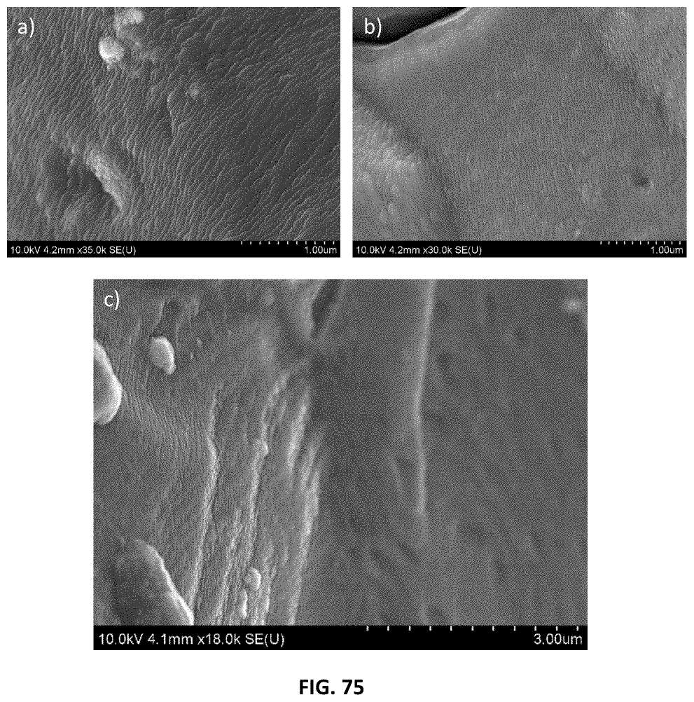

Pore Nano-walls (Ar): an array of (parallel) wall like structures having a height of 3 to 100 nm preferably, with a length ranging from 10 to 40 nm, said ridge composed primarily of Ti (FIGS. 75 a-c). Such structures are formed using following DIS parameters—Gas: Ar, Angle: 0°, Energy: 1000 eV, Fluence: 1×10¹⁸ cgs.

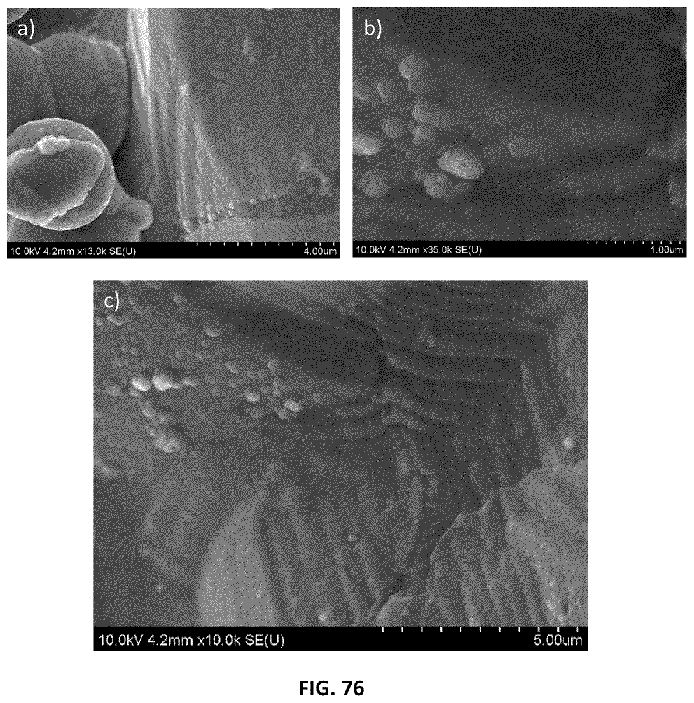

Wall of Pore Nano-walls (Ar): an array of (parallel) wall like structures having a height of 3 to 100 nm preferably, with a length ranging from 10 to 40 nm, said ridge composed primarily of porous Ti (FIGS. 76 a-c). Such structures are formed using following DIS parameters—Gas: Ar, Angle: 0°, Energy: 1000 eV, Fluence: 1×10¹⁸ cgs.

### 6. Sample: 60% Ti_Ar_02

### Angle=60°

Fluence (cm⁻²)=1.00E+18
Energy (eV)=1000

### Parameter: Angle (60°)

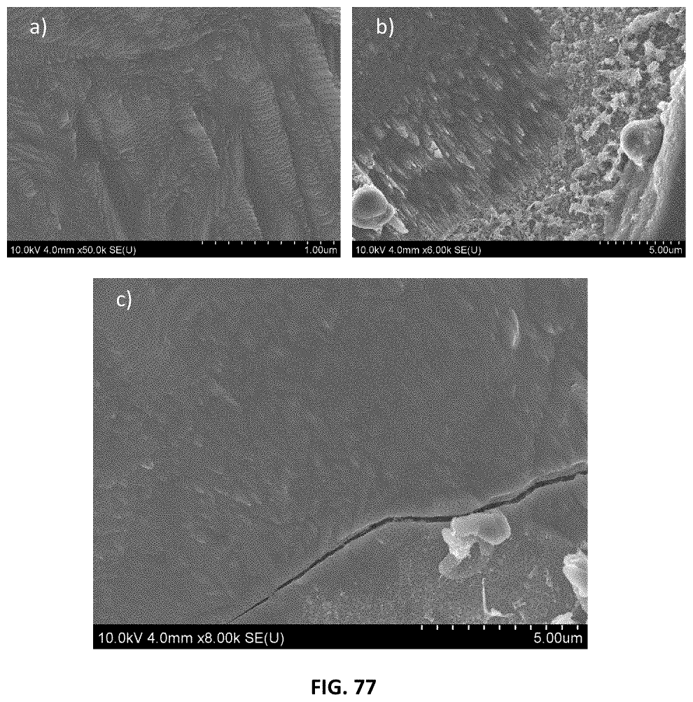

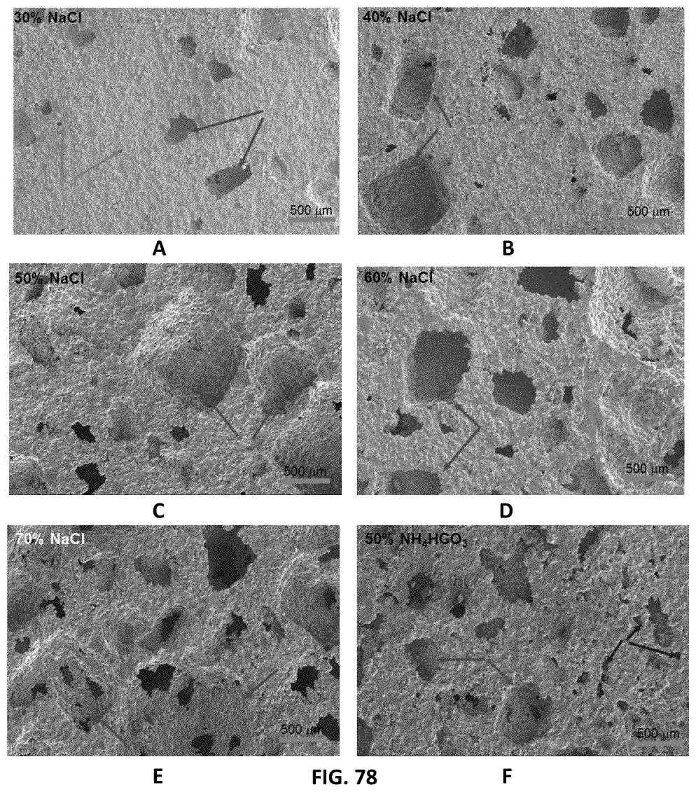

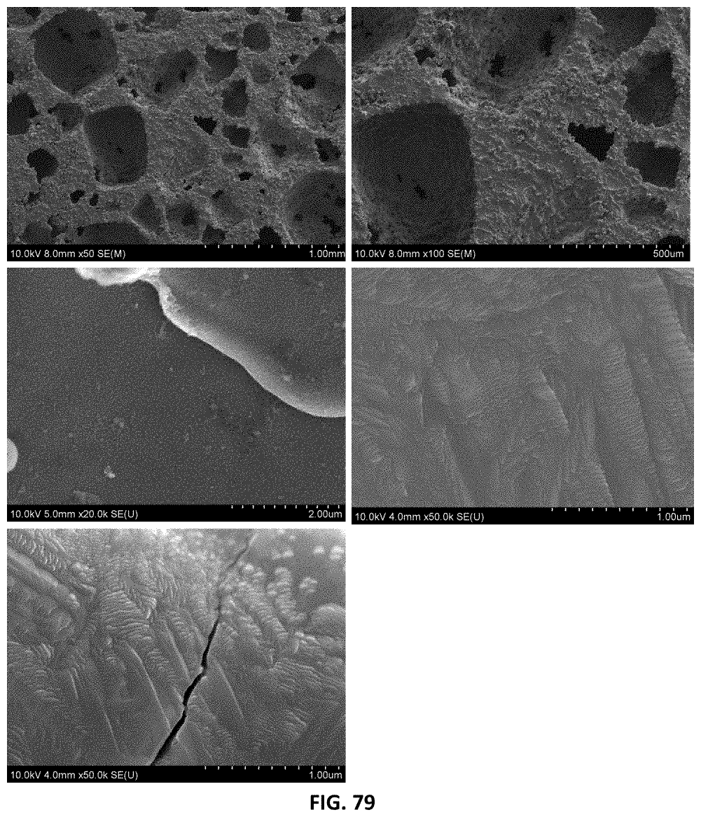

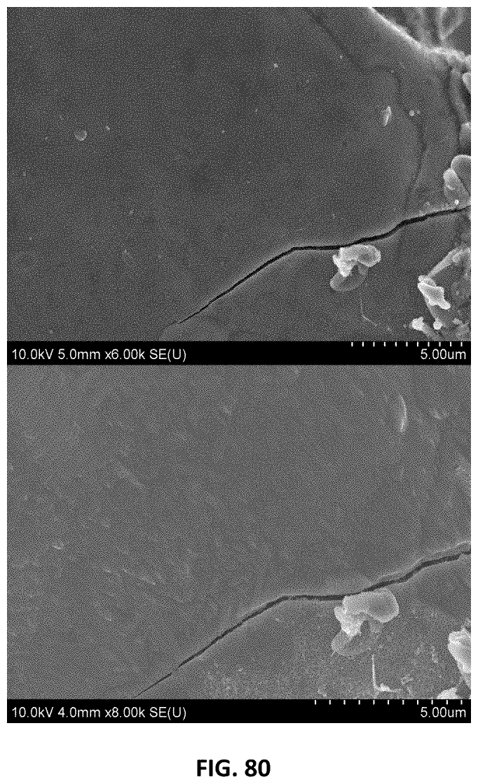

Surface Nano-walls and nano-cones (Ar): On the surface, we observe 2 different kind of nanostructures: 1) an array of (parallel) wall like structures having a height of 3 to 100 nm preferably, with a length ranging from 10 to 40 nm, said ridge composed primarily of Ti (FIG. 77). 2) Wide and narrow nano-cones ranging between 2 to 100 nm preferably, with a length ranging from 10 to 40 nm (FIG. 77). Such structures are formed using following DIS parameters—Gas: Ar, Angle: 60°, Energy: 1000 eV, Fluence: 1×10¹⁸ cgs.

### REFERENCES

Nanostructured biointerfaces (2014), J P Allain, M Echeverry-Rendon, J J Pavon, S L Arias, Nanopatterning and Nanoscale Devices for Biological Applications, 41-72.
Titanium surface modification by directed irradiation synthesis (DIS): nanostructuring for regenerative medicine (2013), J Pavon, O El-Atwani, E Walker, S L Arias, J P Allain, Health Care Exchanges (PAHCE), Pan American, 1-1.

### Stage of Development

Preliminary biological assessment by Comet Assay® testing of porous cpTi irradiated samples has shown that Ar+ions beams has no toxicological detrimental effect on initial biocompatible surface. HASMCs morphology behavior in contact modified surface, and in terms of time, also suggests that these new surfaces will in fact improve in Vivo tissue stimulation. This can be stated from the consistency with several previous results, as well as because those important improvements observed on surface nano-structuring, controlled roughness and reduced free surface energy. In summary, phenomenological relationships and trends determined here allowed us to have a new insight about positive influence of Ar+irradiation on porous cpTi surfaces in order to not only improve osseointegration, but also to effectively promote bone tissue growth and repair.

### Example 5: Nanostructured Ti6Al4V Biointerfaces by Directed Irradiation Synthesis for Improved Cell Adhesion and Proliferation

### Brief Overview

Provided is both a method and material structure transformed by subject method to elicit specific bioactive surface functions. In this invention directed irradiation synthesis (DIS) is used combining a sequenced asymmetric ion-beam that can be combined with a thermal-particle beam to induce surface nanostructures on medical grade Ti6Al4V. The material structures are introduced and vary in morphology and topology. The nanostructure morphology and topology has a correlated dependence on the crystallographic grains of the Ti alloy material. The nanostructure morphology, surface chemistry and topology are controlled independently by incident angle, fluence, energy and species. The ratio of ion to thermal particle flux can also influence morphology and/or topology. The nanostructure morphology and surface morphology in turn can control cell shape directing desired cell lines into specific phenotype behavior. Three primary bioactive properties are induced: 1) cell shape, 2) cell adhesion and proliferation and 3) bactericidal and anti-bacterial physical surface structures. Thus there is an enhancement of cell integration in bone tissue, for example, with a more favorable healing time range. Combining these new properties with the favored intrinsic biomechanical properties of Ti-based alloys makes for a beneficial biomaterials system.

The provided compositions and methods have implications for tissue engineering in endovascular and bone integration applications including as an advanced biointerface for bone implants and stent materials for vascular reconstruction. Other applications include implants for spinal cord injury.

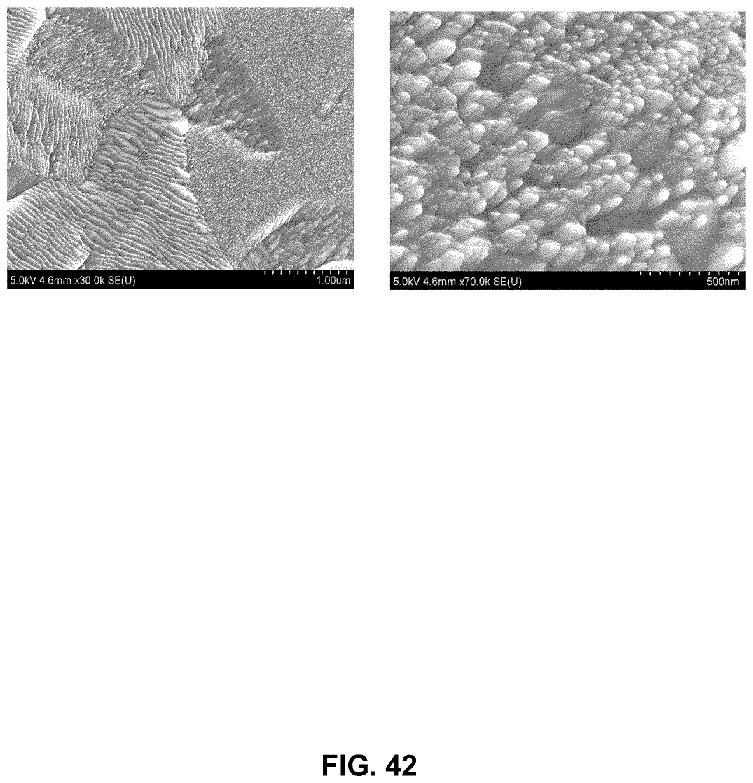

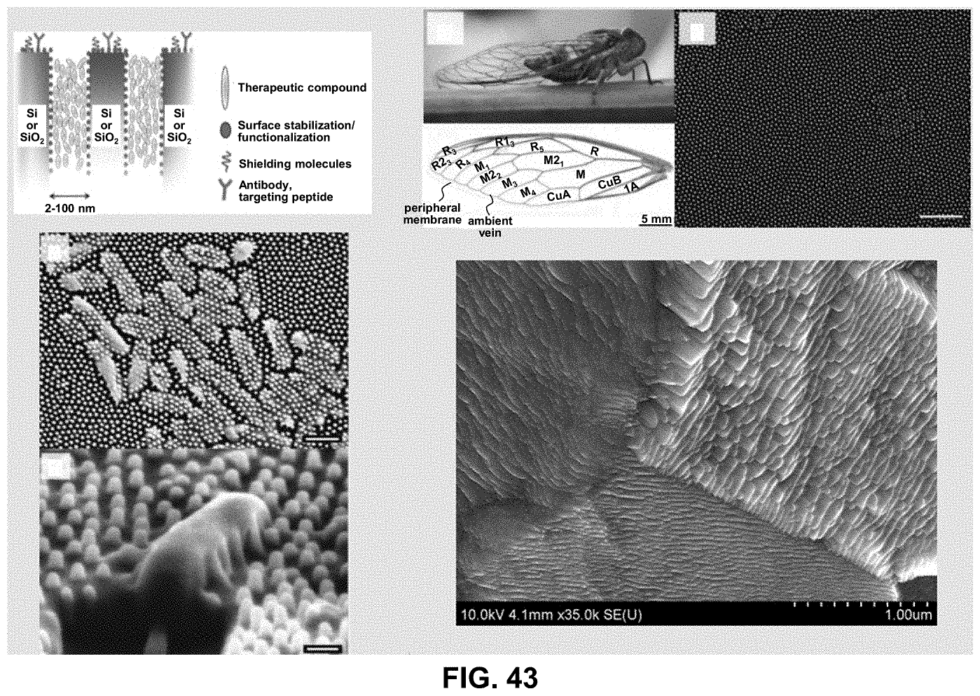

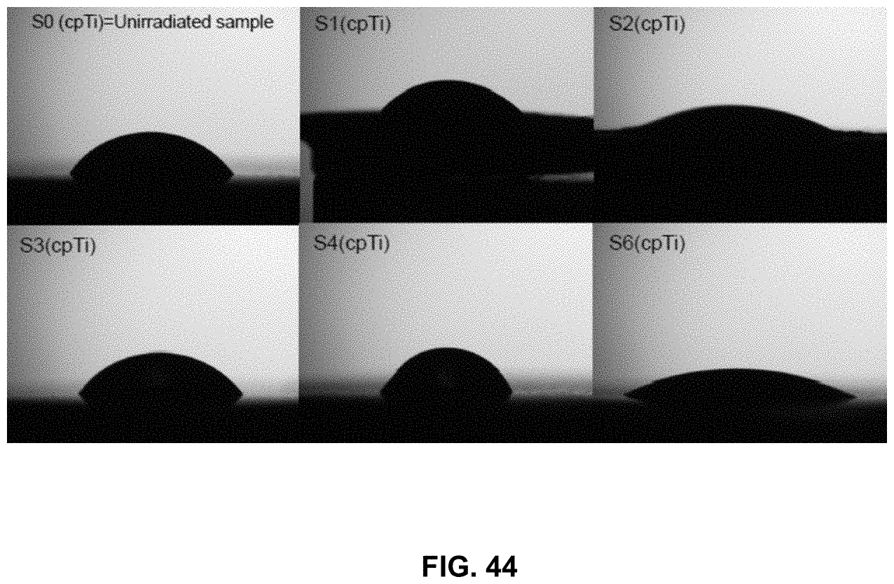

Titanium and its alloys is widely recognized to be the preferred biomaterial for bone replacement due to its excellent balance between biomechanical and biocompatibility response. However, titanium suffers from the intrinsic growth of a thin fibrous tissue interface. FIG. 42 shows the complex interplay between pro-inflammatory and anti-inflammatory behavior after the insertion of an implant in the body. The creation of fibrous tissue prevents the effective integration of bone tissue to the implant. Macrophage adhesion and recruitment of immune cells occurs within several weeks of implant introduction to the body. Therefore, biomaterials that quickly integrate into the tissue (e.g. bone, endothelium, etc. . . . ) would provide for enhanced integration of biomaterial and minimization of complications that compromise patient care.

By transforming the surface of Ti-based biomaterials implants can provide for fast tissue reconstruction by enhancing tissue integration in a time scale that prevents immune-system response to the foreign body biomaterial. Meaning that as a consequence of nanofeatures fabricated on the biomaterial in question it imparts the function to effectively influence surrounding biological environment at a molecular nanoscale level. Several studies demonstrated different morphology configurations at the nanoscale can have a strong and direct influence over cellular behavior; indeed, it is possible to appreciate that cells prefer texturized surfaces in comparison with smooth ones. Biointerface topography and, in particular, nanoscale features can affect cell behavior and integrin-mediated cell adhesion, and is now evident from studies with fabricated topographical features. The extent to which nanotopography influences cell behavior in-vitro remains unclear, and investigation on this phenomenon is still underway. The processes that mediate the cellular reaction with nanoscale surface structures are also not well understood. For example, it is not clear if this influence derives directly from surface topography, or perhaps indirectly with surface structures possibly affecting the composition, orientation, or conformation of the adsorbed ECM (extra-cellular matrix) components. However, current practice is to simply increase the surface roughness of an implant material in the effort to elicit a more favorable cellular response. The compositions and methods to fabricate the same enable a high-fidelity control of cellular behavior that would in principle influence such mechanisms as: cell proliferation, cell differentiation and cell adhesion and migration.

Directed irradiation synthesis (DIS) and directed plasma nanosynthesis (DPNS) address this limitation by introducing a synthesis process that is scalable to high-volume manufacturing by virtue of its intrinsic large-area simultaneous exposure of materials surfaces and interfaces. One subset of DIS is ion-beam sputtering (IBS) and is the methodology used in the work reported here. Advanced in-situ synthesis methods have been recently developed by Allain et al. to elucidate ion-irradiation mechanisms that can manipulate surface chemistry and surface morphology to ultimately synthesize functional coatings for 3D scaffold systems. We vary a limited number of parameters that introduce a specific morphology with a specific cell shape response as demonstrated with HASMC biological assay tests shown below.

A few papers have reported converting Ti into a third generation biomaterial, but few address the modification of Ti-based material surfaces for regenerative medicine applications. For example, the latest technologies involving Ti-based implant systems only address enhancements to the surface roughness or vaguely the surface chemistry but with very little control of either and certainly not of both independently. Most of them have shown improvements of cell adhesion due to interactions with nano-patterned surfaces, as regulators of cellular functions through focal adhesion. Results include: increased adhesion and formation of human mesenchymal stem cells, improvements of rat aortic endothelial cells adhesion on TiO2, upregulation of osteospecific proteins-osteopontin and osteocalcin of human osteoprogenitor cells cultured on Ti. However, nowhere in prior art is there a systematic independent high-fidelity (e.g. structure shape, size, sequence, extent) control of morphology and surface chemistry. This can only be achieved by processing with DIS and/or DPNS approaches pioneered in Prof. Allain's group.

Conventional processing of materials for surface nanostructuring has been dominated by the development of advanced top-down fabrication techniques that include lithography-based techniques. Some of them include focused-beam lithographies using electron or ion energetic particles and scanning probe lithographies. The drawbacks of these conventional techniques have mainly been attributed to their physical limitations in fabricating structures smaller than about 50-nm and also limited to the modification of a few classes of materials Prior art in surface processing is constrained to the use of these lithography-dependent patterning approaches that limits scalability and versatility, which drive industrial scalability strategies. One important limitation in current nanomanufacturing approaches is a dependence on naturally self-ordered processes that balance kinetic and thermodynamic dissipative forces in the absence of irradiation therefore requiring very high temperature processes. Consequently, many of the desired biomaterial properties that require a combination of metal alloy and soft material interfaces cannot be processed with conventional bottom-up techniques or techniques that rely on chemical processes to induce a surface variation.

Directed irradiation synthesis (DIS) and directed plasma nanosynthesis (DPNS) address this limitation by introducing a synthesis process that is scalable to high-volume manufacturing by virtue of its intrinsic large-area simultaneous exposure of materials surfaces and interfaces. One subset of DIS is ion-beam sputtering (IBS) and is the methodology used in the work reported here. Advanced in-situ synthesis methods have been recently developed by Allain et al. to elucidate ion-irradiation mechanisms that can manipulate surface chemistry and surface morphology to ultimately synthesize functional coatings for 3D scaffold systems.

Provided is a new process which produces different nanostructures such as: nanoripples and nanorods depending on the incidence angle of ions. The interactions between the cells and the nanopatterned surfaces exhibited overall improvement of cells behavior and in particular cell shape control and hydrophilic properties.

Further, described is an enhanced bioactive interface for Ti-based systems and a process and arrangement to generate patterned structures and unique topography at the nanoscale while eliminating the shortcomings of the prior approaches related to high-volume manufacturing resulting in a process that is scalable to by virtue of its intrinsic large-area simultaneous exposure of materials surfaces and interfaces. In a first embodiment, a method for fabricating structures on substrate having a substrate surface includes providing a set of control parameters to an ion beam source and thermal source corresponding to a desired nanostructure topology. The method also includes forming a plurality of nanostructures in a first surface area of the substrate by exposing the substrate surface to an ion beam from the ion beam source and thermal energy from the thermal source. The ion beam has a first area of effect on the substrate surface, and the thermal energy has a second area of effect on the substrate surface. Each of the first area and the second area includes the first surface area. In other words, the coincident beams under the set of control parameters produce a plurality of microstructures or nanostructures.

In particular, the technology as described in the present disclosure has important ramifications for biomaterials which are important for introducing design pathways tuning bioactive properties used in multiple applications for biocompatibility and bio-surface material adaptability.

### Technical Description, Details and Supporting Data

DIS conditions of different cpTi samples appear summarized in Table 16 for systems where the fluence and incident particle energy is kept constant and the incident angle is varied.

TABLE 16
Irradiation Parameters (1-keV Ar⁺) on Ti6AI4V Samples

| | Energy | Flux (ions sec | Fluence | Incidence | |
|---|---|---|---|---|---|
| Samples | (keV) | cm⁻²) | (cm⁻²) | angle (°) | Time (s) |
| S1 (Ti6) | 1.0 | 6.51E14 | 2.5E17 | 0° | 384.0 |
| S2 (Ti6) | 1.0 | 3.29E14 | 2.5E17 | 30° | 758.8 |
| S3 (Ti6) | 1.0 | 6.44E14 | 2.5E17 | 60° | 387.9 |
| S4 (Ti6) | 1.0 | 6.55E14 | 2.5E17 | 80° | 381.6 |

Surface structural modifications and nano-structuring due to DIS of commercially pure titanium (cpTi) samples are summarized in FIG. 1. The influence of DIS on cpTi is reflected in a general nanopatterning; it mostly corresponds to short oriented nanorods, preferentially oriented in the same orientation of the ions beam direction. However, some of them appear with a switch of direction, most probably associated to different crystallographic direction of a phase (hcp) grains. This synthetized nano-patterning appears partially as some nano-ripples or with a mixed structure between nano-rods and nano-ripples. The prevalence of nano-rods indicates that the incident angle has the same value of the transition angle or slightly higher (diffusive to erosive regime; rods and ripples to 100% nano-rods). The observed general effect of DIS nano-patterning is the consequence of cpTi microstructure as a monophasic alloy of a phase (hcp); the whole coherency between nano-rods and nano-ripples with respect to ion beam direction would indicate that surface grains are texturized; i.e. grains in the same direction that could be the consequence of the polishing operation which is also verified because of the consistency with polishing scratch directions. It is relevant the high throwing power of this DIS nano-patterning, which is reflected in the capability to create nano-rods inside the pores, and inside the scratches as well (see details in FIG. 1). Not only nano-rods, but also nano-columns can be easily appreciated inside the gaps-scratches, in a similar way as it is normally observed inside a trench during practice FIB technique. Those columns from the bottom of the scratch seem to grow approximately perpendicular to surface; however, those nano-rods far away from the gap, appear more elongated and they tend to be more parallel to the surface.

Variation of Incident Ion Particle Fluence

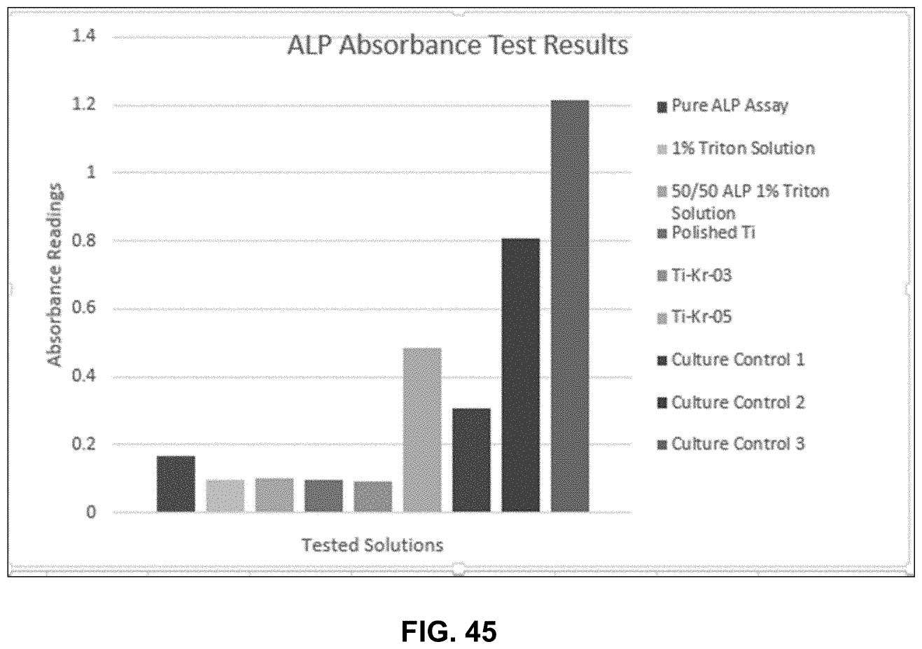

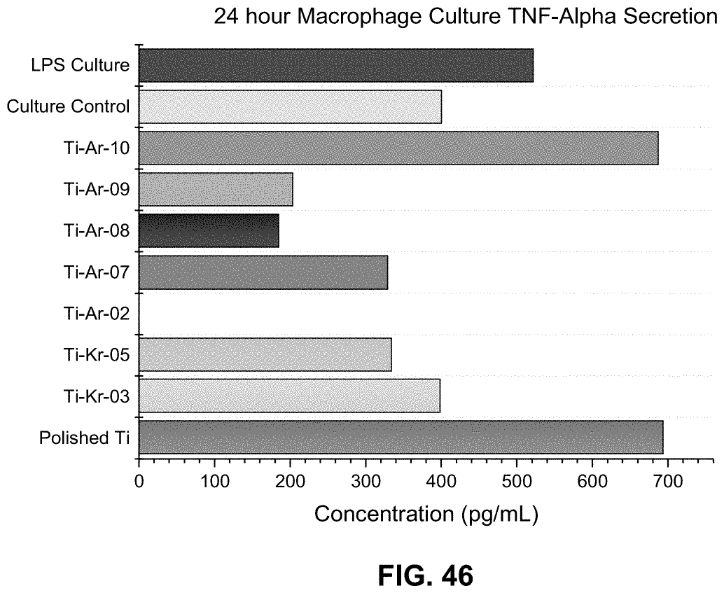

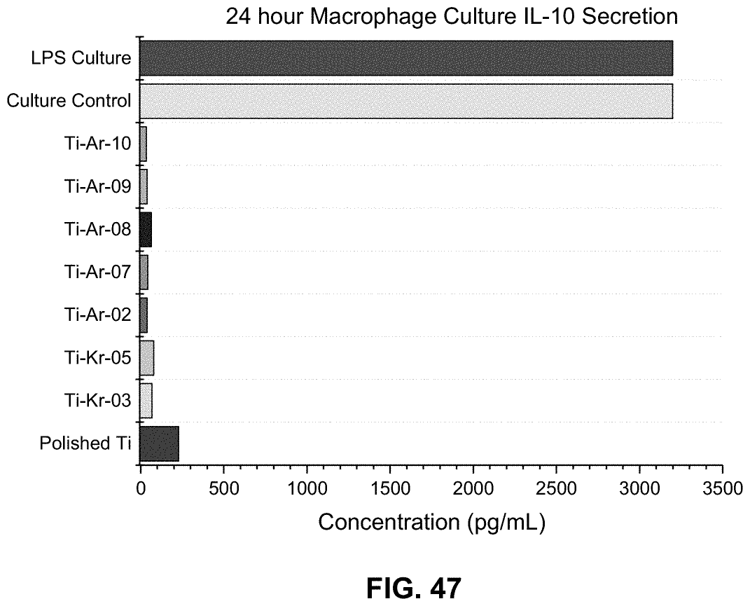

Although high-fidelity control of Ti-based nanostructures is already attained by varying the incident angle we also observe that varying the fluence will result in further control of surface morphology that can be tailored depending on the cellular response needed. Table 17 summarizes the reduced to practice parameters with “oblique incidence at 60-degrees”. In another application of this approach the significantly increased surface-to-volume area also enables the use for peptide attachment and biosensing properties. FIGS. 45-47 show high-resolution SEM images of the striking nanotopography attained with DIS. Note the enhanced surface-to-volume ratio results in surface area increases by factors of 10-100 to conventional anodization or chemical-based surface treatment processes.

TABLE 17
Parameter space for DIS

| | | Energy | Fluence | Incident |
|---|---|---|---|---|
| Sample Name | Gas | (keV) | (cm⁻²) | Angle (°) |
| Ti—Kr-03 | Krypton | 1 | 1.0 × 10¹⁸ | Normal |
| Ti—Kr-05 | Krypton | 1 | 1.0 × 10¹⁸ | Oblique |
| Ti—Ar-07 | Argon | 1 | 1.0 × 10¹⁷ | Oblique |
| Ti—Ar-08 | Argon | 1 | 2.5 × 10¹⁷ | Oblique |
| Ti—Ar-09 | Argon | 1 | 5.0 × 10¹⁷ | Oblique |
| Ti—Ar-10 | Argon | 1 | 7.5 × 10¹⁷ | Oblique |
| Ti—Ar-02 | Argon | 1 | 1.0 × 10¹⁸ | Oblique |

Biological Assessment of as-Received (AR) and DIS Treated cpTi Samples

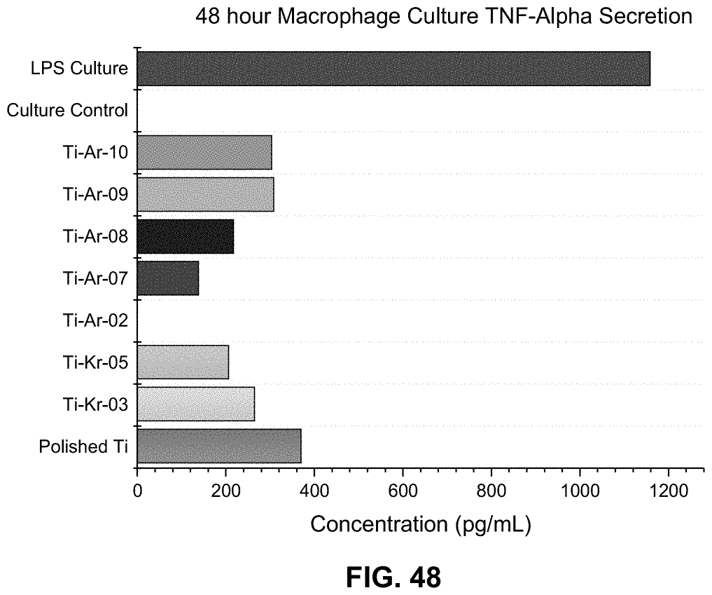

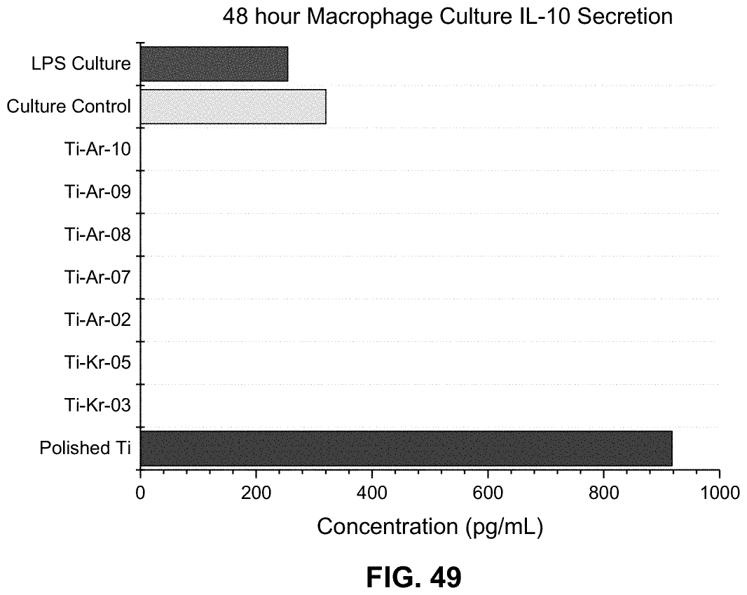

As it is well recognized from biological response of biomaterials surfaces, free surface energy of biomaterials reflected in their contact angle values plays a determinant role in the biological environment response. By considering the effect of multiple surface properties in contact angle results, here we can establish important relationships with surface modifications due to ion irradiation processing of cpTi surfaces. In that context, osseointegration is one of the good examples in which, besides the roughness, the surface tension is a parameter that interferes on it. It permits a higher or smaller scattering of liquid onto the metallic surface. The human blood contains about 90% of water, thus the capability of water adsorption by the surface, known like wettability, is a fundamental parameter to the success of cellular adhesion and, consequently, to the osseointegration. It is commonly accepted that blood compatibility is improved when the hydrophilicity of a surface is increased (unless the surfaces are superhydrophobic) (FIG. 48).

Cytotoxicity assessment of irradiated samples was performed via Comet Assay® testing, in search of evaluating the potential geno-toxicological effect in vitro on human aortic smooth muscle cells (HASMCs) cultured in the presence of the evaluated specimens (FIG. 7). It indicates that irradiated cpTi surfaces did not induce DNA damage on HASMCs cultured in the presence of these materials under the experimental conditions tested in this work. The results depicted in the present work suggest that irradiated cpTi samples with Argon under different conditions do not produce detectable DNA damage in HASMCs.

Biocompatibility is, at least, equal to well-known biocompatibility of conventional bio-inert cpTi surface; with respect to cell behavior parameters, by considering our improvements in nano-structuring, roughness parameters and contact angle, we can reasonably expect that cells factors like adhesion, proliferation, migration, and differentiation will be also improved with the surfaces obtained here. However, it is important to point out at this moment that our results reported here are new in the sense that is the first time is shown relationships between initial surface micro-topography of cpTi, irradiation conditions, surface nano-structure, roughness, surface free energy and basic biocompatibility assessment; they are related not only with their behavior and adhesion, but also with their further potential to stimulate tissue growth.

### Prior Methods, Apparatus, Developments and Publications

Directed irradiation synthesis (DIS) of porous and rough cpTi samples.

Grinding and polishing of cpTi samples were performed, before DIS processing. DIS experiments were performed at Radiation Surface Science and Engineering Laboratory (RSSEL) at the University of Illinois at Urbana Champaign, which is originally developed and set-up by Prof. Jean Paul Allain (FIG. 41). DIS conditions of different cpTi samples are summarized in Table 17.

Structural and Surface Free Energy Characterization of cpTi Samples Modified by DIS.

Surface free energy of irradiated samples was evaluated by contact angle testing with deionized water through a Rame-Hart Goniometer Model 500-Advanced contact angle goniomter/tensiometer with DROPimage Advanced Software. We performed the sessile method of contact angle analysis (where the sample was static and not moving after the drop was placed on it). All measurements were performed with deionized water (to not have any type of interaction with the surface). The water dropper was far enough away from the surface that the water did not touch the sample until it left the dropper. The dropper was kept at the same distance from each sample surface. The water droplets had ˜3 μL of water on each sample. Morphological features of samples were detailed analyzed by Scanning Electron Microscopy (SEM).

Biological Evaluation of cpTi Samples Modified by DIS.

The cells used for biological assessment of treated and un-treated samples were human aortic smooth muscle cells (HASMCs, Lifetechnology Cat # C0075C). To that end, cells morphology changes with culture time, and cytotoxicity assays test on modified surfaces via Comet Assay@ testing, were performed. Changes in cells morphology in terms of culture time was observed by using optical microscopy (OM) analysis. The cell line used was the choose model to validate the potential for tissue growth and regeneration of the new surfaces. Cells were grown at 37° C. with >95% Rh and C02 gas exchange until they were nearly confluent. HASMCs were seeded on the top of the samples for 24 hours under normal culturing conditions (37° C., 95% air, 5% C02, 95% humidity).

### REFERENCES

Nanostructured biointerfaces (2014), J P Allain, M. Echeverry-Rendón, J J Pavon, S L Arias, Nanopatterning and Nanoscale Devices for Biological Applications, 41-72.
Titanium surface modification by directed irradiation synthesis (DIS): nanostructuring for regenerative medicine (2013), J Pavón, O El-Atwani, E Walker, S L Arias, J P Allain, Health Care Exchanges (PAHCE), Pan American, 1-1.
J P Allain, Directed irradiation synthesis of multifunctional nanostructured bioactive surfaces. ABSTRACTS OF PAPERS OF THE AMERICAN CHEMICAL SOCIETY 246, 2013.
J. Pavon, E. Walker, S. Arias, L. M. Reece, J. P. Allain, “New Nanostructured Multifunctional Surfaces of Titanium Alloys Obtained by Directed Irradiation Synthesis (DIS) for Treatments of Spinal Cord Damages,” 10th Annual World Congress of the Society of Brain Mapping and Therapeutics, May 12-14, 2013, Baltimore, Md., USA.
J. Pavon, J. P. Allain, O. El-Atwani, M. Echeverry-Rendon, S. Arias, “Directed Irradiation Synthesis of Titanium-based Biointerfaces for Tissue Regeneration”, MRS Spring Meeting & Exhibit; Apr. 1-5, 2013, San Francisco, Calif.
A. Barnwell, S. L. Arias, A. R. Shetty, J. P. Allain, “Nanostructuring to Improve Osseointegration of Titanium Implants in Spinal Reconstruction”, BMES, Minneapolis, Minn.; Oct. 5-8, 2016.

### Stage of Development

Preliminary biological assessment by Comet Assay® testing of cpTi irradiated samples has shown that Ar+ions beams has no toxicological detrimental effect on initial biocompatible surface. HASMCs morphology behavior in contact modified surface, and in terms of time, also suggests that these new surfaces will in fact improve in vivo tissue stimulation. In summary, phenomenological relationships and trends determined here allowed us to have a new insight about positive influence of Ar+irradiation on both porous and rough cpTi surfaces in order to not only improve osseointegration, but also to effectively promote bone tissue growth and repair. Furthermore, DIS and DPNS exposures with other species such as Kr+, Ne+ and O2+ have also enabled the formation of specific nanostructures and chemistries pertinent to this invention. Cell adhesion, proliferation and cell-shape morphology have all responded with increased levels of activity by over 50% and some cases over 100%. More importantly, the tunability and high-fidelity modulation of the immuno response of macrophage phenotypes suggest enhanced osseointegration an osseoconduction.

### Example 6: Experimental Parameters

Titanium is widely used to produce implants because direct contact occurs between bones and implant surfaces. Titanium has excellent biocompatibility, superior corrosion resistant as well as durable in physiologic mediums. Moreover, it is easily prepared in many different shapes and textures without affecting its biocompatibility. Despite these advantages, some problems of titanium in hard tissue applications are still controversial. The implant fixation to the bone remains an aspect to be improved through alternatives for reducing the stress-shielding phenomenon, which is a consequence of the mismatch between Young's modulus values (titanium is 110 GPa and cortical bone around 20-30 GPa); this difference has been identified as one of the major reasons for implant loosening and bone resorption. Furthermore, it has been suggested that when bone loss is excessive, it can compromise the long-term clinical performance of the prosthesis. This may also be responsible for implant migration, aseptic loosening, fractures around the prosthesis, and can imply technical problems during revision surgery.

Solving the stress-shielding problem requires the development of new implants and prosthesis with a lower stiffness than conventional designs, without any critical detrimental effect on the mechanical strength. Within the effort to obtain implants with a better stiffness match with the cortical bone, there are several important advances already reported. One of those explored routes to obtain porous titanium is the space-holder technique, which is a modification of conventional powder metallurgy. This technique consists of mixing the metal powder with a special additive to be removed before sintering. We have prepared porous Ti using NaCl and NH₄HCO₃ as space holder material, with 30, 40, 50, 60 and 70 vol. % concentration (NaCl) and 50% (NH₄HCO₃).

Porous titanium is considered a promising biomaterial for various applications in orthopedics including bone substitution and total joint replacement surgeries. Using the space holder technique, it is now possible to manufacture porous titanium with mechanical properties in the range of the mechanical properties of bone. Open porous biomaterials have large surface area that could be modified using bio-functionalizing surface treatment techniques for improved performance of the implant. The surface of biomedical materials is often treated using surface engineering techniques to improve the (biological) performance of the materials. Since titanium is bio-inert, surface treatments and coatings are often applied to improve their bioactivity. Surface nanostructuring of conventional biomaterials has emerged as one of the most important and effective manners to convert them in advanced biomaterials; this is the consequence of nano-features capability to effectively have some influence on surrounding biological environment at molecular nano-scale level. Since the cells in their natural environment are surrounded by nanoscale features linked to their extracellular matrix (ECM), the nanotopographical parameters become an essential part in design of biomaterials for tissue formation and repair. Accordingly, several studies suggest that a remarkably small modification in surface nanotopography could result permissive for mesenchymal stem cell growth and development, indicating that changes in such nanotopographical features had a direct influence in the adhesion/tension balance to permit self-renewal or targeted differentiation. Biointerface topography and, in particular, nanoscale features can affect cell behavior and integrin-mediated cell adhesion, and is now evident from studies with fabricated topographical features.

Introduced herein is directed irradiation synthesis (DIS) as a process that can pattern porous Ti samples not only on the surface but also inside the pores and pore walls. Broad-beam ions combined with rastered focused ions and gradient ion-beam profiles are sequenced and/or combined with reactive and/or non-reactive thermal beams that control the surface topography, chemistry and structure at the micro and nano-scale. In this work, we show that porous cpTi samples can be nano-patterned inside and outside the pores in such a way that the biological response of the surface can be favorably influenced. To that end, treated samples were detailed characterized in order to establish relationships with DIS conditions, and with surface energy and structural properties as well. These new surfaces were also biologically evaluated by using human aortic smooth muscle cells (HASMCs) for cytotoxicity assessment. Overall analysis of DIS influence on porous cpTi samples allowed identifying the real potential of our technology for nano-patterning and generates a positive biological response.

TABLE 18
Irradiation parameters on porous Ti samples.

| | Energy | Angle | Fluence |
|---|---|---|---|
| Sample | (keV) | (degree) | (cm⁻²) |
| Angle | | | |
| 30%Ti_Ar_05 | 1000 | 0 | 1.00E+18 |
| 50%Ti_Ar_06 | 1000 | 60 | 1.00E+18 |
| Energy | | | |
| 30%Ti_Ar_03 | 500 | 0 | 1.00E+18 |
| 50%Ti_Ar_04 | 750 | 0 | 1.00E+18 |
| Energy | | | |
| 30%Ti_Ar_07 | 500 | 60 | 1.00E+18 |
| 50%Ti_Ar_08 | 750 | 60 | 1.00E+18 |
| Angle | | | |
| 60%Ti_Ar_01 | 1000 | 0 | 1.00E+18 |
| 60%Ti_Ar_02 | 1000 | 60 | 1.00E+18 |

### Example 7: 3D Complex Geometries of Titanium Dental Implants

Medical devices, which are design to be implanted in living tissues, have to fit perfectly in the tissue defect. There, they will get in contact to host cells and body fluids at the same time, therefore, these biomaterials must have an optimum design of their surface which should promote and facilitate tissue integration. Among the minimum requirements, biocompatibility and non-toxicity, biomaterials should be developed in a controlled manner in order to promote a reduction of bacteria attachment, a reduction or delay of immune response and enhancement of mesenchymal and osteoblast cells adhesion and differentiation.

During the past few years, the surface modification of dental implants has grown and developed new strategies to enhance the surface properties in order to achieve faster osseointegration and a reduce bacteria attachment. In general terms, the survival rates of dental implants are high, however, there is still implants failure of around 2% and 5% after one month and one year respectively. Implant rejection still occurs and therefore, the main focus of the research society is in the development of new strategies to achieve a suitable surface modification of dental implants which decrease the percentage failure.

Many efforts have been centered on the simulation of the bone matrix chemistry using an active coating with biomacromolecules such collagen or ceramics such hydroxyapatite which supports chemical cues to promotes a proper osseointegration. However, dental implants are going to contact with other tissues, not only hard bone, they have to adjust to soft tissue healing. Regarding dental implants environment, the surface has to induce a faster sealing in soft tissue to avoid bacterial colonization and post infections. The different types of tissue in which medical devices are facing made harder the smart design from a native tissue point of view.

This one reason why surface technology has been developing many strategies in order to create bioactive surfaces which can respond specifically in each context. Plasma-based techniques have shown an attractive method to modify the surface of dental implants developing topography and chemistry changes to respond to different environment. In that sense, the developed features at the nano-scale order can mimic the nanofeatures found in native ECM and interact with cytoplasmatic prolongations such filopodia and lamellipodia in the same order. Surface topography as well physicochemical properties have shown to be key factors of the biological responses affecting fundamental processes, such as the protein adsorption and cell adhesion, proliferation and differentiation.

On the other hand, surface modifications techniques have to take into account the complex structures used in the biomedical field such dental implants or catheters. Many of these surface technologies work in limited conditions using 2D substrates or at small-scale, therefore, some of these technologies are not suitable for the biomedical industry.

In this context, DPNS can produce the surface modification of 3D complex structures and in addition, can be the easy scaleable to the industry level, solving the previous limitations. Furthermore, it achieves nanofeatures in a homogeneous form for the whole surface which will enhance cells interaction and subsequently, implants osseointegration. DPNS becomes a powerful tool capable to effectively modify 3D complex structures as it is shown in the next figures.

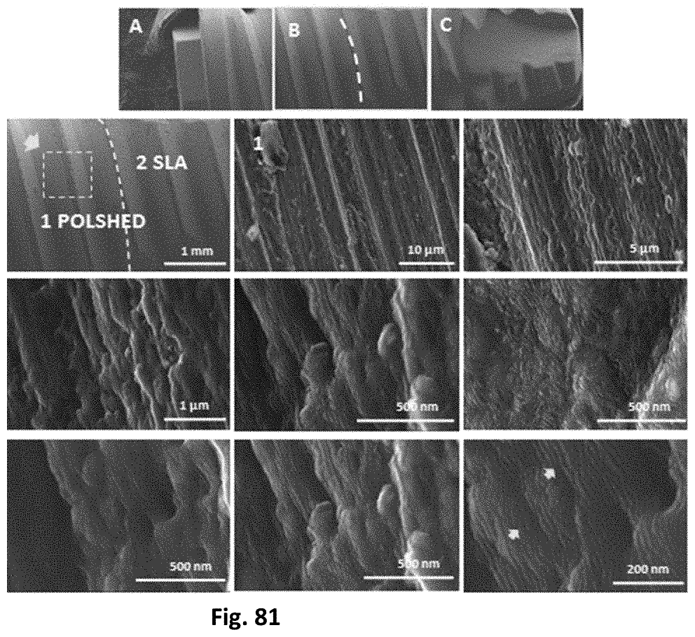

Here, it is presented a clear example of how DPNS can modify any 3D material. As it is observed in FIG. 81 nanofeatures were developed in polished titanium alloy covering the whole implant surface due to the ion bombardment of Argon by DPNS. These homogeneous nanofeatures are presented in similar morphology and size (nanopillars or nanoplatelets of 20 nm).

In these images, it is possible to confirm DPNS as a powerful technology to achieve the modification of complicated geometries with different planes, angles, and topographies.

In addition, these complex structures usually are in the biomedical market with some previous modifications produced by other technologies.

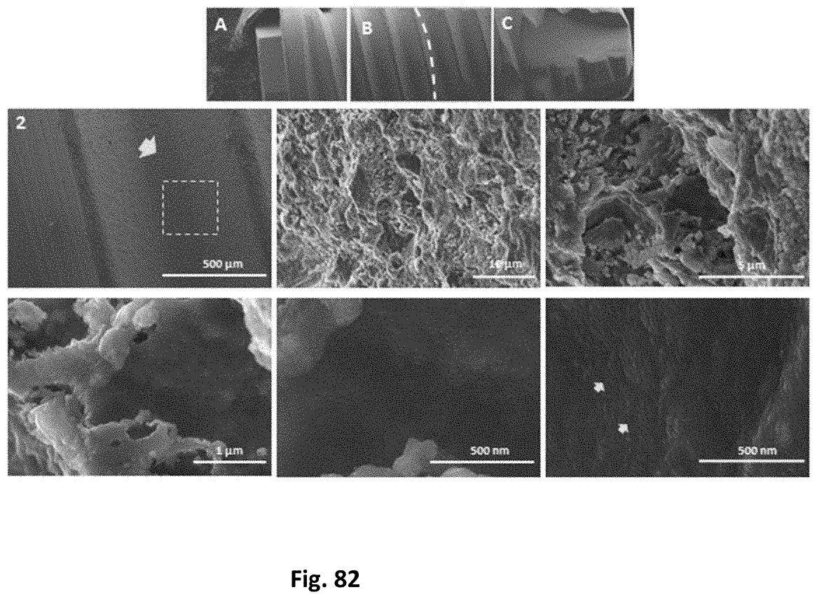

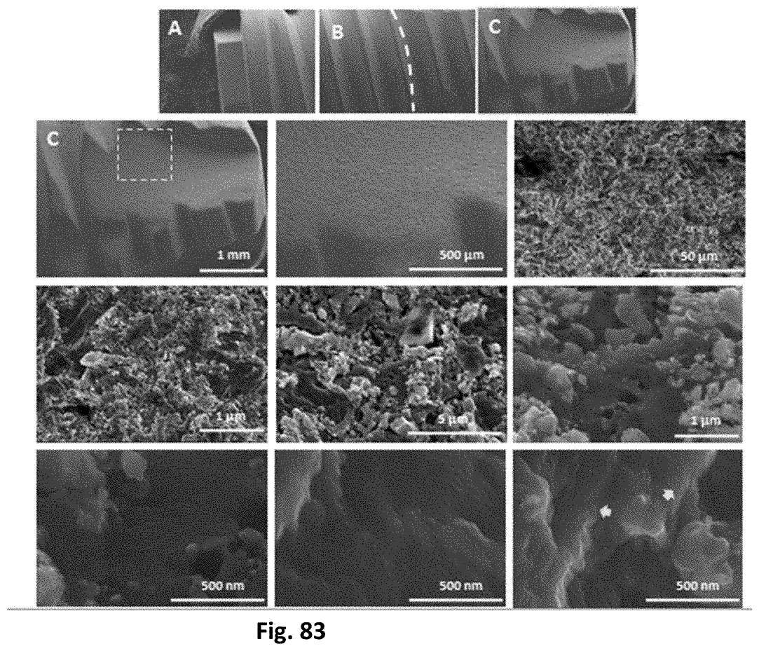

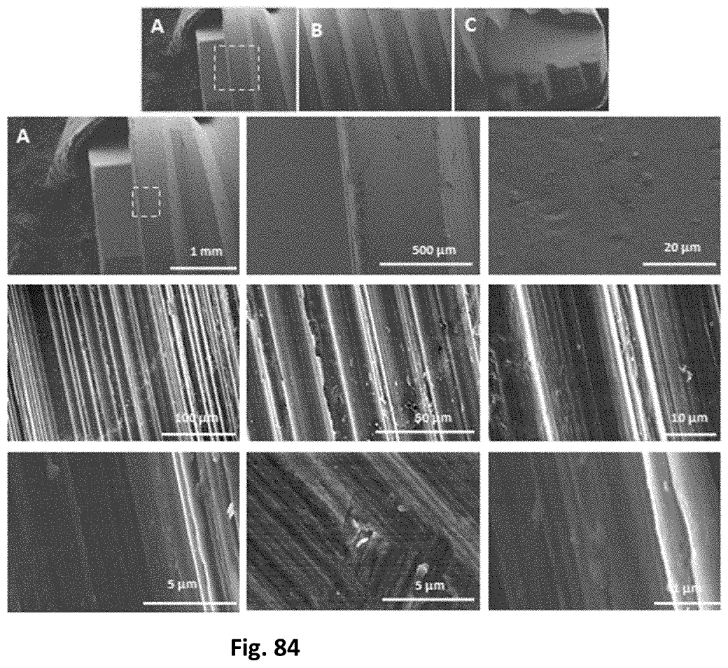

In FIGS. 82 and 83, SEM images revealed a microroughness surface due to the commercial SLA process with new nanofeatures in the middle and lower parts respectively. While the upper part still without any modification (see FIG. 81 polished titanium) which has been shown to reduce bacterial adhesion, the middle and lower parts should increase the osseointegration and long-term fixation. SLA type surface, produced by sandblasting and acid etched process, has shown to promote osteoblast adhesion and differentiation inducing faster osseointegration. The main SLA topography is based on random features at the microscale level, however, using DPNS it has introduced features at the nanoscale level as well. DPNS achieves the surface modification at the nanoscale order of complex devices with other treatments which will improve their osseointegration and increase the success of their clinical application.

### REFERENCES

Smeets, R., Stadlinger, B., Schwarz, F., Beck-Broichsitter, B., Jung, O., Precht, C., . . . & Ebker, T. (2016). Impact of dental implant surface modifications on osseointegration. BioMed Research International, 2016.
Chrcanovic, B. R., Albrektsson, T., & Wennerberg, A. (2014). Reasons for failures of oral implants. Journal of Oral Rehabilitation, 41(6), 443-476.
Ballo, A. M., Omar, O., Xia, W., & Palmquist, A. (2011). Dental implant surfaces-physicochemical properties, biological performance, and trends. In Implant Dentistry-A Rapidly Evolving Practice. InTech.
de Queiroz, J. D. F., de Sousa Leal, A. M., Terada, M., Agnez-Lima, L. F., Costa, I., de Souza Pinto, N. C., & de Medeiros, S. R. B. (2014). Surface modification by argon plasma treatment improves antioxidant defense ability of CHO-k1 cells on titanium surfaces. Toxicology in Vitro, 28(3), 381-387.
Yoshinari, M., Matsuzaka, K., & Inoue, T. (2011). Surface modification by cold-plasma technique for dental implants-Bio-functionalization with binding pharmaceuticals. Japanese Dental Science Review, 47(2), 89-101.
Guastaldi, F. P., Yoo, D., Marin, C., Jimbo, R., Tovar, N., Zanetta-Barbosa, D., & Coelho, P. G. (2013). Plasma treatment maintains surface energy of the implant surface and enhances osseointegration. International journal of biomaterials, 2013.
McNamara, L. E., Sjostrbm, T., Seunarine, K., Meek, R. D., Su, B., & Dalby, M. J. (2014). Investigation of the limits of nanoscale filopodial interactions. Journal of tissue engineering, 5, 2041731414536177.

### Example 8: Irradiation of Phosphate Coating of Titanium Alloy Surfaces by Directed Plamsa Nanosynthesis (DPNS) Towards an Increased Bone Osseointegration

Commercially pure titanium (cpTi) and its medical grade alloy (Ti6Al4V) are some of the most used biomaterials for bone implants fabrication. This is attributed to their high biocompatibility, the good balance of mechanical properties and their osteointegration capability. However, since Ti is a first generation biomaterial (bioinert) it allows the formation of a fibrous tissue around the implants. In order to prevent the failure associated to this fibrous capsule, several surface modification techniques of Ti have been developed: most of them are focused on topographical and top-down chemical changes like thermal plasma spray and electrochemical methods, using HA and Bioglass®. There are not reports about development one-step and bottom-up chemical conversion coatings on Ti, in order to produce bioactive surfaces, but also with nanofeatures which can offer antibiofouling properties. The general hypothesis in the work presented here is that a new phosphating treatment of Ti not only can produce a bioactive layer of calcium phosphate, but also that layer can be the matrix to trap other elements like Silver nanoparticles, Zn, or antimicrobials which will give antimicrobial properties, all in a one-step and bottom-up process. To test the hypothesis, we have used some homemade and commercial solutions: two phosphating solutions (solution A with 2.4 g/L ZnO, 2.8 g/L CaCl, 2% v/v ortho-phosphoric acid H₃PO₄ in distilled water, and solution B with 16 ml/L of H₃PO₄, 1.2 g/L ZnO, 16 g/L NaNO₂ in distilled water), and two commercial solutions (Oxifos® and Oxifos Zn®). Polished and cleaned Ti6Al4V samples were treated by immersion in these solutions, and then were characterized by SEM.

Experimental Procedure

1. Pre-Phosphating Preparation of Samples

Ti6Al4V samples are initially polished (240, 320, 400, 600, 800 and 1200 of SiC abrasive papers, and cloth disc with aluminum suspension until mirror finishing) and cleaned with water and neutral soap. Then, the samples are de-oxidized in a mix of HF and HNO3 during 5 minutes.

2. Preparation of Phosphating Solution

The better phosphating solution from the previous studies (Solution A) is prepared with 2.4 g/L ZnO, 2.8 g/L CaCl, 2% v/v H3PO4 in distilled water, and heated at 75° C., during samples immersion.

3. Phosphating Process

The prepared samples are immersed in solution A at 75° C., during 20 minutes, then washed and dried at room temperature. Some samples are already treated in this manner. Some others have to be polished and phosphated in these conditions, cause they were brought non polished from Group BAMR, UdeA.

4. DIS Procedures.

Unphosphated samples were be irradiated in the optimal conditions described in Table 19. They were selected from initial studies performed in our group using Ti6Al4V surfaces and Mg foams studies. These parameters were adjusted to avoid the delamination or cracking effect and to obtain a specific nano-patterning or an improved surface chemistry.

TABLE 19
Irradiation parameters of phosphates Ti samples

| | | Energy | Fluence | Angle |
|---|---|---|---|---|
| Sample | Gas | (eV) | (cm⁻²) | (degree) |
| P_Ti_18mins_001 | Ar | 1000 | 1*10¹⁸ | 0 |
| P_Ti_20mins_002 | Ar | 1000 | 1*10¹⁸ | 60 |
| P_Ti_22mins_003 | Ar | 1000 | 7*10¹⁷ | 60 |
| P_Ti_24mins_004 | Ar | 1000 | 2.5*10¹⁷ | 60 |
| P_Ti_26mins_005 | Ar | 1000 | 7*10¹⁷ | 0 |
| P_Ti_28mins_006 | Ar | 1000 | 2.5*10¹⁷ | 0 |
| P_Ti_30mins_007 | Ar | 750 | 1*10¹⁸ | 0 |
| P_Ti_32mins_008 | Ar | 500 | 1*10¹⁸ | 0 |
| P_Ti_40mins_009 | Ar | 750 | 1*10¹⁸ | 60 |
| P_Ti_50mins_010 | Ar | 500 | 1*10¹⁸ | 60 |

With respect to phosphate samples, being a ceramic layer, we must consider some previous experiences that Prof. Allain has had irradiating ceramic surfaces, with the purpose to avoid layer cracking, produce some nano-pattering or even an improved surface chemistry for the further testing of bioactivity by immersion in SBF.

Irradiation procedures were carried out at different energies, flux, fluence, incidence angles and times in order to depassivate the titanium alloy and induce diffusion of Aluminum content to the surface of the samples before being immersed in SBF. These procedures can also be used to modify the topography, mechanical properties and chemistry of the surface to obtain the proper bone environment to enhance bone formation and improve bioactivity on the surface of Ti6Al4V.

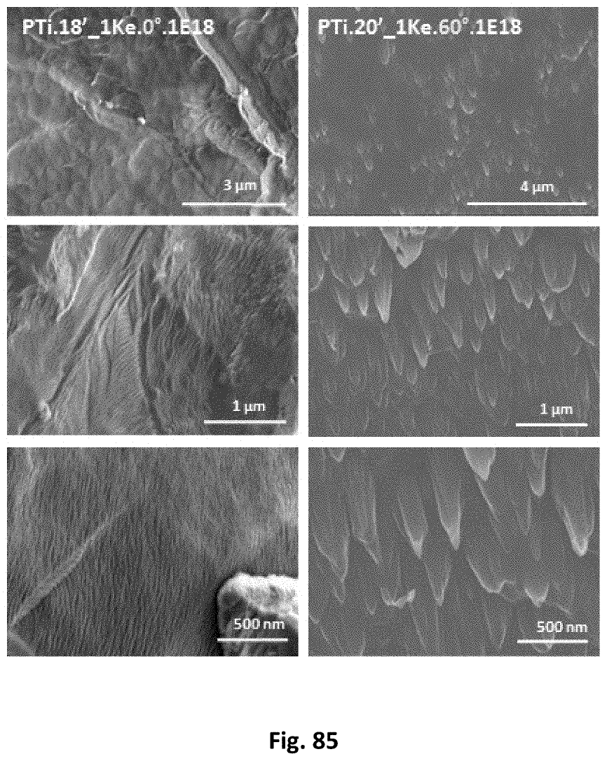

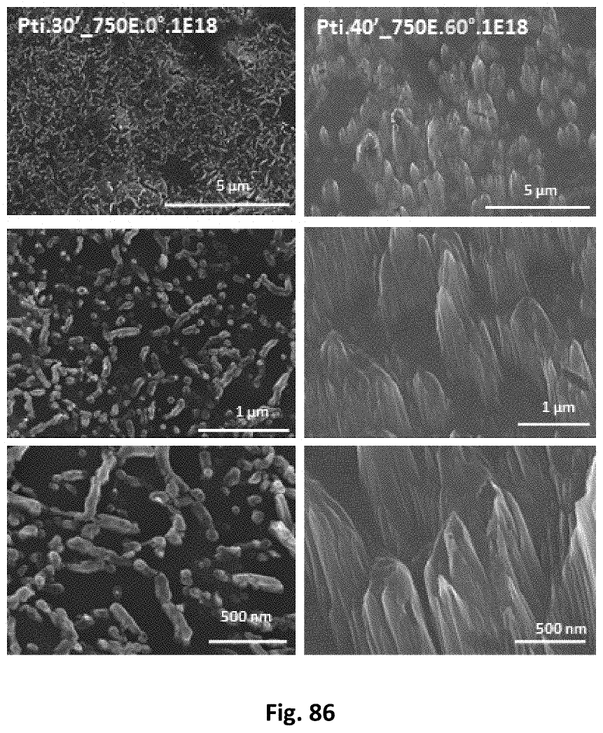

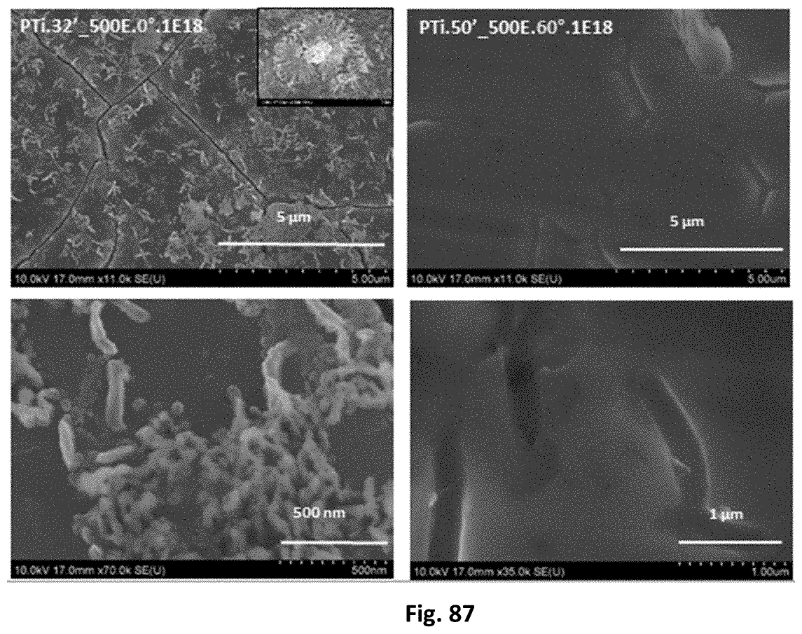

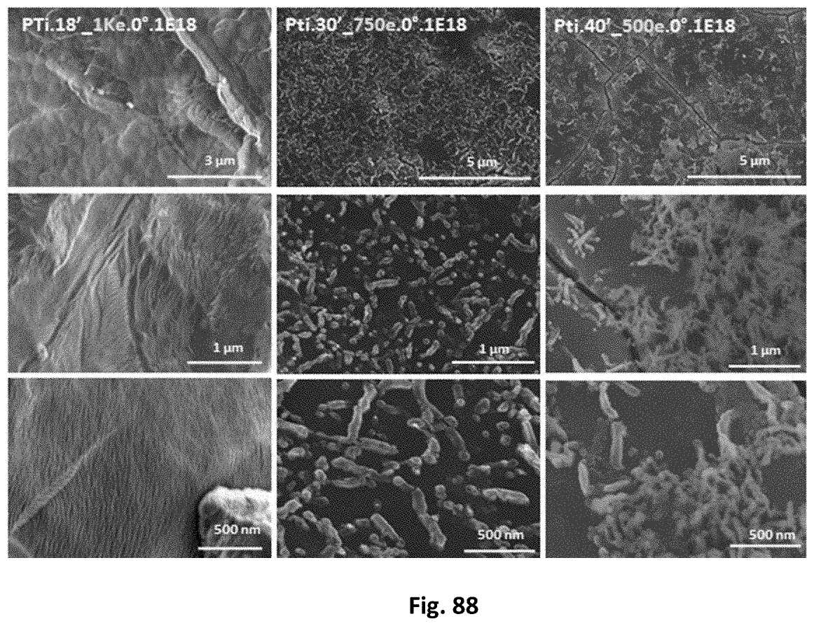

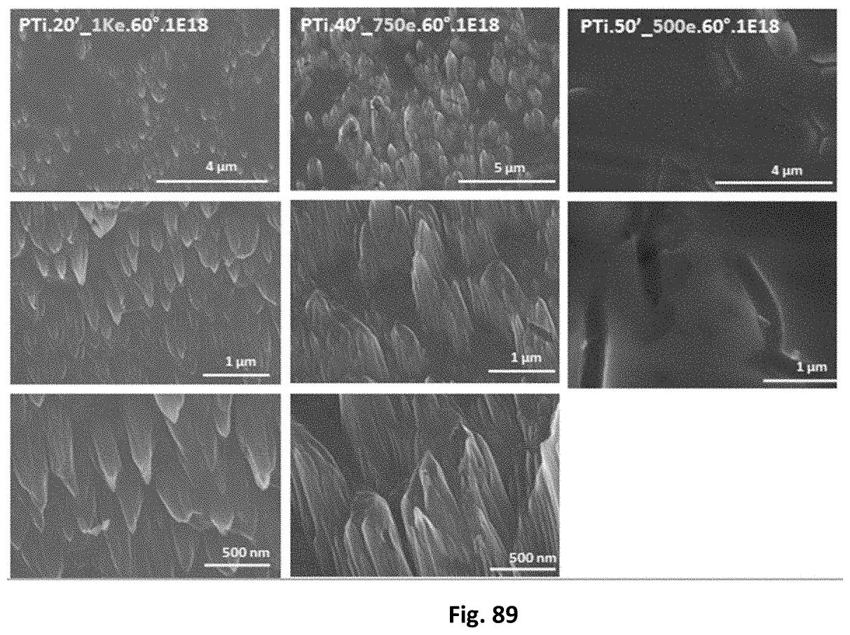

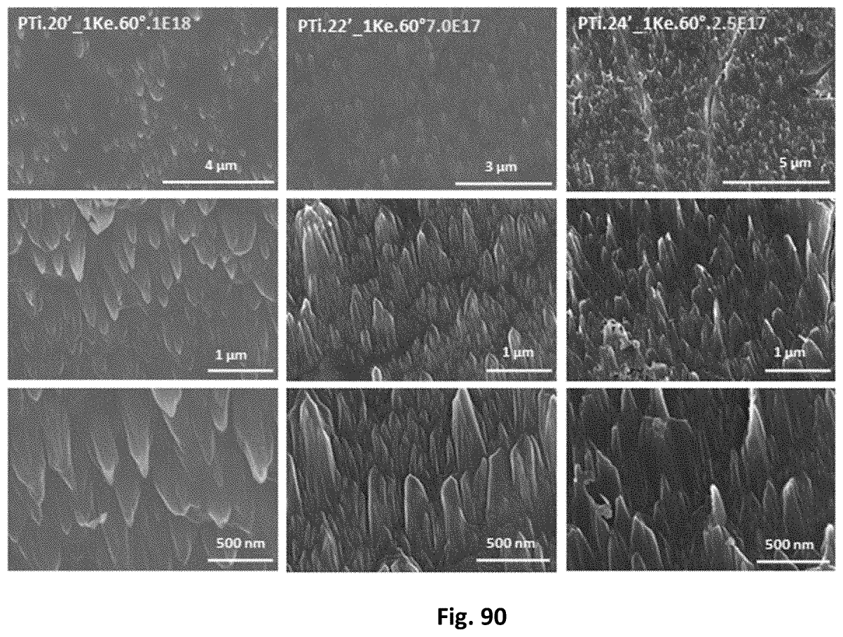

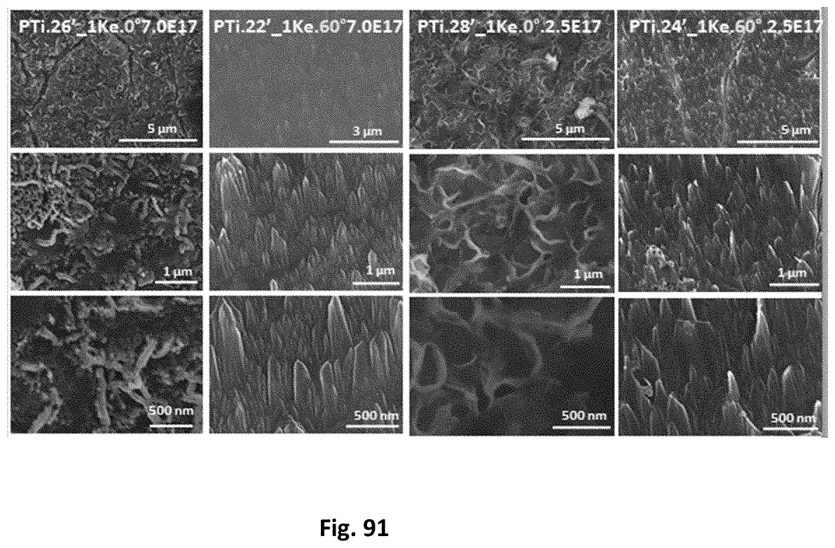

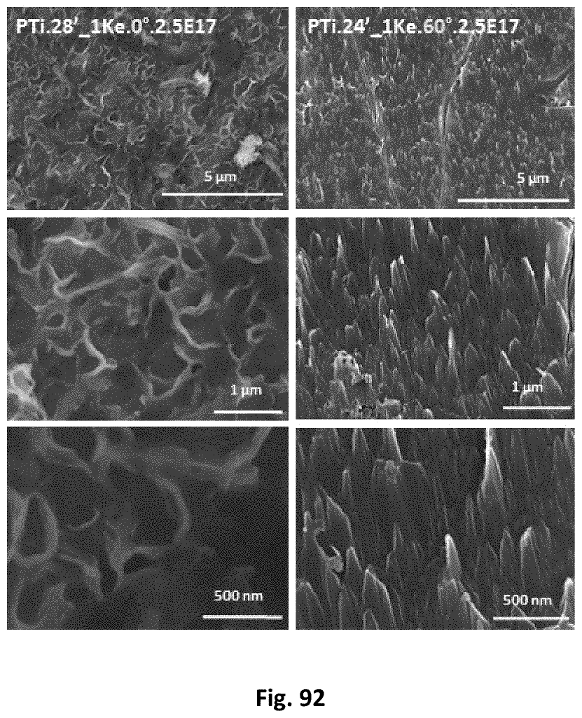

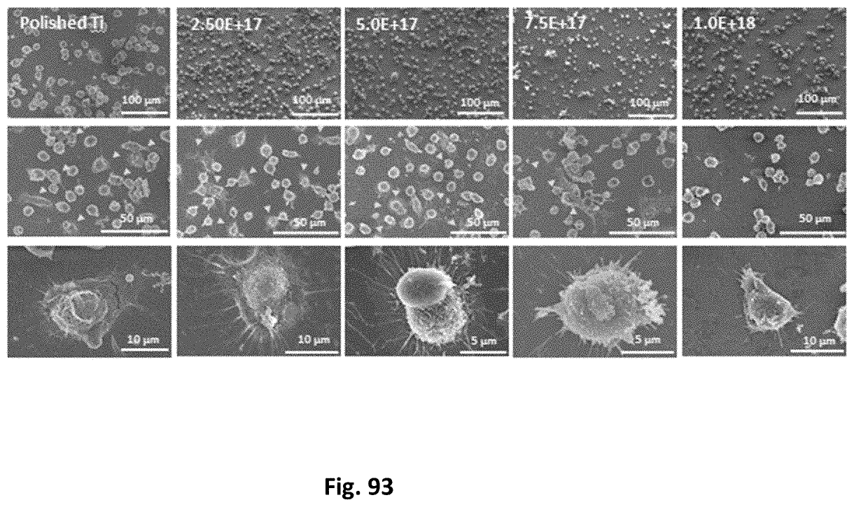

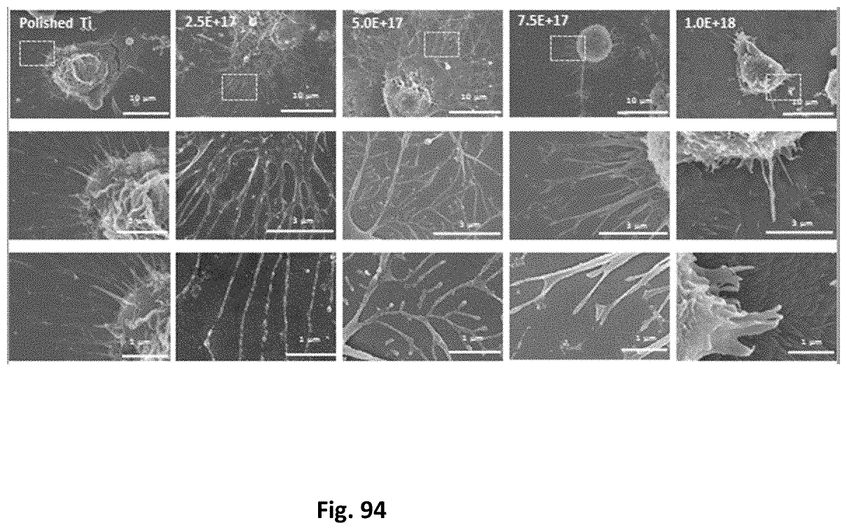

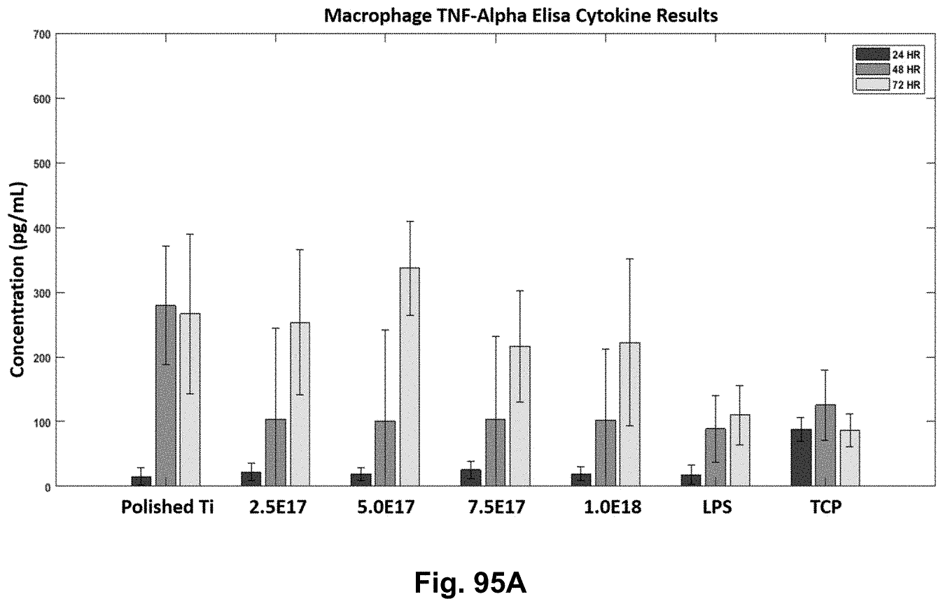

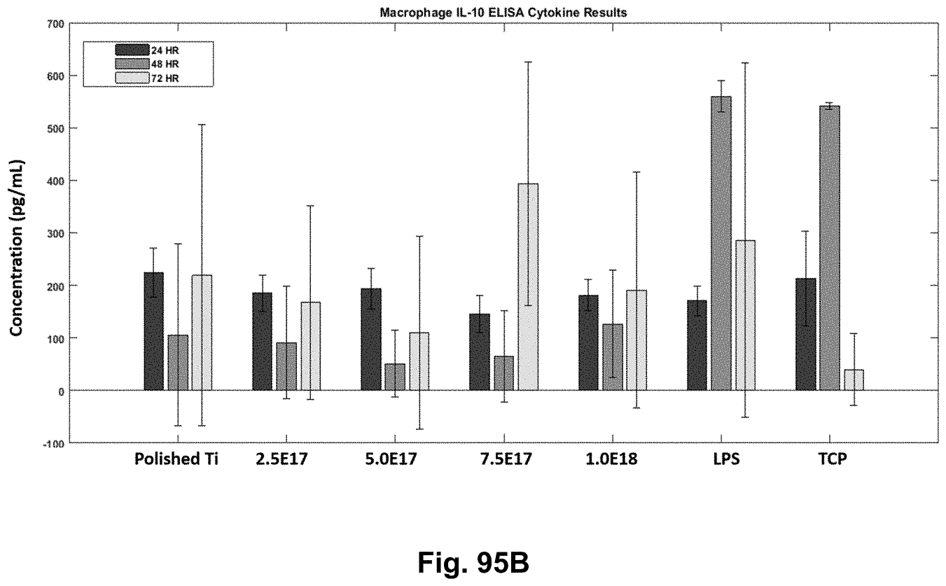

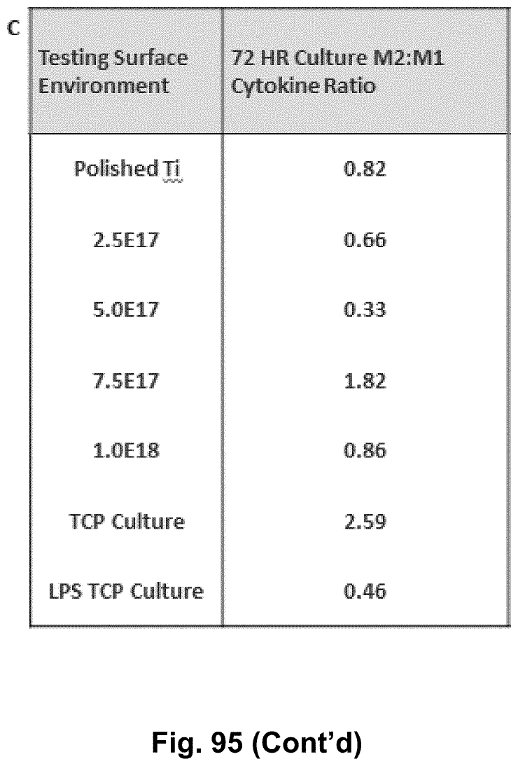

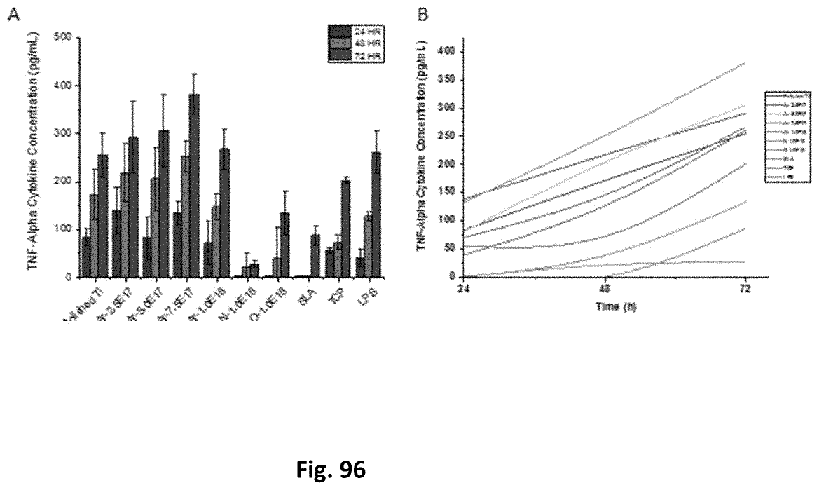

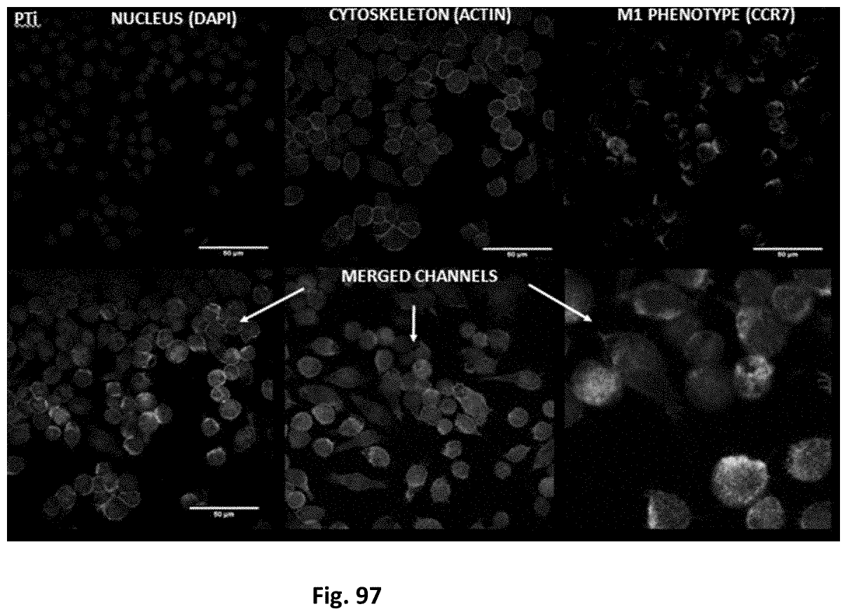

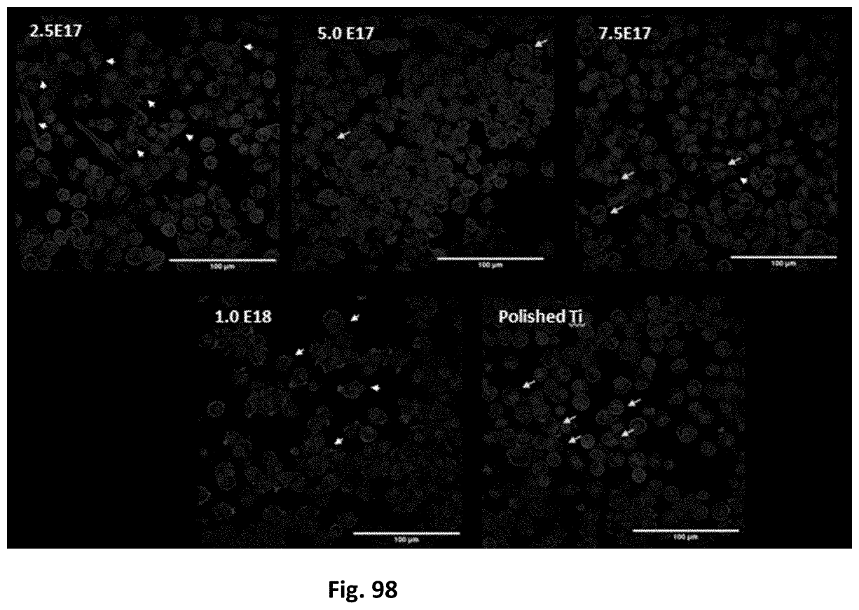

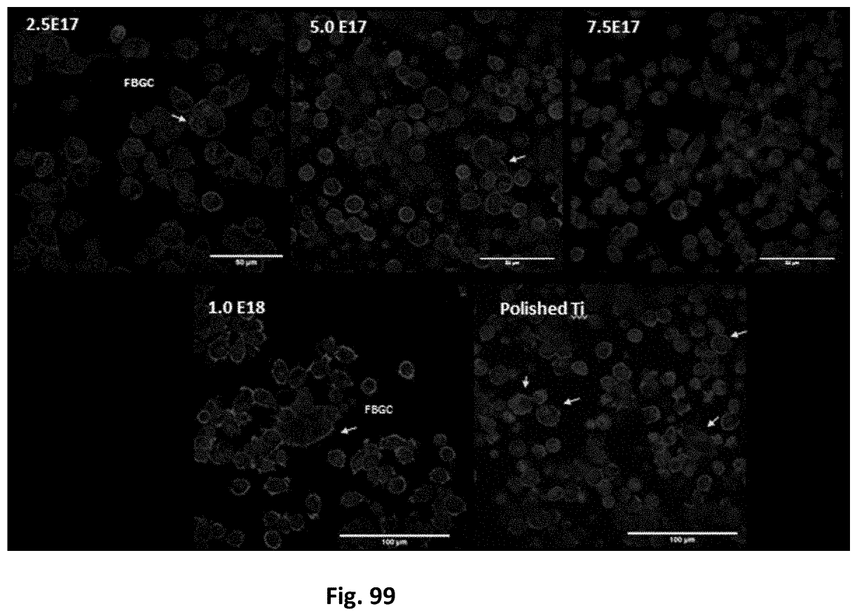

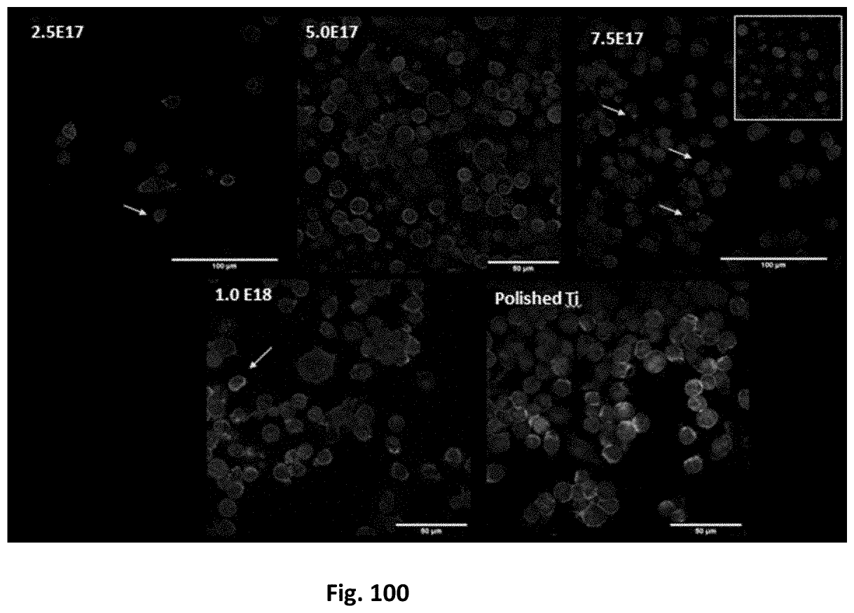

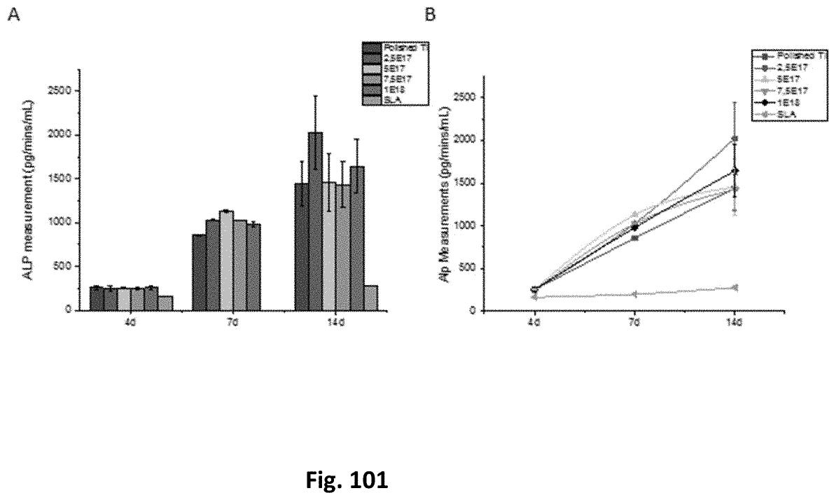

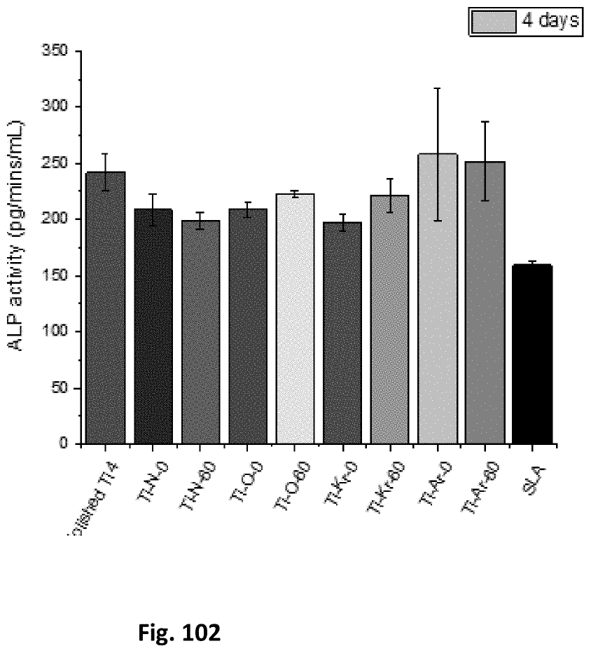

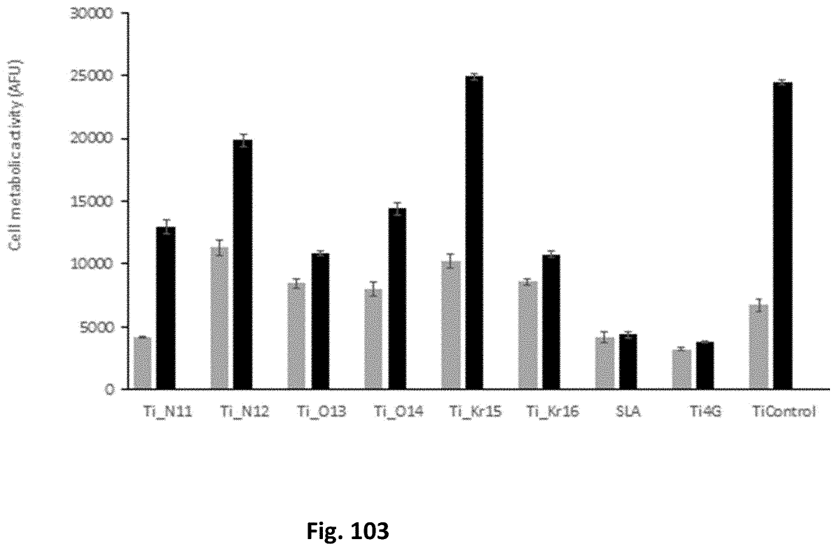

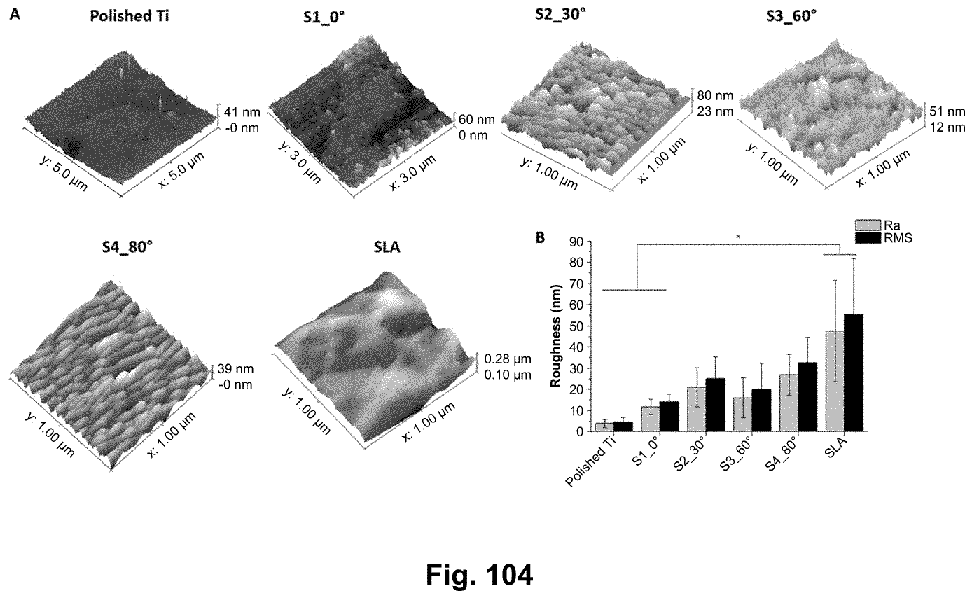

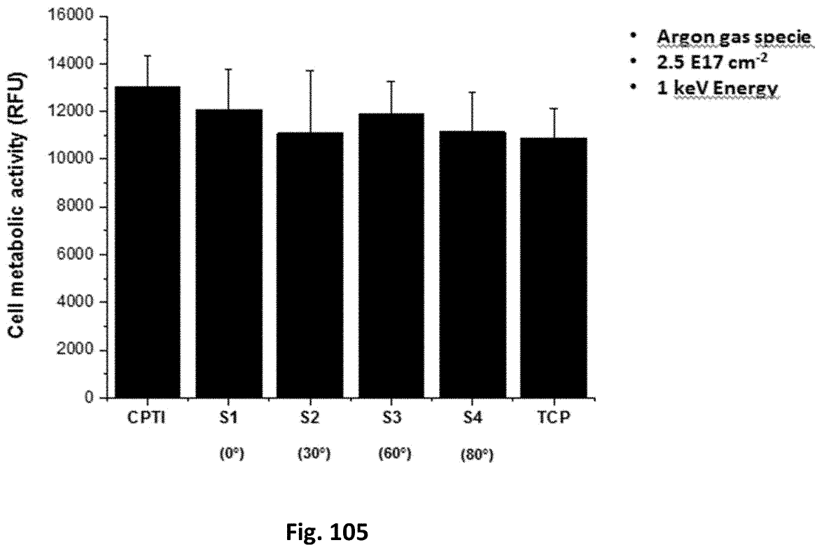

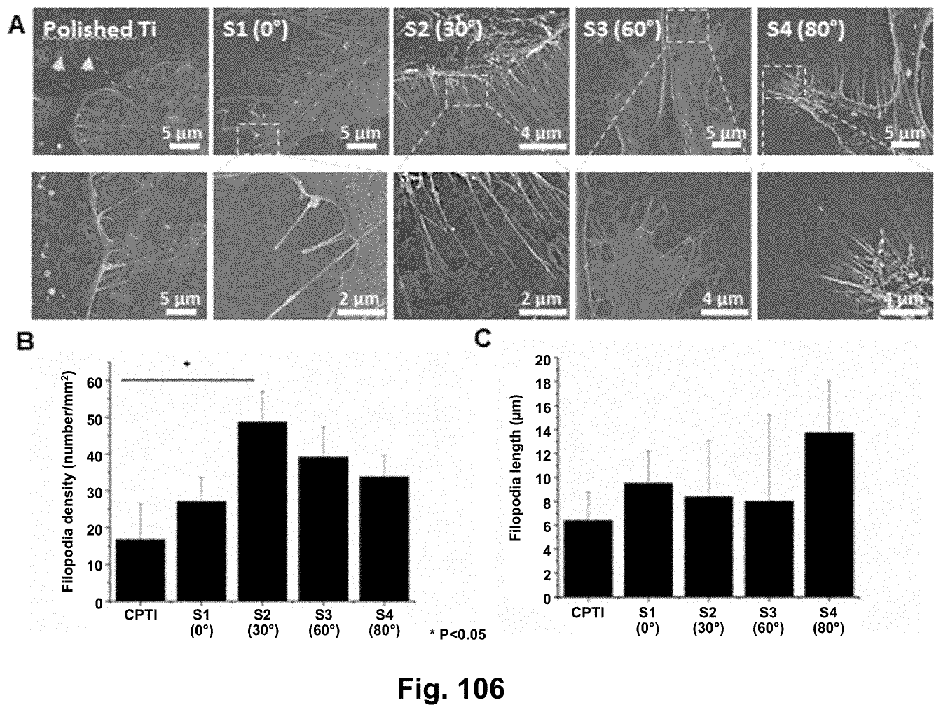

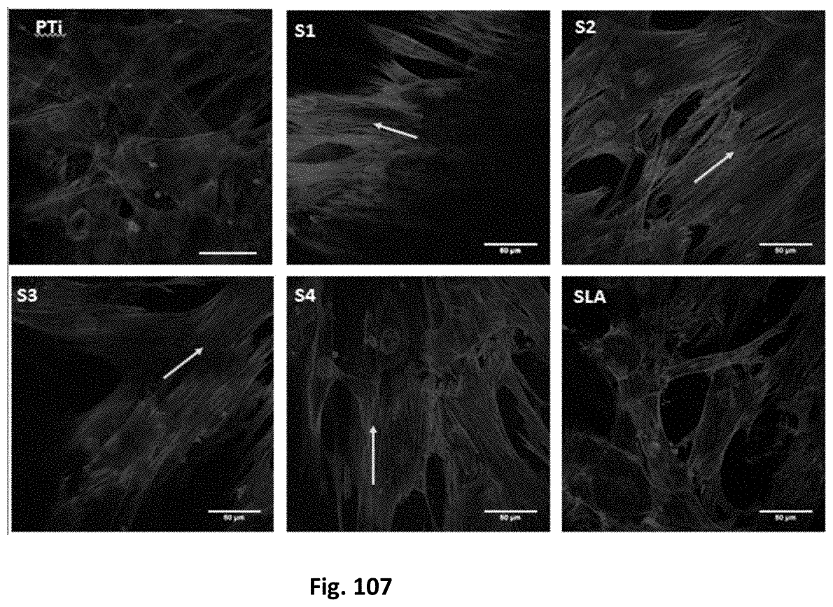

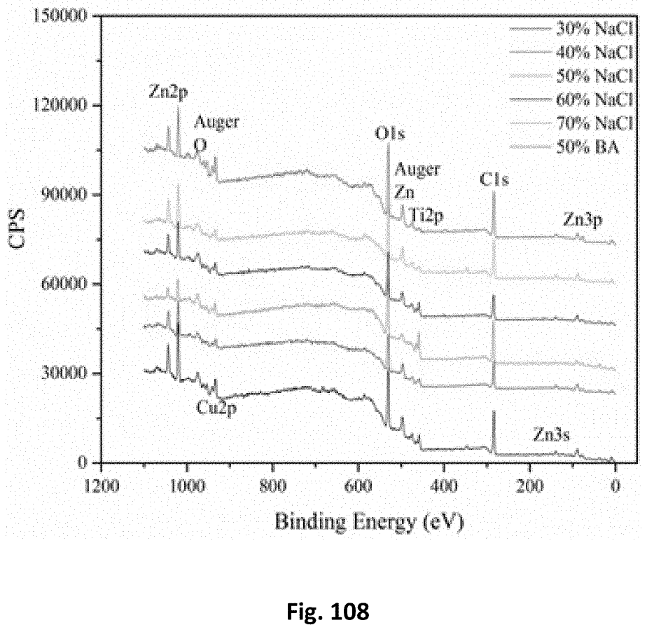

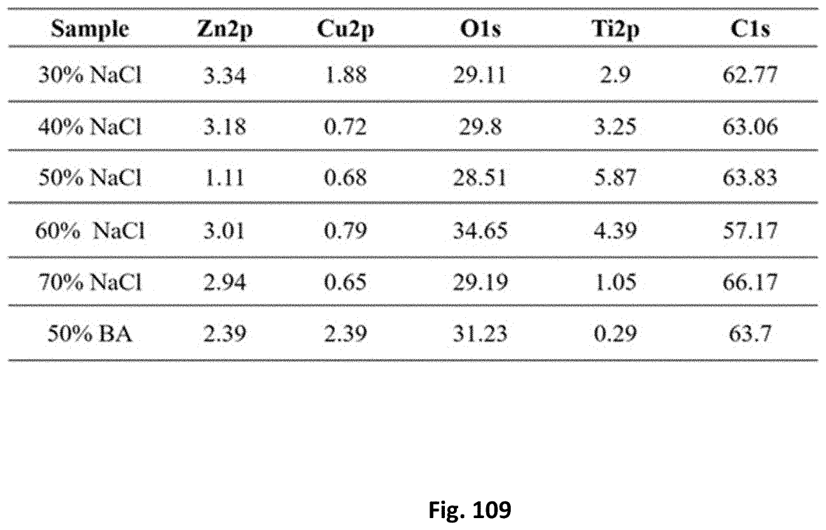

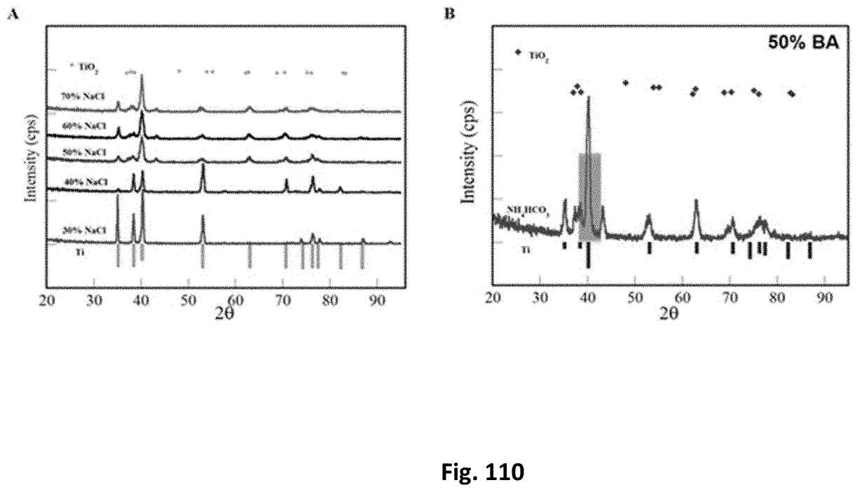

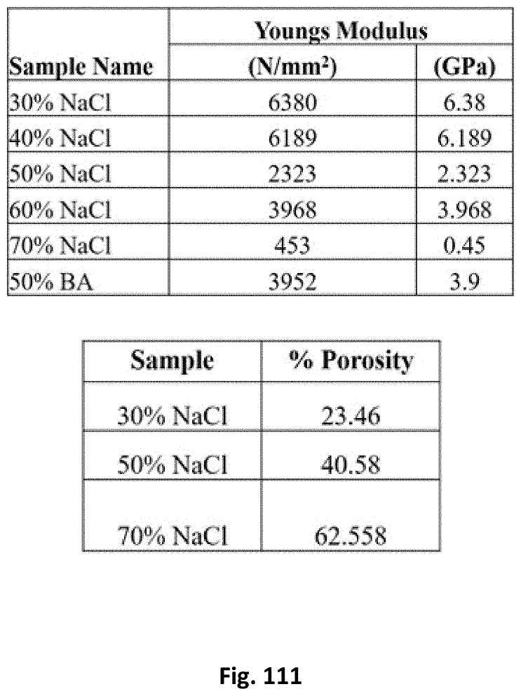

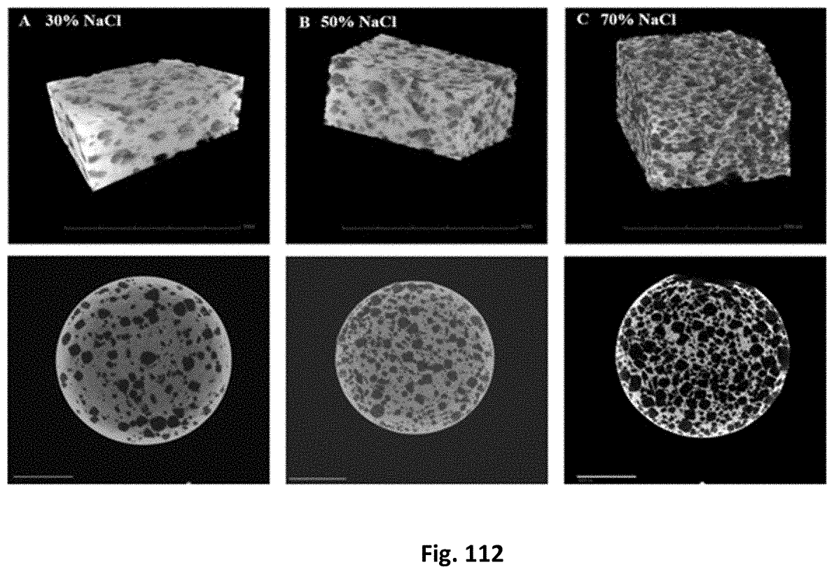

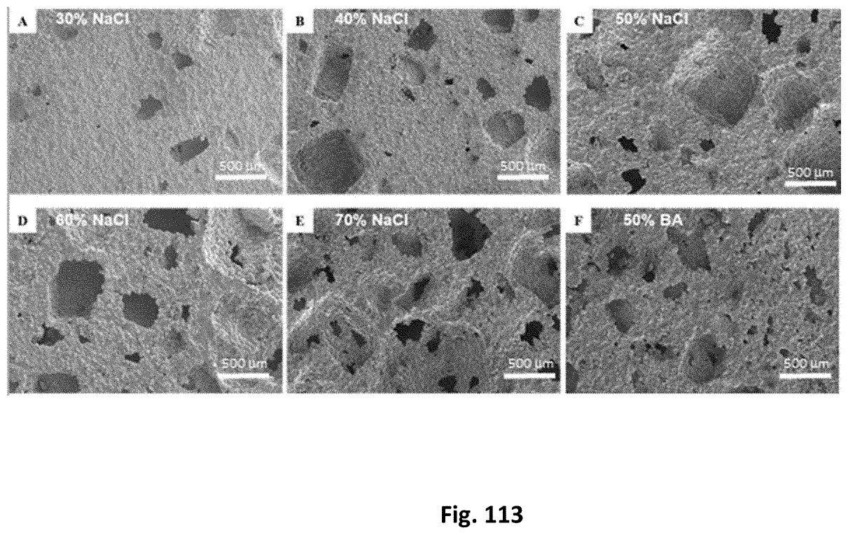

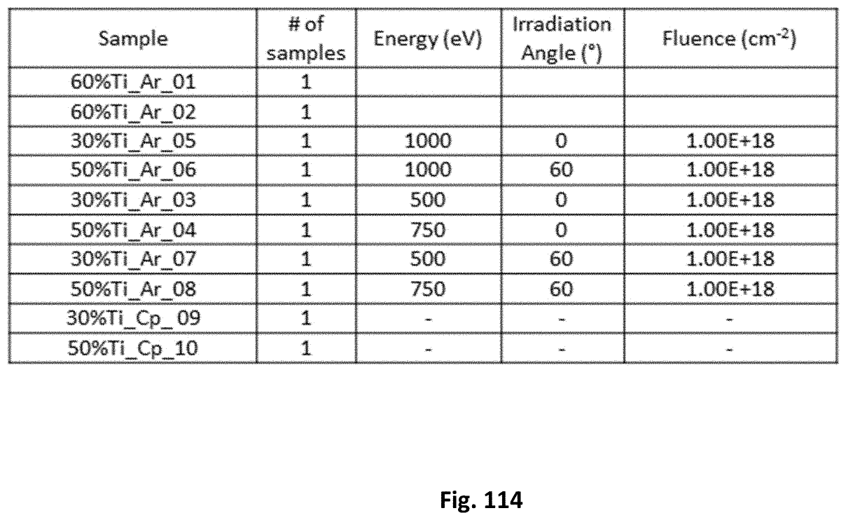

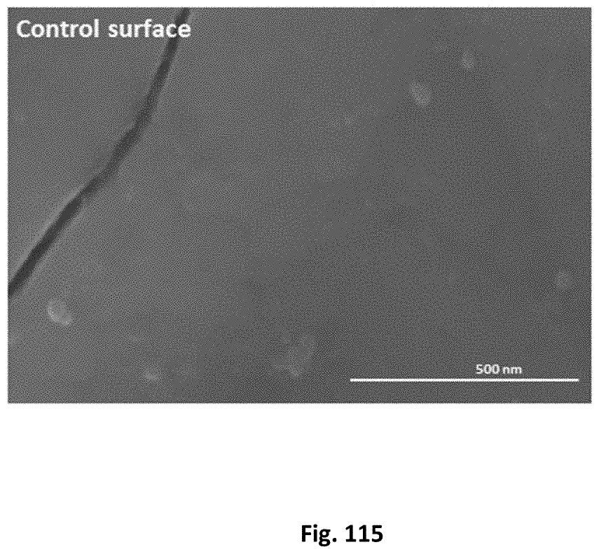

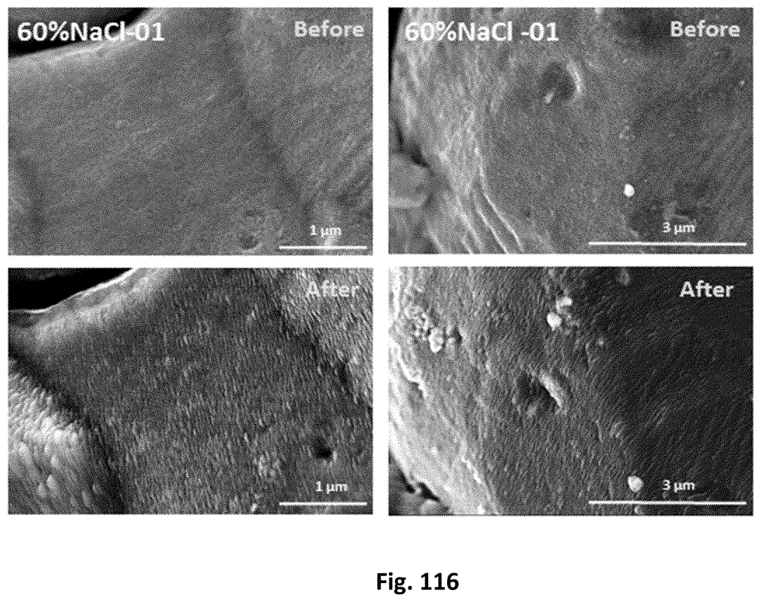

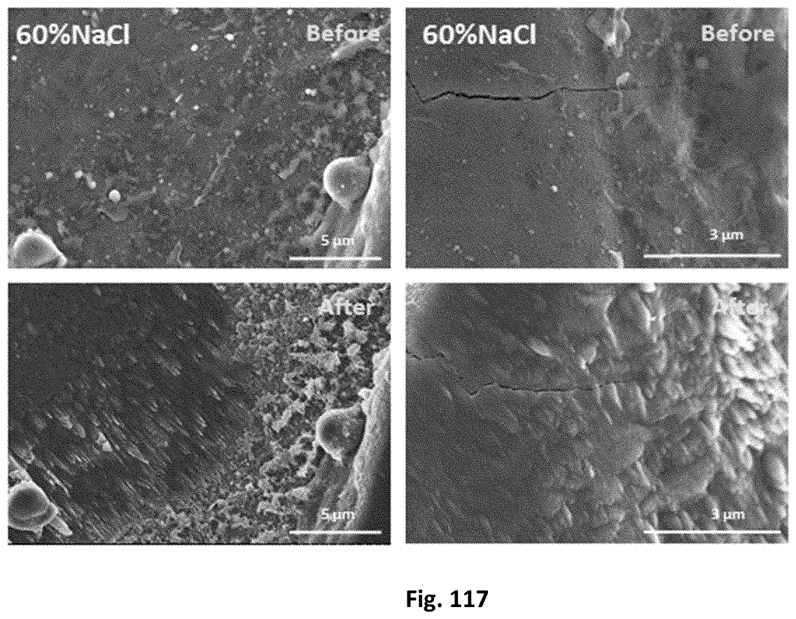

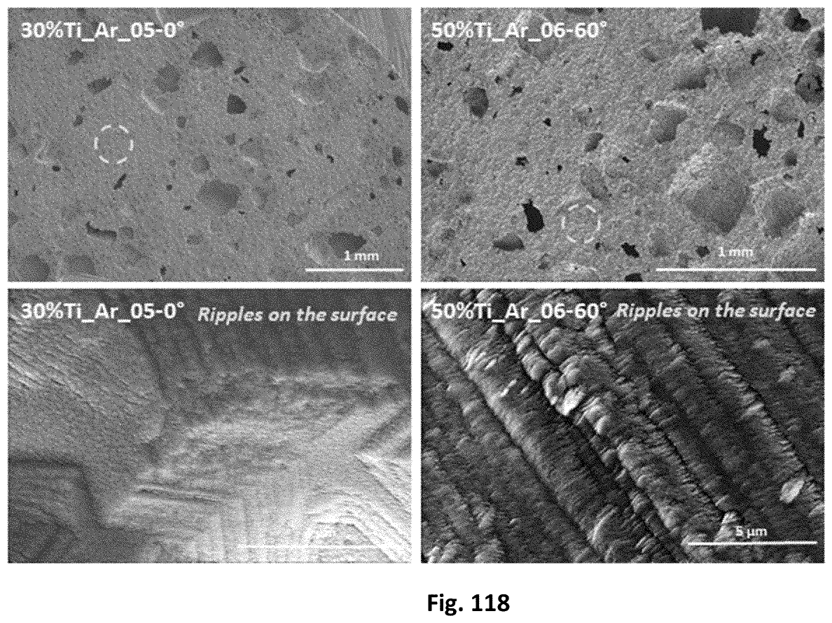

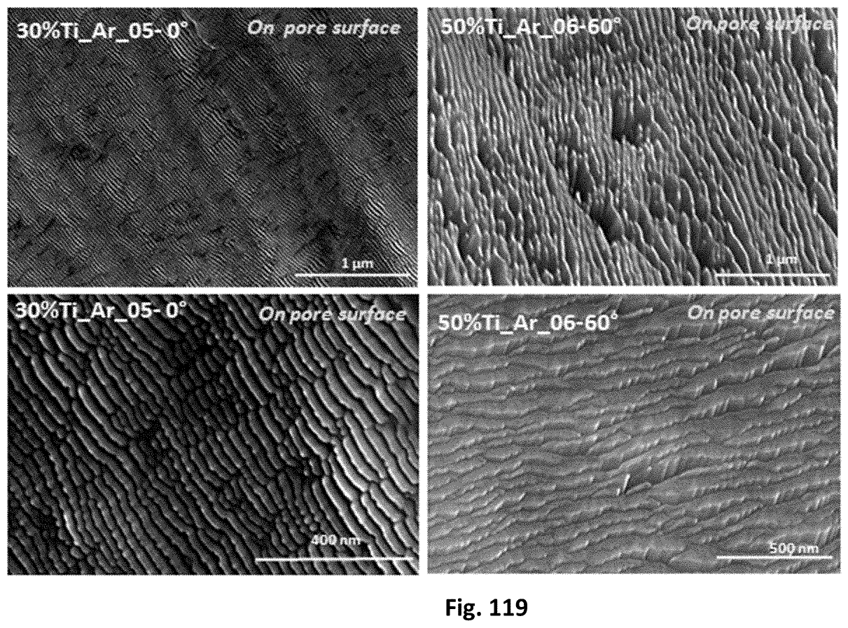

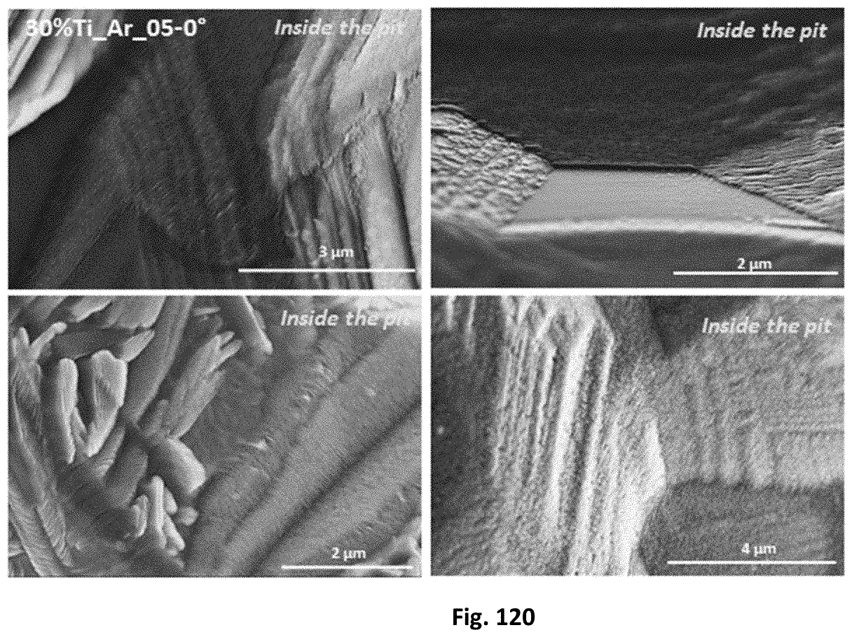

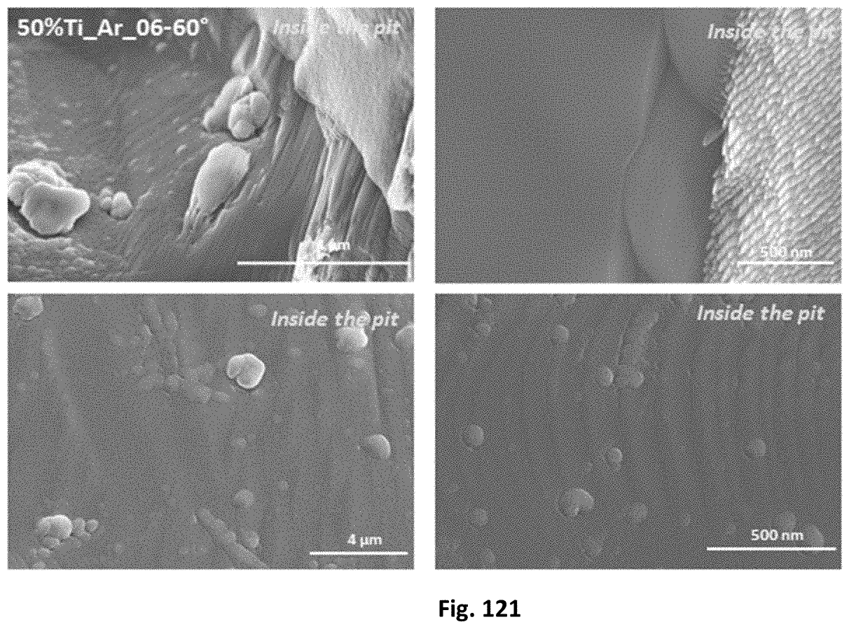

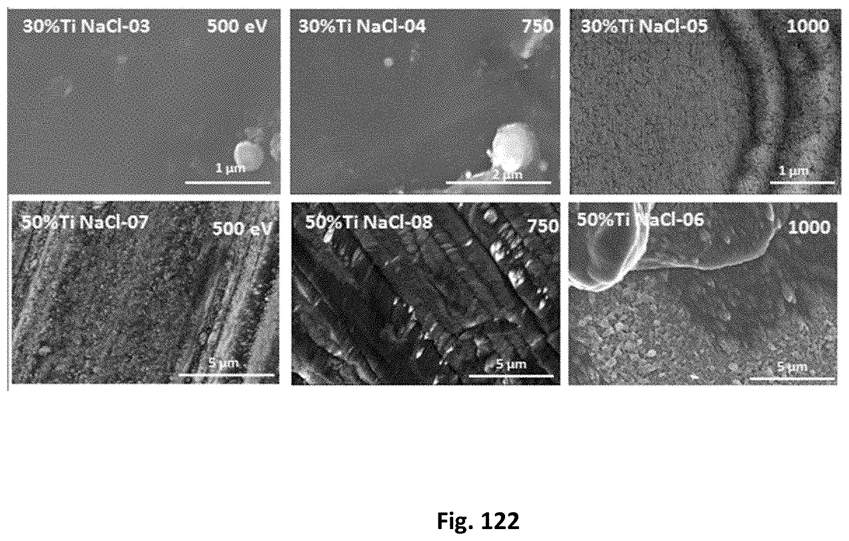

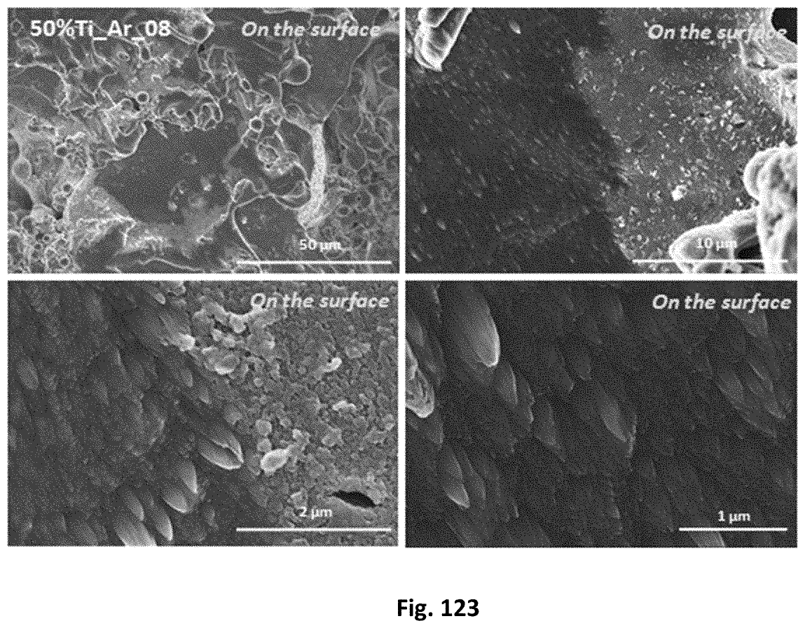

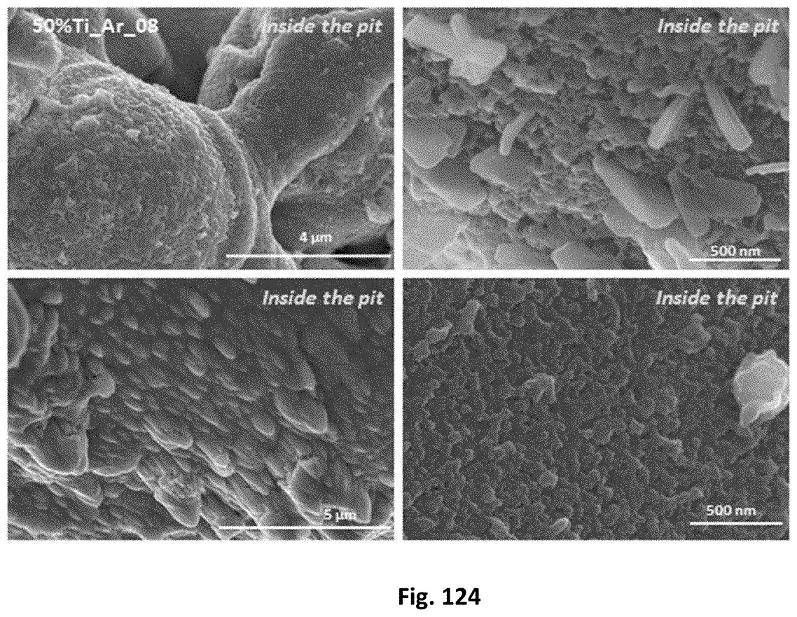

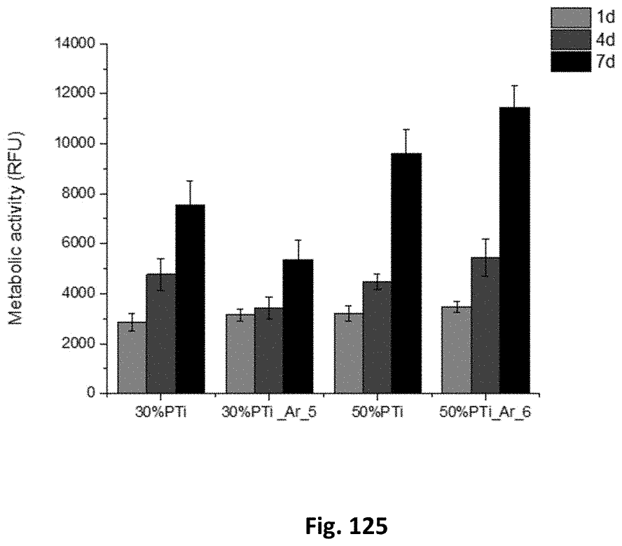

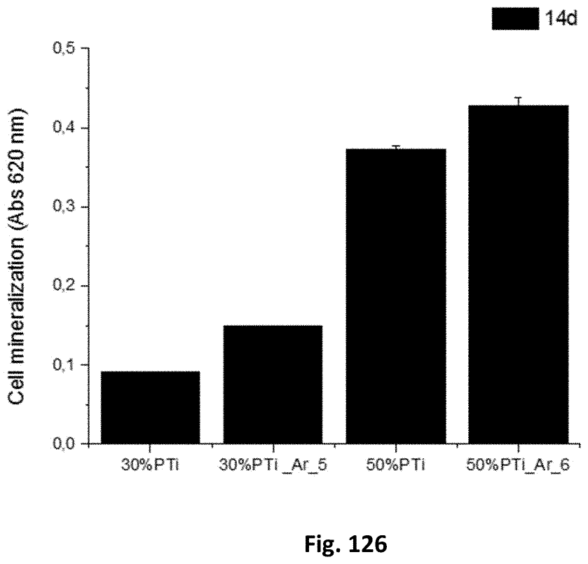

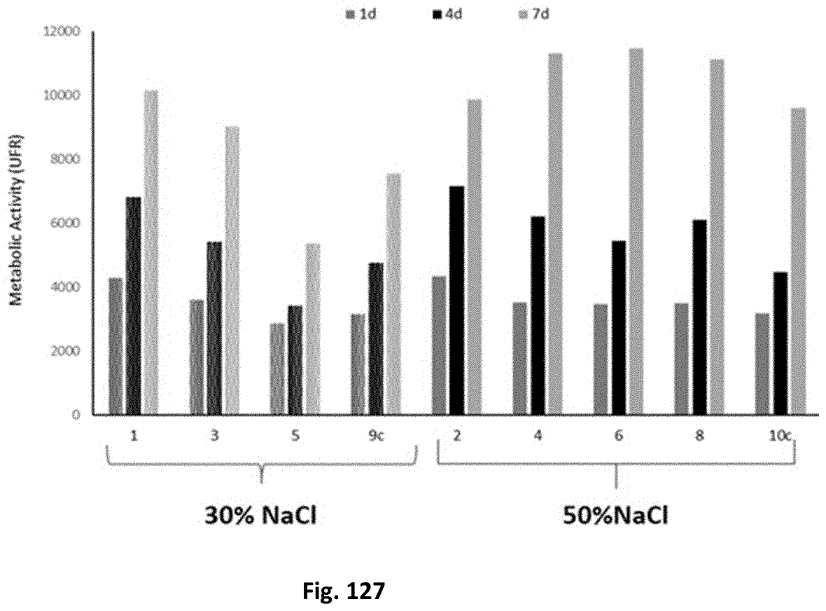

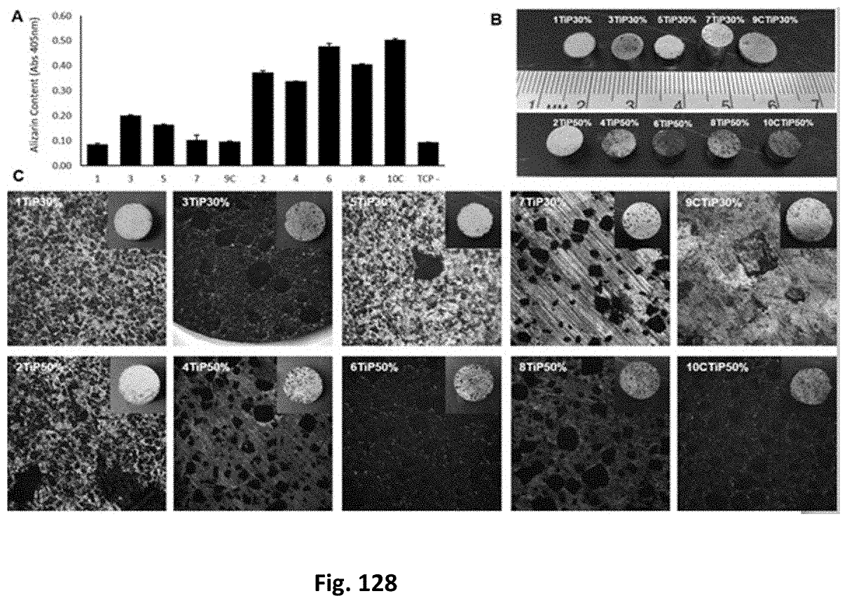

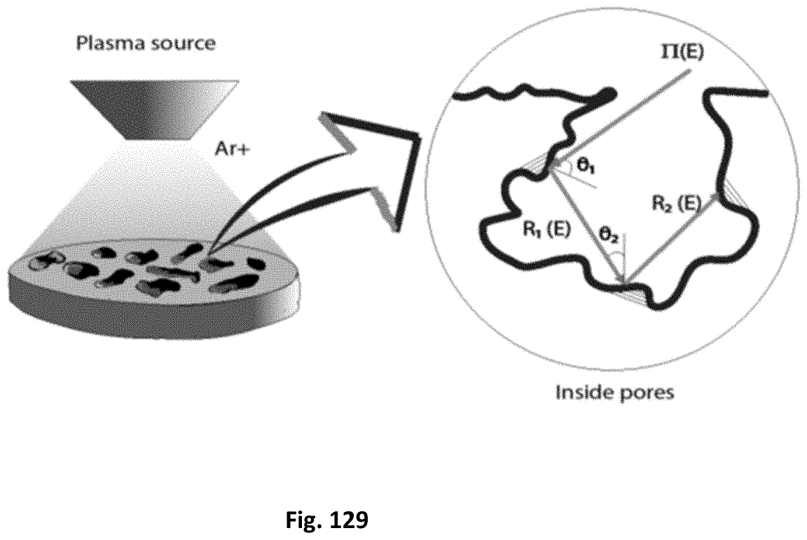

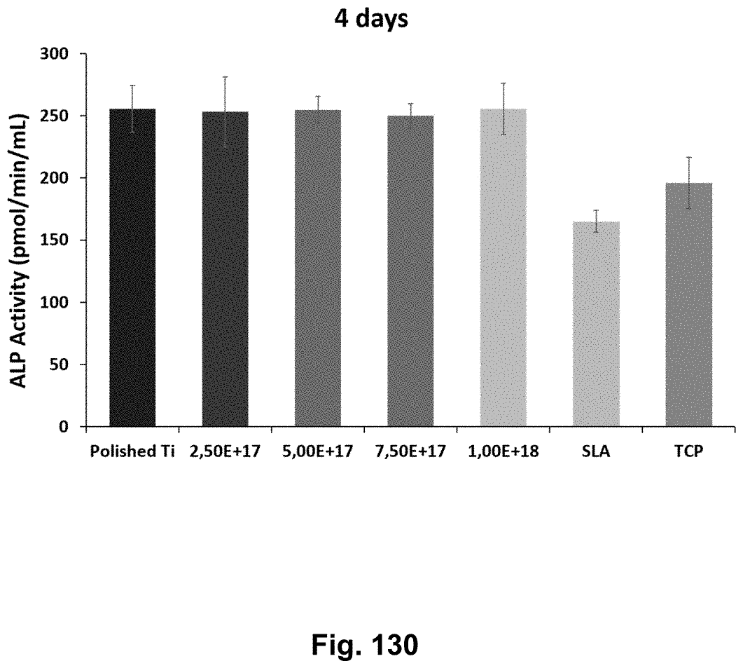

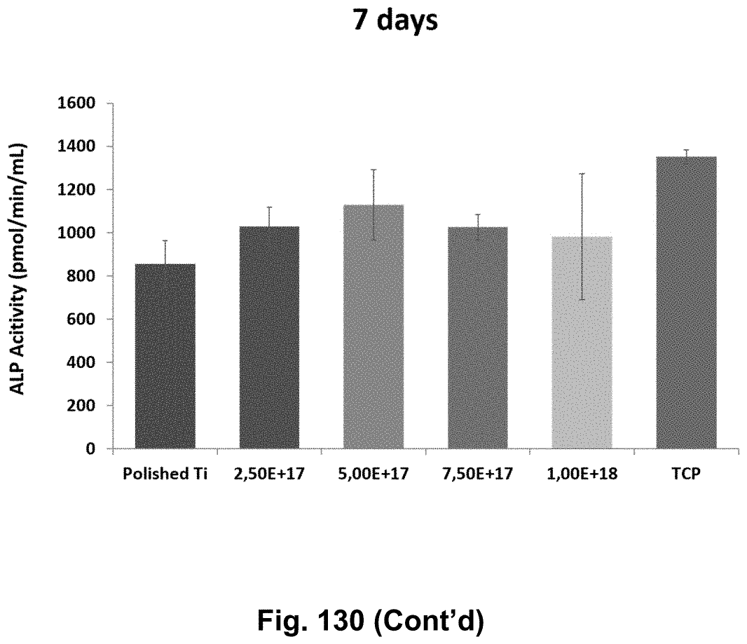

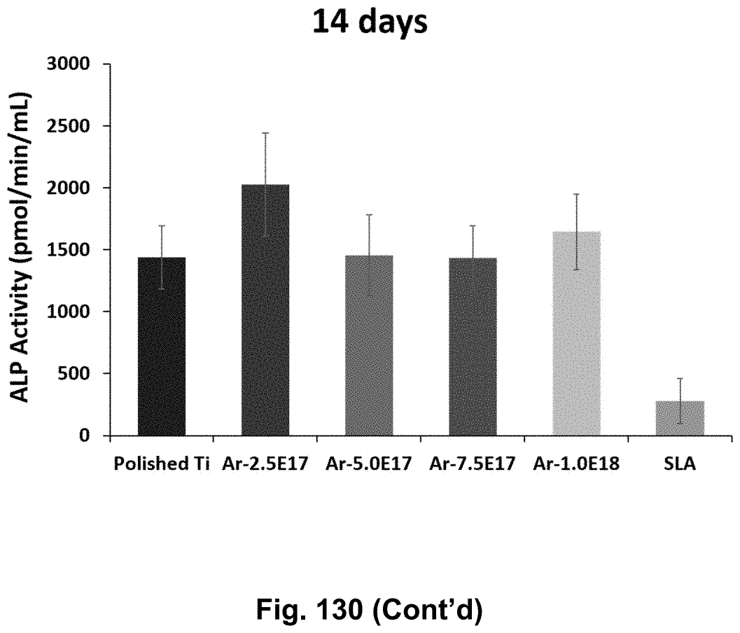

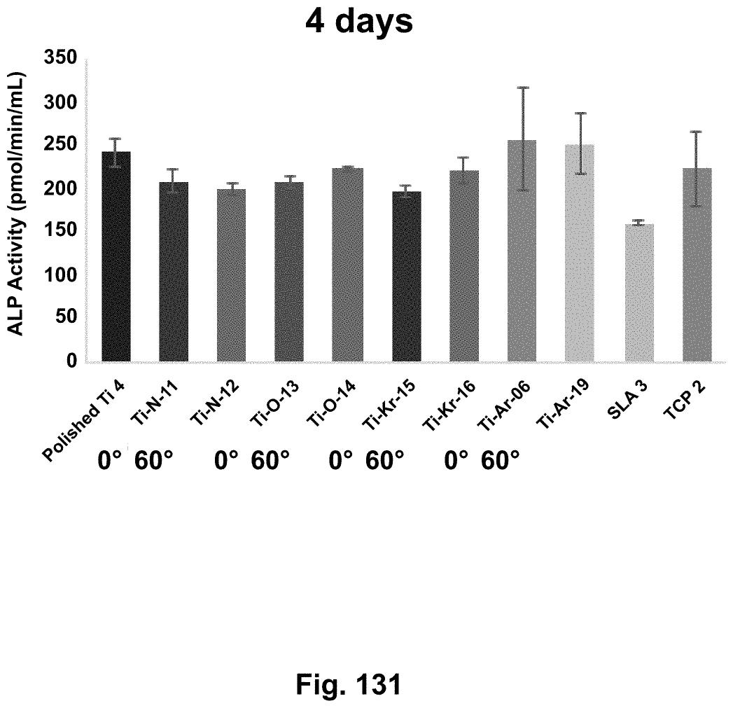

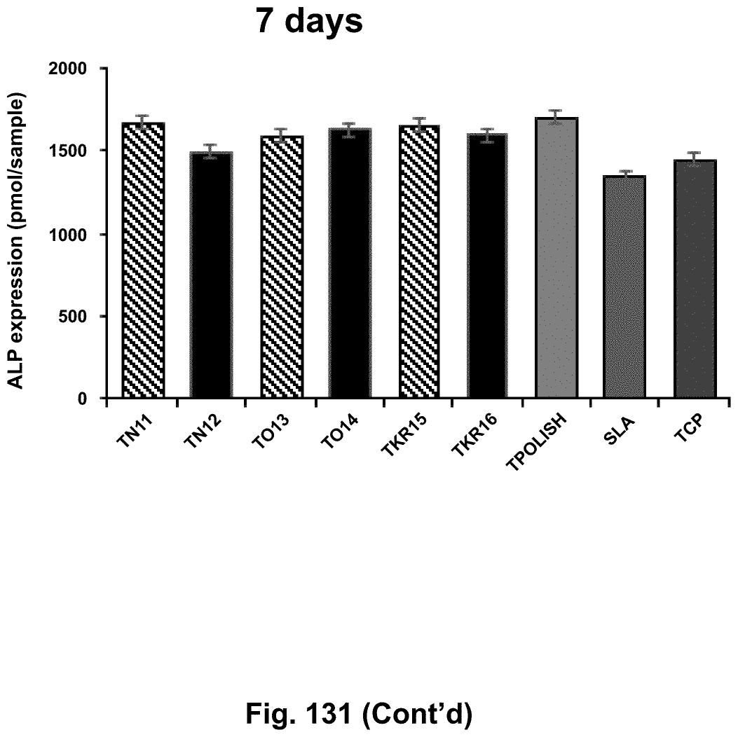

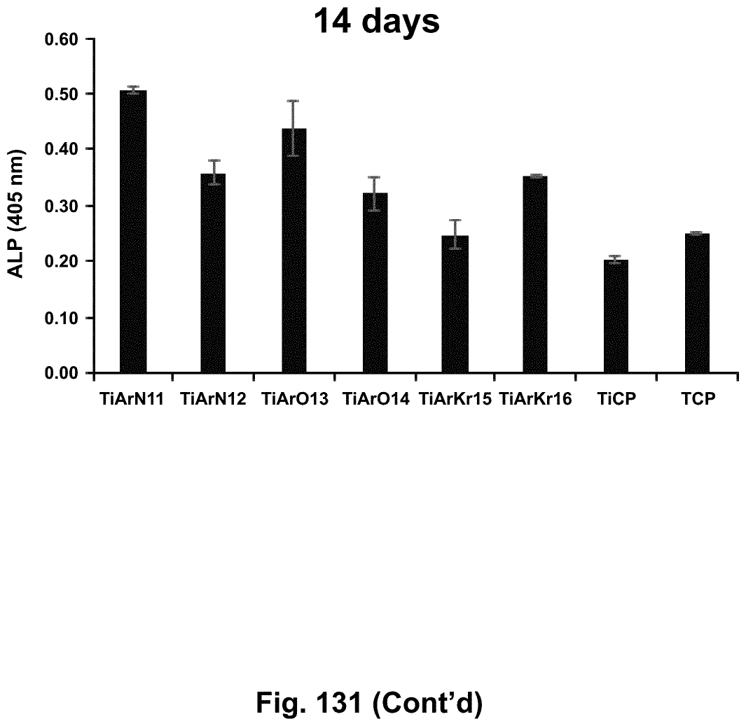

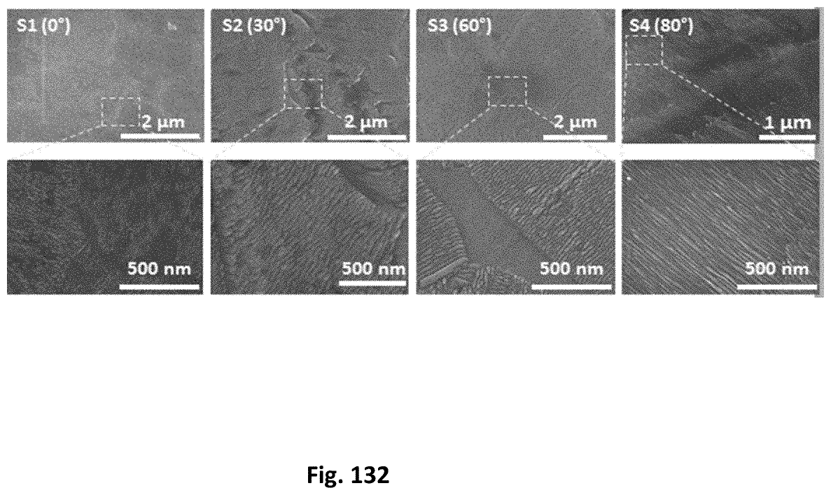

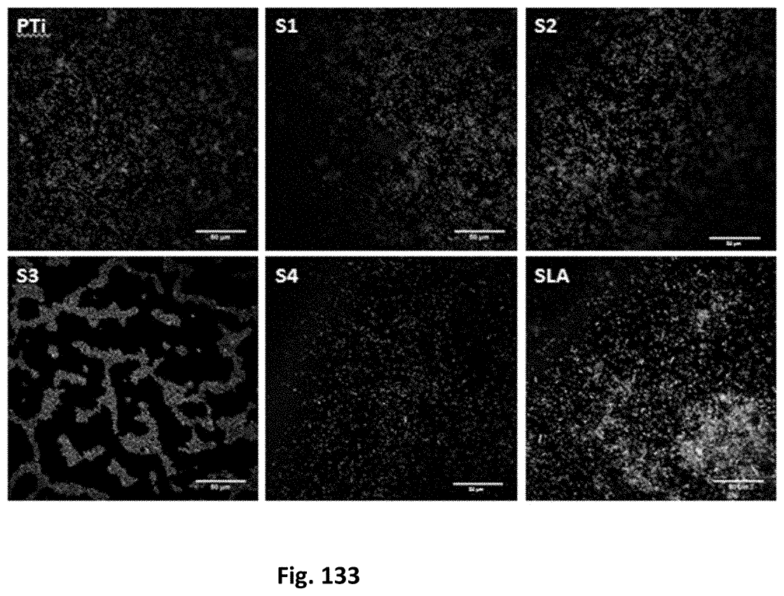

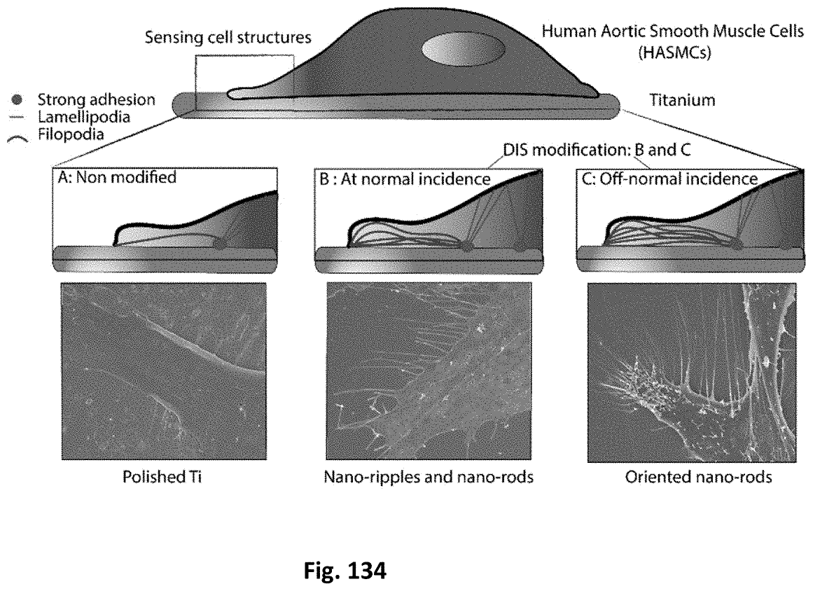

Results are provided in FIGS. 85-92.

### Example 9: Titanium Ion Irradiation Parameters

| | | Energy | Angle | Fluence |
|---|---|---|---|---|
| Sample | Gas | (keV) | (degree) | (cm⁻²) |
| Ti_Ar_07_1 | Ar | 1 | 60 | 1.00E+17 |
| Ti_Ar_08_1 | Ar | 1 | 60 | 2.50E+17 |
| Ti_Ar_09_1 | Ar | 1 | 60 | 5.00E+17 |
| Ti_Ar_10_1 | Ar | 1 | 60 | 7.50E+17 |
| Ti_Ar_02_1 | Ar | 1 | 60 | 1.00E+18 |
| Ti_Ar_19_1 | Ar | 1 | 60 | 1.00E+18 |
| Ti_Ar_20_1 | Ar | 1 | 60 | 1.00E+18 |
| Ti_Ar_21_1 | Ar | 1 | 60 | 1.00E+18 |
| Ti_Ar_22_1 | Ar | 1 | 60 | 1.00E+18 |
| Ti_Ar_23_1 | Ar | 1 | 60 | 7.50E+17 |
| Ti_Ar_24_1 | Ar | 1 | 60 | 7.50E+17 |
| Ti_Ar_25_1 | Ar | 1 | 60 | 7.50E+17 |
| Ti_Ar_26_1 | Ar | 1 | 60 | 7.50E+17 |
| Ti_Ar_27_1 | Ar | 1 | 60 | 5.00E+17 |
| Ti_Ar_28_1 | Ar | 1 | 60 | 5.00E+17 |
| Ti_Ar_29_1 | Ar | 1 | 60 | 5.00E+17 |
| Ti_Ar_30_1 | Ar | 1 | 60 | 5.00E+17 |
| Ti_Ar_31_1 | Ar | 1 | 60 | 2.50E+17 |
| Ti_Ar_32_1 | Ar | 1 | 60 | 2.50E+17 |
| Ti_Ar_33_1 | Ar | 1 | 60 | 2.50E+17 |
| Ti_Ar_34_1 | Ar | 1 | 60 | 2.50E+17 |
| Ti_Kr_03_1 | Kr | 1 | 0 | 1.00E+18 |
| Ti_Kr_05_1 | Kr | 1 | 60 | 1.00E+18 |
| Ti_N_11_1 | N₂ | 1 | 0 | 1.00E+18 |
| Ti_N_12_1 | N₂ | 1 | 60 | 1.00E+18 |
| Ti_O_13_1 | O₂ | 1 | 0 | 1.00E+18 |
| Ti_O_14_1 | O₂ | 1 | 60 | 1.00E+18 |
| Ti_Kr_15_1 | Kr | 1 | 0 | 1.00E+18 |
| Ti_Kr_16_1 | Kr | 1 | 60 | 1.00E+18 |

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A titanium-containing composition comprising:
a titanium or titanium alloy substrate having a surface; wherein said surface has a plurality of nanoscale domains characterized by a surface geometry providing a selected multifunctional bioactivity;
wherein each of said nanoscale domains has at least one lateral spatial dimension selected over the range of 3 nm to 1 μm and a vertical spatial dimension less than 500 nm.

2. A titanium-containing composition comprising:
a titanium or titanium alloy substrate having a surface; wherein said surface has a plurality of nanoscale domains characterized by a surface geometry providing a selected multifunctional bioactivity;
wherein said nanoscale domains are generated by exposing said surface to one or more directed energetic particle beam characterized by one or more beam properties.

3. The composition of claim 1, wherein said selected multifunctional bioactivity is with respect to an in vivo or in vitro activity with respect to a plurality of biological or physical processes relative to a titanium or titanium alloy substrate surface not having said plurality of nanoscale domains characterized by said nanofeatured surface geometry.

4. The composition of claim 3, wherein said in vivo or in vitro activity is an enhancement in cell adhesion activity, cell shape activity, cell proliferation activity, cell migration activity, cell differentiation activity, anti-bacterial activity, bactericidal activity, anti-inflammatory activity, osseointegration activity, biocorrosion activity, cell differentiation activity, immuno-modulating activity during acute or chronic inflammation or any combination of these; or wherein said in vivo or in vitro activity is a decrease in an immune response.

5. The composition of claim 4, wherein said enhancement of in vivo or in vitro activity is equal to or greater than 100%; and wherein decrease in said immune response is equal to or greater than 200% in a period selected from the range of 24 to 48 hours.

6-7. (canceled)

8. The composition of claim 1, wherein said surface geometry is spatial distribution of relief features, recessed features, localized regions characterized by a selected composition, phase, crystallographic texture, or any combination of these.

9. The composition of claim 1, wherein said surface geometry is a periodic or semi-periodic spatial distribution of said nanoscale domains.

10. The composition of claim 1, wherein said surface geometry is provided between and within pores of said substrate.

11. (canceled)

12. The composition of claim 1, wherein each of said nanoscale domains are characterized by a vertical spatial dimension of less than or equal to 50 nm.

13. The composition of claim 1, wherein each of said nanoscale domains are characterized by a vertical spatial dimension selected over the range of 10 nm to 250 nm.

14. The composition of claim 1, wherein said nanoscale domains comprise nanowalls, nanorods, nanoplates, nanoripples or any combination thereof having lateral spatial dimensions selected over the range of 10 to 1000 nm and vertical spatial dimensions of less than or equal to 250 nm.

15. The composition of claim 14, wherein said nanowalls, nanorods, nanoplates or nanoripples are inclined towards a direction oriented along a selected axis relative to said surface.

16. The composition of claim 14, wherein said nanowalls, nanorods, nanoplates or nanoripples are separated from one another by a distance of less than 100 nm.

17. The composition of claim 1, wherein said nanoscale domains comprise discrete crystallographic domains characterized as an α+β annealed alloy.

18. (canceled)

19. The composition of claim 1, wherein said nanoscale domains characterized by a chemical composition different from the bulk phase of said titanium or titanium alloy substrate.

20. The composition of claim 1, wherein said surface geometry provides an enhancement in vivo or in vitro activity with respect to cell adhesion proliferation activity and migration greater than or equal to 100.

21. The composition of claim 1, wherein said surface geometry provides an enhancement in vivo or in vitro activity with respect to anti-bacterial activity and bactericidal activity greater than or equal to 100%.

22. The composition of claim 1, wherein said surface geometry provides an enhancement of a selected physical property of said substrate; wherein said physical property is hydrophilicity, hydrophobicity, surface free energy, surface charge density or any combination of these.

23. (canceled)

24. The composition of claim 22, wherein said enhancement of selected physical property is equal to or greater than 25%.

25. (canceled)

26. The composition of claim 1, wherein said titanium or titanium comprises a mesoporous, microporous, or a nanoporous substrate.

27. The composition of claim 1, wherein said titanium or titanium alloy substrate comprises commercially pure titanium metal (cpTi), Ti6Al4V alloy or a combination thereof.

28. The composition of claim 1 or 2, wherein said titanium or titanium alloy substrate comprises a component of a medical device; wherein said medical device is a dental implant, a joint, hip or shoulder replacement, pedicle screw, syringe, needle, scalpel, or other surgical rod, plate or spinal injury instrument device.

29. (canceled)

30. The composition of claim 2, wherein the directed energetic particle beam is a broad beam, focused beam, asymmetric beam, reactive beam or any combination of these.

31. The composition of claim 2, wherein said one or more beam properties is intensity, fluence, energy, flux, incident angle, ion composition, neutral composition, ion to neutral ratio or any combinations thereof.

32. A method of fabricating a bioactive titanium-containing substrate, said method comprising:
providing said titanium or titanium alloy substrate having a substrate surface; and
directing a directed energetic particle beam onto said substrate surface, thereby generating a plurality of nanoscale domains on said surface;
wherein said directed energetic particle beam has one or more beam properties selected to generate said plurality of nanoscale domains characterized by a surface geometry providing a selected multifunctional bioactivity.

33-40. (canceled)

41. A method of fabricating a bioactive titanium-containing substrate, said method comprising:
providing said titanium or titanium alloy substrate having a substrate surface; and
directing a first directed energetic particle beam and a second directed energy particle beam onto said substrate surface, thereby generating a plurality of nanoscale domains on said surface;
wherein said first directed energetic particle beam has one or more first beam properties and said second directed energetic particle beam has one or more second beam properties; and
wherein at least one of said first beam properties is different than at least one of said second beam properties and said first beam properties and said second beam properties are independently selected to generate said plurality of nanoscale domains characterized by a surface geometry providing a selected multifunctional bioactivity.
